(19) United States
(12) Patent Application Publication
(10) Pub. No.: US 2008/0242666 A1
(43) Pub. Date: Oct. 2, 2008

(54) 1-6-Substituted (3R,6R)-3-(2,3-Dihydro-1H-Inden-2-Yl)-2,5-Piperazinedione Derivatives as Oxytocin Receptor Antagonists For the Treatment of Preterm Labour, Dysmenorrhea and Endometriosis

(76) Inventors: Colin Andrew Leach, King of Prussia, PA (US); John Liddle, Hertfordshire (GB); Simon Peace, Hertfordshire (GB); Joanne Philp, Hertfordshire (GB); Ian Edward David Smith, Hertfordshire (GB); Lamont Roscoe Terrell, King of Prussia, PA (US); Jing Zhang, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/722,732

(22) PCT Filed: Dec. 22, 2005
(86) PCT No.: PCT/GB05/05007

§ 371 Date: Jan. 3, 2008

(30) Foreign Application Priority Data
Dec. 23, 2004 (GB) 0428235.6

(51) Int. Cl. C07D 241/08; A61K 31/496; A61K 31/5377; C07D 413/12; C07D 401/12; A61K 31/495; C07D 403/12; A61K 31/551; A61P 5/00; A61P 43/00

(52) U.S. Cl. 514/235.8; 544/383; 514/255.02; 544/121; 544/360; 514/253.01; 514/252.11; 544/357; 514/253.12; 514/218; 540/575

(57) ABSTRACT

The present invention relates to Compound S of Formula (I).

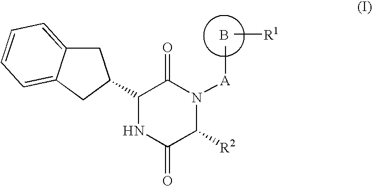

### FIELD OF THE INVENTION

This invention relates to novel diketopiperazine derivatives having a potent and selective antagonist action at the oxytocin receptor, to processes for their preparation, pharmaceutical compositions containing them and to their use in medicine.

### BACKGROUND OF THE INVENTION

U.S. Pat. No. 5,817,751 describes combinatorial and solid phase methods for the synthesis of diverse diketopiperazine derivatives and the use of these methods to create libraries of diverse diketopiperazine derivatives.

WO99/47549 describes diketopiperazine derivatives including 3-benzyl-2,5 diketopiperazine derivatives as inhibitors of fructose 1,6-bisphosphate (FBPase).

WO99/38844 describes a method for preparing N-[(aliphatic or aromatic) carbonyl]-2-aminoacetamide compounds and their cyclisation to give inter alia diketopiperazine derivatives.

WO99/37304 describes oxaheterocyclyl compounds including oxapiperazinyl compounds that are inhibitors of Factor Xa.

WO03/053443 describes diketopiperazine derivatives which exhibit activity as selective antagonists at the oxytocin receptor.

WO2005/000840 describes diketopiperazine derivatives which exhibit activity as selective antagonists at the oxytocin receptor.

The hormone oxytocin is potent contractor of the uterus and is used for the induction or augmentation of labour. Also the density of uterine oxytocin receptors increases significantly by >100 fold during pregnancy and peaks in labour (pre-term and term).

Pre-term births/labour (between 24 and 37 weeks) causes about 60% of infant mortality/morbidity and thus a compound which inhibits the uterine actions of oxytocin e.g. oxytocin antagonists, should be useful for the prevention or control of pre-term labour.

### SUMMARY OF THE INVENTION

We have found a class of diketopiperazine derivatives which exhibit a particularly useful level of activity as selective antagonists at the oxytocin receptor.

### DETAILED DESCRIPTION OF THE INVENTION

The present invention provides at least one chemical entity selected from a compound of Formula (I):

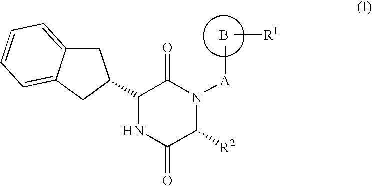

and physiologically acceptable derivatives thereof,
wherein:
A represents a C₁₋₄alkylene group optionally substituted by one or more C₁₋₄alkyl groups;
the ring B represents a mono-, bi- or tricyclic aryl or heteroaryl group containing one or more heteroatoms independently selected from O, S or N, wherein the aryl or heteroaryl group may be optionally substituted by one or more R¹ groups which may be independently selected from C₁₋₆cycloalkyl, C₁₋₆alkyl, C₁₋₆cycloalkoxy, C₁₋₆alkoxy, aryl, aralkyl, heterocyclyl, heteroaryl, —Oheterocyclyl, —Oheteroaryl, —S(O)ₙheterocyclyl or —S(O)ₙheteroaryl (each of which may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴); or R¹ may additionally be independently selected from halo, hydroxyl, —NR³R⁴, nitro, cyano, trifluoroC₁₋₄alkyl, trifluoroC₁₋₄alkoxy, carboxyl, —CONR³R⁴, —COR⁵, —S(O)ₙR⁶, —NR⁷COR⁸, —S(O)ₘNR⁹R¹⁰ or —NR¹¹S(O)ₘR¹²;
R² represents C₃₋₇alkyl, C₃₋₇ cycloalkyl or phenyl, each of which may be further optionally substituted by one or more groups selected from C₁₋₄alkyl or C₃₋₇ cycloalkyl;
R³ and R⁴ independently represent H, C₁₋₆alkyl, C₁₋₆cycloalkyl, aryl, heterocyclyl or heteroaryl wherein the C₁₋₆alkyl, C₁₋₆cycloalkyl, aryl, heterocyclyl or heteroaryl groups may be further optionally substituted by one or more groups independently selected from halo, hydroxyl, carboxyl, C₁₋₆alkyl, C₁₋₆alkoxy, C₁₋₃alkoxyC₁₋₆alkyl, trifluoroC₁₋₄alkyl, trifluoroC₁₋₄alkoxy, —S(O)ₙR⁶, COR⁵, heteroaryl, heterocyclyl, aryl or —NR^{3a}R^{4a};
or R³ and R⁴, together with the interconnecting N-atom to which they are attached form a 5- or 6-membered heteroaryl or a 4- to 7-membered heterocyclyl ring, which ring may additionally contain 1 or 2 heteroatoms independently selected from O, S or N; and wherein the 5- or 6-membered heteroaryl or 4- to 7-membered heterocyclyl ring may be further optionally substituted by one or more groups selected from C₁₋₄alkyl, C₁₋₄alkoxy, trifluoroC₁₋₄alkyl, trifluoroC₁₋₄alkoxy, —NR^{3a}R^{4a}, COR⁵, hydroxyl, aryl, heteroaryl or heterocyclyl (wherein the C₁₋₄alkyl, C₁₋₄alkoxy, aryl, heteroaryl or heterocyclyl groups on the 5- or 6-membered heteroaryl or 4- to 7-membered heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, COR⁵, heteroaryl, heterocyclyl, aryl or —NR^{3a}R^{4a});
R^{3a} and R^{4a} independently represent H, C₁₋₆alkyl, C₁₋₆cycloalkyl, aryl, heterocyclyl or heteroaryl wherein the C₁₋₆alkyl, C₁₋₆cycloalkyl, aryl, heterocyclyl or heteroaryl groups may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, or aryl;
or R^{3a} and R^{4a}, together with the interconnecting N-atom to which they are attached form a 5- or 6-membered heteroaryl or a 4- to 7-membered heterocyclyl ring, which ring may additionally contain 1 or 2 heteroatoms independently selected from O, S or N; and wherein the 5- or 6-membered heteroaryl or 4- to 7-membered heterocyclyl ring may be further optionally substituted by one or more groups selected from C₁₋₄alkyl, C₁₋₄alkoxy, trifluoroC₁₋₄alkyl, trifluoroC₁₋₄alkoxy, hydroxyl, aryl, heteroaryl or heterocyclyl (wherein the C₁₋₄alkyl, C₁₋₄alkoxy, aryl, heteroaryl or heterocyclyl groups on the 5- or 6-membered heteroaryl or 4- to 7-membered heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, or aryl);
R⁵ represents C₁₋₆alkyl, C₁₋₆alkoxy, trifluoroC₁₋₆alkyl, aryl, heteroaryl or heterocyclyl, wherein the C₁₋₆alkyl, C₁₋₆alkoxy, trifluoroC₁₋₆alkyl, aryl, heteroaryl or heterocyclyl groups may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴;
R⁶ represents C₁₋₆alkyl, C₁₋₆cycloalkyl, trifluoroC₁₋₆alkyl, aryl, heteroaryl, or heterocyclyl wherein the C₁₋₆alkyl, C₁₋₆cycloalkyl, trifluoroC₁₋₆alkyl, aryl, heteroaryl, or heterocyclyl groups may be optionally substituted by one or more groups independently selected from halo, hydroxyl, carboxyl, C₁₋₆alkyl, C₁₋₆alkoxy, C₁₋₃alkoxyC₁₋₆alkyl, trifluoroC₁₋₄alkyl, trifluoroC₁₋₄alkoxy, heteroaryl, heterocyclyl, aryl or —NR³R⁴;
R⁷ represents H or C₁₋₄alkyl (optionally substituted by one or more groups independently selected from by halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴);
R₈ represents C₁₋₆alkyl, C₁₋₆cycloalkyl, aryl, heterocyclyl or heteroaryl each of which may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴;
or R⁷ and R⁸ together with the interconnecting atoms to which they are attached form a 4- to 7-membered heterocyclyl ring which ring may additionally contain one or more heteroatoms independently selected from O, S or N, and wherein the heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴;
R⁹ and R¹⁰ independently represent H, C₁₋₆alkyl, C₁₋₆cycloalkyl, aryl, heterocyclyl or heteroaryl wherein the C₁₋₆alkyl, C₁₋₆cycloalkyl, aryl, heterocyclyl or heteroaryl group may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, aryl, —NR³R⁴ or heterocyclyl optionally substituted with C₁₋₆alkyl;
or R⁹ and R¹⁰, together with the interconnecting N-atom to which they are attached form a 5- or 6-membered heteroaryl or a 4- to 7-membered heterocyclyl ring which ring may additionally contain 1 or 2 heteroatoms independently selected from O, S or N; and wherein the 5- or 6-membered heteroaryl or 4- to 7-membered heterocyclyl ring may be further optionally substituted by one or more groups selected from C₁₋₄alkyl, C₁₋₄alkoxy, trifluoroC₁₋₄alkyl, trifluoroC₁₋₄alkoxy, —NR³R⁴, hydroxyl, aryl, heteroaryl or heterocyclyl (wherein the C₁₋₄alkyl, C₁₋₄alkoxy, aryl, heteroaryl or heterocyclyl groups on the 5- or 6-membered heteroaryl or 4- to 7-membered heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴);
R¹¹ represents H or C₁₋₄alkyl (optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴);
R¹² represents C₁₋₆alkyl, C₁₋₆cycloalkyl, aryl, heterocyclyl or heteroaryl each of which may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴;
or R¹¹ and R¹² together with the interconnecting atoms to which they are attached form a 4- to 7-membered heterocyclyl ring which ring may additionally contain one or more heteroatoms independently selected from O, S or N, and wherein the heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴;
n represents 0, 1 or 2;
and m represents 1 or 2.

In one aspect of the invention there is provided at least one chemical entity comprising a compound of Formula (IA) and physiologically acceptable derivatives thereof, wherein the compound of Formula (IA) is a compound of Formula (I) which is other than a compound selected from List 1:

### List 1

(3R,6R)-3-(2,3-dihydro-1H-inden-2-yl)-6-(2-methylpropyl)-1-[2-(2-pyridinyl)ethyl]-2,5-piperazinedione;
(3R,6R)-3-(2,3-dihydro-1H-inden-2-yl)-6-(2-methylpropyl)-1-[2-(4-nitrophenyl)ethyl]-2,5-piperazinedione;
(3R,6R)-3-(2,3-dihydro-1H-inden-2-yl)-6-(2-methylpropyl)-1-[2-(4-pyridinyl)ethyl]-2,5-piperazinedione
(3R,6R)-3-(2,3-dihydro-1H-inden-2-yl)-6-(2-methylpropyl)-1-(4-pyridinylmethyl)-2,5-piperazinedione;
(3R,6R)-3-(2,3-dihydro-1H-inden-2-yl)-6-(2-methylpropyl)-1-[2-(5,6,7,8-tetrahydroimidazo[1,5-a]pyridin-1-yl)ethyl]-2,5-piperazinedione;
(3R,6R)-3-(2,3-dihydro-1H-inden-2-yl)-6-(2-methylpropyl)-1-{[6-(trifluoromethyl)-3-pyridinyl]methyl}-2,5-piperazinedione;
(3R,6R)-3-(2,3-dihydro-1H-inden-2-yl)-1-[2-(1-methyl-1H-imidazol-2-yl)ethyl]-6-(2-methylpropyl)-2,5-piperazinedione;
(3R,6R)-3-(2,3-dihydro-1H-inden-2-yl)-6-(2-methylpropyl)-1-{[4-(3-pyridinyl)phenyl]-methyl}-2,5-piperazinedione;
(3R,6R)-3-(2,3-dihydro-1H-inden-2-yl)-1-[2-(1-methyl-1H-imidazol-5-yl)ethyl]-6-(2-methylpropyl)-2,5-piperazinedione;
(3R,6R)-3-(2,3-dihydro-1H-inden-2-yl)-6-(2-methylpropyl)-1-{2-[4-(methylsulfonyl)phenyl]-ethyl}-2,5-piperazinedione;
N-(3-{2-[(3R,6R)-3-(2,3-dihydro-1H-inden-2-yl)-6-(2-methylpropyl)-2,5-dioxo-1-piperazinyl]ethyl}phenyl)methanesulfonamide;
(3R,6R)-3-(2,3-dihydro-1H-inden-2-yl)-6-(2-methylpropyl)-1-[(1-methyl-1H-pyrazol-4-yl)methyl]-2,5-piperazinedione;
(3R,6R)-3-(2,3-dihydro-1H-inden-2-yl)-6-(2-methylpropyl)-1-{[4-(4-morpholinylmethyl)-phenyl]methyl}-2,5-piperazinedione;
(3R,6R)-3-(2,3-dihydro-1H-inden-2-yl)-6-(2-methylpropyl)-1-[(2-methyl-2H-tetrazol-5-yl)methyl]-2,5-piperazinedione;
(3R,6R)-3-(2,3-dihydro-1H-inden-2-yl)-1-[(1-ethyl-5-methyl-1H-pyrazol-4-yl)methyl]-6-(2-methylpropyl)-2,5-piperazinedione;
(3R,6R)-3-(2,3-dihydro-1H-inden-2-yl)-1-[(1-ethyl-1H-pyrazol-4-yl)methyl]-6-(2-methylpropyl)-2,5-piperazinedione;
(3R,6R)-3-(2,3-dihydro-1H-inden-2-yl)-6-(2-methylpropyl)-1-{[1-methyl-5-(trifluoromethyl)-1H-pyrazol-4-yl]methyl}-2,5-piperazinedione;
(3R,6R)-3-(2,3-dihydro-1H-inden-2-yl)-1-({4-[(dimethylamino)methyl]phenyl}methyl)-6-(1-ethylpropyl)-2,5-piperazinedione;
(3R,6R)-1-{[3,5-bis(trifluoromethyl)phenyl]methyl}-3-(2,3-dihydro-1H-inden-2-yl)-6-(1-ethylpropyl)-2,5-piperazinedione;
(3R,6R)-6-cyclopropyl-3-(2,3-dihydro-1H-inden-2-yl)-1-{[2-(methylsulfonyl)phenyl]-methyl}-2,5-piperazinedione;
(3R,6R)-3-(2,3-dihydro-1H-inden-2-yl)-1-[(3-methylphenyl)methyl]-6-phenyl-2,5-piperazinedione; and
(3R,6R)-3-(2,3-dihydro-1H-inden-2-yl)-1-[(2-{[3-(dimethylamino)propyl]sulfinyl}phenyl)-methyl]-6-(1-ethylpropyl)-2,5-piperazinedione.

In an alternative embodiment of the invention there is provided at least one chemical entity selected from a compound of Formula (A):

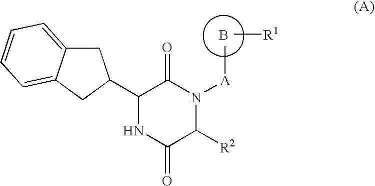

and physiologically acceptable derivatives, salts, solvates and prodrugs thereof,
wherein:
A represents a C₁₋₄alkylene group optionally substituted by one or more C₁₋₄alkyl groups;
the ring B represents a mono-, bi- or tricyclic aryl or heteroaryl group containing one or more heteroatoms independently selected from O, S or N, wherein the aryl or heteroaryl group may be optionally substituted by one or more R¹ groups which may be independently selected from C₁₋₆cycloalkyl, C₁₋₆alkyl, C₁₋₆cycloalkoxy, C₁₋₆alkoxy, aryl, aralkyl, heterocyclyl, heteroaryl, —Oheterocyclyl, —Oheteroaryl, —S(O)ₙheterocyclyl or —S(O)ₙheteroaryl (each of which may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴); or R¹ may additionally be independently selected from H, halo, —NR³R⁴, nitro, trifluoroC₁₋₄alkyl, trifluoroC₁₋₄alkoxy, carboxyl, —CONR³R⁴, —COR⁵, —S(O)ₙR⁶, —NR⁷COR⁸, —S(O)ₘNR⁹R¹⁰ or —NR¹¹S(O)ₘR¹²;
R² represents C₃₋₇alkyl, C₃₋₇ cycloalkyl or phenyl, each of which may be further optionally substituted by one or more groups selected from C₁₋₄alkyl or C₃₋₇ cycloalkyl;
R³ and R⁴ independently represent H, C₁₋₆alkyl, C₁₋₆cycloalkyl, aryl, heterocyclyl or heteroaryl wherein the C₁₋₆alkyl, C₁₋₆cycloalkyl, aryl, heterocyclyl or heteroaryl groups may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or the group —NR^{3a}R^{4a};
or R³ and R⁴, together with the interconnecting N-atom to which they are attached form a 5- or 6-membered heteroaryl or heterocyclyl ring, which ring may additionally contain 1 or 2 heteroatoms independently selected from O, S or N; and wherein the 5- or 6-membered heteroaryl ring may be further optionally substituted by one or more groups selected from C₁₋₄alkyl, C₁₋₄alkoxy, trifluoroC₁₋₄alkyl, trifluoroC₁₋₄alkoxy, —NR^{3a}R^{4a}, hydroxyl, aryl, heteroaryl or heterocyclyl (wherein the C₁₋₄alkyl, C₁₋₄alkoxy, aryl, heteroaryl or heterocyclyl groups on the 5- or 6-membered heteroaryl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR^{3a}R^{4a});
R^{3a} and R^{4a} independently represent H, C₁₋₆alkyl, C₁₋₆cycloalkyl, aryl, heterocyclyl or heteroaryl wherein the C₁₋₆alkyl, C₁₋₆cycloalkyl, aryl, heterocyclyl or heteroaryl groups may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, or aryl;
or R^{3a} and R^{4a}, together with the interconnecting N-atom to which they are attached form a 5- or 6-membered heteroaryl or heterocyclyl ring, which ring may additionally contain 1 or 2 heteroatoms independently selected from O, S or N; and wherein the 5- or 6-membered heteroaryl ring may be further optionally substituted by one or more groups selected from C₁₋₄alkyl, C₁₋₄alkoxy, trifluoroC₁₋₄alkyl, trifluoroC₁₋₄alkoxy, —NR^{3a}R^{4a}, hydroxyl, aryl, heteroaryl or heterocyclyl (wherein the C₁₋₄alkyl, C₁₋₄alkoxy, aryl, heteroaryl or heterocyclyl groups on the 5- or 6-membered heteroaryl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, or aryl);
R⁵ represents H, C₁₋₆alkyl, C₁₋₆alkoxy, trifluoroC₁₋₆alkyl, aryl, heteroaryl or heterocyclyl, wherein the C₁₋₆alkyl, C₁₋₆alkoxy, trifluoroC₁₋₆alkyl, aryl, heteroaryl or heterocyclyl groups may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴;
R⁶ represents H, C₁₋₆alkyl, C₁₋₆cycloalkyl, trifluoroC₁₋₆alkyl, aryl, heteroaryl, or heterocyclyl wherein the C₁₋₆alkyl, C₁₋₆cycloalkyl, trifluoroC₁₋₆alkyl, aryl, heteroaryl, or heterocyclyl groups may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴;
R⁷ represents H or C₁₋₄alkyl (optionally substituted one or more groups independently selected from by halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴);
R₈ represents C₁₋₆alkyl, C₁₋₆cycloalkyl, aryl, heterocyclyl or heteroaryl each of which may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴;
or R⁷ and R⁸ together with the interconnecting atoms to which they are attached form a 5- or 6-membered heterocyclyl ring which ring may additionally contain one or more heteroatoms independently selected from O, S or N, and wherein heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴;
R⁹ and R¹⁰ independently represent H, C₁₋₆alkyl, C₁₋₆cycloalkyl, aryl, heterocyclyl or heteroaryl wherein the C₁₋₆alkyl, C₁₋₆cycloalkyl, aryl, heterocyclyl or heteroaryl group may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴;
or R⁹ and R¹⁰, together with the interconnecting N-atom to which they are attached form a 5- or 6-membered heteroaryl or heterocyclyl ring which ring may additionally contain 1 or 2 heteroatoms independently selected from O, S or N; and wherein the 5- or 6-membered heteroaryl ring may be further optionally substituted by one or more groups selected from C₁₋₄alkyl, C₁₋₄alkoxy, trifluoroC₁₋₄alkyl, trifluoroC₁₋₄alkoxy, —NR³R⁴, hydroxyl, aryl, heteroaryl or heterocyclyl (wherein the C₁₋₄alkyl, C₁₋₄alkoxy, aryl, heteroaryl or heterocyclyl groups on the 5- or 6-membered heteroaryl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴);
R¹¹ represents H or C₁₋₄alkyl (optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴);
R¹² represents C₁₋₆alkyl, C₁₋₆cycloalkyl, aryl, heterocyclyl or heteroaryl each of which may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴;
or R¹¹ and R¹² together with the interconnecting atoms to which they are attached form a 5- or 6-membered heterocyclyl ring which ring may additionally contain one or more heteroatoms independently selected from O, S or N, and wherein the heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocycyl, aryl or the group —NR³R⁴;
n represents 0, 1 or 2;
and m represents 1 or 2.

In one aspect of the invention there is provided at least one chemical entity comprising a compound of Formula (A′) and physiologically acceptable derivatives thereof, wherein the compound of Formula (A′) is a compound of Formula (A) which is other than a compound selected from List 1 as hereinbefore defined.

Certain compounds of Formula (I) or Formula (A) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by Formula (I) or Formula (A) as mixtures with isomers thereof in which one or more chiral centres are inverted. Likewise, it is understood that compounds of Formula (I) or Formula (A) may exist in tautomeric forms other than that shown in the Formula and these are also included within the scope of the present invention.

The compounds of Formula (I) or Formula (A) wherein at least one of the groups R₁ or R₂ contains a basic or acidic grouping may form salts with physiologically acceptable acids or bases and reference to compounds of Formula (I) or Formula (A) herein includes such salts.

### Terms and Definitions

As used herein, the terms “physiologically acceptable derivative” or “pharmaceutically acceptable derivative”, mean any pharmaceutically acceptable salt, solvate, or prodrug e.g. ester or carbamate, or salt or solvate of such a prodrug, of a compound of Formula (I) or Formula (A), which upon administration to the recipient is capable of providing (directly or indirectly) a compound of Formula (I) or Formula (A), or an active metabolite or residue thereof. Preferred pharmaceutically acceptable derivatives are salts and solvates.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. Esters may be active in their own right and/or be hydrolysable under in vivo conditions in the human body. Suitable pharmaceutically acceptable in vivo hydrolysable ester groups include those which break down readily in the human body to leave the parent acid or its salt. Examples of such esters include alkyl and 1-(acetyloxy)ethyl esters.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to methoxy, ethoxy, propoxy, prop-2-oxy, butoxy, but-2-oxy, 2-methylprop-1-oxy, 2-methylprop-2-oxy, pentoxy or hexyloxy.

As used herein, the term “alkylene” as a group or a part of a group refers to a linear or branched saturated hydrocarbon linker group containing the indicated number of carbon atoms. Examples of such groups include methylene, ethylene and the like.

As used herein, the term “aralkyl” as a group or a part of a group refers to an alkyl group as herein defined which contains the indicated number of carbon atoms, the alkyl group being substituted with an aryl group as herein defined.

As used herein, the term “cycloalkyl” as a group or a part of a group refers to a saturated cyclic hydrocarbon group of 3 to 7 carbon atoms. Examples of such groups include cyclopropyl, cyclobutyl, cyclopentyl, cyclohexyl and cycloheptyl groups.

As used herein, the term “cycloalkyloxy” as a group or a part of a group refers to an —O-cycloalkyl group wherein cycloalkyl is as herein defined.

As used herein, the term “halogen” or halo refers to fluorine, chlorine, bromine or iodine.

As used herein, the term “aryl” refers to refers to a cyclic compound made up of one or more benzene rings and includes phenyl, naphthyl, phenanthrenyl and anthracenyl, each of which may be optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴.

As used herein, the term “heteroaryl” as a group or a part of a group refers to an optionally substituted aromatic group comprising one to four heteroatoms selected from N, O and S, the aromatic group containing one, two or three 5- or 6-membered conjugated or fused rings with at least one ring having a conjugated pi-electron system. Heteroaryl groups may be substituted by one or more groups independently selected from halo, oxo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴. Examples of such 5-membered heteroaryl groups include furanyl, thienyl, pyrrolyl, oxazolyl, isoxazolyl, thiazolyl, imidazolyl, pyrazolyl, oxadiazolyl, thiadiazolyl, triazolyl or tetrazolyl and these heterocycles may be substituted as described above. Examples of 6-membered heteroaryl groups include pyridyl, pyridazinyl, pyrimidinyl, pyrazinyl and triazinyl and these heterocycles may be substituted as described above. Examples of fused heteroaryl groups, include benzimidazolyl, benzofuranyl, indolyl, indazolyl, benzoxazolyl, naphthyridinyl, pteridinyl, quinolinyl and these heteroaryl groups may be substituted as described above.

As used herein, the term “heterocyclyl” as a group or a part of a group refers to an optionally substituted, 3- to 7-membered, saturated or partially saturated cyclic hydrocarbon group containing one to four heteroatoms selected from N, O and S. Heterocyclyl groups may be substituted by one or more groups independently selected from halo, oxo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴. Examples of 5-membered heterocyclyl groups include pyrrolinyl, pyrrolidinyl, imidazolinyl, imidazolidinyl, each of which may be substituted as described above. Examples of 6-membered heterocyclyl groups include pyranyl, morpholino, thiomorpholino, piperidinyl, each of which may be substituted as described above. An example of 7-membered heterocyclyl groups includes homopiperazine (hexahydro-1H-1,4-diazepin-1-yl). In addition, the term “heterocyclyl” includes fused heterocyclyl groups, for example benzopiperidinyl, benzopiperazinyl, each of which may be substituted as described above.

As used herein, the term “substituted” refers to substitution with the named substituent or substituents, multiple degrees of substitution being allowed unless otherwise stated.

For the avoidance of doubt, the term “independently” means that where more than one substituent is selected from a number of possible substituents, those substituents may be the same or different.

The compounds of the present invention may be in the form of and/or may be administered as a pharmaceutically acceptable salt. Indeed, in certain embodiments of the invention, pharmaceutically acceptable salts of the compounds according to Formula (I) or Formula (A) may be preferred over the respective free base or free acid because such salts impart greater stability or solubility to the molecule thereby facilitating formulation into a dosage form. Accordingly, the invention is further directed to pharmaceutically acceptable salts of the compounds according to Formula (I) or Formula (A).

As used herein, the term “pharmaceutically acceptable salts” refers to salts that retain the desired biological activity of the subject compound and exhibit minimal undesired toxicological effects. For a review on suitable salts see Berge et al, J. Pharm. Sci., 1977, 66, 1-19. The term “pharmaceutically acceptable salts” includes both pharmaceutically acceptable acid addition salts and pharmaceutically acceptable base addition salts. These pharmaceutically acceptable salts may be prepared in situ during the final isolation and purification of the compound, or by separately reacting the purified compound in its free acid or free base form with a suitable base or acid, respectively. The salt may precipitate from solution and be collected by filtration or may be recovered by evaporation of the solvent.

A pharmaceutically acceptable acid addition salt can be formed by reaction of a compound of formula (I) with a suitable inorganic or organic acid (such as hydrobromic, hydrochloric, sulfuric, sulfamic, nitric, phosphoric, succinic, maleic, hydroxymaleic, acrylic, formic, acetic, hydroxyacetic, phenylacetic, butyric, isobutyric, propionic, fumaric, citric, tartaric, lactic, mandelic, benzoic, o-acetoxybenzoic, chlorobenzoic, methylbenzoic, dinitrobenzoic, hydroxybenzoic, methoxybenzoic salicylic, glutamic, stearic, ascorbic, palmitic, oleic, pyruvic, pamoic, malonic, lauric, glutaric aspartic, p-toluenesulfonic, benzenesulfonic, methanesulfonic, ethanesulfonic, 2-hydroxyethanesulfonic, naphthalenesulfonic (e.g. 2-naphthalenesulfonic), p-aminobenzenesulfonic (i.e. sulfanilic), hexanoic, heptanoic, or phthalic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, hydroiodide, sulfate, bisulfate, nitrate, phosphate, hydrogen phosphate, succinate, maleate, malate, formate, acetate, trifluoroacetate, saccharate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate), methanesulphonic, ethanesulphonic, p-toluenesulphonic, isethionate or hexanoate salt. In one embodiment there is provided the formate and hydrochloride salts of the compounds of the invention.

A pharmaceutically acceptable base addition salt can be formed by reaction of a compound of formula (I) with a suitable inorganic or organic base (e.g. ammonia, triethylamine, ethanolamine, triethanolamine, choline, arginine, lysine or histidine), optionally in a suitable solvent such as an organic solvent, to give the base addition salt which is usually isolated for example by crystallisation and filtration. Pharmaceutically acceptable base salts include ammonium salts and salts with organic bases, including salts of primary, secondary and tertiary amines, including aliphatic amines, aromatic amines, aliphatic diamines, and hydroxy alkylamines, such as methylamine, ethylamine, isopropylamine, diethylamine, ethylenediamine, ethanolamine, trimethylamine, dicyclohexyl amine, diethanolamine, cyclohexylamine and N-methyl-D-glucamine. Other suitable pharmaceutically acceptable base salts include pharmaceutically acceptable metal salts, for example pharmaceutically acceptable alkali-metal or alkaline-earth-metal salts such as hydroxides, carbonates and bicarbonates of sodium, potassium, lithium, calcium, magnesium, aluminium, and zinc; in particular pharmaceutically acceptable metal salts of one or more carboxylic acid moieties that may be present in the compound of Formula (I) or Formula (A).

Other non-pharmaceutically acceptable salts, for example oxalates may be used, for example in the isolation of compounds of the invention, and are included within the scope of this invention.

The invention includes within its scope all possible stoichiometric and non-stoichiometric forms of the salts of the compounds of Formula (I).

As used herein, the term “solvate” refers to a complex of variable stoichiometry formed by a solute (in this invention, a compound of Formula (I) or Formula (A) or a salt thereof) and a solvent. Such solvents for the purpose of the invention may not interfere with the biological activity of the solute. Examples of suitable solvents include water, ethanol and acetic acid. Most preferably the solvent used is water and the solvate may also be referred to as a hydrate.

In one aspect of the invention A represents CH₂, CH(CH₃) or CH₂CH₂. In another aspect, A represents CH₂ or CH(CH₃). In a further aspect, A represents CH₂.

In one aspect of the invention the ring B represents phenyl, pyridyl, pyrimidinyl, quinolinyl or pyrazolyl. In another aspect the ring B represents phenyl, pyridyl, pyrimidinyl or pyrazolyl. In a further aspect, the ring B represents phenyl.

In one aspect, the ring B is optionally substituted by one or two R¹ groups. In another aspect, R¹ groups may be independently selected from C₁₋₆alkyl, C₁₋₆alkoxy, aryl, aralkyl, heterocyclyl, heteroaryl, (each of which may be optionally substituted by one or more groups independently selected from hydroxyl, C₁₋₆alkyl, C₁₋₆alkoxy, heterocyclyl, aryl or —NR³R⁴); or R¹ may additionally be independently selected from halo, hydroxyl, —NR³R⁴, nitro, trifluoroC₁₋₄alkyl, trifluoroC₁₋₄alkoxy, carboxyl, —CONR³R⁴, —COR⁵, —S(O)ₙR⁶, —NR⁷COR⁸, —S(O)ₘNR⁹R¹⁰ or —NR¹¹S(O)ₘR¹². In another aspect, R¹ groups may be may be independently selected from C₁₋₆alkyl, heteroaryl, for example pyrazolyl, (each of which may be optionally substituted by one or more groups independently selected from C₁₋₆alkoxy or —NR³R⁴); or R¹ may additionally be independently selected from —NR³R⁴, —CONR³R⁴, —S(O)ₙR⁶, —NR⁷COR⁸ or —S(O)ₘNR⁹R¹⁰.

In one aspect of the invention R² represents C₃₋₅alkyl, or R² represents C₃₋₅cycloalkyl which may be further optionally substituted by C₁₋₂alkyl, wherein the total number of carbon atoms in the R² group is between 3 and 5.

In one aspect of the invention R³ and R⁴ independently represent H, C₁₋₆alkyl, C₁₋₆cycloalkyl, heterocyclyl or heteroaryl wherein the C₁₋₆alkyl, C₁₋₆cycloalkyl, heterocyclyl or heteroaryl groups may be further optionally substituted by one or more groups independently selected from hydroxyl, C₁₋₆alkyl, C₁₋₆alkoxy, C₁₋₃alkoxyC₁₋₆alkyl, heterocyclyl, aryl or —NR^{3a}R^{4a}; or R³ and R⁴, together with the interconnecting N-atom to which they are attached form a 5- or 6-membered heterocyclyl ring, which ring may additionally contain 1 or 2 heteroatoms independently selected from O, S or N (for example morpholine or piperazine); and wherein the 5- or 6-membered heterocyclyl ring may be further optionally substituted by C₁₋₄alkyl, (wherein the C₁₋₄alkyl group may be further optionally substituted by one or more groups independently selected from heterocyclyl or aryl). In another aspect R³ and R⁴ independently represent H or C₁₋₄alkyl which is optionally substituted by one or more groups independently selected from hydroxyl, C₁₋₂alkyl or —NR^{3a}R^{4a}, or R³ and R⁴, together with the interconnecting N-atom to which they are attached form a 5- or 6-membered heterocyclyl ring, which ring may additionally contain 1 or 2 heteroatoms independently selected from O, S or N (for example morpholine or piperazine); and wherein the 5- or 6-membered heterocyclyl ring may be further optionally substituted by C₁₋₄alkyl.

In one aspect of the invention R^{3a} and R^{4a} independently represent H or C₁₋₆alkyl. In another aspect R^{3a} and R^{4a} independently represent C₁₋₆alkyl.

In one aspect of the invention R⁵ represents C₁₋₆alkoxy which is optionally substituted with hydroxyl, C₁₋₆alkoxy, or —NR³R⁴ (for example NMe₂, morpholine, piperidine, piperazine or pyrrolidine). In another aspect R⁵ respesents C₁₋₃alkoxy.

In one aspect of the invention R⁶ represents C₁₋₆alkyl, trifluoroC₁₋₆alkyl or heterocyclyl, each of which may be optionally substituted by one or more groups independently selected from C₁₋₆alkyl, C₁₋₃alkoxyC₁₋₆alkyl, heterocyclyl or —NR³R⁴ (for example NMe₂, morpholine, piperidine or piperazine). In another aspect R⁶ represents C₁₋₃alkyl.

In one aspect of the invention R⁷ represents H or C₁₋₄alkyl.

In one aspect of the invention R₈ represents C₁₋₆alkyl or heterocyclyl or heteroaryl each of which may be optionally substituted by one or more groups independently selected from C₁₋₆alkyl, or —NR³R⁴.

In one aspect of the invention R⁹ and R¹⁰ independently represent H, C₁₋₆alkyl, heterocyclyl or heteroaryl each of which is optionally substituted by one or more groups independently selected from hydroxyl, carboxyl, C₁₋₆alkyl, aryl —NR³R⁴ or heterocyclyl optionally substituted by C₁₋₆alkyl, or R⁹ and R¹⁰, together with the interconnecting N-atom to which they are attached form a 5-, 6- or 7-membered heterocyclyl ring which ring may additionally contain 1 or 2 heteroatoms independently selected from O, S or N (for example morpholine, piperidine or piperazine); and wherein the 5-, 6- or 7-membered heterocyclyl ring may be further optionally substituted by one or more groups selected from C₁₋₄alkyl, or —NR³R⁴, (wherein the C₁₋₄alkyl group may be further optionally substituted by C₁₋₆alkoxy). In another aspect R⁹ and R¹⁰ both represent CH₃, or R⁹ and R¹⁰, together with the interconnecting N-atom to which they are attached form a morpholine, piperidine, piperazine or pyrrolidine ring.

In one aspect of the invention R¹¹ represents H or C₁₋₄alkyl. In another aspect of the invention R¹² represents C₁₋₆alkyl. In a further aspect R¹¹ and R¹² together with the interconnecting atoms to which they are attached form a 5- or 6-membered heterocyclyl ring which ring may additionally contain one or more heteroatoms independently selected from O, S or N, and wherein the heterocyclyl ring may be further optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl or —NR³R⁴.

In one aspect of the invention n represents 2.

In one aspect of the invention m represents 2.

In one aspect of the invention, for compounds of Formula (A), the stereochemistry of the two chiral centres on the central piperazine-2,5-dione ring is (3R,6R).

It is to be understood that the present invention covers all combinations of aspects of the invention, including suitable, convenient and preferred groups, described hereinabove.

In one aspect, chemical entities useful in the present invention may be chosen from at least one chemical entity of Formula (I) selected from the group consisting of:
(3R,6R)-3-(2,3-dihydro-1H-inden-2-yl)-6-(1-ethylpropyl)-1-[2(hydroxymethyl)benzyl]-piperazine-2,5-dione;
methyl 2-{[(3R,6R)-3-(2,3-dihydro-1H-inden-2-yl)-6-(1-ethylpropyl)-2,5-dioxo-1-piperazinyl]methyl}benzoate;
(3R,6R)-3-(2,3-dihydro-1H-inden-2-yl)-6-(1-ethylpropyl)-1-{[3-(methyloxy)phenyl]methyl}-2,5-piperazinedione;
(3R,6R)-3-(2,3-dihydro-1H-inden-2-yl)-6-(1-ethylpropyl)-1-[(2-methylphenyl)methyl]-2,5-piperazinedione;
(3R,6R)-3-(2,3-dihydro-1H-inden-2-yl)-6-(1-ethylpropyl)-1-{[4-(methyloxy)phenyl]methyl}-2,5-piperazinedione;
(3R,6R)-3-(2,3-dihydro-1H-inden-2-yl)-6-(1-ethylpropyl)-1-{[2-(methyloxy)phenyl]methyl}-2,5-piperazinedione;
(3R,6R)-3-(2,3-dihydro-1H-inden-2-yl)-6-(1-ethylpropyl)-1-[(3-methylphenyl)methyl]-2,5-piperazinedione;
(3R,6R)-3-(2,3-dihydro-1H-inden-2-yl)-6-(1-ethylpropyl)-1-{[3-(trifluoromethyl)phenyl]-methyl}-2,5-piperazinedione;
(3R,6R)-3-(2,3-dihydro-1H-inden-2-yl)-6-(1-ethylpropyl)-1-[(4-methylphenyl)methyl]-2,5-piperazinedione;
(3R,6R)-1-[(3-chlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-[(4-fluorophenyl)methyl]-2,5-piperazinedione;
(3R,6R)-1-[(2-chlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[3-(4-morpholinylmethyl)-phenyl]methyl}-2,5-piperazinedione formate;
(3R,6R)-3-(2,3-dihydro-1H-inden-2-yl)-6-(1-ethylpropyl)-1-{[4-(4-morpholinyl)phenyl]-methyl}-2,5-piperazinedione;
(3R,6R)-3-(2,3-dihydro-1H-inden-2-yl)-6-(1-ethylpropyl)-1-{[3-(1-piperidinylmethyl)-phenyl]methyl}-2,5-piperazinedione formate;
(3R,6R)-3-(2,3-dihydro-1H-inden-2-yl)-1-[(2-{[2-(dimethylamino)ethyl]oxy}phenyl)methyl]-6-(1-ethylpropyl)-2,5-piperazinedione;
(3R,6R)-3-(2,3-dihydro-1H-inden-2-yl)-6-(1-ethylpropyl)-1-[(2-fluorophenyl)methyl]-2,5-piperazinedione;
(3R,6R)-3-(2,3-dihydro-1H-inden-2-yl)-6-(1-ethylpropyl)-1-[(3-fluorophenyl)methyl]-2,5-piperazinedione;
(3R,6R)-3-(2,3-dihydro-1H-inden-2-yl)-6-(1-ethylpropyl)-1-{[2-(trifluoromethyl)phenyl]-methyl}-2,5-piperazinedione;
(3R,6R)-3-(2,3-dihydro-1H-inden-2-yl)-6-(1-ethylpropyl)-1-{[4-(trifluoromethyl)phenyl]-methyl}-2,5-piperazinedione;
(3R,6R)-3-(2,3-dihydro-1H-inden-2-yl)-6-(1-ethylpropyl)-1-({2-[(trifluoromethyl)oxy]-phenyl}methyl)-2,5-piperazinedione;
(3R,6R)-3-(2,3-dihydro-1H-inden-2-yl)-6-(1-ethylpropyl)-1-({3-[(trifluoromethyl)oxy]-phenyl}methyl)-2,5-piperazinedione;
(3R,6R)-1-{[2,6-bis(methyloxy)phenyl]methyl}-3-(2,3-dihydro-1H-inden-2-yl)-6-(1-ethylpropyl)-2,5-piperazinedione;
(3R,6R)-1-[(2,6-dichlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-(2-pyridinylmethyl)-2,5-piperazinedione;
(3R,6R)-3-(2,3-dihydro-1H-inden-2-yl)-6-(2,2-dimethylpropyl)-1-[(3-methylphenyl)methyl]-2,5-piperazinedione;
(3R,6R)-3-(2,3-dihydro-1H-inden-2-yl)-6-(1-ethylpropyl)-1-[(1R)-1-phenylethyl]-2,5-piperazinedione;
(3R,6R)-3-(2,3-dihydro-1H-inden-2-yl)-1-[(2,6-dimethyl-3-pyridinyl)methyl]-6-(1-ethylpropyl)-2,5-piperazinedione;
(3R,6R)-1-{[2,4-bis(methyloxy)phenyl]methyl}-3-(2,3-dihydro-1H-inden-2-yl)-6-(1-ethylpropyl)-2,5-piperazinedione;
(3R,6R)-1-[(2-bromophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[4-(methylsulfonyl)phenyl]-methyl}-2,5-piperazinedione;
4-{[(3R,6R)-3-(2,3-dihydro-1H-inden-2-yl)-6-(1-ethylpropyl)-2,5-dioxo-1-piperazinyl]-methyl}-N,N-dimethyl-benzenesulfonamide;
(3R,6R)-3-(2,3-dihydro-1H-inden-2-yl)-6-(1-ethylpropyl)-1-[(4-hydroxyphenyl)methyl]-2,5-piperazinedione;
(3R,6R)-3-(2,3-dihydro-1H-inden-2-yl)-6-(2-methylpropyl)-1-[(3-nitrophenyl)methyl]-2,5-piperazinedione;
(3R,6R)-3-(2,3-dihydro-1H-inden-2-yl)-6-(2-methylpropyl)-1-[(2-nitrophenyl)methyl]-2,5-piperazinedione;
(3R,6R)-1-({3-[(difluoromethyl)oxy]phenyl}methyl)-3-(2,3-dihydro-1H-inden-2-yl)-6-(2-methylpropyl)-2,5-piperazinedione;
(3R,6R)-3-(2,3-dihydro-1H-inden-2-yl)-6-(2-methylpropyl)-1-{[2-(methylsulfonyl)phenyl]-methyl}-2,5-piperazinedione;
(3R,6R)-3-(2,3-dihydro-1H-inden-2-yl)-6-(2-methylpropyl)-1-{[4-(1,2,3-thiadiazol-4-yl)phenyl]methyl}-2,5-piperazinedione;
(3R,6R)-3-(2,3-dihydro-1H-inden-2-yl)-6-(1-ethylpropyl)-1-[(1-phenyl-1H-pyrazol-4-yl)methyl]-2,5-piperazinedione;
(3R,6R)-1-[(3-chlorophenyl)methyl]-3-(2,3-dihydro-1H-inden-2-yl)-6-(2-methylpropyl)-2,5-piperazinedione;
(3R,6R)-1-[(3,4-dichlorophenyl)methyl]-3-(2,3-dihydro-1H-inden-2-yl)-6-(2-methylpropyl)-2,5-piperazinedione;
(3R,6R)-3-(2,3-dihydro-1H-inden-2-yl)-6-(1-ethylpropyl)-1-{[2-(4-morpholinyl)phenyl]-methyl}-2,5-piperazinedione;
(3R,6R)-1-{[3,5-bis(methyloxy)phenyl]methyl}-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-({4-[(trifluoromethyl)oxy]-phenyl}methyl)-2,5-piperazinedione;
(3R,6R)-3-(2,3-dihydro-1H-inden-2-yl)-1-[(3,5-dimethylphenyl)methyl]-6-(1-ethylpropyl)-2,5-piperazinedione;
(3R,6R)-3-(2,3-dihydro-1H-inden-2-yl)-6-(1-ethylpropyl)-1-(phenylmethyl)-2,5-piperazinedione;
(3R,6R)-1-[(4-chlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(2-methylpropyl)-1-{[3-(2-pyridinyloxy)phenyl]-methyl}-2,5-piperazinedione;
(3R,6R)-3-(2,3-dihydro-1H-inden-2-yl)-6-(1-ethylpropyl)-1-{[2-(methylsulfonyl)phenyl]-methyl}-2,5-piperazinedione;
(3R,6R)-6-cyclohexyl-3-(2,3-dihydro-1H-inden-2-yl)-1-{[2-(methylsulfonyl)phenyl]methyl}-2,5-piperazinedione;
(3R,6R)-3-(2,3-dihydro-1H-inden-2-yl)-6-(2-methylphenyl)-1-{[2-(methylsulfonyl)phenyl]-methyl}-2,5-piperazinedione;
(3R,6R)-3-(2,3-dihydro-1H-inden-2-yl)-6-(1-methylethyl)-1-{[2-(methylsulfonyl)phenyl]-methyl}-2,5-piperazinedione;
(3R,6R)-6-(dicyclopropylmethyl)-3-(2,3-dihydro-1H-inden-2-yl)-1-{[2-(methylsulfonyl)-phenyl]methyl}-2,5-piperazinedione;
(3R,6R)-3-(2,3-dihydro-1H-inden-2-yl)-6-(2,2-dimethylpropyl)-1-{[2-(methylsulfonyl)-phenyl]methyl}-2,5-piperazinedione;
(3R,6R)-3-(2,3-dihydro-1H-inden-2-yl)-6-(1-ethylpropyl)-1-({4-[(trifluoromethyl)-sulfonyl]phenyl}methyl)-2,5-piperazinedione;
(3R,6R)-3-(2,3-dihydro-1H-inden-2-yl)-1-{[2-(methyloxy)-4-(methylsulfonyl)phenyl]-methyl}-6-(2-methylpropyl)-2,5-piperazinedione;
(3R,6R)-3-(2,3-dihydro-1H-inden-2-yl)-6-(1-ethylpropyl)-1-{[2-(methyloxy)-4-(methylsulfonyl)phenyl]methyl}-2,5-piperazinedione;
(3R,6R)-1-{[2-chloro-4-(methylsulfonyl)phenyl]methyl}-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-[(5-methyl-1-phenyl-1H-pyrazol-4-yl)methyl]-2,5-piperazinedione;
(3R,6R)-3-(2,3-dihydro-1H-inden-2-yl)-1-({2-[(1,1-dimethylethyl)thio]phenyl}methyl)-6-(1-ethylpropyl)-2,5-piperazinedione;
5 (3R,6R)-3-(2,3-dihydro-1H-inden-2-yl)-6-(1-ethylpropyl)-1-{[1-methyl-5-(trifluoromethyl)-1H-pyrazol-4-yl]methyl}-2,5-piperazinedione;
(3R,6R)-3-(2,3-dihydro-1H-inden-2-yl)-6-(1-ethylpropyl)-1-{[3-(methylsulfonyl)-2-pyridinyl]methyl}-2,5-piperazinedione;
(3R,6R)-3-(2,3-dihydro-1H-inden-2-yl)-6-(1-ethylpropyl)-1-(2-nitrobenzyl)piperazine-2,5-dione;
2-{[(3R,6R)-3-(2,3-dihydro-1H-inden-2-yl)-6-(1-ethylpropyl)-2,5-dioxo-1-piperazinyl]-methyl}benzoic acid;
2-{[(3R,6R)-3-(2,3-dihydro-1H-inden-2-yl)-6-(1-ethylpropyl)-2,5-dioxo-1-piperazinyl]-methyl}-N-methylbenzamide;
2-{[(3R,6R)-3-(2,3-dihydro-1H-inden-2-yl)-6-(1-ethylpropyl)-2,5-dioxo-1-piperazinyl]-methyl}benzamide;
2-{[(3R,6R)-3-(2,3-dihydro-1H-inden-2-yl)-6-(1-ethylpropyl)-2,5-dioxo-1-piperazinyl]-methyl}-N,N-dimethylbenzamide;
2-{[(3R,6R)-3-(2,3-dihydro-1H-inden-2-yl)-6-(1-ethylpropyl)-2,5-dioxo-1-piperazinyl]-methyl}-N-methyl-N-(1-methyl-4-piperidinyl)benzamide;
(3R,6R)-3-(2,3-dihydro-1H-inden-2-yl)-1-[(2-{[4-(dimethylamino)-1-piperidinyl]-carbonyl}phenyl)methyl]-6-(1-ethylpropyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-(1-methyl-4-piperidinyl)benzamide formate;
2-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]-N-methylbenzamide;
2-{[(3R,6R)-3-(2,3-dihydro-1H-inden-2-yl)-6-(1-ethylpropyl)-2,5-dioxo-1-piperazinyl]-methyl}-N-[3-(dimethylamino)propyl]-N-methylbenzamide formate;
2-{[(3R,6R)-3-(2,3-dihydro-1H-inden-2-yl)-6-(1-ethylpropyl)-2,5-dioxo-1-piperazinyl]-methyl}-N-[3-(dimethylamino)propyl]benzamide;
2-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]benzamide;
(3R,6R)-3-(2,3-dihydro-1H-inden-2-yl)-6-(1-ethylpropyl)-1-({2-[(4-methyl-1-piperazinyl)-carbonyl]phenyl}methyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-[2-(4-morpholinyl)ethyl]benzamide;
2-{[(3R,6R)-3-(2,3-dihydro-1H-inden-2-yl)-6-(1-ethylpropyl)-2,5-dioxo-1-piperazinyl]-methyl}-N-(2-hydroxyethyl)benzamide;
2-{[(3R,6R)-3-(2,3-dihydro-1H-inden-2-yl)-6-(1-ethylpropyl)-2,5-dioxo-1-piperazinyl]-methyl}-N-(2-hydroxyethyl)-N-methylbenzamide;
(3R,6R)-3-(2,3-dihydro-1H-inden-2-yl)-6-(1-ethylpropyl)-1-{[2-(4-morpholinylcarbonyl)-phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-N-4-piperidinylbenzamide;
(3R,6R)-3-(2,3-dihydro-1H-inden-2-yl)-6-(1-ethylpropyl)-1-{[2-(1-piperazinylcarbonyl)-phenyl]methyl}-2,5-piperazinedione;
(3R,6R)-3-(2,3-dihydro-1H-inden-2-yl)-6-(1-ethylpropyl)-1-{[2-(4-morpholinylmethyl)-phenyl]methyl}-2,5-piperazinedione;
(3R,6R)-3-(2,3-dihydro-1H-inden-2-yl)-1-({2-[(dimethylamino)methyl]phenyl}methyl)-6-(1-ethylpropyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-methylbenzenesulfonamide;
2-{[(3R,6R)-3-(2,3-dihydro-1H-inden-2-yl)-6-(1-ethyl-propyl)-2,5-dioxo-1-piperazinyl]-methyl}-N,N-dimethyl-benzenesulfonamide;
(3R,6R)-3-(2,3-dihydro-1H-inden-2-yl)-6-(1-ethylpropyl)-1-{[2-(4-morpholinylsulfonyl)-phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}benzene-sulfonamide;
(3R,6R)-3-(2,3-dihydro-1H-inden-2-yl)-6-(1-ethylpropyl)-1-({2-[(4-methyl-1-piperazinyl)-sulfonyl]phenyl}-methyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-(1-methyl-4-piperidinyl)-benzenesulfonamide;
2-{[(3R,6R)-3-(2,3-dihydro-1H-inden-2-yl)-6-(1-ethyl-propyl)-2,5-dioxo-1-piperazinyl]-methyl}-N-methyl-N-(1-methyl-4-piperidinyl)benzenesulfonamide;
2-{[(3R,6R)-3-(2,3-dihydro-1H-inden-2-yl)-6-(1-ethylpropyl)-2,5-dioxo-1-piperazinyl]-methyl}-N-[2-(4-morpholinyl)ethyl]benzene-sulfonamide;
2-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]-benzenesulfonamide;
(3R,6R)-3-(2,3-dihydro-1H-inden-2-yl)-1-({2-[(4-ethyl-1-piperazinyl)sulfonyl]phenyl}-methyl)-6-(1-ethylpropyl)-2,5-piperazinedione;
(3R,6R)-3-(2,3-dihydro-1H-inden-2-yl)-6-(1-ethylpropyl)-1-{[2-({4-[2-(methyloxy)ethyl]-1-piperazinyl}sulfonyl)phenyl]methyl}-2,5-piperazinedione;
(3R,6R)-3-(2,3-dihydro-1H-inden-2-yl)-6-(1-ethylpropyl)-1-{[2-(1-piperazinylsulfonyl)-phenyl]methyl}-2,5-piperazinedione hydrochloride;
(3R,6R)-3-(2,3-dihydro-1H-inden-2-yl)-6-(1-ethylpropyl)-1-{[2-(4-piperidinylthio)-phenyl]methyl}-2,5-piperazinedione;
(3R,6R)-3-(2,3-dihydro-1H-inden-2-yl)-6-(1-ethylpropyl)-1-{[2-(4-piperidinylsulfonyl)-phenyl]methyl}-2,5-piperazinedione hydrochloride;
(3R,6R)-3-(2,3-dihydro-1H-inden-2-yl)-6-(1-ethylpropyl)-1-{[2-({1-[2-(methyloxy)-ethyl]-4-piperidinyl}sulfonyl)phenyl]methyl}-2,5-piperazinedione;
(3R,6R)-3-(2,3-dihydro-1H-inden-2-yl)-6-(1-ethylpropyl)-1-[(2-{[3-(4-morpholinyl)propyl]-thio}phenyl)methyl]-2,5-piperazinedione;
(3R,6R)-3-(2,3-dihydro-1H-inden-2-yl)-6-(1-ethylpropyl)-1-[(2-{[3-(4-morpholinyl)propyl]-sulfonyl}phenyl)methyl]-2,5-piperazinedione;
(3R,6R)-3-(2,3-dihydro-1H-inden-2-yl)-1-[(2-{[3-(dimethylamino)propyl]thio}phenyl)-methyl]-6-(1-ethylpropyl)-2,5-piperazinedione;
(3R,6R)-3-(2,3-dihydro-1H-inden-2-yl)-6-(1-ethylpropyl)-1-({2-[(1-methyl-4-piperidinyl)-thio]phenyl}methyl)-2,5-piperazinedione formate;
(3R,6R)-3-(2,3-dihydro-1H-inden-2-yl)-6-(1-ethylpropyl)-1-({2-[(1-methyl-4-piperidinyl)-sulfonyl]phenyl}methyl)-2,5-piperazinedione formate;
(3R,6R)-3-(2,3-dihydro-1H-inden-2-yl)-1-({2-[(1-ethyl-4-piperidinyl)sulfonyl]phenyl}-methyl)-6-(1-ethylpropyl)-2,5-piperazinedione;
(3R,6R)-1-[(2-Aminophenyl)methyl]-3-(2,3-dihydro-1H-inden-2-yl)-6-(1-ethylpropyl)-2,5-piperazinedione;
N-(2-{[(3R,6R)-3-(2,3-dihydro-1H-inden-2-yl)-6-(1-ethylpropyl)-2,5-dioxo-1-piperazinyl]methyl}phenyl)methanesulfonamide;
N-(2-{[(3R,6R)-3-(2,3-dihydro-1H-inden-2-yl)-6-(1-ethylpropyl)-2,5-dioxo-1-piperazinyl]-methyl}phenyl)ethane-sulfonamide;
N-(2-{[(3R,6R)-3-(2,3-dihydro-1H-inden-2-yl)-6-(1-ethylpropyl)-2,5-dioxo-1-piperazinyl]-methyl}phenyl)-2-propanesulfonamide;
N-(2-{[(3R,6R)-3-(2,3-dihydro-1H-inden-2-yl)-6-(1-ethylpropyl)-2,5-dioxo-1-piperazinyl]-methyl}phenyl)-N-methylmethanesulfonamide;
(3R,6R)-3-(2,3-dihydro-1H-inden-2-yl)-1-{[2-(1,1-dioxido-2-isothiazolidinyl)phenyl]-methyl}-6-(1-ethylpropyl)-2,5-piperazinedione;
N-(2-{[(3R,6R)-3-(2,3-dihydro-1H-inden-2-yl)-6-(1-ethylpropyl)-2,5-dioxo-1-piperazinyl]-methyl}phenyl)acetamide;
N-(2-{[(3R,6R)-3-(2,3-dihydro-1H-inden-2-yl)-6-(1-ethylpropyl)-2,5-dioxo-1-piperazinyl]-methyl}phenyl)-N³,N³-dimethyl-β-alaninamide formate;
N-(2-{[(3R,6R)-3-(2,3-dihydro-1H-inden-2-yl)-6-(1-ethylpropyl)-2,5-dioxo-1-piperazinyl]-methyl}-phenyl)-4-(dimethyl-amino)butanamide formate;
(formic acid —N-(2-{[(3R,6R)-3-(2,3-dihydro-1H-inden-2-yl)-6-(1-ethylpropyl)-2,5-dioxo-1-piperazinyl]methyl}phenyl)-1-methyl-4-piperidinecarboxamide;
(3R,6R)-3-(2,3-dihydro-1H-inden-2-yl)-6-(1-ethylpropyl)-1-{[2-(2-oxo-1-pyrrolidinyl)-phenyl]methyl}-2,5-piperazinedione;
4-{[(3R,6R)-3-(2,3-dihydro-1H-inden-2-yl)-6-(2-methylpropyl)-2,5-dioxo-1-piperazinyl]-methyl}-N,N-dimethylbenzenesulfonamide;
4-{[(3R,6R)-3-(2,3-dihydro-1H-inden-2-yl)-6-(2-methylpropyl)-2,5-dioxo-1-piperazinyl]-methyl}-N,N-dimethylbenzenesulfonamide;
(3R,6R)-3-(2,3-dihydro-1H-inden-2-yl)-1-[(3-{[ethyl(methyl)amino]methyl}phenyl)methyl]-6-(1-ethylpropyl)-2,5-piperazinedione;
(3R,6R)-3-(2,3-dihydro-1H-inden-2-yl)-6-(2-methylpropyl)-1-[(1-phenyl-1H-pyrazol-4-yl)methyl]-2,5-piperazinedione;
(3R,6R)-3-(2,3-dihydro-1H-inden-2-yl)-1-{[1-(3-methylphenyl)-1H-pyrazol-4-yl]methyl}-6-(2-methylpropyl)-2,5-piperazinedione;
(3R,6R)-3-(2,3-dihydro-1H-inden-2-yl)-6-(2-methylpropyl)-1-[2-(3-pyridinyl)ethyl]-2,5-piperazinedione;
N-(3-{[(3R,6R)-3-(2,3-dihydro-1H-inden-2-yl)-6-(2-methylpropyl)-2,5-dioxo-1-piperazinyl]-methyl}phenyl)methanesulfonamide;
N-(2-{[(3R,6R)-3-(2,3-dihydro-1H-inden-2-yl)-6-(2-methylpropyl)-2,5-dioxo-1-piperazinyl]-methyl}phenyl)-N-methylacetamide;
(3R,6R)-3-(2,3-dihydro-1H-inden-2-yl)-1-{2-[4-(methyloxy)-3-(methylsulfonyl)phenyl]-ethyl}-6-(2-methylpropyl)-2,5-piperazinedione;
(3R,6R)-3-(2,3-dihydro-1H-inden-2-yl)-6-(1-ethylpropyl)-1-[(1-methyl-1H-pyrazol-4-yl)methyl]-2,5-piperazinedione;
(3R,6R)-3-(2,3-dihydro-1H-inden-2-yl)-6-(1-ethylpropyl)-1-[(1S)-1-phenylethyl]-2,5-piperazinedione;
(3R,6R)-3-(2,3-dihydro-1H-inden-2-yl)-6-(1-ethylpropyl)-1-(3-pyridinylmethyl)-2,5-piperazinedione;
(3R,6R)-3-(2,3-dihydro-1H-inden-2-yl)-6-(1-ethylpropyl)-1-(4-pyridinylmethyl)-2,5-piperazinedione;
2-{[(3R,6R)-3-(2,3-dihydro-1H-inden-2-yl)-6-(1,1-dimethylpropyl)-2,5-dioxo-1-piperazinyl]methyl}-N-methylbenzamide;
(3R,6R)-3-(2,3-dihydro-1H-inden-2-yl)-6-(1-ethylpropyl)-1-[(2-oxo-1,2-dihydro-3-pyridinyl)methyl]-2,5-piperazinedione;
(3R,6R)-3-(2,3-dihydro-1H-inden-2-yl)-6-(1-ethylpropyl)-1-[(2-hydroxyphenyl)methyl]-2,5-piperazinedione;
2-{[(3R,6R)-3-(2,3-dihydro-1H-inden-2-yl)-6-(1-methylethyl)-2,5-dioxo-1-piperazinyl]methyl}-N-methylbenzamide;
(3R,6R)-3-(2,3-dihydro-1H-inden-2-yl)-6-(1-ethylpropyl)-1-{[1-(phenylmethyl)-1H-pyrazol-4-yl]methyl}-2,5-piperazinedione;
(3R,6R)-6-cyclopentyl-3-(2,3-dihydro-1H-inden-2-yl)-1-{[2-(methylsulfonyl)phenyl]-methyl}-2,5-piperazinedione;
(3R,6R)-3-(2,3-dihydro-1H-inden-2-yl)-6-(1-ethylpropyl)-1-{[2-(methylthio)phenyl]methyl}-2,5-piperazinedione;
(3R,6R)-3-(2,3-dihydro-1H-inden-2-yl)-6-(2-methylpropyl)-1-{[2-(methylthio)phenyl]-methyl}-2,5-piperazinedione;
(3R,6R)-1-[(2,4-dichlorophenyl)methyl]-3-(2,3-dihydro-1H-inden-2-yl)-6-(1-ethylpropyl)-2,5-piperazinedione;
(3R,6R)-1-(2-biphenylylmethyl)-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-[2-(3-pyridinyl)ethyl]-2,5-piperazinedione;
(3R,6R)-6-(dicyclopropylmethyl)-3-(2,3-dihydro-1H-inden-2-yl)-1-[(3-methylphenyl)-methyl]-2,5-piperazinedione;
(3R,6R)-1-{[3,4-bis(methyloxy)phenyl]methyl}-3-(2,3-dihydro-1H-inden-2-yl)-6-(2-methylpropyl)-2,5-piperazinedione;
(3R,6R)-3-(2,3-dihydro-1H-inden-2-yl)-1-[(4-{[2-(dimethylamino)ethyl]oxy}phenyl)methyl]-6-(1-ethylpropyl)-2,5-piperazinedione;
(3R,6R)-3-(2,3-dihydro-1H-inden-2-yl)-6-(1-ethylpropyl)-1-{[3-(1-pyrrolidinylmethyl)-phenyl]methyl}-2,5-piperazinedione;
(3R,6R)-3-(2,3-dihydro-1H-inden-2-yl)-6-(1-ethylpropyl)-1-{[4-(4-morpholinylmethyl)-phenyl]methyl}-2,5-piperazinedione;
formic acid-(3R,6R)-3-(2,3-dihydro-1H-inden-2-yl)-1-({3-[(dimethylamino)methyl]-phenyl}methyl)-6-(1-ethylpropyl)-2,5-piperazinedione (1:1);
(3R,6R)-3-(2,3-dihydro-1H-inden-2-yl)-6-(1-methylethyl)-1-[(3-methylphenyl)methyl]-2,5-piperazinedione;
N-cyclopropyl-4-{[(3R,6R)-3-(2,3-dihydro-1H-inden-2-yl)-6-(2-methylpropyl)-2,5-dioxo-1-piperazinyl]methyl}benzamide;
(3R,6R)-3-(2,3-dihydro-1H-inden-2-yl)-6-(2-methylpropyl)-1-{[3-(3-pyridinyl)phenyl]-methyl}-2,5-piperazinedione;
(3R,6R)-3-(2,3-dihydro-1H-inden-2-yl)-6-(1-ethylpropyl)-1-{[4-fluoro-2-(hydroxymethyl)-phenyl]methyl}-2,5-piperazinedione;
(3R,6R)-3-(2,3-dihydro-1H-inden-2-yl)-6-(1-ethylpropyl)-1-{[4-(2-pyrazinylamino)-phenyl]methyl}-2,5-piperazinedione;
(3R,6R)-3-(2,3-dihydro-1H-inden-2-yl)-6-(1-ethylpropyl)-1-{[4-(2-pyrimidinylamino)-phenyl]methyl}-2,5-piperazinedione;
(3R,6R)-3-(2,3-dihydro-1H-inden-2-yl)-6-(2-methylpropyl)-1-{[4-(2-pyrimidinylamino)-phenyl]methyl}-2,5-piperazinedione;
(3R,6R)-3-(2,3-dihydro-1H-inden-2-yl)-6-(1-ethylpropyl)-1-({4-[(1-methyl-1H-imidazol-2-yl)amino]phenyl}methyl)-2,5-piperazinedione;
(3R,6R)-3-(2,3-dihydro-1H-inden-2-yl)-1-({4-[(1-methyl-1H-imidazol-2-yl)amino]phenyl}-methyl)-6-(2-methylpropyl)-2,5-piperazinedione;
(3R,6R)-3-(2,3-dihydro-1H-inden-2-yl)-6-(1-ethylpropyl)-1-({4-[(5-methyl-1,3,4-thiadiazol-2-yl)amino]phenyl}methyl)-2,5-piperazinedione;
(3R,6R)-3-(2,3-dihydro-1H-inden-2-yl)-6-(2-methylpropyl)-1-({4-[(5-methyl-1,3,4-thiadiazol-2-yl)amino]phenyl}methyl)-2,5-piperazinedione;
(3R,6R)-3-(2,3-dihydro-1H-inden-2-yl)-6-(1-ethylpropyl)-1-({4-[(5-methyl-1,3-thiazol-2-yl)amino]phenyl}methyl)-2,5-piperazinedione;
(3R,6R)-3-(2,3-dihydro-1H-inden-2-yl)-6-(2-methylpropyl)-1-({4-[(5-methyl-1,3-thiazol-2-yl)amino]phenyl}methyl)-2,5-piperazinedione;
(3R,6R)-3-(2,3-dihydro-1H-inden-2-yl)-6-(1-ethylpropyl)-1-{[4-(1H-pyrazol-1-yl)phenyl]methyl}-2,5-piperazinedione;
(3R,6R)-3-(2,3-dihydro-1H-inden-2-yl)-6-(1-ethylpropyl)-1-{[4-(1H-1,2,3-triazol-1-yl)phenyl]methyl}-2,5-piperazinedione;
(3R,6R)-3-(2,3-dihydro-1H-inden-2-yl)-6-(1-ethylpropyl)-1-{[4-(1H-1,2,4-triazol-1-yl)phenyl]methyl}-2,5-piperazinedione;
(3R,6R)-3-(2,3-dihydro-1H-inden-2-yl)-6-(1-ethylpropyl)-1-{[4-(2H-1,2,3-triazol-2-yl)phenyl]methyl}-2,5-piperazinedione;
(3R,6R)-3-(2,3-dihydro-1H-inden-2-yl)-6-(2-methylpropyl)-1-{[2-(1H-tetrazol-1-yl)phenyl]methyl}-2,5-piperazinedione;
(3R,6R)-3-(2,3-dihydro-1H-inden-2-yl)-6-(1-ethylpropyl)-1-{[2-(1H-tetrazol-1-yl)phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-5-fluorobenzoic acid;
2-{[(3R,6R)-3-(2,3-dihydro-1H-inden-2-yl)-6-(1-ethylpropyl)-2,5-dioxo-1-piperazinyl]methyl}-5-fluoro-N,N-dimethylbenzamide;
(3R,6R)-3-(2,3-dihydro-1H-inden-2-yl)-6-(1-ethylpropyl)-1-{[4-fluoro-2-(4-morpholinylcarbonyl)phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-5-fluoro-N-(2-hydroxyethyl)benzamide;
2-{[(3R,6R)-3-(2,3-dihydro-1H-inden-2-yl)-6-(2-methylpropyl)-2,5-dioxo-1-piperazinyl]-methyl}benzoic acid;
2-{[(3R,6R)-3-(2,3-dihydro-1H-inden-2-yl)-6-(2-methylpropyl)-2,5-dioxo-1-piperazinyl]-methyl}benzamide;
2-{[(3R,6R)-3-(2,3-dihydro-1H-inden-2-yl)-6-(2-methylpropyl)-2,5-dioxo-1-piperazinyl]-methyl}-N,N-dimethylbenzamide;
(3R,6R)-3-(2,3-dihydro-1H-inden-2-yl)-6-(2-methylpropyl)-1-{[2-(4-morpholinylcarbonyl)-phenyl]methyl}-2,5-piperazinedione;
2-{[(3R,6R)-3-(2,3-dihydro-1H-inden-2-yl)-6-(2-methylpropyl)-2,5-dioxo-1-piperazinyl]-methyl}-N-methylbenzamide;
3-{[(3R,6R)-3-(2,3-dihydro-1H-inden-2-yl)-6-(1-ethylpropyl)-2,5-dioxo-1-piperazinyl]-methyl}benzoic acid;
3-{[(3R,6R)-3-(2,3-dihydro-1H-inden-2-yl)-6-(1-ethylpropyl)-2,5-dioxo-1-piperazinyl]-methyl}benzamide;
3-{[(3R,6R)-3-(2,3-dihydro-1H-inden-2-yl)-6-(1-ethylpropyl)-2,5-dioxo-1-piperazinyl]methyl}-N-methylbenzamide;
(3R,6R)-3-(2,3-dihydro-1H-inden-2-yl)-6-(1-ethylpropyl)-1-{[3-(4-morpholinylcarbonyl)-phenyl]methyl}-2,5-piperazinedione;
4-{[(3R,6R)-3-(2,3-dihydro-1H-inden-2-yl)-6-(1-ethylpropyl)-2,5-dioxo-1-piperazinyl]-methyl}benzoic acid;
4-{[(3R,6R)-3-(2,3-dihydro-1H-inden-2-yl)-6-(1-ethylpropyl)-2,5-dioxo-1-piperazinyl]-methyl}benzamide;
4-{[(3R,6R)-3-(2,3-dihydro-1H-inden-2-yl)-6-(1-ethylpropyl)-2,5-dioxo-1-piperazinyl]-methyl}-N-methylbenzamide;
(3R,6R)-3-(2,3-dihydro-1H-inden-2-yl)-6-(1-ethylpropyl)-1-{[4-(4-morpholinylcarbonyl)-phenyl]methyl}-2,5-piperazinedione;
4-{[(3R,6R)-3-(2,3-dihydro-1H-inden-2-yl)-6-(1-ethylpropyl)-2,5-dioxo-1-piperazinyl]-methyl}-N-(2-hydroxyethyl)benzamide;
4-{[(3R,6R)-3-(2,3-dihydro-1H-inden-2-yl)-6-(1-ethylpropyl)-2,5-dioxo-1-piperazinyl]-methyl}-N-methyl-N-[2-(methyloxy)ethyl]benzamide;
4-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]-N-methylbenzamide;
4-{[(3R,6R)-3-(2,3-dihydro-1H-inden-2-yl)-6-(1-ethylpropyl)-2,5-dioxo-1-piperazinyl]methyl}-N-(2-hydroxyethyl)-N-methylbenzamide;
(3R,6R)-3-(2,3-dihydro-1H-inden-2-yl)-1-({4-[(4-methyl-1-piperazinyl)carbonyl]-phenyl}methyl)-6-(2-methylpropyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-{1-[2-(methyloxy)ethyl]-4-piperidinyl}benzamide;
(3R,6R)-1-{[2,4-bis(hydroxymethyl)phenyl]methyl}-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[2-(hydroxymethyl)-4-(methylthio)phenyl]methyl}-2,5-piperazinedione;
(3R,6R)-1-{[2,4-bis(1-pyrrolidinylcarbonyl)phenyl]methyl}-3-(2,3-dihydro-1H-inden-2-yl)-6-(1-ethylpropyl)-2,5-piperazinedione;
(3R,6R)-1-{[2,4-bis(4-morpholinylcarbonyl)phenyl]methyl}-3-(2,3-dihydro-1H-inden-2-yl)-6-(1-ethylpropyl)-2,5-piperazinedione;
4-{[(3R,6R)-3-(2,3-dihydro-1H-inden-2-yl)-6-(1-ethylpropyl)-2,5-dioxo-1-piperazinyl]-methyl}-N,N,N′,N′-tetramethyl-1,3-benzenedicarboxamide;
4-{[(3R,6R)-3-(2,3-dihydro-1H-inden-2-yl)-6-(1-ethylpropyl)-2,5-dioxo-1-piperazinyl]-methyl}-N,N′-bis(2-hydroxyethyl)-N,N′-dimethyl-1,3-benzenedicarboxamide;
4-{[(3R,6R)-3-(2,3-dihydro-1H-inden-2-yl)-6-(1-ethylpropyl)-2,5-dioxo-1-piperazinyl]-methyl}-N,N′-bis[2-(dimethylamino)ethyl]-N,N′-dimethyl-1,3-benzenedicarboxamide;
4-{[(3R,6R)-3-(2,3-dihydro-1H-inden-2-yl)-6-(1-ethylpropyl)-2,5-dioxo-1-piperazinyl]-methyl}-N,N′-dimethyl-N,N′-bis[2-(methyloxy)ethyl]-1,3-benzenedicarboxamide;
(3R,6R)-1-({2,4-bis[(4-methyl-1-piperazinyl)carbonyl]phenyl}methyl)-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[2-(hydroxymethyl)-4-(methylsulfonyl)phenyl]methyl}-2,5-piperazinedione;
(3R,6R)-3-(2,3-dihydro-1H-inden-2-yl)-6-(1-ethylpropyl)-1-{[4-(methylsulfonyl)-2-(4-morpholinylcarbonyl)phenyl]methyl}-2,5-piperazinedione;
(3R,6R)-3-(2,3-dihydro-1H-inden-2-yl)-6-(1-ethylpropyl)-1-{[2-[(4-methyl-1-piperazinyl)-carbonyl]-4-(methylsulfonyl)phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-N-methyl-N-[2-(methyloxy)ethyl]-5-(methylsulfonyl)benzamide;
N-[2-(diethylamino)ethyl]-2-{[(3R,6R)-3-(2,3-dihydro-1H-inden-2-yl)-6-(1-ethylpropyl)-2,5-dioxo-1-piperazinyl]methyl}-N-methyl-5-(methylsulfonyl)benzamide;
(3R,6R)-3-(2,3-dihydro-1H-inden-2-yl)-6-(1-ethylpropyl)-1-[(4-(methylsulfonyl)-2-{[(2S)-2-(1-pyrrolidinylmethyl)-1-pyrrolidinyl]carbonyl}phenyl)methyl]-2,5-piperazinedione;
2-{[(3R,6R)-3-(2,3-dihydro-1H-inden-2-yl)-6-(1-ethylpropyl)-2,5-dioxo-1-piperazinyl]methyl}-N-(2-hydroxyethyl)benzenesulfonamide;
(3R,6R)-3-(2,3-dihydro-1H-inden-2-yl)-6-(1-ethylpropyl)-1-({2-[(4-methylhexahydro-1H-1,4-diazepin-1-yl)sulfonyl]phenyl}methyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-{[(2R)-1-ethyl-2-pyrrolidinyl]methyl}benzenesulfonamide;
(3R,6R)-3-(2,3-dihydro-1H-inden-2-yl)-1-[(2-{[(3S)-3-(dimethylamino)-1-pyrrolidinyl]-sulfonyl}phenyl)methyl]-6-(1-ethylpropyl)-2,5-piperazinedione;
(3R,6R)-3-(2,3-dihydro-1H-inden-2-yl)-1-[(2-{[(3R)-3-(dimethylamino)-1-pyrrolidinyl]-sulfonyl}phenyl)methyl]-6-(1-ethylpropyl)-2,5-piperazinedione;
2-{[(3R,6R)-3-(2,3-dihydro-1H-inden-2-yl)-6-(1-methylethyl)-2,5-dioxo-1-piperazinyl]-methyl}benzenesulfonamide;
N-[(2-{[(3R,6R)-3-(2,3-dihydro-1H-inden-2-yl)-6-(1-ethylpropyl)-2,5-dioxo-1-piperazinyl]-methyl}phenyl)sulfonyl]glycine;
(3R,6R)-3-(2,3-dihydro-1H-inden-2-yl)-6-(1-ethylpropyl)-1-({2-[(1-methylethyl)sulfonyl]-phenyl}methyl)-2,5-piperazinedione;
(3R,6R)-1-[(2-aminophenyl)methyl]-3-(2,3-dihydro-1H-inden-2-yl)-6-(2-methylpropyl)-2,5-piperazinedione;
formic acid-N-(2-{[(3R,6R)-3-(2,3-dihydro-1H-inden-2-yl)-6-(1-ethylpropyl)-2,5-dioxo-1-piperazinyl]methyl}phenyl)-N²,N²-dimethylglycinamide (1:1);
(3R,6R)-3-(2,3-dihydro-1H-inden-2-yl)-6-(1-ethylpropyl)-1-{[2-(1H-imidazol-1-yl)phenyl]-methyl}-2,5-piperazinedione;
formic acid-(3R,6R)-3-(2,3-dihydro-1H-inden-2-yl)-1-[(2-{[3-(dimethylamino)propyl]-sulfonyl}phenyl)methyl]-6-(1-ethylpropyl)-2,5-piperazinedione (1:1);
(3R,6R)-6-cyclopentyl-3-(2,3-dihydro-1H-inden-2-yl)-1-{[4-(methylsulfonyl)phenyl]-methyl}-2,5-piperazinedione;
(3R,6R)-3-(2,3-dihydro-1H-inden-2-yl)-1-[(1,5-dimethyl-1H-pyrazol-4-yl)methyl]-6-(1-ethylpropyl)-2,5-piperazinedione;
(3R,6R)-3-(2,3-dihydro-1H-inden-2-yl)-6-(1-ethylpropyl)-1-{[2-(methylsulfinyl)phenyl]-methyl}-2,5-piperazinedione;
(3R,6R)-3-(2,3-dihydro-1H-inden-2-yl)-6-(1-ethylpropyl)-1-{[2-(methylsulfinyl)phenyl]-methyl}-2,5-piperazinedione;
(3R,6R)-3-(2,3-dihydro-1H-inden-2-yl)-6-(2-methylpropyl)-1-{[2-(methylsulfinyl)phenyl]-methyl}-2,5-piperazinedione;
(3R,6R)-3-(2,3-dihydro-1H-inden-2-yl)-6-(2-methylpropyl)-1-{[2-(methylsulfinyl)phenyl]-methyl}-2,5-piperazinedione;
(3R,6R)-3-(2,3-dihydro-1H-inden-2-yl)-6-(1-ethylpropyl)-1-{[2-(1H-pyrazol-1-yl)phenyl]-methyl}-2,5-piperazinedione;
   and physiologically acceptable derivatives thereof.

The ability of the compounds of Formula (I) or Formula (A) to inhibit the actions of oxytocin may be determined using a variety of conventional procedures.

Thus, compounds of Formula (I) or Formula (A) have a high affinity for the oxytocin receptors on the uterus of rats and humans and this may be determined using conventional procedure. For example the affinity for the oxytocin receptors on the rat uterus may be determined by the procedure of Pettibone et al, Drug Development Research, 1993 (30) pp 129-142. The compounds of the invention also exhibit high affinity at the human recombinant oxytocin receptor in CHO cells and this may be conveniently demonstrated using the procedure described by Wyatt et al. Bioorganic & Medicinal Chemistry Letters, 2001 (11) pp 1301-1305.

The compounds of the invention are therefore useful in the treatment or prevention of diseases and/or conditions mediated through the action of oxytocin. Examples of such diseases and/or conditions include pre-term labour, dysmenorrhea, endometriosis and benign prostatic hyperplasia.

The compounds may also be useful to delay labour prior to elective caesarean section or transfer of the patient to a tertiary care centre, treatment of sexual dysfunction (male and female), particularly premature ejaculation, obesity, eating disorders, congestive heart failure, arterial hypertension, liver cirrhosis, nephritic or ocular hypertension, obsessive-compulsive disorder and neuropsychiatric disorders. The compounds of the invention may also be useful for improving fertility rates in animals, e.g. farm animals.

The invention therefore provides for the use of at least one chemical entity comprising a compound of Formula (IA) or Formula (A′) and physiologically acceptable derivatives thereof for use in therapy and in particular use as medicine for antagonising the effects of oxytocin upon the oxytocin receptor and for use in the treatment or prevention of diseases or conditions mediated through the action of oxytocin.

The invention also provides for the use of at least one chemical entity comprising a compound of Formula (IA) or Formula (A′) and physiologically acceptable derivatives thereof in the manufacture of a medicament for antagonising the effects of oxytocin on the oxytocin receptor. In one embodiment, the invention provides for the use of at least one chemical entity comprising a compound of Formula (IA) or Formula (A′) and physiologically acceptable derivatives thereof in the manufacture of a medicament for the treatment of one or more diseases or conditions selected from pre-term labour, dysmenorrhea and endometriosis.

According to a further aspect, the invention also provides for a method for antagonising the effects of oxytocin upon the oxytocin receptor, comprising administering to a patient in need thereof an antagonistic amount of a at least one chemical entity comprising at least one chemical entity comprising a compound of Formula (IA) or Formula (A′) and physiologically acceptable derivatives thereof.

According to another aspect, the invention also provides for a method of treating or preventing diseases or conditions mediated through the action of oxytocin, which comprises administering to a mammal in need thereof an effective amount of at least one chemical entity comprising a compound of Formula (IA) or Formula (A′) and physiologically acceptable derivatives thereof. In one aspect, the disease or condition is selected from pre-term labour, dysmenorrhea and endometriosis.

It will be appreciated by those skilled in the art that reference herein to treatment extends to prophylactics as well as the treatment of established diseases or symptoms.

It will further be appreciated that the amount of a compound of the invention required for use in treatment will vary with the nature of the condition being treated, the route of administration and the age and the condition of the patient and will be ultimately at the discretion of the attendant physician. In general however doses employed for adult human treatment will typically be in the range of 2 to 800 mg per day, dependent upon the route of administration.

Thus for parenteral administration a daily dose will typically be in the range 2 to 50 mg, preferably 5 to 25 mg per day. For oral administration a daily dose will typically be within the range 10 to 800 mg, e.g. 20 to 150 mg per day.

The desired dose may conveniently be presented in a single dose or as divided doses administered at appropriate intervals, for example as two, three, four or more sub-doses per day.

### Compositions

While it is possible that, for use in therapy, a compound of the invention may be administered as the raw chemical, it is preferable to present the active ingredient as a pharmaceutical formulation.

The invention thus further provides a pharmaceutical composition comprising at least one chemical entity comprising a compound of Formula (IA) or Formula (A′) and physiologically acceptable derivatives thereof and a pharmaceutically acceptable carrier or diluent. The formulation may optionally contain other therapeutic and/or prophylactic ingredients. The carrier(s) must be ‘acceptable’ in the sense of being compatible with the other ingredients of the formulation and not deleterious to the recipient thereof.

The compositions of the invention include those in a form especially formulated for oral, buccal, parenteral, inhalation or insufflation, implant or rectal administration.

Tablets and capsules for oral administration may contain conventional excipients such as binding agents, for example, syrup, acacia, gelatin, sorbitol, tragacanth, mucilage of starch or polyvinylpyrrolidone; fillers, for example, lactose, sugar, microcystalline cellulose, maize-starch, calcium phosphate or sorbitol; lubricants, for example, magnesium stearate, stearic acid, talc, polyethylene glycol or silica; disintegrants, for example, potato starch or sodium starch glycollate, or wetting agents such as sodium lauryl sulphate. The tablets may be coated according to methods well known in the art. Oral liquid preparations may be in the form of, for example, aqueous or oily suspensions, solutions emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxyethylcellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example, almond oil, fractionated coconut oil, oily esters, propylene glycol or ethyl alcohol; solubilizers such as surfactants for example polysorbates or other agents such as cyclodextrins; and preservatives, for example, methyl or propyl p-hydroxybenzoates or ascorbic acid. The compositions may also be formulated as suppositories, e.g. containing conventional suppository bases such as cocoa butter or other glycerides.

For buccal administration the composition may take the form of tablets or lozenges formulated in conventional manner.

The composition according to the invention may be formulated for parenteral administration by injection or continuous infusion. Formulations for injection may be presented in unit dose form in ampoules, or in multi-dose containers with an added preservative. The compositions may take such forms as suspensions, solutions, or emulsions in oily or aqueous vehicles, and may contain formulatory agents such as suspending, stabilising and/or dispersing agents. Alternatively the active ingredient may be in powder form for constitution with a suitable vehicle, e.g. sterile, pyrogen-free water, before use.

The compositions according to the invention may contain between 0.1-99% of the active ingredient, conveniently from 30-95% for tablets and capsules and 3-50% for liquid preparations.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

### PHARMACY EXAMPLES

### Tablets

| | | |
|---|---|---|
| a) | Compound of the invention | 50.0 mg |
| | Lactose | 70.0 mg |
| | Microcrystalline Cellulose | 70.0 mg |
| | Cross-linked polyvinylpyrrolidone | 8.0 mg |
| | Magnesium Stearate | 2.0 mg |
| | Compression weight | 200.0 mg |

The compound of the invention, microcrystalline cellulose, lactose and cross-linked polyvinylpyrrolidone are sieved through a 500 micron sieve and blended in a suitable mixer. The magnesium stearate is sieved through a 250 micron sieve and blended with the active blend. The blend is compressed into tablets using suitable punches.

| | | |
|---|---|---|
| b) | Compound of the invention | 50.0 mg |
| | Lactose | 120.0 mg |
| | Pregelatinised Starch | 20.0 mg |
| | Cross-linked polyvinylpyrrolidone | 8.0 mg |
| | Magnesium Stearate | 2.0 mg |
| | Compression weight | 200.0 mg |

The compound of the invention, lactose and pregelatinised starch are blended together and granulated with water. The wet mass is dried and milled. The magnesium stearate and cross-linked polyvinylpyrrolidone are screened through a 250 micron sieve and blended with the granule. The resultant blend is compressed using suitable tablet punches.

### Capsules

| | | |
|---|---|---|
| a) | Compound of the invention | 50.0 mg |
| | Lactose | 148.0 mg |
| | Magnesium Stearate | 2.0 mg |
| | Fill weight | 200.0 mg |

The compound of the invention and pregelatinised starch are screened through a 500 micron mesh sieve, blended together and lubricated with magnesium stearate, (meshed through a 250 micron sieve). The blend is filled into hard gelatine capsules of a suitable size.

| | | |
|---|---|---|
| b) | Compound of the invention | 50.0 mg |
| | Lactose | 132.0 mg |
| | Polyvinylpyrrolidone | 8.0 mg |
| | Cross-linked polyvinylpyrrolidone | 8.0 mg |
| | Magnesium Stearate | 2.0 mg |
| | Fill weight | 200.0 mg |

The compound of the invention and lactose are blended together and granulated with a solution of polyvinylpyrrolidone. The wet mass is dried and milled. The magnesium stearate and cross-linked polyvinylpyrrolidone are screened through a 250 micron sieve and blended with the granules. The resultant blend is filled into hard gelatine capsules of a suitable size.

Injection Formulation

| | % w/v |
|---|---|
| Compound of the invention | 0.10 |
| Water for injections B.P. to | 100.00 |

Sodium chloride may be added to adjust the tonicity of the solution and the pH may be adjusted to that of maximum stability and/or to facilitate solution of the compound of the invention using dilute acid or alkali or by the addition of suitable buffer salts. Solubilisers, such as cosolvents, may also be added to facilitate solution of the compound of the invention. Antioxidants and metal chelating salts may also be included. The solution is clarified, made up to final volume with water and the pH remeasured and adjusted if necessary, to provide 1 mg/ml of the compound of Formula (I) or Formula (A). The solution may be packaged for injection, for example by filling and sealing in ampoules, vials or syringes. The ampoules, vials or syringes may be aseptically filled (e.g. the solution may be sterilised by filtration and filled into sterile ampoules under aseptic conditions) and/or terminally sterilised (e.g. by heating in an autoclave using one of the acceptable cycles). The solution may be packed under an inert atmosphere of nitrogen.

Preferably the solution is filled into ampoules, sealed by fusion of the glass and terminally sterilised.

Further sterile formulations are prepared in a similar manner containing 0.05, 0.20 and 0.5% w/v of the compound of the invention, so as to provide respectively 0.5, 2 and 5 mg/ml of the compound of the invention.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with tocolytics or prophylactic medicines. These include, but are not limited to, beta-agonists such as terbutaline or ritodrine, calcium channel blockers, e.g. nifedepine, non-steroidal anti-inflammatory drugs, such as indomethacin, salts of magnesium, such as magnesium sulphate, other oxytocin antagonists, such as atosiban, and progesterone agonists and formulations. In addition the compounds of the present invention may be used in combination with antenatal steroids including betamethasone and dexamethasone, prenatal vitamins especially folate supplements, antibiotics, including but not limited to ampicillin, amoxicillin/clavulanate, metronidazole, clindamycin, and anxiolytics.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the present invention or the second therapeutic agent may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manner as are known for such compounds in the art.

### Abbreviations

In describing the invention, chemical elements are identified in accordance with the Periodic Table of the Elements. Abbreviations and symbols utilized herein are in accordance with the common usage of such abbreviations and symbols by those skilled in the chemical arts. The following abbreviations are used herein:
BOC tert-butyloxycarbonyl
Cbz benzyloxycarbonyl
CDCl₃ deuterated chloroform
DCM dichloromethane
DIPEA diisopropylamine
dioxan 1,4-dioxan
DMF N,N-dimethylformamide
DMSO dimethylsulfoxide
DMSO-d6 deuterated dimethylsulfoxide
EDC.HCl 1-(3-dimethylaminopropyl)-3-ethylcarboiimide hydrochloride
ES+ MS Positive Electrospray mass spectrometry
h hours
ES− MS Negative Electrospray mass spectrometry
HATU O-(7-azabenzotriazol-1-yl)-N,N,N′,N′-tetramethyluronium hexafluorophosphate
HPLC high pressure liquid chromatography
LCMS Liquid Chromatography Mass Spectrometry
mesylate methylsulfonate
min minutes
NMP N-methylpyrrolidone
NMR Nuclear Magnetic Resonance spectroscopy
Pd/C palladium on carbon
Rt retention time
RT room temperature
SPE solid phase extraction
TBDMSO tert-butyldimethylsilyloxy
TFA trifluoroacetic acid
THF tetrahydrofuran
tosylate 4-toluenesulfonate
xantphos 4,5-bis(diphenylphosphino)-9,9-dimethylxanthene

### Compound Preparation

Compounds of the invention may be prepared, in known manner in a variety of ways. In the following reaction Schemes and hereafter, unless otherwise stated R¹ to R¹², A, n and m are as defined above for Formula (I) or Formula (A), and the ring B represents a mono-, bi- or tricyclic aryl or heteroaryl group containing one or more heteroatoms independently selected from O, S or N, for example a phenyl, pyrazole or pyridinyl ring. These processes form further aspects of the invention.

Throughout the specification, general formulae are designated by Roman numerals (I), (II), (III), (IV), etc, with the exception of Formula (A). Subsets of compounds of Formulae (I) and (A) are defined as (Ia), (Aa), (Ib), (Ab), (Ic), (Ac), (Id), (Ad), (Ie), (Ae), (If), (Af), (Ig), (Ag), (If), (Af), (Ii), (Ai), (Ij), (Aj), (Ik), (Ak), (Im), (Am), (In), (An), (Io), (Ao), (Ip), (Ap), (Iq) and (Aq).

Compounds of Formula (I) or Formula (A), may be prepared according to the general reaction Scheme 1 by the following steps as indicated in the Scheme:

### Step (a)

A reaction between a chiral N-protected carboxylic acid of Formula (II), wherein P represents a suitable nitrogen protecting group, for example alkoxycarbonyl (e.g. tert-butoxycarbonyl), or Cbz; a suitable amine of Formula (III), an aldehyde (IV) and an isonitrile (V), wherein J is an optional substituent, for example, J is a chloro, benzyloxy or TBDMSO substituent or is absent, in a suitable solvent such as methanol, trifluoroethanol or chloroform, to give compounds of Formula (VI), wherein J is an optional substituent, for example, J is a chloro, benzyloxy or TBDMSO substituent or is absent.

It will be apparent to the person skilled in the art that compounds of Formula (II), (III), (IV) and (V) may be added together in varying order, for example an imine may be formed between aldehyde (IV) and amine (III) before the addition of carboxylic acid (II) and isonitrile (IV), or the reagents may be added together in a “one-pot” mixture.

It will be further apparent to the skilled person that the amine (III) may be added to the reaction in the form of a salt, such as a hydrochloride salt; in such a case a base may be added to the reaction mixture, for example triethylamine or DIPEA.

### Step (b)

A deprotection reaction to remove nitrogen protecting group P from compounds of Formula (VI) to provide compounds of Formula (VII), wherein J is an optional substituent, for example, J is a chloro, benzyloxy or TBDMSO substituent or is absent. When P is an alkoxycarbonyl group (e.g. tert-butoxycarbonyl), deprotection may be carried out using a suitable acid, for example TFA or HCl in a suitable solvent such as 1,4-dioxan or methanol, or in the presence of acetyl chloride in methanol. When P is a Cbz group, deprotection may be carried out by hydrogenation in the presence of a suitable catalyst, for example Pd/C, in a suitable solvent such as acetic acid or methanol.

### Step (c)

A cyclisation reaction of compounds of Formula (VII) to provide compounds of Formula (I) or Formula (A). Cyclisation may be carried out in the presence of a suitable acid such as glacial acetic acid, in a suitable solvent such as chloroform. Alternatively, cyclisation may be carried out in the presence of a suitable base, such as sodium bicarbonate, or a mixture of sodium bicarbonate and triethylamine. Alternatively, cyclisation may be carried out in the absence of acid or base in a suitable solvent.

It will be apparent to the person skilled in the art that the cis-diastereoisomer, i.e. the compound of Formula (I) or Formula (A), may be separated from the trans diastereoisomer (both isomers shown in Scheme 1) by conventional purification techniques, for example by chromatography. Alternatively, the mixture of cis- and trans-diastereoisomers may be subjected to functional group interconversion(s), for example those depicted in the reaction Schemes 3 to 12 hereinbelow, and separated by conventional techniques thereafter.

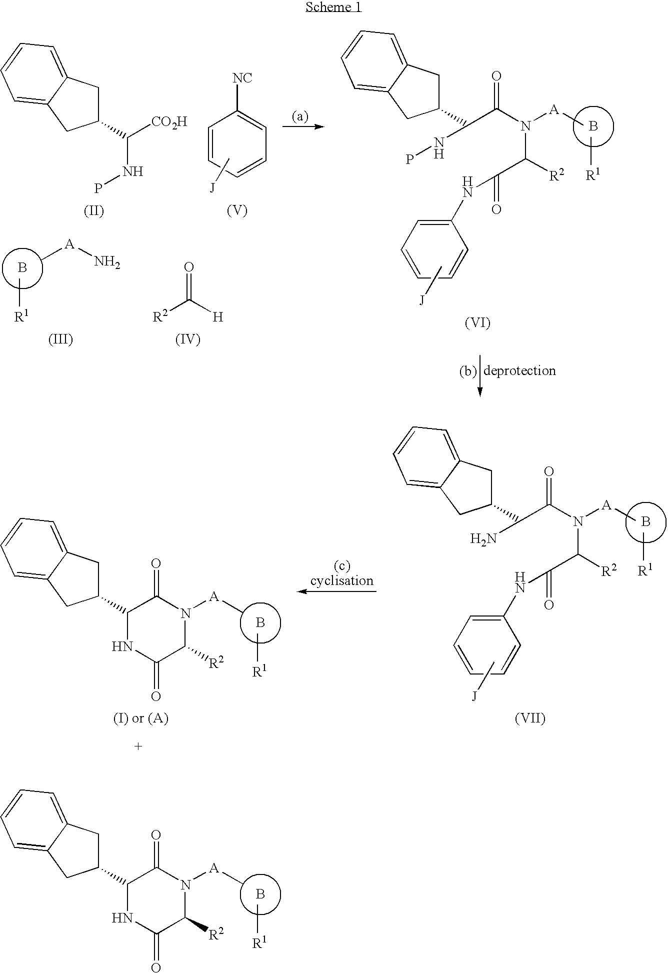

Alternatively, when P is an alkoxycarbonyl group (e.g. tert-butoxycarbonyl), the deprotection step and the cyclisation step may be carried out in a one-step reaction as shown in Scheme 2, in the presence of a suitable acid, for example HCl, in a suitable solvent, for example a mixture of 1,4-dioxan and DCM.

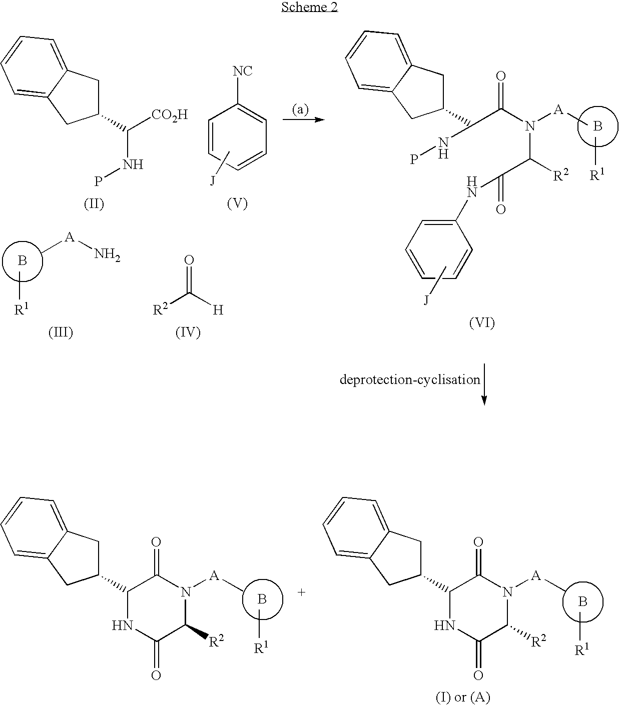

Compounds of Formula (Ib) or Formula (Ab), wherein R¹ represents —CONR³R⁴, may be prepared from compounds of Formula (Ia) or Formula (Aa), wherein R¹ represents —CH₂OH, according to reaction Scheme 3. Compounds (Ia) or (Aa) may be oxidised at the R¹ position to the carboxylic acid group —CO₂H. This can be carried out for example in a two-step process by reacting compounds of Formula (Ia) or (Aa) with 4-methylmorpholine N-oxide (NMNO) with tetrapropylammonium perruthenate (TPAP) in a suitable solvent such as dichloromethane, followed by oxidation of the resulting aldehyde using a suitable oxidising agent, such as sodium chlorite, to provide the carboxylic acid. The carboxylic acid may then be reacted with a suitable amine HNR³R⁴, for example in the presence of a coupling agent, such as 2-(1H-benzotriazol-1-yl)-1,1,3,3-tetramethyluronium tetrafluoroborate in the presence of a suitable base such as triethylamine, in a solvent e.g. dichloromethane, to form the compounds of Formula (Ib) or Formula (Ab).

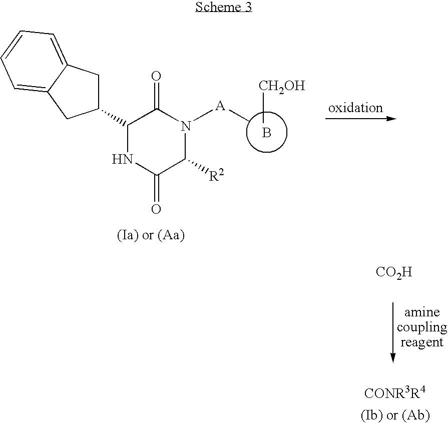

Compounds of Formula (Ib) or Formula (Ab), wherein R¹ represents the group —CONR³R⁴, wherein either one of R³ or R⁴ represents a group which contains an NH moiety, for example a nitrogen-containing heterocyclyl group, e.g. piperidine, may be prepared according to reaction Scheme 4 (piperidine shown by way of example) by deprotecting the corresponding N-protected compound of Formula (Ic) or (Ac), wherein P is a suitable nitrogen protecting group. Where P is, for example, an alkoxycarbonyl group (e.g. tert-butoxycarbonyl), deprotection may be carried out in the presence of an acid, e.g. HCl, in a suitable solvent such as 1,4-dioxan.

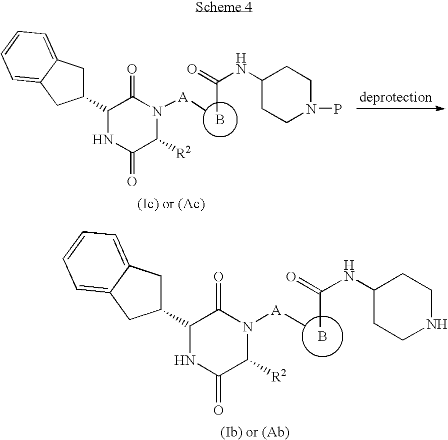

Compounds of Formula (Id) or (Ad), wherein R¹ represents C₁₋₆alkyl substituted by the group —NR³R⁴ may be prepared according to reaction Scheme 5 by reacting an aldehyde compound of Formula (VIII) with a suitable coupling agent, such as sodium triacetoxyborohydride, in the presence of a suitable amine HNR³R⁴.

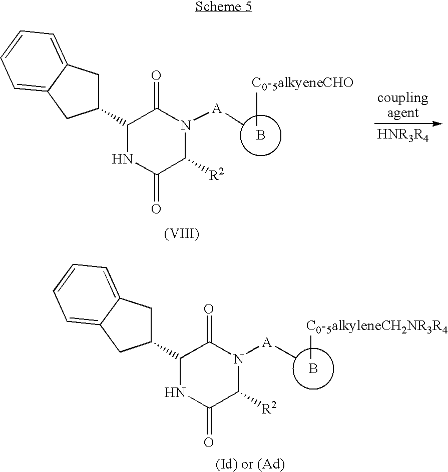

Compounds of Formula (Ie) or Formula (Ae), wherein R¹ represents the group —S(O)ₘNR⁹R¹⁰ may be prepared according to reaction Scheme 6 by deprotecting sulfanyl compounds of Formula (If) or (Af) to form the thiol compound IX. This may be carried out for example using a nitroaryl sulfenyl chloride, for example 2-nitrobenzenesulfenyl chloride, in the presence of a suitable base, such as triethylamine, and a suitable solvent, for example DMF, and tris(carboxyethyl)phosphine hydrochloride. Compound IX may be oxidised, e.g. with sulfuryl chloride in the presence of a suitable base, such as potassium nitrate, to form a sulfonyl chloride compound of Formula (II) or (Ai) which is subsequently reacted with a suitable amine HNR⁹R¹⁰ to form the amide (Ie) or (Ae).

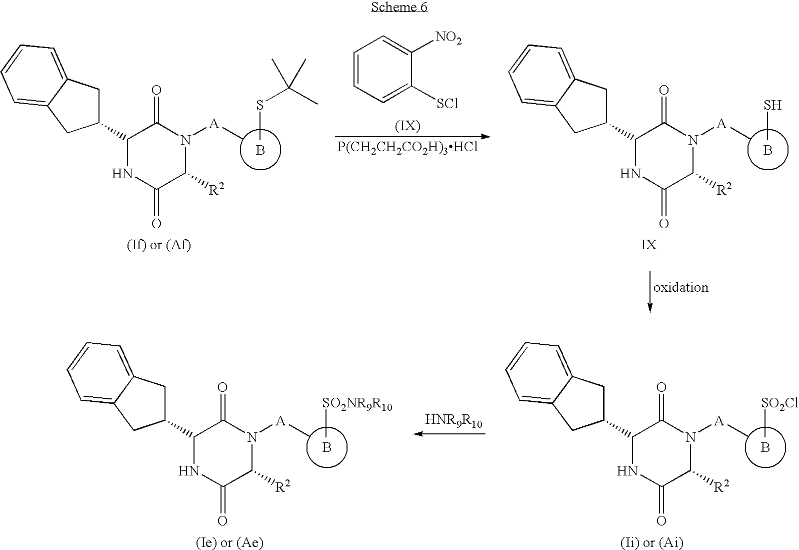

Compounds of Formula (I) or Formula (A), wherein R¹ represents the group —S(O)ₙR⁶ may be prepared according to reaction Scheme 7 starting from the thiol compound of Formula IX. This may be reacted with a suitable N-protected amine R⁶X, wherein X is a suitable leaving group, for example mesylate, tosylate or halo, to form a sulfanyl compound of Formula (Ij) or (Aj), wherein P is a suitable nitrogen protecting group, which can either be oxidised to the sulfone (Im) or (Am) using a suitable oxidising agent such as 3-chloroperoxybenzoic acid, or simply deprotected, for example using an acid, where P is a tert-butoxycarbonyl group, to form a sulfanyl compound of Formula (Ik) or (Ak). The sulfone (Im) or (Am) may be deprotected, for example using an acid, where P is a tert-butoxycarbonyl group, and may be further modified, if desired, to introduce a suitable group R⁹ on the amine, by treatment with R⁹Y, wherein Y is a suitable leaving group, for example mesylate, tosylate or halo, in the presence of a suitable base, for example potassium carbonate, in a suitable solvent, such as DMF.

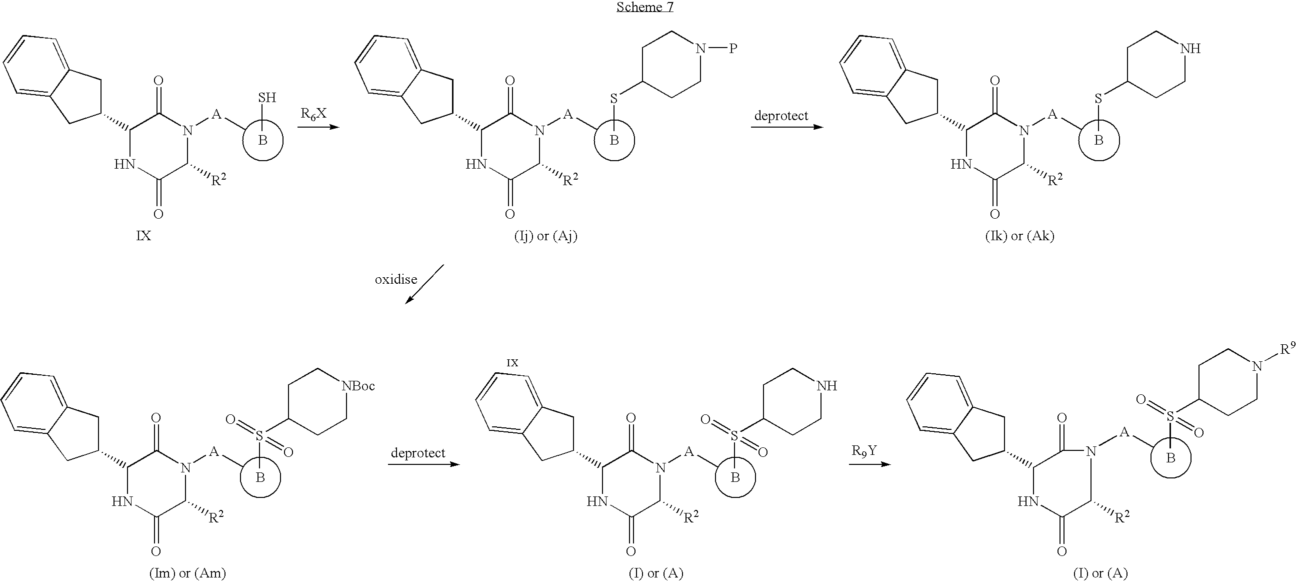

Compounds of Formula (I) or Formula (A), wherein R¹ represents the group —NR¹¹S(O)ₘR¹², wherein R¹¹ represents H, may be prepared according to reaction Scheme 8 starting from the nitro compound (In) or (An) which may be hydrogenated using standard conditions, for example in the presence of a Pd/C catalyst, to form the amine (Io) or (Ao) which may be reacted with a suitable sulfonyl chloride compound of Formula (X) in the presence of a suitable base, such as triethylamine and dimethylaminopyridine, to form the sulphonamide compound (Ip) or (Ap).

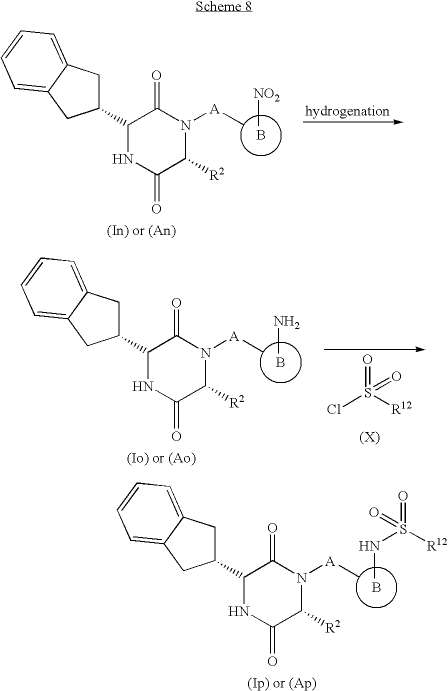

Alternatively, compounds of Formula (I) or Formula (A), wherein R¹ represents the group —NR¹¹S(O)ₘR¹², wherein R¹¹ represents an optionally substituted C₁₋₄alkyl group may be prepared according to reaction Scheme 9 by reacting the sulphonamide (Ip) or (Ap) with a suitable alkyl halide R¹¹Z, wherein Z is a leaving group such as halogen, for example R¹¹Z is iodomethane, in the presence of a suitable base, such as potassium carbonate in a suitable solvent such as dimethylformamide (DMF).

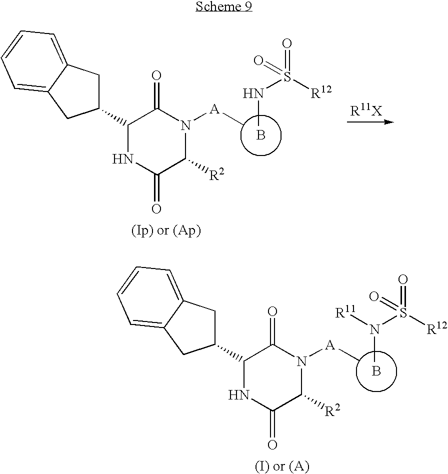

Compounds of Formula (I) or Formula (A), wherein R¹ represents the group —NR¹¹S(O)ₘR¹² which forms a cyclised sulphonamide may be prepared according to reaction Scheme 10 starting from the corresponding amine compound (Io) or (Ao) and reacting this with the appropriate chloroalkyl sulfonyl chloride in the presence of a suitable catalyst such as tetrabutyl ammonium iodide.

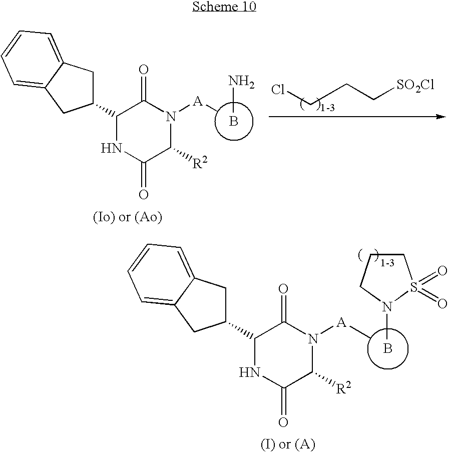

Compounds of Formula (I) or Formula (A), wherein R¹ represents the group —NR⁷COR⁸ may be prepared according to reaction Scheme 11 by treating the primary amine compound of Formula (Io) or (Ao) with a suitable acid chloride, such as acetyl chloride, in the presence of a suitable solvent, such as dichloromethane, and a base, for example pyridine.

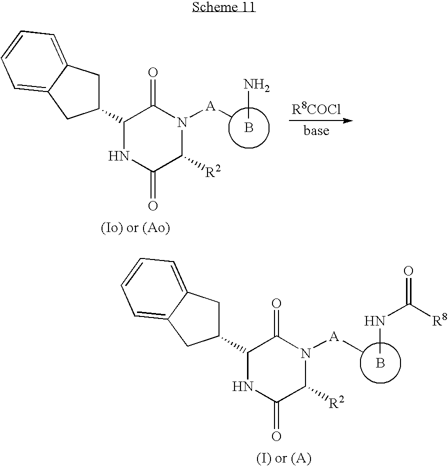

Compounds of Formula (I) or Formula (A), wherein R¹ represents the group —NR⁷COR⁸, wherein R⁷ and R⁸ together with the carbonyl group to which they are attached form a cyclic group, may be prepared according to reaction Scheme 12 reacting the bromo compound (Iq) or (Aq) with a suitable amide, HNR⁷COR⁸, wherein R⁷ and R⁸ together with the carbonyl group to which they are attached form a cyclic group, in the presence of a suitable catalyst such as copper iodide in a suitable solvent, such as 1,4-dioxan and the mixture subjected to microwave irradiation to form the resulting cyclic amide.

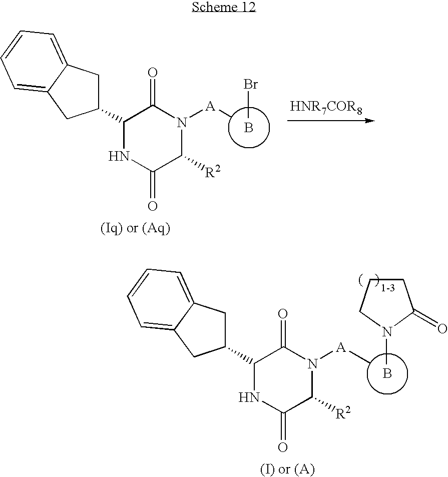

Those skilled in the art will appreciate that where a compound of Formula (I) or (A) possesses an amide group anywhere in the molecule, for example the group —CONR³R⁴ at the R¹ position, this group may be synthesised for example from a coupling reaction between the corresponding carboxylic acid —CO₂H and an amine HNR³R⁴, using a variety of standard methods. The carboxylic acid may be synthesized by oxidation of the corresponding aldehyde —CHO or the corresponding alcohol —CH₂OH, or by hydrolysis of the corresponding ester —CO₂R^{X}, wherein R^{X} is for example a C₁₋₄alkyl group, or from the corresponding halo compound, for example by treatment with a Grignard reagent in the presence of carbon dioxide.

Those skilled in the art will appreciate that where a compound of Formula (I) or (A) possesses a sulfonamide anywhere in the molecule, for example the group —SO₂NR⁹R¹⁰, this group may be synthesised for example from a reaction between the corresponding sulfonyl chloride —SO₂Cl and an amine, using a variety of standard methods. The sulfonyl chloride —SO₂Cl may be synthesised by oxidation of the corresponding thiol compound —SH using standard conditions, e.g. reaction with sulfuryl chloride in the presence of a suitable base. The thiol compound —SH is accessible from the corresponding sulfanyl compounds —SC₁₋₄alkyl by carrying out a deprotection reaction under standard conditions.

Those skilled in the art will further appreciate that where a compound of Formula (I) or (A) possesses a sulfoxide or sulfone anywhere in the molecule, for example the group —S(O)₁₋₂R⁶, this group may be may be synthesised for example by an oxidation reaction of the corresponding sulfanyl compound —SR⁶ under standard conditions. For example, oxidation of the sulfanyl compound —SR⁶ to provide the sulfone compound —SO₂R⁶ may be carried out using a suitable oxidising agent such as 3-chloroperoxybenzoic acid. The sulfanyl compound —SR⁶ is accessible by alkylation of the corresponding thiol compound —SH with an alkylating agent R⁶X, wherein X is a suitable leaving group, for example mesylate, tosylate or halo, under standard conditions.

Those skilled in the art will also appreciate that in the preparation of the compound of Formula I or a solvate thereof, it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate.

### EXAMPLES

The following examples illustrate the invention. These examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made without departing from the spirit and scope of the invention.

### General Purification and Analytical Methods

Analytical HPLC was conducted by one of the following methods:
A) On a Supelcosil LCABZ+PLUS column (3.3 cm×4.6 mm ID), eluting with 0.1% HCO₂H and 0.01 M ammonium acetate in water (solvent A), and 0.05% HCO₂H and 5% water in acetonitrile (solvent B), using the following elution gradient 0-0.7 minutes 0% B, 0.7-4.2 minutes 0%-100% B, 4.2-5.3 minutes 100% B, 5.3-5.5 minutes 0% B at a flow rate of 3 ml/minute.
B) On an Acquity Ultra Performance LC equipped with a Acquity HPLC BEH C18 1.7 μm column (50×2.1 mm, i.d.), eluting with 0.05% TFA in water (solvent A) and 0.05% TFA in acetonitrile (solvent B), using the following elution gradient 5%-95% (solvent B) over 0.65 minutes and holding at 95% for 0.35 minutes at a flow rate of 0.8 ml/minute.
C) On a Shimadzu LC-2010 HPLC equipped with a YMC-AQ (50×2.0 mm ZD) column, eluting with 0.1% TFA in water (solvent A) and 0.05% TFA in acetonitrile (solvent B), using the following elution gradient 10%-80% (solvent B) over 3.0 minutes and holding at 80% for 0.5 minutes at a flow rate of 0.8 ml/minute.
D) On an Agilent Zorbax SB-C₁₈ 5.0 μm column (2.1 mm×50 mm, i.d.), eluting with 0.05% TFA in water (solvent A) and 0.05% TFA in acetonitrile (solvent B), using the following elution gradient 10%-99% (solvent B) over 3.0 minutes and holding at 99% for 1.0 minutes at a flow rate of 1.0 mL minutes⁻¹.

The mass spectra (MS) were recorded on a Fisons VG Platform or Waters micromass ZQ spectrometer or Finnigan LCQ-Advantage using electrospray positive [(ES+ve to give MH⁺ and M(NH₄)⁺ molecular ions] or electrospray negative [(ES−ve to give (M−H)⁻ molecular ion] modes on a Micromass series 2 or a Waters ZQ mass spectrometer.

¹H NMR spectra were recorded using a Bruker DPX or Avance 300 MHz or Avance 400 MHz spectrometer using tetramethylsilane as the external standard.

Silica column chromatography refers to purification carried out using prepackaged silica normal phase (RediSep®) cartridges sold by Isco or SPE (solid phase extraction) cartridges sold by International Sorbent Technology Ltd. Aminopropyl SPE and SCX-SPE refer to the aminopropyl and flash SCX-2 cartridges sold by International Sorbent Technology Ltd. Gilson purification refers to purification carried out by high performance liquid chromatography on a Xterra® Prep RP18 5 μm column (30 mm×100 mm i.d.) eluting with 0.1% TFA in water and 0.1% TFA in acetonitrile utilizing gradient elution at a flow rate of 25 ml/minute.

Mass directed autoprep refers to methods where the material was purified by high performance liquid chromatography on a HPLCABZ+ 5 μm column (5 cm×10 mm i.d.) with 0.1% HCO₂H in water and 95% MeCN, 5% water (0.5% HCO₂H) utilising gradient elution at a flow rate of 8 ml minutes⁻¹. The Gilson 202-fraction collector was triggered by a VG Platform Mass Spectrometer on detecting the mass of interest.

Hydrophobic frits refer to filtration tubes sold by Whatman.

Preparative layer chromatography refers to the use of TLC plates sold by Merck coated with silica gel 60 F₂₅₄.

### Intermediate 1

### 1-[2-(Methylsulfonyl)phenyl]methanamine

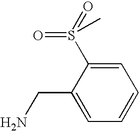

To a vigorously stirred solution of 1-[2-(methylthio)phenyl]methanamine (5 g) in dichloromethane (250 ml) at 5° C. was added the m-chloroperoxybenzoic acid (22.7 g) portionwise. The reaction was stirred at 0° C. for 30 minutes then at 20° C. for 18 hours. The reaction was loaded on to 3×20 g SCX-SPE cartridges, washed with methanol and eluted in 2M ammonia/methanol. Concentration of the latter gave the product as a yellow oil (3.9 g, 64%).

LCMS (A) Rt=0.38 minutes; m/z [M+H]⁺=186

¹H NMR (CDCl₃) δ 8.13 (d, 1H), 7.65 (t, 1H), 7.54 (d, 1H), 7.44 (t, 1H), 4.27 (s, 2H), 3.22 (s, 3H), 1.68 (br s, 2.5H (includes water)).

### Intermediate 2

### 1-{4-[(Trifluoromethyl)sulfonyl]phenyl}methanamine

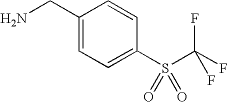

To 4-[(trifluoromethyl)sulfonyl]benzonitrile (1.6 g) in THF (20 mL) at 20° C. was added borane-tetrahydrofuran complex (1.0M, 13.4 mL) via a cannula. The mixture was stirred for 1 hour before warming to reflux for 18 hours, cooled to 0° C. then quenched with methanol (5 mL). The mixture was heated to 70° C., and then cooled and the solvent removed in vacuo. The resulting residue was dissolved in methanol and passed through a 20 g SCX-SPE cartridge. The cartridge was washed with methanol and the product amine eluted with 2.0M ammonia/methanol. Removal of solvent in vacuo afforded the amine as a yellow oil (1.4 g, 89%).

LCMS (A) Rt=1.66 minutes; m/z [M+H]⁺=240

¹H NMR (CDCl₃) δ 7.98 (d, 2H), 7.68 (d, 2H), 4.09 (s, 2H).

### Intermediate 3

### {[2-(Methoxy)-4-(methylsulfonyl)phenyl]methyl}amine hydrochloride

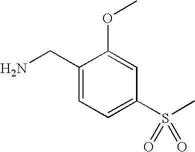

To a solution of 2-(methoxy)-4-(methylthio)benzoic acid (4.0 g) in diethyl ether (10 mL) at 0° C. was added a solution of lithium aluminium hydride in ethyl ether (1.0M, 30 mL), The mixture was stirred at 20° C. for 3 days and quenched with water (1.2 mL), aqueous NaOH (15%, 1.2 mL) and water (3.4 mL). Sodium sulfate (anhydrous, 2 g) was added and the mixture stirred for 5 minutes whereupon the solid was removed by filtration, washed with ether and the filtrate reduced in vacuo. A portion of the residue (0.92 g) was dissolved in chloroform (10 mL) and diisopropylethylamine (10.1 mL) and methane sulfonylchloride (0.46 mL) added regulating the temperature below 35° C. The mixture was stirred for 48 hours, water (5 mL) added, the aqueous layer extracted with chloroform (2×5 mL) and the combined organics reduced in vacuo. The resulting residue was dissolved in dimethylformamide (3 mL) and added to a mixture of bis(1,1-dimethylethyl) imidodicarbonate (1.3 g) and potassium tert-butoxide (0.67 g) in dimethylformamide (12 mL). The mixture was heated to 100° C. for 3 hours, cooled to 0° C. and quenched with saturated ammonium chloride (15 mL). The resulting mixture was reduced by ca. 80% in vacuo and water (50 mL) and ethyl acetate (100 mL) added. The supernatant layer was washed with water (2×50 mL) and saturated brine (50 mL) and dried over sodium sulfate before being reduced in vacuo. Silica column chromatography (ethyl acetate/cyclohexane) of the residue gave the imido-carbonate (2.2 g), 0.49 g of which was then dissolved in dichloromethane (7 mL) and water (6 mL) and sodium hydrogen carbonate (0.49 g) and 3-chloroperoxybenzoic acid (1.5 g) added. The mixture was stirred for 24 hours and the mixture partitioned between saturated sodium hydrogen carbonate (10 mL) and chloroform (10 mL). The combined organics were reduced in vacuo and the residue purified by silica column chromatography (ethyl acetate/cyclohexane). The resulting sulfone (0.35 g) was then dissolved in methanol/dichloromethane (4:1, 10 mL) and acetyl chloride (0.35 mL) added at 0° C. The mixture was stirred for 24 hours and reduced in vacuo to give the title compound (210 mg).

LCMS (A) Rt=0.38 minutes; m/z [M+H]⁺=216

¹H NMR (CDCl₃) δ 8.41 (br s, 3H), 7.68 (d, 1H), 7.56 (d, 1H), 7.50 (s, 1H), 4.08 (br q, 2H), 3.95 (s, 3H), 3.31 (s, 3H).

Prior to inclusion in the Ugi reaction the free base of the amine was obtained by means of an aminopropyl-SPE cartridge or by treatment with one equivalent of triethylamine.

### Intermediate 4

### {[2-Chloro-4-(methylsulfonyl)phenyl]methyl}amine hydrochloride

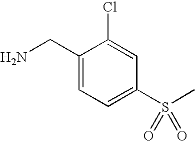

To a solution of 2-chloro-4-(methylsulfonyl)benzoic acid (4.5 g) in diethyl ether (10 mL) at 0° C. was added a solution of lithium aluminium hydride in ethyl ether (1.0M, 30 mL), The mixture was stirred at 20° C. for 3 days and quenched with water (1.2 mL), aqueous NaOH (15%, 1.2 mL) and water (3.4 mL). Sodium sulfate (anhydrous, 2 g) was added and the mixture stirred for 5 minutes whereupon the solid was removed by filtration, washed with ether and the filtrate reduced in vacuo. The residue (2.8 g) was dissolved in tetrahydrofuran (10 mL) and diisopropylethylamine (2.7 mL) and methane sulfonylchloride (1.2 mL) added regulating the temperature below 35° C. The mixture was stirred for 48 hours, water (5 mL) added, the aqueous layer extracted with chloroform (2×5 mL) and the combined organics reduced in vacuo. The resulting residue was dissolved in dimethylformamide (10 mL) and added to a mixture of bis(1,1-dimethylethyl) imidodicarbonate (3.4 g) and potassium tert-butoxide (1.8 g) in dimethylformamide (30 mL). The mixture was heated to 100° C. for 2 hours, cooled to 0° C. and quenched with saturated ammonium chloride (30 mL). The resulting mixture was reduced by ca. 80% in vacuo and water (100 mL) and ethyl acetate (200 mL) added. The supernatant layer was washed with water (2×50 mL) and saturated brine (50 mL) and dried over sodium sulfate before being reduced in vacuo. Silica column chromatography (ethyl acetate/cyclohexane) of the residue gave the imido-carbonate (2.3 g), which was then dissolved in methanol/dichloromethane (4:1, 10 mL) and acetyl chloride (0.35 mL) added at 0° C. The mixture was stirred for 24 hours and reduced in vacuo to give the title compound (210 mg).

LCMS (A) Rt=0.3 minutes; m/z [M+H]⁺=220

¹H NMR (CDCl₃) δ 8.76 (br s., 3H), 8.10 (s, 1H), 7.97 (d, 1H), 7.87 (d, 1H), 4.23 (s, 2H), 3.34 (s, 3H).

Prior to inclusion in the Ugi reaction the free base of the amine was obtained by means of an aminopropyl-SPE cartridge or by treatment with one equivalent of triethylamine.

### Intermediate 5

### [(5-Methyl-1-phenyl-1H-pyrazol-4-yl)methyl]amine

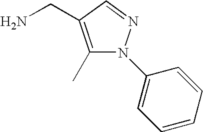

A solution of ethyl 5-methyl-1-phenyl-1H-pyrazole-4-carboxylate (1 g, 4.3 mmol) in dry ether (4 ml) under N₂ was cooled to −75° C. Diisobutyl aluminium hydride (1M in hexane, 8.6 ml) was added over 10 minutes and the reaction stirred at −70° C. for 2 hours. Dry methanol (0.85 ml) was then added and the reaction stirred for a further hour. Hydrochloric acid (2M, 4.5 ml) was added and the reaction allowed to warm to room temperature and stirred for 20 minutes. The organic phase was separated and the aqueous phase extracted with dichloromethane (2×50 ml). The combined organics were dried (Na₂SO₄) and concentrated to yield (5-methyl-1-phenyl-1H-pyrazol-4-yl)methanol as a pale yellow solid (816 mgs, 100%).

LCMS (A) Rt=2.15 minutes; m/z [M+H]⁺=189

Tetrapropylammonium perruthenate (76 mg) was added to a mixture of (5-methyl-1-phenyl-1H-pyrazol-4-yl)methanol (816 mg, 4.3 mmol), 4-methylmorpholine N-oxide (754 mg, 6.45 mmol) and molecular sieves (4 Å) in dry dichloromethane (12.6 ml). The reaction was stirred for 40 minutes then filtered through a silica plug washing with dichloromethane. Concentration yielded 5-methyl-1-phenyl-1H-pyrazole-4-carbaldehyde as a pale yellow solid (763 mg, 95%).

LCMS (A) Rt=2.39 minutes; m/z [M+H]⁺=187

5-methyl-1-phenyl-1H-pyrazole-4-carbaldehyde (763 mg, 4.1 mmol) was dissolved in ethanol (3 ml) and pyridine (3 ml) and hydroxylamine hydrochloride (441 mg, 4.8 mmol) added. The mixture was heated at reflux for 2 hours. The reaction was allowed to cool to room temperature and partitioned between water and chloroform (2×20 ml). The organic phase was dried (Na₂SO₄) and concentrated to yield 5-methyl-1-phenyl-1H-pyrazole-4-carbaldehyde oxime as a mixture of isomers, pale yellow solid, (858 mg).

LCMS (A) Rt=2.38 minutes; m/z [M+H]⁺=202

To a solution of 5-methyl-1-phenyl-1H-pyrazole-4-carbaldehyde oxime (588 mg, 2.93 mmol) in dry tetrahydrofuran (4.8 ml) at 0° C. was added lithium aluminium hydride (1 M solution in tetrahydrofuran, 4.4 ml). The reaction was stirred for 1 hour at room temperature and then heated at reflux for 18 hours. The reaction was cooled to 0° C. and quenched by the addition of water (0.167 ml), 15% sodium hydroxide (0.167 ml) and water (0.5 ml). Na₂SO₄ (0.33 g) was then added. The reaction was filtered and the filtrate concentrated to give [(5-methyl-1-phenyl-1H-pyrazol-4-yl)methyl]amine as a yellow oil (615 mg, 112% contains some tetrahydrofuran).

LCMS (A) Rt=1.62 minutes; m/z [M+H]⁺=188

¹H NMR (CDCl₃) [symbol] 7.61 (s, 1H) 7.5-7.37 (m, 5H), 3.75 (s, 2H), 2.32 (s, 3H), 1.67 (s, 2H).

### Intermediate 6

### ({2-[(1,1-Dimethylethyl)thio]phenyl}methyl)amine

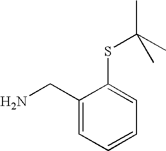

Prepared similarly to [(5-methyl-1-phenyl-1H-pyrazol-4-yl)methyl]amine (Intermediate 5) from the commercially available 2-[(1,1-dimethylethyl)thio]benzaldehyde.

¹H NMR (CDCl3) δ 7.55 (dd, 1H), 7.41 (dd, 1H), 7.35 (dt, 1H), 7.23 (dt, 1H), 4.07 (s, 2H), 1.57 (br s, 2H), 1.31 (s, 9H).

### Intermediate 7

### {[1-Methyl-5-(trifluoromethyl)-1H-pyrazol-4-yl]methyl}amine

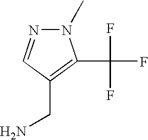

Prepared similarly to [(5-methyl-1-phenyl-1H-pyrazol-4-yl)methyl]amine (Intermediate 5) from commercially available ethyl 1-methyl-5-(trifluoromethyl)-1H-pyrazole-4-carboxylate.

LCMS (A) Rt=0.42 minutes; m/z [M+H]⁺=180

¹H NMR (CDCl₃) δ 7.41 (s, 1H), 3.92 (s, 3H), 3.83 (s, 2H), 1.56 (br s, 2H).

### Intermediate 8

### 4-{[2-(Dimethylamino)ethyl]oxy}-2-fluorobenzonitrile

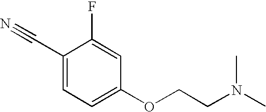

A 1.0M solution of potassium t-butoxide in tetrahydrofuran (7 mL) was added slowly to a solution of N,N-dimethylethanolamine (0.75 mL, 7.19 mmol) in tetrahydrofuran (5 mL) at 0-5° C. and the solution was stirred for 30 minutes. This mixture was added to a solution of 2,4-difluorobenzonitrile (1.00 g, 7.19 mmol) in THF (5 mL) at −65° C. and the resulting mixture was stirred at −65° C. for 3 hours, then allowed to warm to room temperature and stirred overnight. The mixture was then cooled to 5° C. and quenched with water (40 mL) then diluted with diethyl ether (150 ml). The organic phase was washed with water (50 mL), then with brine (50 mL), dried over anhydrous magnesium sulphate and concentrated under reduced pressure to give the title compound as a yellow oil (83%).

¹H NMR (CDCl₃) δ 7.47 (m, 1H), 6.65 (m, 2H), 4.09 (t, 2H), 2.74 (t, 2H), 2.30 (s, 6H)

### Intermediate 9

### 4-{[2-(Dimethylamino)ethyl]oxy}-2-(methylthio)benzonitrile

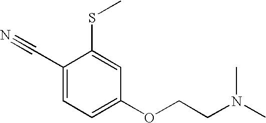

Sodium methanethiolate (0.46 g) was dissolved in dry THF (30 mL); this solution was treated with a solution of 4-{[2-(dimethylamino)ethyl]oxy}-2-fluorobenzonitrile (1.25 g) (Intermediate 8) in dry THF (40 mL) added over 90 minutes. The mixture was stirred at room temperature overnight. Then was treated with aqueous ammonium chloride (17 mL) followed after 10 minutes by aqueous sodium hydrogen carbonate (40 mL). The aqueous phase was extracted with dichloromethane (DCM) and the organic phase was washed with aqueous sodium hydrogen carbonate then with brine, dried over anhydrous sodium sulphate then concentrated under reduced pressure to give a ca. 2:1 mixture of 4-{[2-(dimethylamino)ethyl]oxy}-2-fluorobenzonitrile and 4-{[2-(dimethylamino)ethyl]oxy}-2-(methylthio)benzonitrile (1.65 g). This was dissolved in dry DMF (5 mL) and added to a solution of sodium methanethiolate (0.72 g) in dry DMF under nitrogen over 1 hour. This mixture was stirred at room temperature overnight then quenched with aqueous ammonium chloride (30 mL) and aqueous sodium hydrogen carbonate (50 mL). The aqueous phase was extracted with DCM and the organic phase was washed with aqueous sodium hydrogen carbonate then with brine, dried over anhydrous sodium sulphate then concentrated under reduced pressure to give the title compound as a yellow oil (57%).

LCMS (A) Rt=1.82 mins, [M+H]⁺=237

¹H NMR (CDCl₃) δ 7.43 (d, 1H), 6.82-6.77 (m, 2H), 4.17 (t, 2H), 2.82 (t, 2H), 2.51 (s, 3H), 2.37 (s, 6H)

### Intermediate 10

### 4-{[2-(Dimethylamino)ethyl]oxy}-2-(methylsulfonyl)benzonitrile

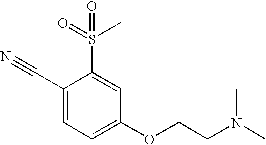

4-{[2-(Dimethylamino)ethyl]oxy}-2-(methylthio)benzonitrile (Int. 9) (1.06 g) was dissolved in dry DCM (10 mL) under nitrogen and the solution was treated with mCPBA (3.35 g). The resulting mixture was stirred overnight. DCM (100 mL) was added, followed by water (2 mL) The aqueous phase was saturated with sodium sulphite and sodium carbonate; the mixture was then stirred for 30 minutes and the phases were separated. The aqueous phase was washed with DCM (×3) and the organic phase was washed with saturated aqueous sodium carbonate. The organic extracts were concentrated under reduced pressure to give the title compound as an oil (47%).

¹H NMR (CDCl₃) δ 7.72 (d, 1H), 7.56-7.49 (m, 2H), 4.26 (broad s, 2H), 3.05 (s, 3H), 2.83 (broad s, 2H), 2.35 (broad s, 6H)

### Intermediate 11

### 2-{[4-(Aminomethyl)-3-(methylsulfonyl)phenyl]oxy}-N,N-dimethylethanamine

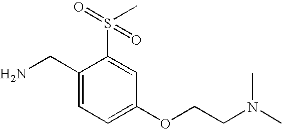

A solution of 4-{[2-(dimethylamino)ethyl]oxy}-2-(methylsulfonyl)benzonitrile (Int. 10) (0.52 g) in glacial acetic acid (40 mL) was hydrogenated over 10% palladium on charcoal (0.4 g) at 1 atmosphere of hydrogen for 3 hours. After purging the system with nitrogen, the catalyst was removed by filtration under nitrogen and was washed with a small volume of DCM. The combined filtrates were concentrated under reduced pressure to give the crude product as a yellow oil; this was dissolved in 2M hydrochloric acid and the solution was concentrated under reduced pressure, then dissolved in a mixture of methanol and toluene and this solution was concentrated under reduced pressure to afford the dihydrochloride of the title compound. This was converted to the free base using a 10 g aminopropyl SPE cartridge eluted with methanol. Product-containing fractions were combined and evaporated under reduced pressure to give the title compound as a yellow oil (80%).

¹H NMR (CDCl₃) δ 7.37 (m, 2H), 7.27 (s, 1H), 4.06 (t, 2H), 3.78 (s, 2H), 2.94 (s, 3H), 2.67 (t, 2H), 2.22 (s, 6H)

### Intermediate 12

### 1-[3-(3-Pyridinyl)phenyl]methanamine

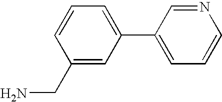

A mixture of 3-bromopyridine (0.12 ml, 1.2 mmol), [3-(aminomethyl)phenyl]boronic acid (568 mg, 3.2 mmol) and tetrakis(triphenylphosphine)palladium(0) (39 mg, 0.033 mmol) in aqueous sodium carbonate (1M, 4 ml) and dimethoxyethane (8 ml) was heated at reflux for 3 hours and then cooled. The mixture was treated with 50 ml of water and extracted with 3×30 ml of dichloromethane. The combined organic phase was dried over magnesium sulfate, filtered and evaporated to reveal a yellow oil. The product was purified by chromatography using a 5 g bond elute SPE cartridge and eluting with a mixture of dichloromethane, ethanol and aqueous ammonia (200:8:1) to give 100 mg (45%) of the title compound as a colourless oil.

LCMS MH+ 185.

¹H NMR (400 MHz, CDCl₃) δ 3.97 (s, 2H), 7.34-7.38 (m, 2H), 7.45-7.48 (m, 2H), 7.54 (bs, 1H), 7.87-7.91 (m, 1H), 8.58-8.61 (m, 1H), 8.84-8.87 (m, 1H).

### Intermediate 13

### [2-(Aminomethyl)-5-fluorophenyl]methanol

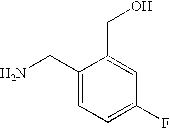

To a solution of 2-bromo-4-fluorobenzonitrile (5 g, 25 mmol) in anhydrous tetrahydrofuran (17 ml) at −30° C. was added isopropylmagnesium chloride (2M in tetrahydrofuran, 15 ml, 30 mmol) and the reaction stirred for 3 hours. Dimethylformamide (5.79 ml, 75 mmol) was then added and the reaction allowed to warm to room temperature and stirred for 1 hour. The reaction was then cooled to −10° C. and hydrochloric acid (2M, 37 ml) added and the reaction stirred for 20 minutes. The reaction was then reduced to ˜⅓ original volume and partitioned with ethyl acetate. The organics were then dried and concentrated to give 4-fluoro-2-formylbenzonitrile as a brown solid, 2.78 g.

¹H NMR (CDCl₃, 400 MHz): δ (ppm)=10.35 (1H, s); 7.9-7.86 (1H, m); 7.75 (1H, dd, J=8.16, 2.64 Hz), 7.49-7.44 (1H, m).

LCMS: Ret time 2.25, no ES⁺ observed.

To a solution of 4-fluoro-2-formylbenzonitrile (2.78 g, 18.6 mmol) in anhydrous tetrahydrofuran (34 ml) at 0° C. was added lithium aluminiumhydride (1M in tetrahydrofuran, 37.2 ml, 37.2 ml) and the reaction stirred for 1 hour. Water (1.14 ml), 15% sodium hydroxide (1.14 ml) and water (3.42 ml) were sequentially added followed by the addition of sodium sulphate (1.4 g). The reaction was then filtered and concentrated to yield [2-(aminomethyl)-5-fluorophenyl]methanol as a brown oil, 2.8 g.

¹H NMR (CDCl₃, 400 MHz): δ (ppm)=7.24-7.20 (1H, m); 7.11-7.08 (1H, m); 6.97-6.92 (1H, m); 4.6 (2H, m); 4.0 (2H, m).

### Intermediate 14

### (3R,6R)-3-(2,3-Dihydro-1H-inden-2-yl)-1-{[2-(hydroxymethyl)-phenyl]methyl}-6-(2-methylpropyl)-2,5-piperazinedione

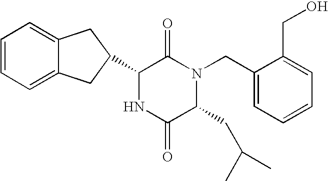

[2-(Aminomethyl)phenyl]methanol (3.509 g, 25.58 mmol) was dissolved in methanol (25 ml) and 3-methylbutanal (2.75 ml, 25.63 mmol) added followed by (2R)-2,3-dihydro-1H-inden-2-yl({[(1,1-dimethylethyl)oxy]carbonyl}amino)ethanoic acid (7.453 g, 25.58 mmol). The mixture was stirred for 15 minutes before 2-[(phenylmethyl)oxy]phenyl isocyanide (5.35 g, 25.58 mmol) was added. The mixture was stirred for 1.3 hours and then left to stand at room temperature over 7 nights before it was cooled in an ice/water bath. Then acetyl chloride (10.9 ml, 153.4 mmol) was added. Then the mixture was stirred in the cooling bath for a further 10 minutes before it was stirred at room temperature. After 4.25 hours the mixture was evaporated under reduced pressure to leave a dark brown foam. The foam was stirred in chloroform (50 ml) and saturated aqueous sodium bicarbonate solution (40 ml) for 60 minutes before it was diluted with chloroform (100 ml) and the phases separated. The aqueous phase was extracted with chloroform (3×50 ml). The combined organic phase was dried (MgSO₄) and concentrated under reduced pressure to ca. 80 ml. The chloroform solution was treated with glacial acetic acid (2 ml) and left to stand, at room temperature for five nights. Then the reaction mixture was washed with 2M hydrochloric acid (70 ml) diluted with chloroform (140 ml) and filtered. The filtered organic phase was washed with saturated aqueous sodium bicarbonate solution (70 ml). The organic phase was dried (MgSO₄), evaporated under reduced pressure and dried in vacuo to leave a dark brown solid. The solid was loaded in dichloromethane onto a 330 g flash silica chromatography column (pre-eluted with 20% ethyl acetate in cyclohexane). The column was eluted with 20% to 100% ethyl acetate in cyclohexane to afford (3R,6R)-3-(2,3-dihydro-1H-inden-2-yl)-1-{[2-(hydroxymethyl)phenyl]methyl}-6-(2-methylpropyl)-2,5-piperazinedione (1.563 g) as a pale brown foam.

LCMS (A) Rt=3.17 minutes; m/z [M+H]⁺=407.

### Intermediate 15

### 1,1-Dimethylethyl 3-{[(3R,6R)-3-(2,3-dihydro-1H-inden-2-yl)-6-(1-ethylpropyl)-2,5-dioxo-1-piperazinyl]methyl}benzoate

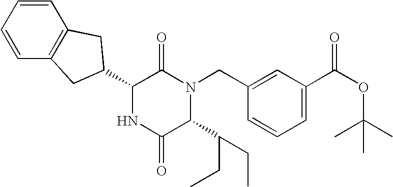

1,1-Dimethylethyl-3-(aminomethyl)benzoate (2.69 g, 12.98 mmol) was dissolved in methanol (15 ml) and 2-ethylbutanal (1.6 ml, 13 mmol) was added followed by (2R)-2,3-dihydro-1H-inden-2-yl({[(phenylmethyl)oxy]carbonyl}amino)ethanoic acid (4.225 g, 12.99 mmol). The mixture was stirred for 11 minutes before 2-[(phenylmethyl)oxy]phenyl isocyanide (2.73 g, 13 mmol) was added. The mixture was stirred at room temperature for 1.8 hours and then left to stand over the weekend (65 hours) before the solvent was evaporated under reduced pressure to leave a sandy foam. The foam in solution in ethanol (90 ml) containing acetic acid (1.5 ml) was hydrogenated at room temperature and pressure over 10% Pd/carbon (1.42 g) for 18.5 hours. The reaction was filtered through glass fibre filters and the solvent removed in vacuo to give a pale brown foam. The foam was stirred in chloroform (50 ml) and treated with glacial acetic acid (2 ml). The mixture was stirred overnight (21.5 hours) at room temperature. Then the reaction mixture was diluted with chloroform (100 ml) and washed with 2M hydrochloric acid (40 ml) followed by saturated aqueous sodium bicarbonate solution (40 ml). The phases were separated by hydrophobic frit and the organic phase was evaporated under reduced pressure and dried in vacuo to leave a brown solid. The solid was loaded in dichloromethane onto a 120 g flash silica chromatography column (pre-eluted with 10% ethyl acetate in cyclohexane). The column was eluted with 10% to 100% ethyl acetate in cyclohexane to afford 1,1-dimethylethyl 3-{[(3R,6R)-3-(2,3-dihydro-1H-inden-2-yl)-6-(1-ethylpropyl)-2,5-dioxo-1-piperazinyl]methyl}benzoate as a pale yellow solid (2.115 g).

LCMS (A) Rt=3.77 minutes; m/z [M+H]⁺=508.

### Intermediate 16

### Phenylmethyl N-[(2-{[(3R,6R)-3-(2,3-dihydro-1H-inden-2-yl)-6-(1-ethylpropyl)-2,5-dioxo 1-piperazinyl]methyl}phenyl)sulfonyl]glycinate

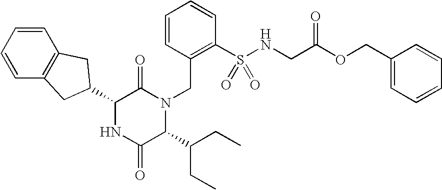

To a solution of 2-{[(3R,6R)-3-(2,3-dihydro-1H-inden-2-yl)-6-(1-ethylpropyl)-2,5-dioxo-1-piperazinyl]methyl}benzenesulfonyl chloride (Int. 20) (100 mg) in dichloromethane (2 ml) was added diisopropylethylamine (78 ul) and phenylmethyl glycinate hydrochloride (39 mg) and the mixture stirred for 3 hours at 20° C. Methanol (2 ml) was added and the mixture passed through a 2 g aminopropyl-SPE column and the solvent removed. The resulting residue was purified using a 2 g Si-SPE column eluting with ethyl acetate/cyclohexate (40-50%) to provide the title compound (69 mg) as an oil.

LCMS (A) Rt=3.67 minutes; m/z [M+H]⁺=618, [M]⁻=616.

Intermediates 17-18 were prepared by methods analogous to that described for Example 66 from 2-{[(3R,6R)-3-(2,3-dihydro-1H-inden-2-yl)-6-(1-ethylpropyl)-2,5-dioxo-1-piperazinyl]methyl}benzoic acid (Ex. 65)

| Int | | | Rt/ | +ve; | |
|---|---|---|---|---|---|
| No | Structure | Mwt | min | −ve | Name |
| 17 | 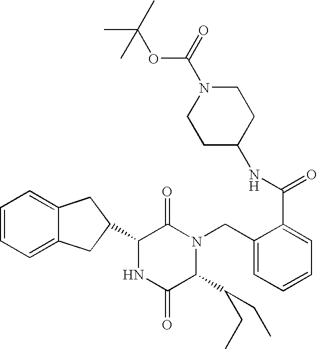 | 616.8 | 3.48(A) | 617;615 | 1,1-dimethylethyl 4-{[(2-{[(3R,6R)-3-(2,3-dihydro-1H-inden-2-yl)-6-(1-ethylpropyl)-2,5-dioxo-1-piperazinyl]-methyl}phenyl)carbonyl]amino}-1-piperidinecarboxylate |
| 18 | 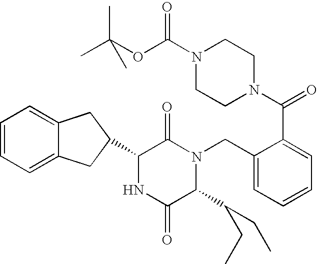 | 602.7 | 3.62(A) | 603;601 | 1,1-dimethylethyl 4-[(2-{[(3R,6R)-3-(2,3-dihydro-1H-inden-2-yl)-6-(1-ethylpropyl)-2,5-dioxo-1-piperazinyl]-methyl}phenyl)carbonyl]-1-piperazine-carboxylate |

### Intermediate 19

### (3R,6R)-3-(2,3-Dihydro-1H-inden-2-yl)-6-(1-ethylpropyl)-1-[(2-mercaptophenyl)-methyl]-2,5-piperazinedione

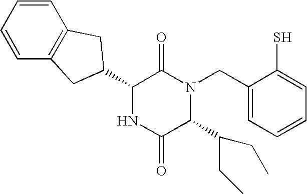

To a solution of (3R,6R)-3-(2,3-dihydro-1H-inden-2-yl)-1-({2-[(1,1-dimethylethyl)thio]-phenyl}methyl)-6-(1-ethylpropyl)-2,5-piperazinedione (Example 60) (8.0 g) in acetic acid (80 mL) was added 2-nitrobenzenesulfenyl chloride (3.3 g) and the mixture stirred at 20° C. for 24 hours. The acetic acid was reduced in vacuo, the resulting yellow residue dissolved in dimethylformamide (36 mL) and the mixture de-gassed by means of a stream of nitrogen gas for 20 minutes. Triethylamine (3.3 mL) and tris(carboxyethyl)phosphine hydrochloride (6.7 g) were then added and the mixture stirred at 20° C. for 1.5 hours under nitrogen. The mixture was reduced in vacuo by ca. 50% whereupon ethyl acetate (400 mL) was added and the mixture washed with de-gassed water (2×250 mL), brine (200 mL) and dried over sodium sulfate. Removal of the solvent in vacuo and silica column chromatography (ethyl acetate/cyclohexane) gave the title compound (5.8 g, 82%).

LCMS (A) Rt=3.7 minutes; m/z [M+H]⁺=423

¹H NMR δ 7.15-7.35 (m, 8H), 7.01 (br d, 1H), 5.33 (d, 1H), 4.23 (d, 1H), 4.15 (dd, 1H), 3.52 (br s, 1H), 3.18 (m, 3H), 2.97 (m, 1H), 2.82 (dd, 1H), 1.52-1.80 (m, 4H), 1.32 (m, 1H), 0.92 (m, 6H).

### Intermediate 20

### 2-{[(3R,6R)-3-(2,3-Dihydro-1H-inden-2-yl)-6-(1-ethylpropyl)-2,5-dioxo-1-piperazinyl]-methyl}benzenesulfonyl chloride

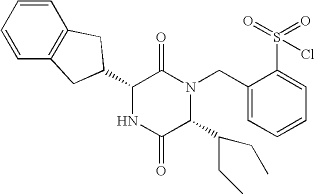

To a solution of (3R,6R)-3-(2,3-dihydro-1H-inden-2-yl)-6-(1-ethylpropyl)-1-[(2-mercapto-phenyl)methyl]-2,5-piperazinedione (Int. 19) (2.4 g) in acetonitrile (60 ml) at 0° C. was added potassium nitrate (1.7 g) followed by dropwise addition of sulfuryl chloride (1.4 ml). The mixture was stirred at 0-10° C. for 2.5 hours. Sodium carbonate (4 g) was dissolved in water (100 ml) and added to the reaction at 0° C., the mixture stirred for 2 minutes and partitioned between ethyl acetate (200 ml) and water (50 ml). The aqueous layer was extracted with ethyl acetate (2×50 ml) and the combined organics washed with saturated brine (150 ml) and dried over sodium sulfate. Removal of the solvent in vacuo and silica column chromatography (dichloromethane, chloroform, diethyl ether and ethyl acetate) gave the title compound (1.5 g, 54%).

LCMS (A) Rt=3.7 minutes; m/z [M+H]⁺=489

¹H NMR δ 8.14 (d, 1H), 7.70 (t, 1H), 7.55 (t, 1H), 7.31 (d, 1H), 7.2 (m, 5H), 5.43 (d, 1H), 5.04 (d, 1H), 4.20 (dd, 1H), 4.11 (d, 1H), 3.20 (m, 3H), 3.06 (m, 1H), 2.88 (dd, 1H), 1.68 (m, 4H), 1.38 (m, 1H), 0.95 (t, 3H), 0.87 (t, 3H).

### Intermediate 21

### 1,1-Dimethylethyl 4-[(2-{[(3R,6R)-3-(2,3-dihydro-1H-inden-2-yl)-6-(1-ethylpropyl)-2,5-dioxo-1-piperazinyl]methyl}phenyl)thio]-1-piperidinecarboxylate

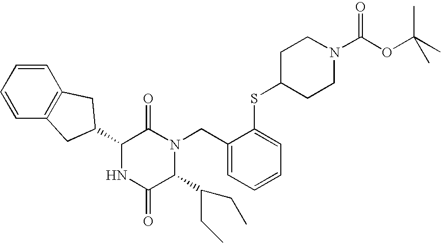

To a solution of (3R,6R)-3-(2,3-dihydro-1H-inden-2-yl)-6-(1-ethylpropyl)-1-[(2-mercapto-phenyl)methyl]-2,5-piperazinedione (Int. 19) (800 mg) in acetonitrile (3.2 ml) was added 1,1-dimethylethyl 4-[(methylsulfonyl)oxy]-1-piperidinecarboxylate (477 mg). The reaction was cooled to 0° C. and de-gassed with a stream of nitrogen gas for 20 minutes. Potassium carbonate (330 mg) was added and the reaction was heated to 80° C. for 3 hours. The reaction was partitioned between water (12 ml) and ethyl acetate (12 ml) and the aqueous layer extracted with ethyl acetate (2×12 ml). The combined organics were concentrated and the residue purified by silica column chromatography (ethyl acetate/cyclohexane) to give the title compound (670 mg, 65%).

LCMS (A) Rt=4.0 minutes; m/z [M+H]⁺=606

¹H NMR δ 7.48 (m, 1H), 7.20 (m, 7H), 6.52 (br d, 1H), 5.28 (d, 1H), 4.51 (d, 1H), 4.13 (m, 1H), 3.97 (m, 3H), 3.17 (m, 4H), 2.80-3.00 (m, 4H), 1.92 (br d, 2H), 1.62 (m, 5H), 1.48 (s, 9H), 1.29 (m, 1H), 0.88 (m, 1H).

### Intermediate 22

### 1,1-Dimethylethyl 4-[(2-{[(3R,6R)-3-(2,3-dihydro-1H-inden-2-yl)-6-(1-ethylpropyl)-2,5-dioxo-1-piperazinyl]methyl}phenyl)sulfonyl]-1-piperidinecarboxylate

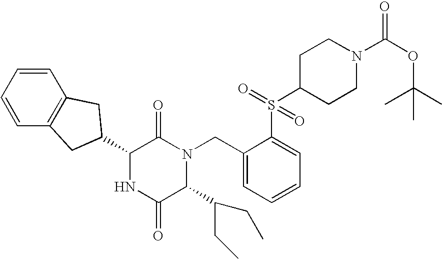

To a solution of 1,1-dimethylethyl 4-[(2-{[(3R,6R)-3-(2,3-dihydro-1H-inden-2-yl)-6-(1-ethylpropyl)-2,5-dioxo-1-piperazinyl]methyl}phenyl)thio]-1-piperidinecarboxylate (Int. 21) (670 mg) in dichloromethane (7.4 ml) was added 3-chloroperoxybenzoic acid (570 mg) and the mixture stirred for 2 hours. The mixture was quenched by the dropwise addition to 10% sodium sulfite solution (5 ml) and the mixture stirred for 5 minutes. The organic layer was separated and the aqueous phase washed with dichloromethane (2×5 ml) and the combined organics reduced in vacuo. The residue was dissolved in methanol and purified by aminopropyl-SPE, eluting the product in methanol. Evaporation in vacuo gave the title compound (690 mg, 98%).

LCMS (A) Rt=3.7 minutes; m/z [M+H]⁺=638

¹H NMR (CDCl₃) δ 7.98 (d, 1H), 7.57 (t, 1H), 7.46 (t, 1H), 7.28 (d, 1H), 7.2 (m, 4H), 6.5 (br d, 1H), 5.32 (d, 1H), 4.80 (d, 1H), 4.25 (br m, 2H), 4.11 (m, 2H), 3.36 (br t, 1H), 3.15 (m, 3H), 2.98 (m, 1H), 2.75 (br m, 3H), 2.08 (br d, 1H), 1.60-1.80 (m, 6H), 1.53 (s, 9H), 1.40 (m, 1H), 1.03 (t, 3H), 0.95 (t, 3H).

### Intermediate 23

### 4-(Pyrazin-2-yl)aminobenzylamine

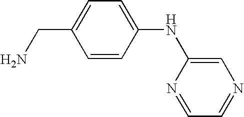

A mixture of 4-bromobenzonitrile (3.76 g, 20.8 mmol), aminopyrazine (2.4 g, 25.3 mmol), xantphos (0.26 g, 0.45 mmol), Pd₂(dba)₃ (0.25 g, 0.43 mmol) and caesium carbonate (9.25 g, 28.4 mmol) in dioxane (50 ml) was heated at reflux under nitrogen for 24 hours. The mixture was cooled, diluted with THF, filtered, concentrated in vacuo then chromatographed (silica, ethyl acetate/petroleum ether 1:3) to give crude 4-(pyrazin-2-yl)aminobenzonitrile (2.64 g). Without further purification, this material (2.38 g) was hydrogenated in methanolic ammonia (120 ml) over Raney nickel (0.36 g) at 50 psi for 3 hours. The catalyst was filtered off and the solution evaporated to obtain 4-(pyrazin-2-yl)aminobenzylamine (2.4 g).

LCMS (C) Rt=0.64 minutes; m/z [M-NH₂]⁺=184

¹H NMR (CDCl₃) δ 8.21 (d, 1H), 8.10 (m, 1H), 7.96 (d, 1H), 7.38 (d, 2H), 7.30 (d, 2H), 6.65 (br s, 1H), 3.85 (s, 2H).

Intermediates 24-25 were prepared by methods analogous to that described for Intermediate 23

| Int. | | | Rt/ | +ve; | |
|---|---|---|---|---|---|
| No. | Structure | Mwt | min | −ve | Name |
| 24 | 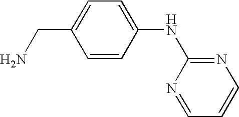 | 200.2 | 0.61(C) | 184 | 4-(pyrimid-2-yl)aminobenzylamine |
| 25 | 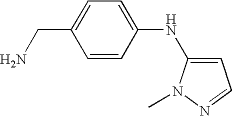 | 202.3 | 0.53(C) | 203 | 4-(1-methylimidazol-2-yl)amino-benzylamine |

### Intermediate 26

### 4-(5-Methyl-1,4,5-thiadiazol-2-yl)aminobenzylamine

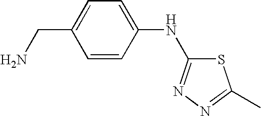

5-Methyl-1,3,4-thiadiazol-2-amine (19 g, 165 mmol) and potassium tert-butoxide (24 g, 206 mmol) was dissolved in DMSO (100 mL) and stirred at room temperature for 1 hour. A solution of 4-fluorobenzonitrile (10.8 g, 88.5 mmol) in DMSO (30 mL) was added dropwise over 15 minutes. The reaction was heated to 50° C. and stirred for 30 minutes. The reaction mixture was poured into water (1000 mL) and a precipitate formed. The solution was adjusted to pH=5 with 2N HCl (100 mL) and then filtered. The precipitate was washed with water and petroleum ether. The solid was dissolved in MeOH and purified by flash chromatography on silica gel to give 10.2 g of 4-[(5-methyl-1,3,4-thiadiazol-2-yl)amino]benzonitrile. This material (2.2 g, 10.2 mmol) was hydrogenated in methanolic ammonia (125 mL) over Raney nickel (0.40 g, 6.8 mmol) at 50 psi for 3 hours at room temperature. The catalyst was filtered off and the filtrate evaporated to obtain 1.9 g of N-[4-(aminomethyl)phenyl]-5-methyl-1,3,4-thiadiazol-2-amine.

¹H NMR (CD₃OD) δ 7.58 (d, 2H), 7.37 (d, 2H), 3.94 (s, 2H), 2.59 (s, 3H)

HPLC (C) Rt=0.80 minutes; m/z [M+H]⁺=221

Intermediate 27 was prepared by methods analogous to that described for Intermediate 23 except sodium carbonate was used as the base.

| | | | | | |
|---|---|---|---|---|---|
| 27 | 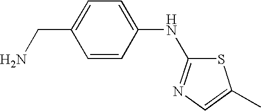 | 219.3 | 0.57(C) | 220 | 4-(5-methylthiazol-2-yl)amino-benzylamine |

### Intermediates 28 and 29

### 4-(2H-1,2,3-Triazol-2-yl)benzonitrile and 4-(1H-1,2,3-triazol-1-yl)benzonitrile

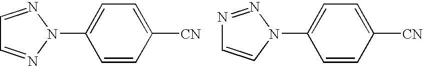

To a solution of 4-bromobenzonitrile (10 g, 54.9 mmol) in dry DMF (20 ml) was added successively 1H-1,2,3-triazole (3.81 g, 54.9 mmol), copper(I) iodide (0.5 g, 2.6 mmol), (1S,2S)—N,N′-dimethyl-1,2-cyclohexanediamine (1 ml, 6.25 mmol) and potassium carbonate (16 g, 116 mmol). The mixture was stirred under nitrogen at 110° C. overnight. The solvent was evaporated in vacuo, ethyl acetate was added to the residue and the resulting solution was filtered, dried and evaporated to give a mixture of the two regioisomers. Chromatography gave 4-(2H-1,2,3-triazol-2-yl)benzonitrile (Intermediate 28) (3.5 g)

¹H NMR (CDCl₃) [symbol] 7.79 (2H, d), 7.88 (2H, s), 8.23 (2H, d)

and 4-(1H-1,2,3-triazol-1-yl)benzonitrile (Intermediate 29) (2.3 g)

¹H NMR (CDCl₃) [symbol] 7.84-8.07 (6H, m)

### Intermediate 30

### {[4-(1H-1,2,3-Triazol-1-yl)phenyl]methyl}amine

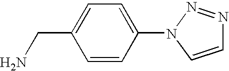

4-(1H-1,2,3-Triazol-1-yl)benzonitrile (Intermediate 29) (2.3 g, 13.5 mmol) in THF (12 ml) was cooled in ice and lithium aluminium hydride (2.4 g, 63.2 mmol) was added portionwise. The mixture was stirred at toom temperature for 1.5 h then recooled in ice and aqueous sodium hydroxide (10 ml) added dropwise. The resulting solution was filtered, and the THF layer dried and evaporated to obtain {[4-(1H-1,2,3-triazol-1-yl)phenyl]methyl}amine (2.5 g)

LCMS (C) Rt=0.45 minutes; m/z [M+H]⁺=175

¹H NMR (CDCl₃) [symbol] 3.96 (2H, s), 7.49 (2H, d), 7.71 (2H, d), 7.84 (1H, narrow d), 7.98 (1H, narrow d).

### Intermediate 31

### 4-(1,2,3-Triazol-2-yl)benzylamine

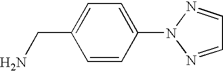

This compound was prepared from Intermediate 28 by a method analogous to that described for Intermediate 30.

¹H NMR (CDCl₃) [symbol] 3.92 (2H, s), 7.43 (2H, d), 7.79 (2H, s), 8.03 (2H, d).

Intermediates 32-33 were prepared in two steps by methods analogous to those described for Intermediates 29 and 30 except that in these cases a single regioisomer was obtained

| Int. | | | Rt/ | +ve; | |
|---|---|---|---|---|---|
| No. | Structure | Mwt | min | −ve | Name |
| 32 | 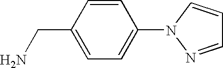 | 173.2 | 0.66(C) | 157 | 4-(pyrazol-1-yl)benzylamine |
| 33 | 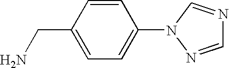 | 174.2 | 0.44(C) | 158 | 4-(1,2,4-triazol-1-yl)benzylamine |

### Intermediate 34

### 2-(1H-Tetrazol-1-yl)benzoic acid

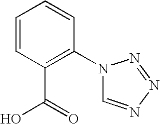

A solution of anthranilic acid (10.0 g, 73 mmol), sodium azide (14.0 g, 217 mmol) and trimethyl orthoformate (23.6 ml, 220 mmol) in glacial acetic acid (250 ml) was stirred at room temperature for 2 hours. The resulting solid was filtered off and dried to obtain the desired product (8.73 g).

¹H NMR (CDCl₃) [symbol] 7.46-8.06 (3H, m), 8.26 (1H, m), 9.79 (1H, s)

### Intermediate 35

### 2-(1H-Tetrazol-1-yl)benzamide

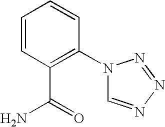

A mixture of 2-(1H-tetrazol-1-yl)benzoic acid (Int. 34) (3.0 g), ammonium chloride (1.68 g), EDC.HCl (6.0 g), diisopropylethylamine (10.8 ml) and 1-hydroxy-7-azabenzotriazole (4.2 g) in DMF (45 ml) was stirred at room temperature under argon for 15 hours. Water was added and the mixture was extracted with ethyl acetate. The combined organic layers were dried and evaporated to yield the desire product (1.8 g).

¹H NMR (CD₃OD) [symbol] 7.63-7.79 (4H, m), 9.43 (1H, s)

### Intermediate 36

### 2-(1H-Tetrazol-1-yl)benzonitrile

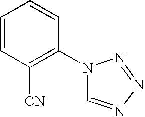

Triethylamine (0.4 ml) was added dropwise to a stirred solution of 2-(1H-tetrazol-1-yl)-benzamide (Int. 35) (110 mg) in phosphorus oxychloride (10 ml) at room temperature. After 30 min the mixture was poured into ice-water and extracted with ethyl acetate. Drying and evaporation of the organic layers yielded the desired product (80 mg).

¹H NMR (CDCl₃) [symbol] 7.69-7.94 (4H, m), 9.26 (1H, s)

### Intermediate 37

### {[2-(1H-Tetrazol-1-yl)phenyl]methyl}amine

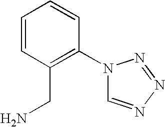

A solution of 2-(1H-tetrazol-1-yl)benzonitrile (Int. 36) (120 mg) in methanolic ammonia (20 ml) was hydrogenated over Raney nickel (0.5 g) at 40 psi for 2 hours. The catalyst was filtered off and the solution evaporated to obtain the desired product (80 mg)

LCMS (C) Rt=0.47 minutes; m/z [M+H]⁺=176

### Intermediate 38

### 4-Bromoisophthalic acid dimethyl ester

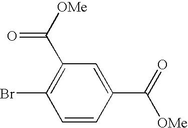

A suspension of 4-bromoisophthalic acid (10 g, 40.8 mmol) in MeOH (150 mL) was cooled to 0° C. Thionyl chloride (10 mL, 140 mmol) was added dropwise over 10 minutes and the reaction stirred at RT overnight. The solvent was then removed. The yellow solid was taken up in 100 mL dichloromethane and washed with 30 mL saturated sodium bicarbonate. The organic phase was separated, dried over Na₂SO₄ and concentrated to yield 4-bromoisophthalic acid dimethyl ester as a white solid (11.0 g, 97%).

LCMS (D) Rt=2.60 minutes; m/z [M+H]⁺=273

### Intermediate 39

### Dimethyl 4-cyano-1,3-benzenedicarboxylate

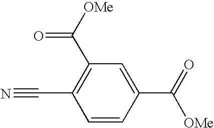

4-Bromoisophthalic acid dimethyl ester (Int. 38) (11 g, 40.3 mmol), copper(I) cyanide (14.4 g, 161.2 mmol), tetraethyl ammonium cyanide (6.38 g, 40.3 mmol), tris(dibenzylideneactone)dipalladium (2.96 g, 3.2 mmol), 1,1′-bis(diphenylphosphino)-ferrocene (7.14 g, 12.9 mmol) and 100 mL dioxane were mixed and refluxed for 2 hours under N₂. The mixture was then cooled down to RT, diluted with 400 mL ethyl acetate and filtered through celite. The filtrate was then washed with 100 mL saturated sodium bicarbonate, dried over MgSO₄ and concentrated. The crude material was purified via combi flash silica gel column eluting with 10-50% ethyl acetate in hexanes to give dimethyl 4-cyano-1,3-benzenedicarboxylate as a pale yellow solid (7.1 g, 80%).

LCMS (D) Rt=2.23 minutes; m/z [M+H]⁺=220

¹H NMR (CDCl₃) [symbol] 8.79 (s, 1H), 8.32 (d, 1H), 7.93 (d, 1H), 4.06 (s, 3H), 4.01 (s, 3H).

### Intermediate 40

### [4-(Aminomethyl)benzene-1,3-diyl]dimethanol

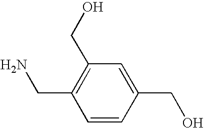

To a suspension of lithium aluminum hydride (2.42 g, 63.9 mmol) in 200 mL dry THF at 0° C. was added dimethyl 4-cyano-1,3-benzenedicarboxylate (Int. 39) (7.0 g, 31.9 mmol) in several batches. The suspension was then warmed up to RT and stirred for 6 hours. After cooling down to 0° C., 15 mL MeOH was added carefully to quench the reaction, followed by 15 mL water. The resulting mixture was stirred overnight. The suspension was then filtered through 100 g celite and washed with MeOH (5×60 mL). The combined filtrate was concentrated and dried in vacuo to give [4-(aminomethyl)benzene-1,3-diyl]dimethanol as a brown oil (5.0 g, 94%).

LCMS (D) Rt=0.26 minutes; m/z [M+H]⁺=168

¹H NMR (CD₃OD) [symbol] 7.29-7.46 (m, 3H), 4.68 (s, 2H), 4.63 (s, 2H), 3.33 (s, 2H).

### Intermediate 41

### Methyl 2-chloro-5-(methylthio)benzoate

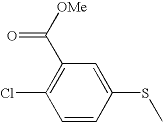

A solution of 2-chloro-5-(methylthio)benzoic acid (20 g, 94.7 mmol) in MeOH (150 mL) was cooled to 0° C. Thionyl chloride (10 mL, 140 mmol) was added dropwise over 10 minutes and the reaction stirred at RT overnight. The solvent was then removed. The yellow solid was taken up in 250 mL ethyl acetate and washed with 30 mL saturated sodium bicarbonate. The organic phase was separated, dried over Na₂SO₄ and concentrated to yield methyl 2-chloro-5-(methylthio)benzoate as a white solid (23.2 g, 100%).

LCMS (D) Rt=2.76 minutes; m/z [M+H]⁺=217

### Intermediate 42

### Methyl 2-cyano-5-(methylthio)benzoate

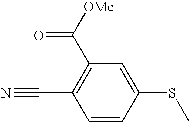

Methyl 2-chloro-5-(methylthio)benzoate (Int. 41) (23.2 g, 106.9 mmol) and copper(I) cyanide (19.2 g, 213.8 mmol) were dissolved in 100 mL N-methylpyrrolidinone. The resulting mixture was heated at 160° C. for 72 hours. LCMS showed the presence of the cyanated ester and hydrolyzed acid in 1:1 ratio. After cooling down to RT, the reaction mixture was treated with 250 mL water and 300 mL ethyl acetate and filtered through celite. The phases were separated. The aqueous phase was extracted with 2×250 mL ethyl acetate. The combined organics were washed with 250 mL water, dried over MgSO₄ and concentrated to give an oil. The crude material was purified via combi flash silica gel column eluting with 0-20% ethyl acetate in hexanes to give methyl 2-cyano-5-(methylthio)benzoate (6.14 g, 28%) as a white solid.

LCMS (D) Rt=2.42 minutes; m/z [M+H]⁺=208

### Intermediate 43

### [2-(Aminomethyl)-5-(methylthio)phenyl]methanol

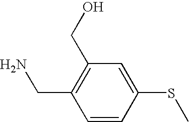

To a suspension of lithium aluminum hydride (3.37 g, 88.9 mmol) in 70 mL dry THF at 0° C. was added methyl 2-cyano-5-(methylthio)benzoate (Int. 42) (6.14 g, 29.6 mmol) in several batches. The suspension was then warmed up to RT and stirred for 18 hours. After cooling down to 0° C., 15 mL MeOH was added carefully to quench the reaction, followed by 15 mL water. The resulting mixture was stirred at RT for 3 hours. The suspension was then filtered through 50 g celite and washed with MeOH (5×60 mL). The combined filtrate was concentrated and dried in vacuo to give [2-(aminomethyl)-5-(methylthio)phenyl]methanol (3.80 g, 70%).

LCMS (D) Rt=0.40 minutes; m/z [M-NH₃]⁺=167

¹H NMR (CD₃OD) [symbol] 7.27 (d, 1H), 7.26 (s, 1H), 4.50 (s, 2H), 3.70 (s, 2H), 3.34 (bs, 1H), 3.17 (s, 3H), 1.92 (bs, 2H).

### Intermediate 44

### 2-{[(3R,6R)-3-(2,3-Dihydro-1H-inden-2-yl)-6-(1-ethylpropyl)-2,5-dioxo-1-piperazinyl]methyl}-5-(methylsulfonyl)benzaldehyde

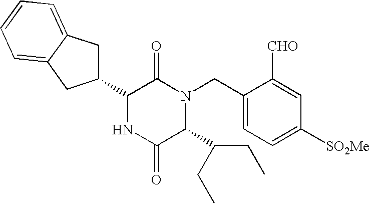

(3R,6R)-3-(2,3-dihydro-1H-inden-2-yl)-6-(1-ethylpropyl)-1-{[2-(hydroxymethyl)-4-(methylsulfonyl)phenyl]methyl}-2,5-piperazinedione (Ex. 206) (880 mg, 1.76 mmol) was dissolved in dry dichloromethane (5 ml). Dess-Martin Periodinane (1.13 g, 2.64 mmol) was added. The resulting suspension was stirred at RT for 1 hour. The crude reaction mixture was purified directly via combi flash silica gel column eluting with 0-75% ethyl acetate in hexanes to give 2-{[(3R,6R)-3-(2,3-dihydro-1H-inden-2-yl)-6-(1-ethylpropyl)-2,5-dioxo-1-piperazinyl]methyl}-5-(methylsulfonyl)benzaldehyde (710 mg, 81%) as a white solid.

LCMS (D) Rt=2.73 minutes; m/z [M+H]⁺=497

¹H NMR (CD₃OD) δ 10.26 (s, 1H), 8.19 (s, 1H), 7.92 (d, 1H), 7.43 (d, 1H), 7.22 (m, 2H), 7.14 (m, 2H), 5.75 (d, 1H), 5.15 (dd, 1H), 4.75 (dd, 1H), 4.10 (m, 1H), 4.01 (m, 1H), 3.13 (s, 3H), 3.09-3.13 (m, 2H), 2.92 (m, 2H), 1.60-1.75 (m, 4H), 1.35 (m, 1H), 0.96 (t, 3H), 0.93 (t, 3H).

Intermediate 45 was prepared by a method analogous to Example 1, using ({2-[(1,1-dimethylethyl)thio]phenyl}methyl)amine and 2-methylpropionaldehyde.

| Int. | | | Rt/ | +ve; | |
|---|---|---|---|---|---|
| No. | Structure | Mwt | min | −ve | Name |
| 45 | 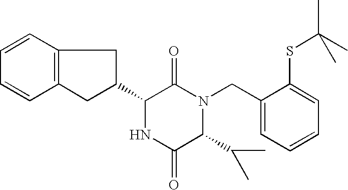 | 450 | 3.8(A) | 451;— | (3R,6R)-3-(2,3-dihydro-1H-inden-2-yl)-1-({2-[(1,1-dimethylethyl)-thio]phenyl}methyl)-6-(1-methyl-ethyl)-2,5-piperazinedione |

Intermediate 46 was prepared from Intermediate 45 by a method analogous to Intermediate 19

| Int. | | | Rt/ | +ve; | |
|---|---|---|---|---|---|
| No. | Structure | Mwt | min | −ve | Name |
| 46 | 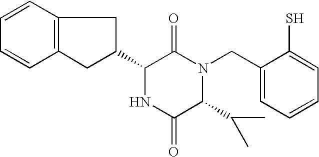 | 394 | 3.4(A) | 495;— | (3R,6R)-3-(2,3-dihydro-1H-inden-2-yl)-1-[(2-mercaptophenyl)methyl]-6-(1-methylethyl)-2,5-piperazinedione |

Intermediate 47 was prepared from Intermediate 46 by a method analogous to Intermediate 20.

| Int. | | | Rt/ | +ve; | |
|---|---|---|---|---|---|
| No. | Structure | Mwt | min | −ve | Name |
| 47 | 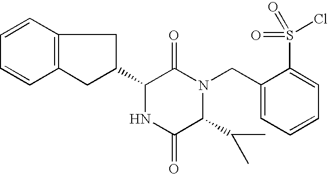 | 460 | 3.5(A) | 461;— | 2-{[(3R,6R)-3-(2,3-dihydro-1H-inden-2-yl)-6-(1-methylethyl)-2,5-dioxo-1-piperazinyl]methyl}benzenesulfonylchloride |

### Intermediate 48

### 4-{[(3R,6R)-3-(2,3-Dihydro-1H-inden-2-yl)-6-(1-ethylpropyl)-2,5-dioxo-1-piperazinyl]-methyl}-1,3-benzenedicarbaldehyde

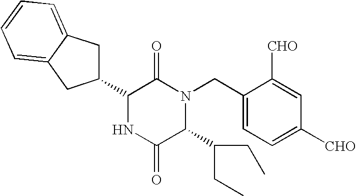

(3R,6R)-1-{[2,4-Bis(hydroxymethyl)phenyl]methyl}-3-(2,3-dihydro-1H-inden-2-yl)-6-(1-ethylpropyl)-2,5-piperazinedione (Example 196) (490 mg, 1.1 mmol) was dissolved in dry dichloromethane (5 ml). Dess-Martin Periodinane (1.38 g, 3.3 mmol) was added. The resulting suspension was stirred at RT for 1 hour. The crude reaction mixture was purified directly via combi flash silica gel column eluting with 0-75% ethyl acetate in hexanes to give 4-{[(3R,6R)-3-(2,3-dihydro-1H-inden-2-yl)-6-(1-ethylpropyl)-2,5-dioxo-1-piperazinyl]methyl}-1,3-benzenedicarbaldehyde (485 mg, 100%) as a white solid.

LCMS (D) Rt=2.82 minutes; m/z [M+H]⁺=447

¹H NMR (CDCl₃) δ 10.26 (s, 1H), 10.12 (s, 1H), 8.39 (s, 1H), 8.10 (d, 2H), 7.51 (d, 1H), 7.21-7.28 (m, 3H), 5.43 (d, 1H), 4.98 (d, 1H), 4.11-4.19 (m, 1H), 4.05 (s, 1H), 3.18 (m, 3H), 2.96-3.02 (m, 1H), 2.82-2.88 (m, 1H), 1.50-1.70 (m, 4H), 1.36-0.141 (m, 1H), 0.93 (t, 3H), 0.88 (t, 3H).

### Intermediate 49

### Diastereomeric mixture of 4-{[(3R,6S)-3-(2,3-dihydro-1H-inden-2-yl)-6-(1-ethylpropyl)-2,5-dioxo-1-piperazinyl]methyl}benzoic acid and 4-{([(3R,6R)-3-(2,3-dihydro-1H-inden-2-yl)-6-(1-ethylpropyl)-2,5-dioxo-1-piperazinyl]methyl}benzoic acid

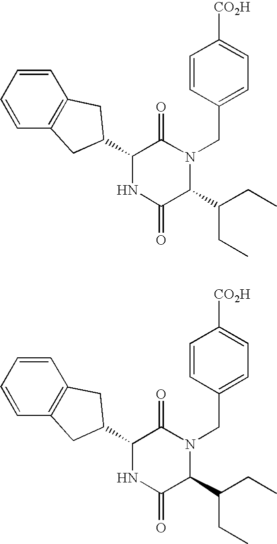

To a solution of 1,1-dimethylethyl 4-(aminomethyl)benzoate (0.79 g, 3.81 mmol) in trifluoroethanol (4 mL) was added 2-ethylbutanal (0.47 mL, 3.81 mmol) and stirred at room temperature for 30 minutes. (2R)-2,3-dihydro-1H-inden-2-yl({[(1,1-dimethylethyl)-oxy]carbonyl}amino)ethanoic acid (1.11 g, 3.81 mmol) was added. The reaction mixture was gently heated to dissolve the indanyl glycine. After stirring at room temperature for 30 minutes, 4-chlorophenylisonitrile (0.52 g, 3.81 mmol) was added. The reaction was stirred at room temperature overnight (22 hours) and then cooled in an ice/water bath. Acetyl chloride (1.6 mL, 22.86 mmol) was added dropwise over 30 minutes. The ice bath was removed and the reaction stirred at room temperature over the weekend. The reaction was concentrated under reduced pressure to give a brown solid. Methylene chloride (20 mL) and a saturated aqueous sodium bicarbonate solution (20 mL) were added to the crude residue and then stirred for 30 minutes. The phases were separated and the aqueous phase extracted with EtOAc (3×). The combined organic phase was washed with brine, dried (MgSO₄) and concentrated under reduced pressure to give 1.0 g of a sticky brown solid. Chloroform (20 mL) was added to the residue and the resulting solution was treated with glacial acetic acid (0.6 mL) and stirred at room temperature overnight (16 hours). The reaction was concentrated to give a brown oil. Ethyl acetate was added to the residue and then extracted with a saturated aqueous NaHCO₃ solution (3×). The combined aqueous extracts were acidified with 2N HCl (pH=2-3) and back extracted with ethyl acetate (3×). The combined organic extracts were washed with brine, dried (MgSO₄), filtered and concentrated to give 0.42 g of the title compounds as a yellow solid.

HPLC (D) Rt=2.70 minutes; m/z [M+H]⁺=435.

### Intermediate 50

### Diastereomeric mixture of 4-{[(3R,6S)-3-(2,3-dihydro-1H-inden-2-yl)-6-(2-methyl-propyl)-2,5-dioxo-1-piperazinyl]methyl}benzoic acid and 4-{[(3R,6R)-3-(2,3-dihydro-1H-inden-2-yl)-6-(2-methylpropyl)-2,5-dioxo-1-piperazinyl]methyl}benzoic acid

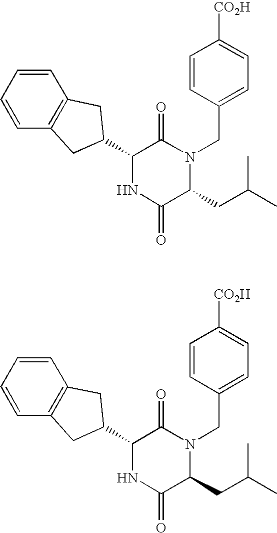

To a solution of 1,1-dimethylethyl 4-(aminomethyl)benzoate (0.70 g, 3.38 mmol) in methanol (4 mL) were added (2R)-2,3-dihydro-1H-inden-2-yl({[(1,1-dimethylethyl)oxy]-carbonyl}amino)ethanoic acid (0.98 g, 3.38 mmol), followed by 4-chlorophenylisonitrile (0.46 g, 3.38 mmol) and then isovaleraldehyde (0.37 mL, 3.38 mmol). The reaction was stirred at room temperature overnight (18 hours) and then concentrated under reduced pressure to give a yellow solid. Methylene chloride (3 mL) was added to the residue and then cooled to 0° C. with an ice bath. 4M HCl dioxane (5 mL, 20.3 mmol) was added dropwise over 30 minutes. The ice bath was removed and the reaction stirred at room temperature over the weekend. The reaction was concentrated under reduced pressure to give a dark brown oil. Methylene chloride (20 mL) and a saturated aqueous sodium bicarbonate solution (10 mL) were added to the crude residue and then stirred for 30 minutes. The phases were separated. The organic phase was furthered extracted with a saturated aqueous NaHCO₃ solution (3×). The combined aqueous extracts were acidified with 2N HCl (pH=2-3) and back extracted with ethyl acetate (3×). The combined organic extracts were washed with brine, dried (MgSO₄), filtered and concentrated to give 1.01 g of the title compounds as a yellow solid.

HPLC (D) Rt=2.61 minutes; m/z [M+H]⁺=421.

### Intermediate 51

### 2-{[(3R,6R)-3-(2,3-Dihydro-1H-inden-2-yl)-6-(1-ethylpropyl)-2,5-dioxopiperazin-1-yl]-methyl}benzaldehyde

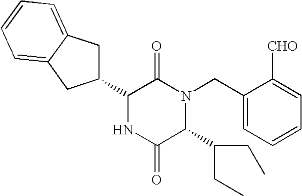

(3R,6R)-3-(2,3-Dihydro-1H-inden-2-yl)-6-(1-ethylpropyl)-1-[2-(hydroxymethyl)-benzyl]piperazine-2,5-dione (Ex. 1) (4.04 g, 9.6 mmol) was dissolved in dry dichloromethane (25 ml) containing 4 Å molecular sieves (3.43 g). 4-Methylmorpholine N-oxide (1.6 g, 13.6 mmol) was added to the stirred mixture followed by tetrapropylammonium perruthenate (101 mg, 0.29 mmol). The mixture was stirred at room temperature for 90 minutes before it was loaded onto a 40 g flash silica chromatography column (pre-eluted with cyclohexane). The column was eluted with 0% to 100% ethyl acetate in cyclohexane to afford 2-{[(3R,6R)-3-(2,3-dihydro-1H-inden-2-yl)-6-(1-ethylpropyl)-2,5-dioxopiperazin-1-yl]methyl}benzaldehyde (2.5 g) as a pale cream solid.

HPLC (A) Rt=3.35 minutes; m/z [M+H]⁺=419.

¹H NMR (CDCl₃) [symbol] 10.15 (s, 1H), 7.86 (dd, 1H), 7.59 (dt, 1H), 7.52 (br t, 1H), 7.32 (d, 1H), 7.22 (m, 5H), 5.47 (d, 1H), 4.90 (d, 1H), 4.15 (dd, 1H), 4.00 (d, 1H), 3.16 (m, 3H), 2.97 (m, 1H), 2.83 (dd, 1H), 1.63 (m, 4H), 1.34 (m, 1H), 0.88 (m, 6H).

### Intermediate 52

### 4-{[(3R,6R)-3-(2,3-dihydro-1H-inden-2-yl)-6-(1-ethylpropyl)-2,5-dioxo-1-piperazinyl]methyl}-1,3-benzenedicarboxylic acid

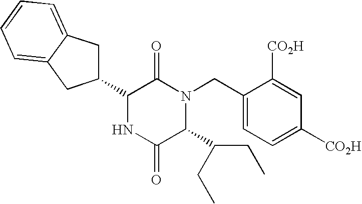

A solution of sulfamic acid (277 mg, 2.86 mmol) in water (2 mL) was added dropwise over 5 minutes to a stirred solution of 4-{[(3R,6R)-3-(2,3-dihydro-1H-inden-2-yl)-6-(1-ethylpropyl)-2,5-dioxo-1-piperazinyl]methyl}-1,3-benzenedicarbaldehyde (Int. 48) (490 mg, 1.10 mmol) in acetonitrile (20 mL), followed by the dropwise addition of a solution of sodium chlorite (298 mg, 3.30 mmol) in water (3 mL). After the mixture had been stirred at room temperature for 90 minutes it was evaporated under reduced pressure to remove the organic solvent. The aqueous residue was diluted with 5 mL water and extracted with ethyl acetate (3×10 mL). The combined organics were washed with saturated aqueous sodium chloride solution (10 mL), dried over MgSO₄, evaporated under reduced pressure and dried in vacuo to afford 4-{[(3R,6R)-3-(2,3-dihydro-1H-inden-2-yl)-6-(1-ethylpropyl)-2,5-dioxo-1-piperazinyl]methyl}-1,3-benzenedicarboxylic acid as a white solid (490 mg, 98%).

LCMS (D) Rt=2.47 minutes; m/z [M+H]⁺=479

¹H NMR (DMSO) [symbol] 8.54 (s, 1H), 8.43 (s, 1H), 8.09 (d, 1H), 7.35 (d, 1H), 7.22 (s, 2H), 7.12 (s, 2H), 5.10 (d, 1H), 4.87 (d, 1H), 4.00-4.05 (m, 1H), 3.90 (s, 1H), 3.10-3.50 (bs, 1H), 2.80-3.10 (m, 5H), 1.30-1.60 (m, 4H), 1.10-0.125 (m, 1H), 0.80 (t, 3H), 0.70 (t, 3H).

### Intermediate 53

### 2-{[(3R,6R)-3-(2,3-Dihydro-1H-inden-2-yl)-6-(1-ethylpropyl)-2,5-dioxo-1-piperazinyl]-methyl}-5-(methylsulfonyl)benzoic acid

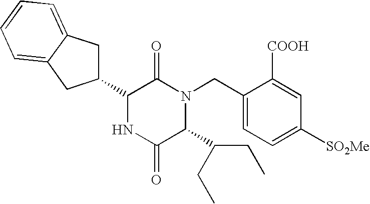

A solution of sulfamic acid (130 mg, 1.30 mmol) in water (2 mL) was added dropwise over 5 minutes to a stirred solution of 2-{[(3R,6R)-3-(2,3-dihydro-1H-inden-2-yl)-6-(1-ethylpropyl)-2,5-dioxo-1-piperazinyl]methyl}-5-(methylsulfonyl)benzaldehyde (Int. 44) (500 mg, 1.00 mmol) in acetonitrile (15 mL), followed by the dropwise addition of a solution of sodium chlorite (136 mg, 1.50 mmol) in water (2 mL). After the mixture had been stirred at room temperature for 90 minutes it was evaporated under reduced pressure to remove the organic solvent. The aqueous residue was diluted with 10 mL water and extracted with ethyl acetate (3×10 mL). The combined organics were dried over MgSO₄, evaporated under reduced pressure and dried in vacuo to afford 2-{[(3R,6R)-3-(2,3-dihydro-1H-inden-2-yl)-6-(1-ethylpropyl)-2,5-dioxo-1-piperazinyl]methyl}-5-(methylsulfonyl)benzoic acid as a white solid (520 mg, 99%).

LCMS (D) Rt=2.54 minutes; m/z [M+H]⁺=513

The following intermediate was prepared by a method analogous to Intermediate 15

| Int. | | | Rt/ | +ve; | |
|---|---|---|---|---|---|
| No. | Structure | Mwt | min | −ve | Name |
| 54 | 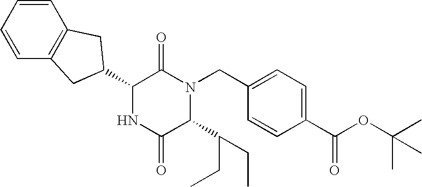 | 490.6 | 3.79(A) | 491 | 1,1-dimethylethyl 4-{[(3R,6R)-3-(2,3-dihydro-1H-inden-2-yl)-6-(1-ethylpropyl)-2,5-dioxo-1-piperazinyl]methyl}benzoate |

### Example 1

### (3R,6R)-3-(2,3-Dihydro-1H-inden-2-yl)-6-(1-ethylpropyl)-1-[2(hydroxymethyl)-benzyl]piperazine-2,5-dione

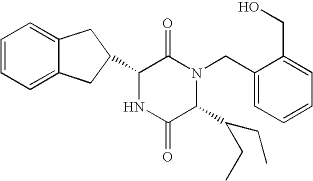

[2-(Aminomethyl)phenyl]methanol (4.12 g, 30 mmol) was dissolved in methanol (30 ml) and 2-ethylbutanal (3.7 ml, 30 mmol) added followed by (2R)-2,3-dihydro-1H-inden-2-yl({[(1,1-dimethylethyl)oxy]carbonyl}amino)ethanoic acid (8.74 g, 30 mmol). The mixture was stirred for 15 minutes before 4-chlorophenylisonitrile (4.13 g, 30 mmol) was added. The mixture was stirred for 2.25 hours and then left to stand at room temperature overnight (16.3 hours) before it was cooled in an ice/water bath. Then acetyl chloride (12.75 ml, 179.5 mmol) was added dropwise, keeping the reaction temperature below 20° C. Then the mixture was stirred in the cooling bath for a further 10 minutes before it was stirred at room temperature. After 5 hours the mixture was evaporated under reduced pressure to leave a dark brown gum. The gum was stirred in chloroform (75 ml) and saturated aqueous sodium bicarbonate solution (75 ml) for 20 minutes before it was diluted with chloroform (75 ml) and the phases separated. The aqueous phase was extracted with chloroform (3×75 ml). The combined organic phase was dried (MgSO₄) and concentrated under reduced pressure to ca. 75 ml. The chloroform solution was treated with glacial acetic acid (3 ml) and left to stand, at room temperature over the weekend. Then the reaction mixture was washed with 2M hydrochloric acid (75 ml), followed by saturated aqueous sodium bicarbonate solution (75 ml). The organic phase was dried (MgSO₄) and evaporated under reduced pressure and dried to leave a brown foam. The foam was loaded in dichloromethane onto a 330 g flash silica chromatography column (pre-eluted with 20% ethyl acetate in cyclohexane). The column was eluted with 20% to 100% ethyl acetate in cyclohexane to afford (3R,6R)-3-(2,3-dihydro-1H-inden-2-yl)-6-(1-ethylpropyl)-1-[2-(hydroxymethyl)benzyl]piperazine-2,5-dione (4.12 g) as a pale brown solid.

HPLC (A) Rt=3.26 minutes; m/z [M+H]⁺=421.

¹H NMR (CDCl₃) δ 7.37 (m, 1H), 7.30 (m, 2H), 7.21 (m, 5H), 6.84 (br d, 1H), 5.45 (d, 1H), 4.74 and 4.63 (d, 2H), 4.16 (d, 1H), 4.08 (dd, 1H), 4.04 (d, 1H), 3.15 (m, 3H), 2.92 (m, 1H), 2.78 (m, 2H), 1.76 (m, 1H), 1.62 (m, 3H), 1.31 (m, 1H), 0.92 (m, 6H).

Examples 2-12, 17-31, 33, 43-47 were prepared by methods analogous to that described for Example 1 using 4-chlorophenylisonitrile, optionally with the addition of a base such as triethylamine or DIPEA if the hydrochloride salts of amines were used. In a modification of this method, Examples 13-16, 32, 34-42, 48 were prepared in a manner analogous to Example 124, using 2-{[(1,1-dimethylethyl)(dimethyl)silyl]oxy}-phenyl isocyanide, optionally with the addition of a base such as triethylamine or DIPEA if the hydrochloride salts of amines were used.

| Ex | | | Rt/ | +ve; | |
|---|---|---|---|---|---|
| No | Structure | Mwt | min | −ve | Name |
| 2 | 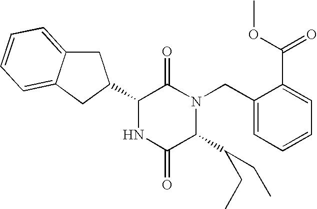 | 448.5 | 3.47(A) | 449;447 | methyl 2-{[(3R,6R)-3-(2,3-dihydro-1H-inden-2-yl)-6-(1-ethylpropyl)-2,5-dioxo-1-piperazinyl]methyl}-benzoate |
| 3 | 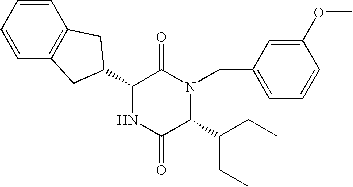 | 420.6 | 3.50(A) | 421;419 | (3R,6R)-3-(2,3-dihydro-1H-inden-2-yl)-6-(1-ethylpropyl)-1-{[3-methoxy-phenyl]methyl}-2,5-piperazinedione |
| 4 | 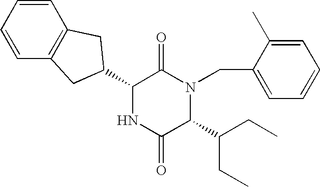 | 404.6 | 3.59(A) | 405;403 | (3R,6R)-3-(2,3-dihydro-1H-inden-2-yl)-6-(1-ethylpropyl)-1-[(2-methylphenyl)methyl]-2,5-piperazinedione |
| 5 | 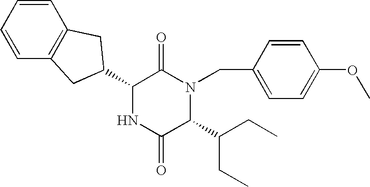 | 420.6 | 3.48(A) | 421;419 | (3R,6R)-3-(2,3-dihydro-1H-inden-2-yl)-6-(1-ethylpropyl)-1-{[4-methoxy-phenyl]methyl}-2,5-piperazinedione |
| 6 | 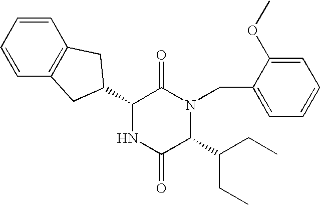 | 420.6 | 3.54(A) | 421;— | (3R,6R)-3-(2,3-dihydro-1H-inden-2-yl)-6-(1-ethylpropyl)-1-{[2-(methoxy)phenyl]methyl}-2,5-piperazinedione |
| 7 | 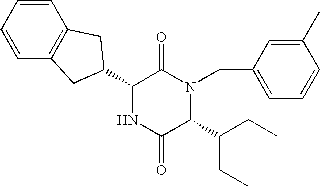 | 404.6 | 3.60(A) | 405;— | (3R,6R)-3-(2,3-dihydro-1H-inden-2-yl)-6-(1-ethylpropyl)-1-[(3-methylphenyl)methyl]-2,5-piperazinedione |
| 8 | 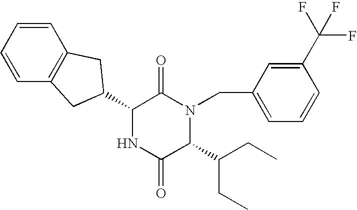 | 458.5 | 3.63(A) | 459;— | (3R,6R)-3-(2,3-dihydro-1H-inden-2-yl)-6-(1-ethylpropyl)-1-{[3-(trifluoromethyl)phenyl]methyl}-2,5-piperazinedione |
| 9 | 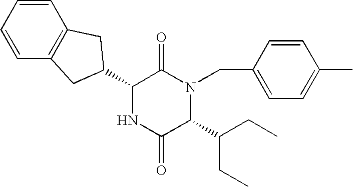 | 404.6 | 3.62(A) | 405;403 | (3R,6R)-3-(2,3-dihydro-1H-inden-2-yl)-6-(1-ethylpropyl)-1-[(4-methylphenyl)methyl]-2,5-piperazinedione |
| 10 | 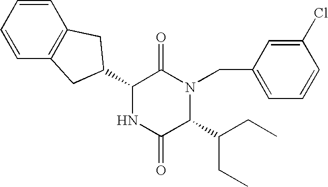 | 425.0 | 3.65(A) | 425;423 | (3R,6R)-1-[(3-chlorophenyl)methyl]-3-(2,3-dihydro-1H-inden-2-yl)-6-(1-ethylpropyl)-2,5-piperazinedione |
| 11 | 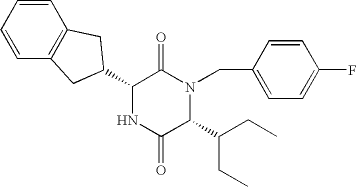 | 408.5 | 3.51(A) | 409;407 | (3R,6R)-3-(2,3-dihydro-1H-inden-2-yl)-6-(1-ethylpropyl)-1-[(4-fluorophenyl)methyl]-2,5-piperazinedione |
| 12 | 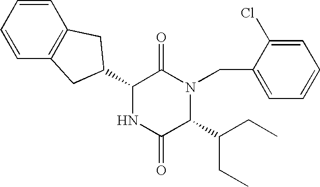 | 425.0 | 3.66(A) | 425;423 | (3R,6R)-1-[(2-chlorophenyl)methyl]-3-(2,3-dihydro-1H-inden-2-yl)-6-(1-ethylpropyl)-2,5-piperazinedione |
| 13 | 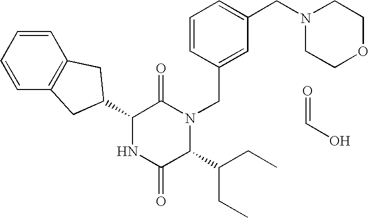 | 535.7 | 2.58(A) | 490;488 | (3R,6R)-3-(2,3-dihydro-1H-inden-2-yl)-6-(1-ethylpropyl)-1-{[3-(4-morpholinylmethyl)-phenyl]methyl}-2,5-piperazinedione formate |
| 14 | 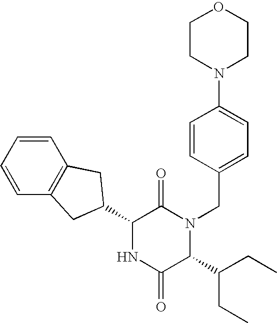 | 475.6 | 3.38(A) | 476;474 | (3R,6R)-3-(2,3-dihydro-1H-inden-2-yl)-6-(1-ethylpropyl)-1-{[4-(4-morpholinyl)phenyl]methyl}-2,5-piperazinedione |
| 15 | 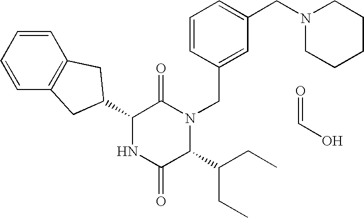 | 533.7 | 2.65(A) | 488;486 | (3R,6R)-3-(2,3-dihydro-1H-inden-2-yl)-6-(1-ethylpropyl)-1-{[3-(1-piperidinylmethyl)-phenyl]methyl}-2,5-piperazinedione formate |
| 16 | 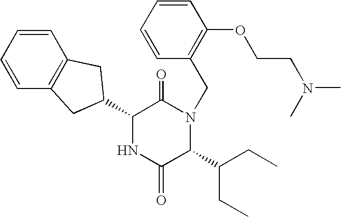 | 477.7 | 2.67(A) | 478;— | (3R,6R)-3-(2,3-dihydro-1H-inden-2-yl)-1-[(2-{[2-(dimethylamino)-ethyl]oxy}phenyl)methyl]-6-(1-ethylpropyl)-2,5-piperazinedione |
| 17 | 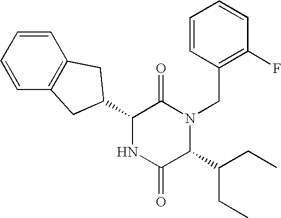 | 408.5 | 3.54(A) | 409;407 | (3R,6R)-3-(2,3-dihydro-1H-inden-2-yl)-6-(1-ethylpropyl)-1-[(2-fluorophenyl)methyl]-2,5-piperazinedione |
| 18 | 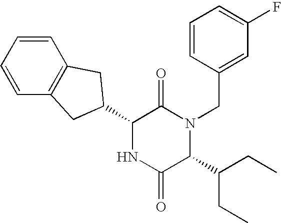 | 408.5 | 3.54(A) | 409;407 | (3R,6R)-3-(2,3-dihydro-1H-inden-2-yl)-6-(1-ethylpropyl)-1-[(3-fluorophenyl)methyl]-2,5-piperazinedione |
| 19 | 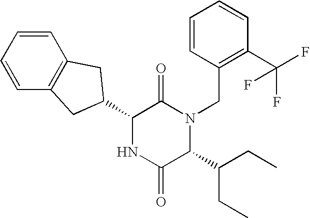 | 458.5 | 3.73(A) | 459;457 | (3R,6R)-3-(2,3-dihydro-1H-inden-2-yl)-6-(1-ethylpropyl)-1-{[2-(trifluoromethyl)phenyl]methyl}-2,5-piperazinedione |
| 20 | 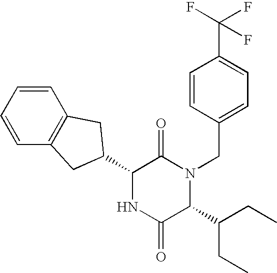 | 458.5 | 3.70(A) | 459;457 | (3R,6R)-3-(2,3-dihydro-1H-inden-2-yl)-6-(1-ethylpropyl)-1-{[4-(trifluoromethyl)phenyl]methyl}-2,5-piperazinedione |
| 21 | 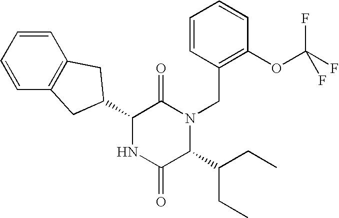 | 474.5 | 3.73(A) | 475;473 | (3R,6R)-3-(2,3-dihydro-1H-inden-2-yl)-6-(1-ethylpropyl)-1-({2-[(trifluoromethyl)oxy]phenyl}methyl)-2,5-piperazinedione |
| 22 | 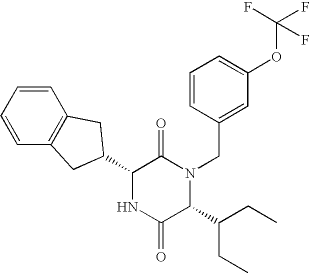 | 474.5 | 3.73(A) | 475;473 | (3R,6R)-3-(2,3-dihydro-1H-inden-2-yl)-6-(1-ethylpropyl)-1-({3-[(trifluoromethyl)oxy]phenyl}methyl)-2,5-piperazinedione |
| 23 | 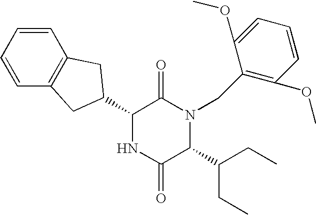 | 450.6 | 3.50(A) | 451;— | (3R,6R)-1-{[2,6-bis(methoxy)-phenyl]methyl}-3-(2,3-dihydro-1H-inden-2-yl)-6-(1-ethylpropyl)-2,5-piperazinedione |
| 24 | 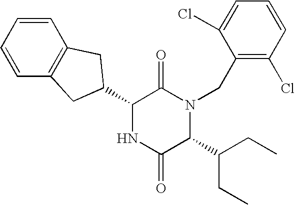 | 459.4 | 3.74(A) | 459/461;455/457 | (3R,6R)-1-[(2,6-dichlorophenyl)-methyl]-3-(2,3-dihydro-1H-inden-2-yl)-6-(1-ethylpropyl)-2,5-piperazinedione |
| 25 | 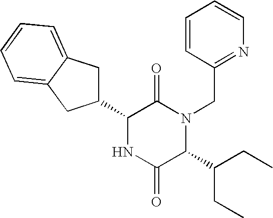 | 391.5 | 3.09(A) | 392;— | (3R,6R)-3-(2,3-dihydro-1H-inden-2-yl)-6-(1-ethylpropyl)-1-(2-pyridinylmethyl)-2,5-piperazinedione |
| 26 | 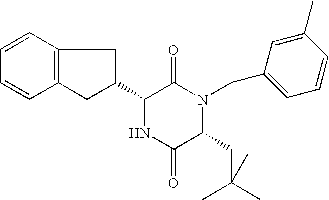 | 404.6 | 3.69(A) | 405;403 | (3R,6R)-3-(2,3-dihydro-1H-inden-2-yl)-6-(2,2-dimethylpropyl)-1-[(3-methylphenyl)methyl]-2,5-piperazinedione |
| 27 | 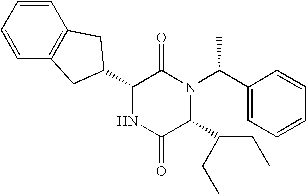 | 404.6 | 3.58(A) | 405;403 | (3R,6R)-3-(2,3-dihydro-1H-inden-2-yl)-6-(1-ethylpropyl)-1-[(1R)-1-phenylethyl]-2,5-piperazinedione |
| 28 | 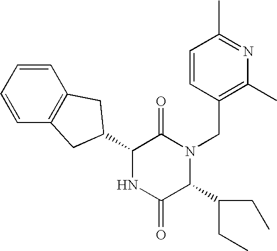 | 419.6 | 2.51(A) | 421;418 | (3R,6R)-3-(2,3-dihydro-1H-inden-2-yl)-1-[(2,6-dimethyl-3-pyridinyl)-methyl]-6-(1-ethylpropyl)-2,5-piperazinedione |
| 29 | 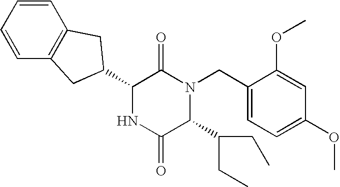 | 450.6 | 3.53(A) | 451;— | (3R,6R)-1-{[2,4-bis(methoxy)-phenyl]methyl}-3-(2,3-dihydro-1H-inden-2-yl)-6-(1-ethylpropyl)-2,5-piperazinedione |
| 30 | 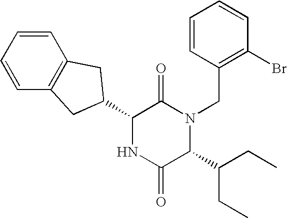 | 469.4 | 3.78(A) | 469/471;467/469 | (3R,6R)-1-[(2-bromophenyl)methyl]-3-(2,3-dihydro-1H-inden-2-yl)-6-(1-ethylpropyl)-2,5-piperazinedione |
| 31 | 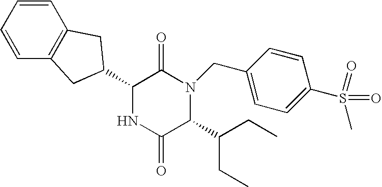 | 468.6 | 3.0 (A) | 469;467 | (3R,6R)-3-(2,3-dihydro-1H-inden-2-yl)-6-(1-ethylpropyl)-1-{[4-(methylsulfonyl)phenyl]methyl}-2,5-piperazinedione |
| 32 | 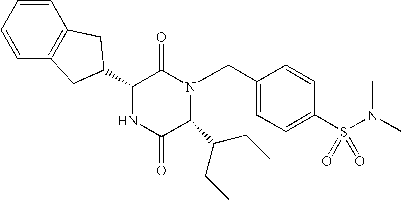 | 497.7 | 3.3 (A) | 498;496 | 4-{[(3R,6R)-3-(2,3-dihydro-1H-inden-2-yl)-6-(1-ethylpropyl)-2,5-dioxo-1-piperazinyl]-methyl}-N,N-dimethyl-benzenesulfonamide |
| 33 | 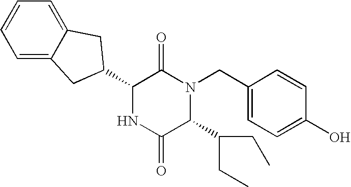 | 406.5 | 3.29(A) | 407;405 | (3R,6R)-3-(2,3-dihydro-1H-inden-2-yl)-6-(1-ethylpropyl)-1-[(4-hydroxyphenyl)methyl]-2,5-piperazinedione |
| 34 | 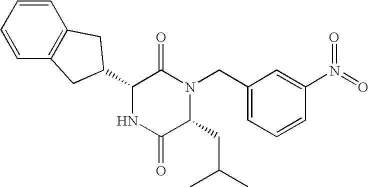 | 421.5 | 3.34(A) | 422;420 | (3R,6R)-3-(2,3-dihydro-1H-inden-2-yl)-6-(2-methylpropyl)-1-[(3-nitrophenyl)methyl]-2,5-piperazinedione |
| 35 | 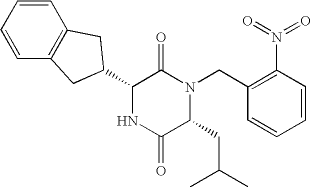 | 421.5 | 3.35(A) | 422;420 | (3R,6R)-3-(2,3-dihydro-1H-inden-2-yl)-6-(2-methylpropyl)-1-[(2-nitrophenyl)methyl]-2,5-piperazinedione |
| 36 | 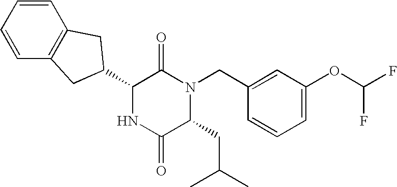 | 442.5 | 3.42(A) | 443;441 | (3R,6R)-1-({3-[(difluoromethyl)oxy]phenyl}methyl)-3-(2,3-dihydro-1H-inden-2-yl)-6-(2-methylpropyl)-2,5-piperazinedione |
| 37 | 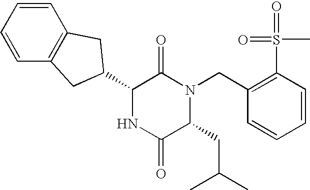 | 454.6 | 3.25(A) | 455;453 | (3R,6R)-3-(2,3-dihydro-1H-inden-2-yl)-6-(2-methylpropyl)-1-{[2-(methylsulfonyl)phenyl]methyl}-2,5-piperazinedione |
| 38 | 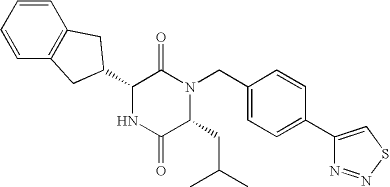 | 460.6 | 3.49(A) | 461;459 | (3R,6R)-3-(2,3-dihydro-1H-inden-2-yl)-6-(2-methylpropyl)-1-{[4-(1,2,3-thiadiazol-4-yl)phenyl]methyl}-2,5-piperazinedione |
| 39 | 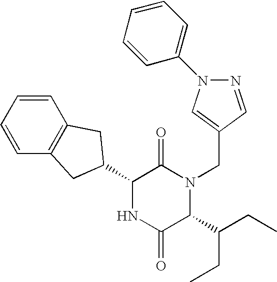 | 456.6 | 3.55(A) | 457;455 | (3R,6R)-3-(2,3-dihydro-1H-inden-2-yl)-6-(1-ethylpropyl)-1-[(1-phenyl-1H-pyrazol-4-yl)methyl]-2,5-piperazinedione |
| 40 | 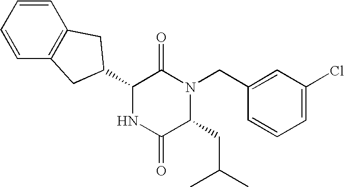 | 410.9 | 3.52(A) | 411;409 | (3R,6R)-1-[(3-chlorophenyl)methyl]-3-(2,3-dihydro-1H-inden-2-yl)-6-(2-methylpropyl)-2,5-piperazinedione |
| 41 | 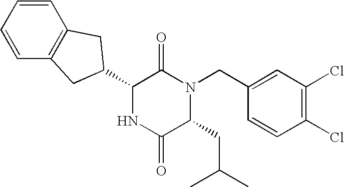 | 445.4 | 3.65(A) | 445;443 | (3R,6R)-1-[(3,4-dichlorophenyl)-methyl]-3-(2,3-dihydro-1H-inden-2-yl)-6-(2-methylpropyl)-2,5-piperazinedione |
| 42 | 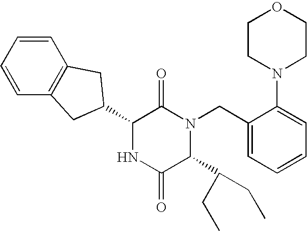 | 475.6 | 3.47(A) | 476;474 | (3R,6R)-3-(2,3-dihydro-1H-inden-2-yl)-6-(1-ethylpropyl)-1-{[2-(4-morpholinyl)phenyl]methyl}-2,5-piperazinedione |
| 43 | 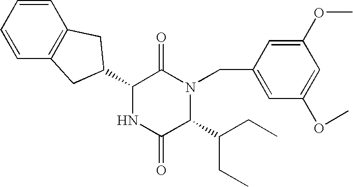 | 450.6 | 3.48(A) | 451;449 | (3R,6R)-1-{[3,5-bis(methoxy)-phenyl]methyl}-3-(2,3-dihydro-1H-inden-2-yl)-6-(1-ethylpropyl)-2,5-piperazinedione |
| 44 | 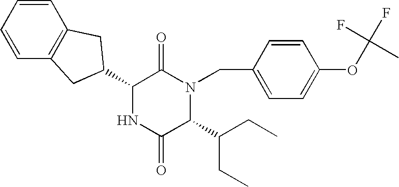 | 474.5 | 3.75(A) | 475;473 | (3R,6R)-3-(2,3-dihydro-1H-inden-2-yl)-6-(1-ethylpropyl)-1-({4-[(trifluoromethyl)oxy]phenyl}methyl)-2,5-piperazinedione |
| 45 | 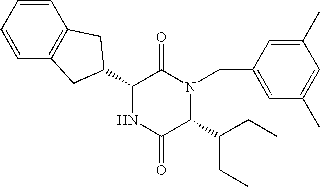 | 418.6 | 3.74(A) | 419;— | (3R,6R)-3-(2,3-dihydro-1H-inden-2-yl)-1-[(3,5-dimethylphenyl)methyl]-6-(1-ethylpropyl)-2,5-piperazinedione |
| 46 | 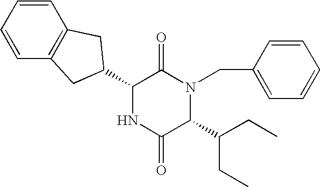 | 390.5 | 3.51(A) | 391;— | (3R,6R)-3-(2,3-dihydro-1H-inden-2-yl)-6-(1-ethylpropyl)-1-(phenyl-methyl)-2,5-piperazinedione |
| 47 | 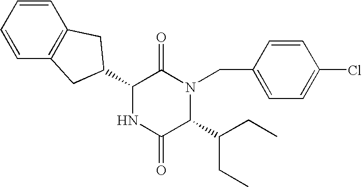 | 425.0 | 3.66(A) | 425;423 | (3R,6R)-1-[(4-chlorophenyl)methyl]-3-(2,3-dihydro-1H-inden-2-yl)-6-(1-ethylpropyl)-2,5-piperazinedione |
| 48 | 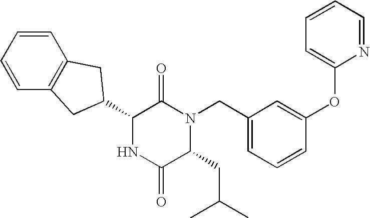 | 469.9 | 3.37(A) | 470;468 | (3R,6R)-3-(2,3-dihydro-1H-inden-2-yl)-6-(2-methylpropyl)-1-{[3-(2-pyridinyloxy)phenyl]methyl}-2,5-piperazinedione |

Examples 49-61 were prepared by methods analogous to that described for Example 1, using the Intermediates indicated, optionally with the addition of a base such as triethylamine or DIPEA if the hydrochloride salts of amines were used

| Ex | Int | | | Rt/ | +ve; | |
|---|---|---|---|---|---|---|
| No | No | Structure | Mwt | min | −ve | Name |
| 49 | 1 | 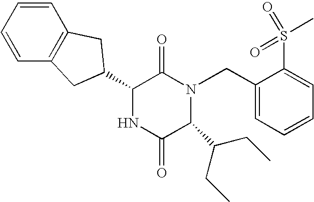 | 468.6 | 3.3 (A) | 469;467 | (3R,6R)-3-(2,3-dihydro-1H-inden-2-yl)-6-(1-ethylpropyl)-1-{[2-(methylsulfonyl)phenyl]-methyl}-2,5-piperazinedione |
| 50 | 1 | 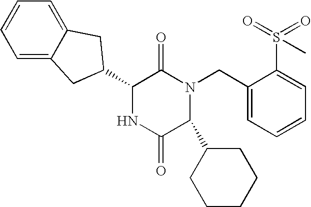 | 480.6 | 3.37(A) | 481;479 | (3R,6R)-6-cyclohexyl-3-(2,3-dihydro-1H-inden-2-yl)-1-{[2-(methylsulfonyl)phenyl]methyl}-2,5-piperazinedione |
| 51 | 1 | 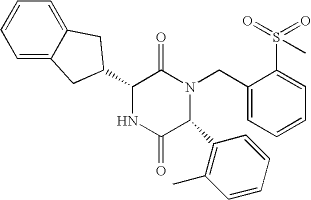 | 488.6 | 3.19(A) | 489;487 | (3R,6R)-3-(2,3-dihydro-1H-inden-2-yl)-6-(2-methylphenyl)-1-{[2-(methylsulfonyl)phenyl]-methyl}-2,5-piperazinedione |
| 52 | 1 | 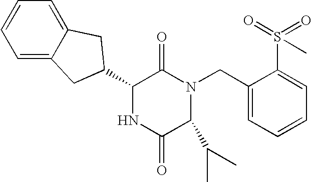 | 376.5 | 3.36(A) | 377;375 | (3R,6R)-3-(2,3-dihydro-1H-inden-2-yl)-6-(1-methylethyl)-1-{[2-(methylsulfonyl)phenyl]-methyl}-2,5-piperazinedione |
| 53 | 1 | 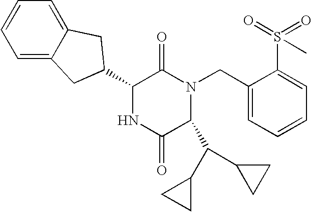 | 492.6 | 3.55(A) | 429;427 | (3R,6R)-6-(dicyclopropylmethyl)-3-(2,3-dihydro-1H-inden-2-yl)-1-{[2-(methylsulfonyl)phenyl]-methyl}-2,5-piperazinedione |
| 54 | 1 | 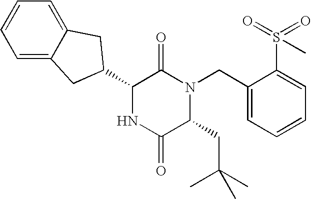 | 468.6 | 3.32(A) | 469;467 | (3R,6R)-3-(2,3-dihydro-1H-inden-2-yl)-6-(2,2-dimethyl-propyl)-1-{[2-(methylsulfonyl)-phenyl]methyl}-2,5-piperazinedione |
| 55 | 2 | 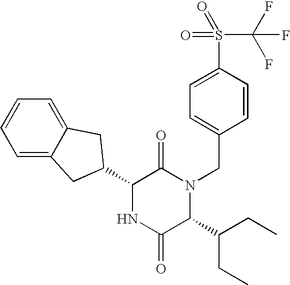 | 522.3 | 3.5 (A) | 523;— | (3R,6R)-3-(2,3-dihydro-1H-inden-2-yl)-6-(1-ethylpropyl)-1-({4-[(trifluoromethyl)-sulfonyl]-phenyl}methyl)-2,5-piperazinedione |
| 56 | 3 | 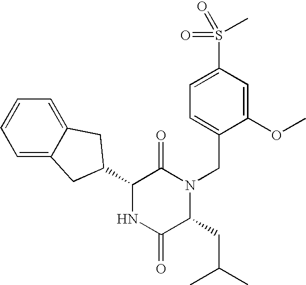 | 484.6 | 3.3 (A) | 485;— | (3R,6R)-3-(2,3-dihydro-1H-inden-2-yl)-1-{[2-methoxy-4-(methylsulfonyl)phenyl]methyl}-6-(2-methylpropyl)-2,5-piperazinedione |
| 57 | 3 | 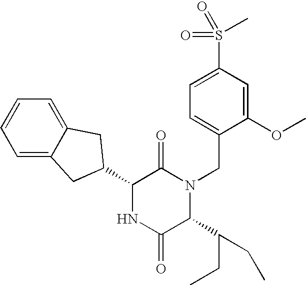 | 498.7 | 3.2 (A) | 499;— | (3R,6R)-3-(2,3-dihydro-1H-inden-2-yl)-6-(1-ethylpropyl)-1-{[2-methoxy-4-(methylsulfonyl)-phenyl]methyl}-2,5-piperazinedione |
| 58 | 4 | 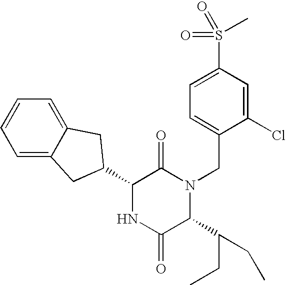 | 503.1 | 3.2 (A) | 503;— | (3R,6R)-1-{[2-chloro-4-(methyl-sulfonyl)phenyl]methyl}-3-(2,3-dihydro-1H-inden-2-yl)-6-(1-ethylpropyl)-2,5-piperazinedione |
| 59 | 5 | 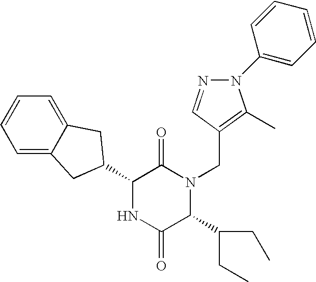 | 470.6 | 3.31(A) | 471;515 | 3R,6R)-3-(2,3-dihydro-1H-inden-2-yl)-6-(1-ethylpropyl)-1-[(5-methyl-1-phenyl-1H-pyrazol-4-yl)methyl]-2,5-piperazinedione |
| 60 | 6 | 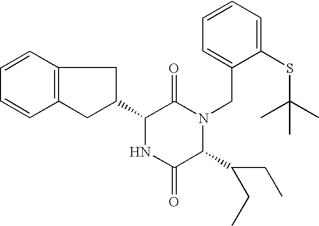 | 478.7 | 4.0 (A) | 479;— | (3R,6R)-3-(2,3-dihydro-1H-inden-2-yl)-1-({2-[(1,1-dimethyl-ethyl)thio]phenyl}methyl)-6-(1-ethylpropyl)-2,5-piperazinedione |
| 61 | 7 | 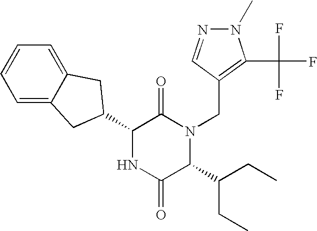 | 462.5 | 3.47(A) | 463;461 | (3R,6R)-3-(2,3-dihydro-1H-inden-2-yl)-6-(1-ethylpropyl)-1-{[1-methyl-5-(trifluoromethyl)-1H-pyrazol-4-yl]methyl}-2,5-piperazinedione |

### Example 62

### (3R,6R)-3-(2,3-Dihydro-1H-inden-2-yl)-6-(1-ethylpropyl)-1-{[3-(methylsulfonyl)-2-pyridinyl]methyl}-2,5-piperazinedione

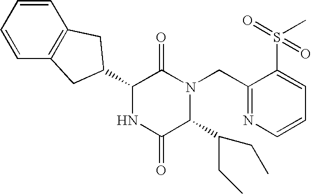

1-[3-(Methylsulfonyl)-2-pyridinyl]methanamine (186 mg, 1 mmol) and 2-ethylbutanal (124 uL, 1 mmol) were dissolved in chloroform (5 mL) and the mixture was left at room temperature for 63 hours. (2R)-2,3-Dihydro-1H-inden-2-yl({[(1,1-dimethylethyl)oxy]-carbonyl}amino)ethanoic acid (291 mg, 1 mmol) was added, followed by 4-chlorophenylisonitrile (138 mg, 1 mmol) and the mixture was stirred for 30 minutes at room temperature, then left for 48 hours. The solvent was blown off with nitrogen, the residue taken up in methanol (10 mL) and the solution was cooled to 0° C., then acetyl chloride (0.5 mL) was cautiously added dropwise. The mixture was left at room temperature overnight, then the solvent was blown off with nitrogen and the residue taken up in dichloromethane (10 mL) and stirred with saturated aqueous sodium hydrogen carbonate (5 mL), with solid sodium hydrogen carbonate being added with caution until effervescence ceased. The organic phase was separated using a hydrophobic frit, and treated with glacial acetic acid (0.1 mL). The mixture was left at room temperature overnight. It was then stirred with saturated aqueous sodium hydrogen carbonate (5 mL), with solid sodium hydrogen carbonate being added with caution until effervescence ceased, then the organic phase was separated using a hydrophobic frit. The solvent was removed under reduced pressure and the crude product was purified by mass-directed autoprep followed by preparative layer chromatography on silica (20×20 cm plates, 2 mm thickness) eluted×3 with 2.5% isopropanol in dichloromethane to give (3R,6R)-3-(2,3-dihydro-1H-inden-2-yl)-6-(1-ethylpropyl)-1-{[3-(methylsulfonyl)-2-pyridinyl]methyl}-2,5-piperazinedione (33 mg) as an off-white solid.

HPLC (A) Rt=3.18 mins, [M+H]⁺=470

¹H NMR (CDCl₃) δ 8.72 (dd, 1H), 8.30 (dd, 1H), 7.72 (br d, 1H), 7.42 (dd, 1H), 7.23-7.14 (m, 4H), 5.41 (d, 1H), 5.03 (d, 1H), 4.47 (d, 1H), 4.00 (dd, 1H), 3.42 (s, 3H), 3.18-2.80 (m, 5H), 1.85-1.61 (m, 4H), 1.48-1.35 (m, 1H), 1.04-0.95 (m, 6H)

### Example 63

### (3R,6R)-3-(2,3-Dihydro-1H-inden-2-yl)-6-(1-ethylpropyl)-1-(2-nitrobenzyl)piperazine-2,5-dione

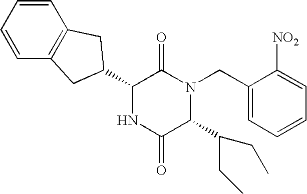

[(2-Nitrophenyl)methyl]amine hydrochloride (5.02 g, 26.6 mmol) was dissolved in ethyl acetate (25 ml) and sodium hydrogen carbonate (25 ml) added. The organic phase was separated, dried (Na₂SO₄) and concentrated to give [(2-nitrophenyl)methyl]amine as a yellow oil (2.48 g, 61.4%).

HPLC Rt=0.46 minutes; m/z [M+H]⁺=153.

To a solution of [(2-nitrophenyl)methyl]amine (2.48 g, 16.2 mmol) in methanol (50 ml) was added (2R)-2,3-dihydro-1H-inden-2-yl({[(1,1-dimethylethyl)oxy]carbonyl}-amino)-ethanoic acid (4.72 g, 16.2 mmol), 2-{[(1,1-dimethylethyl)(dimethyl)silyl]oxy}phenyl isocyanide (3.78 g, 16.2 mmol) and 2-ethylbutanal (2 ml, 16.2 mmol). The reaction was stirred at room temperature for 18 hours then cooled to 0° C. and acetyl chloride (6.9 ml) added. The reaction was stirred at room temperature for 72 hours. The solvent was removed in vacuo and the residue dissolved in chloroform (125 ml). Sodium bicarbonate was added (125 ml) and the biphasic mixture stirred for 3 hours. The organic phase was separated, dried (Na₂SO₄) and concentrated. The residue was dissolved in chloroform (120 ml) and acetic acid (1.2 ml) added and the reaction stirred at room temperature for 18 hours and then at 50° C. for 1.5 hours. The reaction was then washed with hydrochloric acid (2M, 3×100 ml). The organic phase was separated, dried (Na₂SO₄) and concentrated to give a brown solid which was purified by silica column chromatography to give (3R,6R)-3-(2,3-dihydro-1H-inden-2-yl)-6-(1-ethylpropyl)-1-(2-nitrobenzyl)piperazine-2,5-dione as a light brown solid (2.88 g, 44.4%).

HPLC (A) Rt=3.39 minutes, m/z [M+H]⁺=436

¹H NMR (CDCl₃) δ 8.07 (dd, 1H), 7.62 (dt, 1H), 7.48 (dt, 1H), 7.33 (d, 1H), 7.24 (m, 2H), 7.19 (m, 2H), 6.47 (d, 1H), 5.3 (d, 1H), 4.72 (d, 1H), 4.14 (m, 1H), 4.02 (d, 1H), 3.17 (m, 3H), 2.97 (m, 1H), 2.8 (m, 1H), 1.64 (m, 4H), 1.35 (m, 1H), 0.93 (t, 3H), 0.86 (t, 3H).

### Example 65

### 2-{[(3R,6R)-3-(2,3-Dihydro-1H-inden-2-yl)-6-(1-ethylpropyl)-2,5-dioxo-1-piperazinyl]methyl}benzoic acid

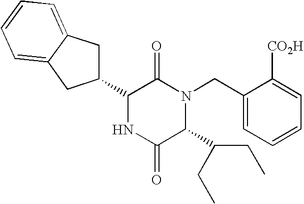

A solution of sulfamic acid (750 mg, 7.72 mmol) in water (30 ml) was added dropwise over 5 minutes to a stirred solution of 2-{[(3R,6R)-3-(2,3-dihydro-1H-inden-2-yl)-6-(1-ethylpropyl)-2,5-dioxopiperazin-1-yl]methyl}benzaldehyde (Int. 51) (2.48 g, 5.92 mmol) in acetonitrile (300 ml), followed by the dropwise addition of a solution of sodium chlorite (775 mg, 8.57 mmol) in water (30 ml). After the mixture had been stirred at room temperature for 70 minutes it was evaporated under reduced pressure to remove the organic solvent. The aqueous residue was partitioned between ethyl acetate (150 ml) and water (10 ml). The organic phase was washed with saturated aqueous sodium chloride solution (50 ml), dried (MgSO₄), evaporated under reduced pressure and dried in vacuo to afford 2-{[(3R,6R)-3-(2,3-dihydro-1H-inden-2-yl)-6-(1-ethylpropyl)-2,5-dioxo-1-piperazinyl]methyl}benzoic acid as a white solid (2.5 g).

HPLC (A) Rt=3.39 minutes; m/z [M+H]⁺=435

¹H NMR (CDCl₃) δ 8.27 (d, 1H), 7.98 (d, 1H), 7.53 (dt, 1H), 7.38 (br t, 1H), 7.35 (d, 1H), 7.23 (m, 2H), 7.16 (m, 2H), 5.30 (d, 1H), 4.68 (d, 1H), 4.15 (d, 1H), 4.12 (dd, 1H), 3.16 (m, 3H), 2.94 (m, 1H), 2.87 (m, 1H), 1.68 (m, 2H), 1.58 (m, 2H), 1.34 (m, 1H), 0.93 (t, 3H), 0.87 (t, 3H).

### Example 66

### 2-{[(3R,6R)-3-(2,3-Dihydro-1H-inden-2-yl)-6-(1-ethylpropyl)-2,5-dioxo-1-piperazinyl]methyl}-N-methylbenzamide

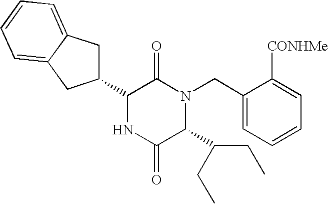

2-{[(3R,6R)-3-(2,3-dihydro-1H-inden-2-yl)-6-(1-ethylpropyl)-2,5-dioxo-1-piperazinyl]-methyl}benzoic acid (Ex. 65) (100 mg, 0.23 mmol) was dissolved in dry dichloromethane (3 ml) and triethylamine (64 ul, 0.46 mmol) added, followed after 7 minutes by 2-(1H-benzotriazol-1-yl)-1,1,3,3-tetramethyluronium tetrafluoroborate (88 mg, 0.27 mmol). The mixture was stirred at room temperature for 5 hours before methylamine (0.6 ml of a 2M solution in tetrahydrofuran, 1.2 mmol) was added. The mixture was left to stand at room temperature overnight (17 hours) before it was diluted with dichloromethane (2 ml) and washed with saturated aqueous sodium bicarbonate solution (2 ml). The organic phase was dried (hydrophobic frit) and evaporated to leave a yellow gum. The gum was purified using mass directed autoprep to give 2-{[(3R,6R)-3-(2,3-dihydro-1H-inden-2-yl)-6-(1-ethylpropyl)-2,5-dioxo-1-piperazinyl]methyl}-N-methylbenzamide as a white solid (73 mg).

HPLC (A) Rt=3.09 minutes; m/z [M+H]⁺=448

¹H NMR (CDCl₃) [symbol] 7.54 (br s, 1H), 7.40 (m, 2H), 7.28 (m, 2H), 7.20 (m, 4H), 6.60 (br q, 1H), 5.13 (d, 1H), 4.47 (d, 1H), 4.11 (d, 1H), 4.03 (dd, 1H), 3.12 (m, 3H), 3.00 (d, 3H), 2.91 (m, 1H), 2.80 (m, 1H), 1.73 (m, 1H), 1.61 (m, 3H), 1.34 (m, 1H), 0.96 (t, 3H), 0.89 (t, 3H).

Examples 67-80 were prepared by methods analogous to that described for Example 66 from 2-{[(3R,6R)-3-(2,3-dihydro-1H-inden-2-yl)-6-(1-ethylpropyl)-2,5-dioxo-1-piperazinyl]-methyl}benzoic acid (Ex. 65)

| Ex | | | Rt/ | +ve; | |
|---|---|---|---|---|---|
| No | Structure | Mwt | min | −ve | Name |
| 67 | 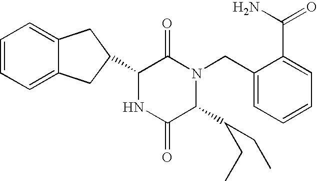 | 433.5 | 3.05(A) | 434;432 | 2-{[(3R,6R)-3-(2,3-dihydro-1H-inden-2-yl)-6-(1-ethylpropyl)-2,5-dioxo-1-piperazinyl]methyl}benzamide |
| 68 | 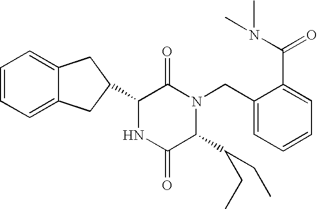 | 461.6 | 3.2 (A) | 462;— | 2-{[(3R,6R)-3-(2,3-dihydro-1H-inden-2-yl)-6-(1-ethylpropyl)-2,5-dioxo-1-piperazinyl]methyl}-N,N-dimethylbenzamide |
| 69 | 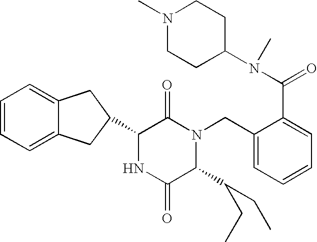 | 544.7 | 2.63(A) | 545;— | 2-{[(3R,6R)-3-(2,3-dihydro-1H-inden-2-yl)-6-(1-ethylpropyl)-2,5-dioxo-1-piperazinyl]methyl}-N-methyl-N-(1-methyl-4-piperidinyl)benzamide |
| 70 | 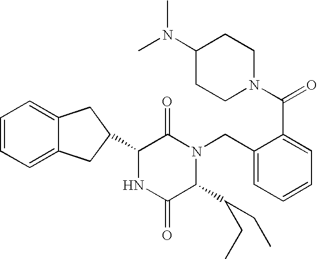 | 544.7 | 2.57(A) | 545;543 | (3R,6R)-3-(2,3-dihydro-1H-inden-2-yl)-1-[(2-{[4-(dimethylamino)-1-piperidinyl]carbonyl}phenyl)methyl]-6-(1-ethylpropyl)-2,5-piperazinedione |
| 71 | 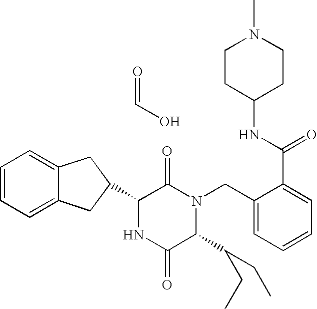 | 576.7 | 2.52(A) | 531;529 | 2-{[(3R,6R)-3-(2,3-dihydro-1H-inden-2-yl)-6-(1-ethylpropyl)-2,5-dioxo-1-piperazinyl]methyl}-N-(1-methyl-4-piperidinyl)benzamide formate |
| 72 | 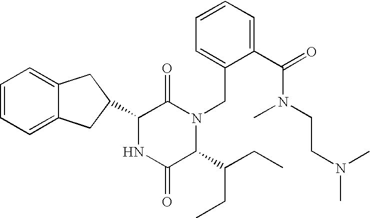 | 518.7 | 2.65(A) | 519;— | 2-{[(3R,6R)-3-(2,3-dihydro-1H-inden-2-yl)-6-(1-ethylpropyl)-2,5-dioxo-1-piperazinyl]methyl}-N-[2-(dimethyl-amino)ethyl]-N-methylbenzamide |
| 73 | 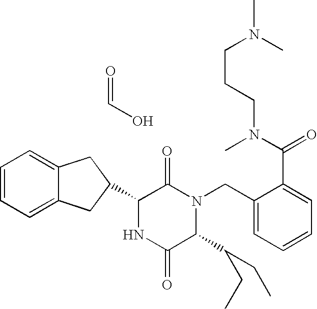 | 578.7 | 2.65(A) | 533;— | 2-{[(3R,6R)-3-(2,3-dihydro-1H-inden-2-yl)-6-(1-ethylpropyl)-2,5-dioxo-1-piperazinyl]methyl}-N-[3-(dimethyl-amino)propyl]-N-methylbenzamideformate |
| 74 | 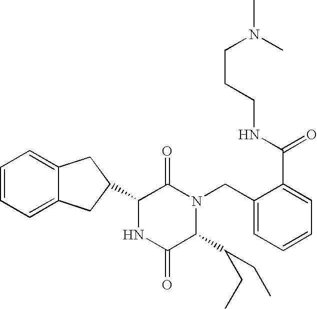 | 518.7 | 2.57(A) | 519;517 | 2-{[(3R,6R)-3-(2,3-dihydro-1H-inden-2-yl)-6-(1-ethylpropyl)-2,5-dioxo-1-piperazinyl]methyl}-N-[3-(dimethylamino)propyl]benzamide |
| 75 | 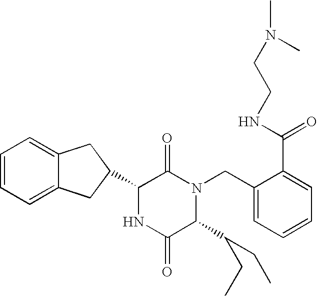 | 504.6 | 2.50(A) | 505;503 | 2-{[(3R,6R)-3-(2,3-dihydro-1H-inden-2-yl)-6-(1-ethylpropyl)-2,5-dioxo-1-piperazinyl]methyl}-N-[2-(dimethylamino)ethyl]benzamide |
| 76 | 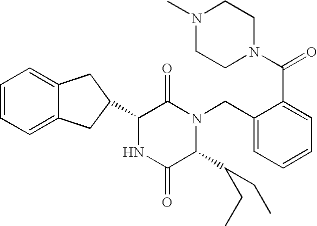 | 516.6 | 2.54(A) | 517;515 | (3R,6R)-3-(2,3-dihydro-1H-inden-2-yl)-6-(1-ethylpropyl)-1-({2-[(4-methyl-1-piperazinyl)carbonyl]phenyl}-methyl)-2,5-piperazinedione |
| 77 | 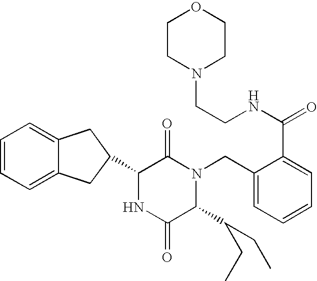 | 546.7 | 2.57(A) | 547;545 | 2-{[(3R,6R)-3-(2,3-dihydro-1H-inden-2-yl)-6-(1-ethylpropyl)-2,5-dioxo-1-piperazinyl]methyl}-N-[2-(4-morpholinyl)ethyl]benzamide |
| 78 | 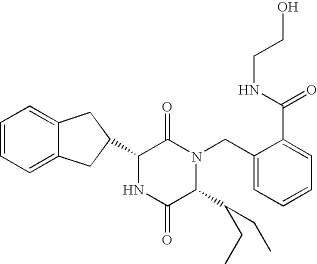 | 477.6 | 2.91(A) | 478;476 | 2-{[(3R,6R)-3-(2,3-dihydro-1H-inden-2-yl)-6-(1-ethylpropyl)-2,5-dioxo-1-piperazinyl]methyl}-N-(2-hydroxyethyl)benzamide |
| 79 | 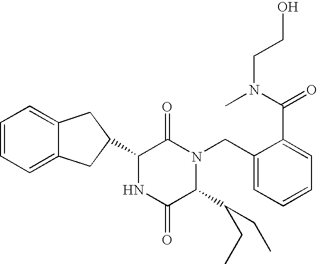 | 491.6 | 3.06(A) | 492;490 | 2-{[(3R,6R)-3-(2,3-dihydro-1H-inden-2-yl)-6-(1-ethylpropyl)-2,5-dioxo-1-piperazinyl]methyl}-N-(2-hydroxy-ethyl)-N-methylbenzamide |
| 80 | 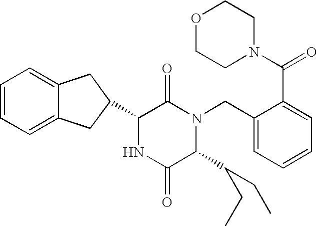 | 503.6 | 3.19(A) | 504;502 | (3R,6R)-3-(2,3-dihydro-1H-inden-2-yl)-6-(1-ethylpropyl)-1-{[2-(4-morpholinylcarbonyl)phenyl]methyl}-2,5-piperazinedione |

### Example 83

### 2-{[(3R,6R)-3-(2,3-Dihydro-1H-inden-2-yl)-6-(1-ethylpropyl)-2,5-dioxo-1-piperazinyl]methyl}-N-4-piperidinylbenzamide

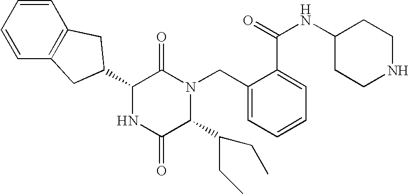

1,1-Dimethylethyl 4-{[(2-{[(3R,6R)-3-(2,3-dihydro-1H-inden-2-yl)-6-(1-ethylpropyl)-2,5-dioxo-1-piperazinyl]methyl}phenyl)carbonyl]amino}-1-piperidinecarboxylate (Int. 17) (845 mg, 1.37 mmol) was treated with 4M hydrogen chloride in dioxan (3 ml, 12 mmol). The mixture was stirred at room temperature for 1 hour before it was evaporated under reduced pressure to leave a yellow foam. The foam was loaded in 1:1 methanol: dichloromethane onto an SCX-SPE column (pre-eluted with methanol). The column was eluted with methanol, followed by 2M ammonia in methanol. The ammonia in methanol fractions afforded 2-{[(3R,6R)-3-(2,3-dihydro-1H-inden-2-yl)-6-(1-ethylpropyl)-2,5-dioxo-1-piperazinyl]methyl}-N-4-piperidinylbenzamide as a pale yellow solid (547 mg).

HPLC (A) Rt=2.54 minutes; m/z [M+H]⁺=517

¹H NMR (CDCl₃) [symbol] 7.44 (brd, 1H), 7.38 (brt, 1H), 7.31 (brt, 1H), 7.24 (m, 2H), 7.18 (m, 4H), 6.78 (m, 1H), 5.12 (d, 1H), 4.45 (d, 1H), 4.13 (d, 1H), 4.07 (m, 1H), 4.03 (dd, 1H), 3.12 (m, 5H), 2.91 (m, 1H), 2.78 (m, 3H), 2.06 (m, 2H), 1.73 (m, 1H), 1.61 (m, 3H), 1.45 (m, 2H), 1.35 (m, 1H), 0.96 (t, 3H), 0.89 (t, 3H).

Example 84 was prepared from Int. 18 by a method analogous to that described for Example 83

| Ex | | | Rt/ | +ve; | |
|---|---|---|---|---|---|
| No | Structure | Mwt | min | −ve | Name |
| 84 | 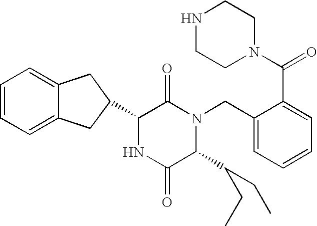 | 502.6 | 2.59(A) | 503;501 | (3R,6R)-3-(2,3-dihydro-1H-inden-2-yl)-6-(1-ethylpropyl)-1-{[2-(1-piperazinylcarbonyl)phenyl]-methyl}-2,5-piperazinedione |

### Example 85

### (3R,6R)-3-(2,3-Dihydro-1H-inden-2-yl)-6-(1-ethylpropyl)-1-{[2-(4-morpholinylmethyl)-phenyl]methyl}-2,5-piperazinedione

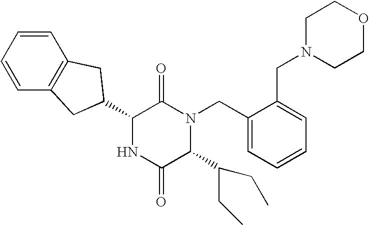

2-{[(3R,6R)-3-(2,3-dihydro-1H-inden-2-yl)-6-(1-ethylpropyl)-2,5-dioxopiperazin-1-yl]-methyl}benzaldehyde (Int. 51) (164 mg, 0.39 mmol) was dissolved in dry tetrahydrofuran (2.5 ml), under nitrogen, and morpholine (34.5 ul, 0.39 mmol) added. The stirred mixture was cooled in an ice/water bath and sodium triacetoxyborohydride (118 mg, 0.55 mmol) added portionwise. The mixture was stirred for 1.3 hours in the cooling bath before it was stirred at room temperature over 3 days. Then the mixture was partitioned between saturated aqueous ammonium chloride solution (2 ml) and dichloromethane (5 ml). The organic phase was washed with saturated aqueous sodium bicarbonate solution (2 ml), dried (hydrophobic frit) and evaporated to leave a fawn solid. The solid was loaded in dichloromethane onto an SCX-SPE column (5 g cartridge, pre-eluted with methanol). The column was eluted with methanol, followed by 2M ammonia in methanol. The ammonia in methanol fractions afforded (3R,6R)-3-(2,3-dihydro-1H-inden-2-yl)-6-(1-ethylpropyl)-1-{[2-(4-morpholinylmethyl)phenyl]methyl}-2,5-piperazinedione as an orange/brown solid (145 mg).

HPLC (A) Rt=2.70 minutes; m/z [M+H]⁺=490

¹H NMR (CDCl₃) δ 8.65 (br d, 1H), 7.27 (m, 4H), 7.19 (m, 4H), 5.41 (d, 1H), 4.47 (d, 1H), 4.15 (dd, 1H), 3.98 (d, 1H), 3.70 (m, 4H), 3.50 (ABq, 2H), 3.21 (m, 2H), 3.14 (m, 1H), 2.97 (m, 1H), 2.89 (m, 1H), 2.44 (m, 4H), 1.69 (m, 4H), 1.35 (m, 1H), 0.92 (t, 3H), 0.90 (t, 3H).

Example 86 was prepared by a method analogous to that described for Example 85.

| Ex | | | Rt/ | +ve; | |
|---|---|---|---|---|---|
| No | Structure | Mwt | min | −ve | Name |
| 86 | 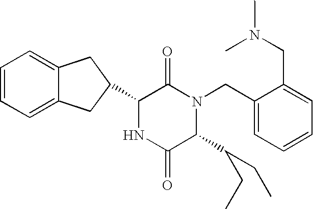 | 447.6 | 2.59(A) | 448;— | (3R,6R)-3-(2,3-dihydro-1H-inden-2-yl)-1-({2-[(dimethylamino)methyl]phenyl}-methyl)-6-(1-ethylpropyl)-2,5-piperazinedione |

### Example 89

### 2-{[(3R,6R)-3-(2,3-Dihydro-1H-inden-2-yl)-6-(1-ethylpropyl)-2,5-dioxo-1-piperazinyl]-methyl}-N-methylbenzenesulfonamide

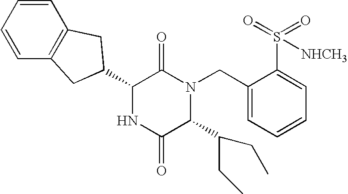

To a solution of 2-{[(3R,6R)-3-(2,3-dihydro-1H-inden-2-yl)-6-(1-ethylpropyl)-2,5-dioxo-1-piperazinyl]methyl}benzenesulfonyl chloride (Int. 20) (100 mg) in dichloromethane (2 ml) was added diisopropylethylamine (39 ul) and methylamine (2M, 0.31 ml, 3 eq., NB generally 3 eq. of volatile amines, 1 eq. of non-volatile) and the mixture stirred for 3 hours at 20° C. Methanol (2 ml) was added and the mixture passed through a 2 g aminopropyl-SPE column to afford the product (85 mg, 83%).

HPLC (A) Rt=3.4 minutes; m/z [M+H]⁺=484

¹H NMR δ 8.03 (d, 1H), 7.54 (t, 1H), 7.45 (t, 1H), 7.32 (d, 1H), 7.22 (m, 4H), 6.47 (br d, 1H), 5.68 (q, 3H), 5.58 (d, 1H), 4.44 (d, 1H), 4.1 (m, 2H), 3.18 (m, 3H), 2.96 (m, 1H), 2.81 (dd, 1H), 2.68 (d, 3H), 1.4-1.8 (m, 5H), 0.96 (2t, 6H).

Examples 90-100 were prepared by methods analogous to that described for Example

| Ex | | | Rt/ | +ve; | |
|---|---|---|---|---|---|
| No | Structure | Mwt | min | −ve | Name |
| 90 | 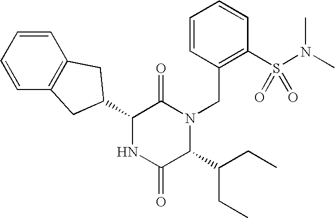 | 497.7 | 3.4(A) | 498;— | 2-{[(3R,6R)-3-(2,3-dihydro-1H-inden-2-yl)-6-(1-ethyl-propyl)-2,5-dioxo-1-piper-azinyl]methyl}-N,N-dimethyl-benzenesulfonamide |
| 91 | 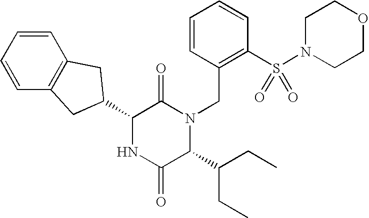 | 539.7 | 3.4(A) | 540;— | (3R,6R)-3-(2,3-dihydro-1H-inden-2-yl)-6-(1-ethylpropyl)-1-{[2-(4-morpholinylsulfonyl)phenyl]-methyl}-2,5-piperazinedione |
| 92 | 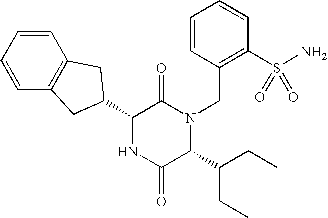 | 469.6 | 3.2(A) | 470;468 | 2-{[(3R,6R)-3-(2,3-dihydro-1H-inden-2-yl)-6-(1-ethylpropyl)-2,5-dioxo-1-piperazinyl]methyl}-benzene-sulfonamide |
| 93 | 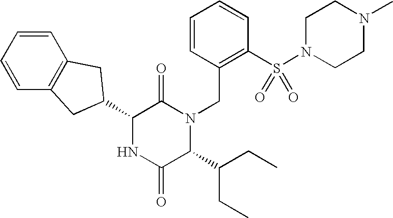 | 552.7 | 2.6(A) | 553;— | (3R,6R)-3-(2,3-dihydro-1H-inden-2-yl)-6-(1-ethylpropyl)-1-({2-[(4-methyl-1-piperazinyl)sulfonyl]-phenyl}-methyl)-2,5-piperazine-dione |
| 94 | 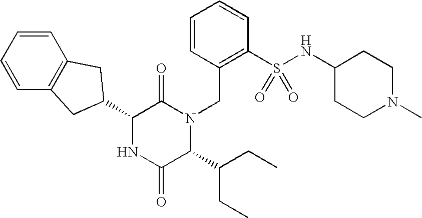 | 566.8 | 2.6(A) | 567;565 | 2-{[(3R,6R)-3-(2,3-dihydro-1H-inden-2-yl)-6-(1-ethylpropyl)-2,5-dioxo-1-piperazinyl]methyl}-N-(1-methyl-4-piperidinyl)-benzene-sulfonamide |
| 95 | 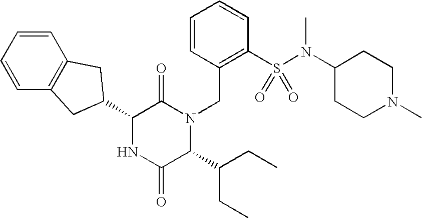 | 580.8 | 2.7(A) | 581;— | 2-{[(3R,6R)-3-(2,3-dihydro-1H-inden-2-yl)-6-(1-ethyl-propyl)-2,5-dioxo-1-piperazinyl]methyl}-N-methyl-N-(1-methyl-4-piperidinyl)-benzenesulfonamide |
| 96 | 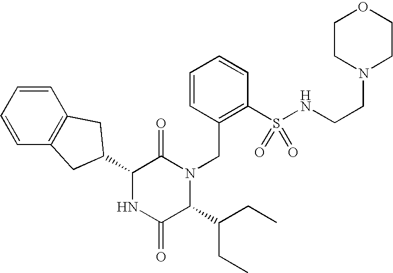 | 582.8 | 2.7(A) | 583;581 | 2-{[(3R,6R)-3-(2,3-dihydro-1H-inden-2-yl)-6-(1-ethylpropyl)-2,5-dioxo-1-piperazinyl]methyl}-N-[2-(4-morpholinyl)ethyl]benzene-sulfonamide |
| 97 | 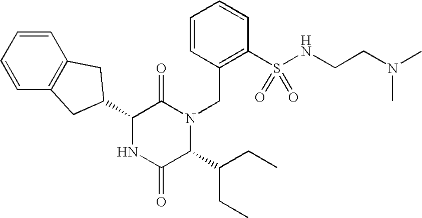 | 540.7 | 2.7(A) | 541;— | 2-{[(3R,6R)-3-(2,3-dihydro-1H-inden-2-yl)-6-(1-ethylpropyl)-2,5-dioxo-1-piperazinyl]methyl}-N-[2-(dimethylamino)ethyl]benzene-sulfonamide |
| 98 | 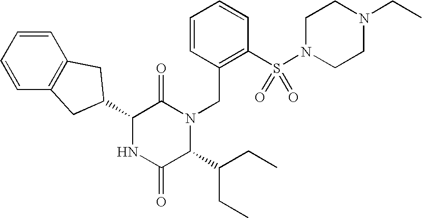 | 566.8 | 2.8(A) | 567;— | (3R,6R)-3-(2,3-dihydro-1H-inden-2-yl)-1-({2-[(4-ethyl-1-piperazinyl)-sulfonyl]phenyl}methyl)-6-(1-ethyl-propyl)-2,5-piperazinedione |
| 99 | 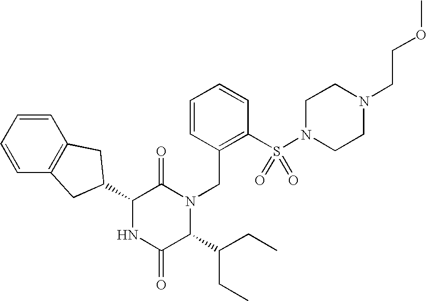 | 596.8 | 2.8(A) | 597;— | (3R,6R)-3-(2,3-dihydro-1H-inden-2-yl)-6-(1-ethylpropyl)-1-{[2-({4-[2-(methoxy)ethyl]-1-piperazinyl}-sulfonyl)phenyl]methyl}-2,5-piperazinedione |
| 100 | 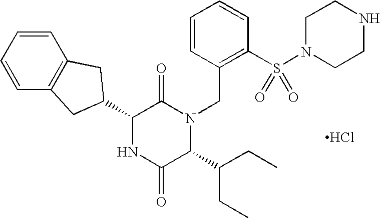 | 575.2 | 2.7(A) | 539;— | (3R,6R)-3-(2,3-dihydro-1H-inden-2-yl)-6-(1-ethylpropyl)-1-{[2-(1-piperazinylsulfonyl)phenyl]methyl}-2,5-piperazinedionehydrochloride |

### Example 102

### (3R,6R)-3-(2,3-Dihydro-1H-inden-2-yl)-6-(1-ethylpropyl)-1-{[2-(4-piperidinylthio)-phenyl]methyl}-2,5-piperazinedione

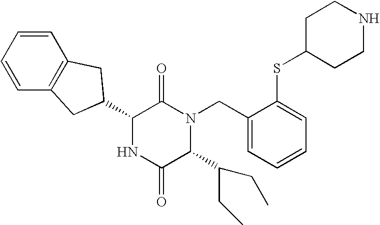

To a solution of 1,1-dimethylethyl 4-[(2-{[(3R,6R)-3-(2,3-dihydro-1H-inden-2-yl)-6-(1-ethylpropyl)-2,5-dioxo-1-piperazinyl]methyl}phenyl)thio]-1-piperidinecarboxylate (Int. 21) (45 mg) in dichloromethane (0.2 mL) was added 4M hydrogen chloride in dioxan (0.1 mL) and the mixture stirred for 2 hours. The mixture was reduced in vacuo and purified by aminopropyl-SPE and mass-directed autoprep to give the title compound (12.2 mg).

HPLC (A) Rt=2.8 minutes; m/z [M+H]⁺=506

¹H NMR δ 7.45 (m, 1H), 7.20 (m, 7H), 6.54 (br s, 1H), 5.32 (d, 1H), 4.51 (d, 1H), 4.17 (dd, 1H), 4.02 (d, 1H), 3.20 (m, 5H), 3.00 (m, 1H), 2.84 (dd, 1H), 2.75 (t, 2H), 1.95-2.20 (br m, 5H), 1.70 (m, 5H), 1.32 (m, 1H), 0.92 (m, 6H).

### Example 104

### (3R,6R)-3-(2,3-Dihydro-1H-inden-2-yl)-6-(1-ethylpropyl)-1-{[2-(4-piperidinylsulfonyl)-phenyl]methyl}-2,5-piperazinedione hydrochloride

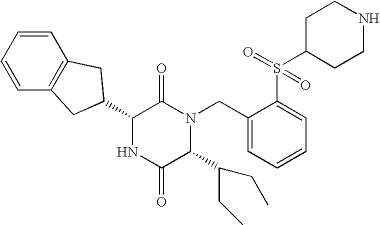

To a solution of 1,1-dimethylethyl 4-[(2-{[(3R,6R)-3-(2,3-dihydro-1H-inden-2-yl)-6-(1-ethylpropyl)-2,5-dioxo-1-piperazinyl]methyl}phenyl)sulfonyl]-1-piperidinecarboxylate (Int. 22) (94 mg) in dichloromethane (0.4 mL) was added 4M hydrogen chloride in dioxan (0.27 mL) and the mixture stirred for 3 days. The solvent was removed in vacuo to give the title compound (80 mg).

HPLC (A) Rt=2.7 minutes; m/z [M+H]⁺=538

¹H NMR (CDCl₃) δ 8.80 (br s, 2H), 8.53 (br d, 1H), 7.88 (d, 1H), 7.78 (t, 1H), 7.59 (t, 1H), 7.34 (d, 1H), 7.20 (m, 2H), 7.11 (m, 2H), 5.16 (d, 1H), 4.87 (d, 1H), 4.03 (dd, 1H), 3.96 (d, 1H), 3.74 (tt, 1H), 3.34 (m, 2H (obscured by water)), 2.80-3.05 (m, 6H), 2.07 (br d, 1H), 1.90 (m, 3H), 1.45-1.66 (m, 4H), 1.26 (m, 1H), 0.89 (t, 3H), 0.78 (t, 3H).

### Example 105

### (3R,6R)-3-(2,3-Dihydro-1H-inden-2-yl)-6-(1-ethylpropyl)-1-{[2-({1-[2-methoxy-ethyl]-4-piperidinyl}sulfonyl)phenyl]methyl}-2,5-piperazinedione

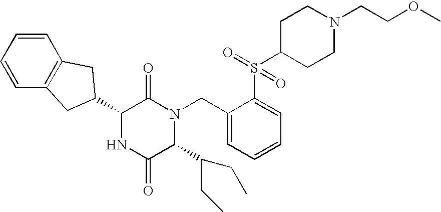

A solution of (3R,6R)-3-(2,3-dihydro-1H-inden-2-yl)-6-(1-ethylpropyl)-1-{[2-(4-piperidinyl-sulfonyl)phenyl]methyl}-2,5-piperazinedione hydrochloride (Ex. 104) (150 mg, 0.26 mmol) in methanol was loaded onto an aminopropyl SPE column, washing with methanol. Concentration yielded (3R,6R)-3-(2,3-dihydro-1H-inden-2-yl)-6-(1-ethylpropyl)-1-{[2-(4-piperidinylsulfonyl)phenyl]methyl}-2,5-piperazinedione as a green oil (131 mg, 93%).

HPLC (A) Rt=2.66 minutes; m/z [M+H]⁺=538

To a solution of (3R,6R)-3-(2,3-dihydro-1H-inden-2-yl)-6-(1-ethylpropyl)-1-{[2-(4-piperidinylsulfonyl)phenyl]methyl}-2,5-piperazinedione (131 mg, 0.24 mmol) in dimethylformamide (0.6 ml) was added potassium carbonate (40 mg, 0.29 mmol), 2-bromoethylmethylether (22.6 μL, 0.24 mmol) and tetrabutylammoniumiodide (18 mg, 0.05 mmol). The reaction was heated to 50° C. for 3 hours. Further 2-bromoethylmethylether (22.6 μL, 0.24 mmol), tetrabutylammoniumiodide (18 mg, 0.05 mmol) and potassium carbonate (40 mg, 0.29 mmol) were added and the reaction was heated at 50° C. for 1 hour. The reaction was loaded onto an SCX-SPE column, washed with methanol then eluted with 2M ammonia/methanol. Concentration gave (3R,6R)-3-(2,3-dihydro-1H-inden-2-yl)-6-(1-ethylpropyl)-1-{[2-({1-[2-(methoxy)ethyl]-4-piperidinyl}sulfonyl)phenyl]methyl}-2,5-piperazinedione as a clear oil (117 mg, 82%).

HPLC (A) Rt=2.72 minutes; m/z [M+H]⁺=596

¹H NMR (CDCl₃) δ 8.05 (d, 1H), 7.6 (t, 1H), 7.5 (t, 1H), 7.35-7.2 (m, 5H), 6.5 (br s, 1H), 5.35 (d, 1H), 4.95 (d, 1H), 4.15 (dd, 1H), 4.05 (d, 1H), 3.5 (m, 2H), 3.35 (s, 3H), 3.2-2.95 (m, 7H), 2.85 (m, 1H), 2.58 (m, 2H), 2.05-1.8 (m, 6H), 1.75-1.5 (m, 4H), 1.33 (m, 1H), 0.95 (t, 3H), 0.85 (t, 3H).

Examples 106-111 were prepared by methods analogous to that described for Intermediate 21 (sulfides) and Intermediate 22 (sulfones).

| Ex | | | Rt/ | +ve; | |
|---|---|---|---|---|---|
| No | Structure | Mwt | min | −ve | Name |
| 106 | 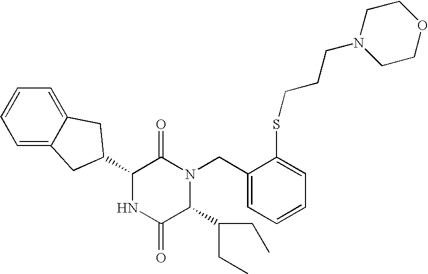 | 549.7 | 2.79(A) | 550;— | (3R,6R)-3-(2,3-dihydro-1H-inden-2-yl)-6-(1-ethylpropyl)-1-[(2-{[3-(4-morpholinyl)-propyl]thio}phenyl)methyl]-2,5-piperazinedione |
| 107 | 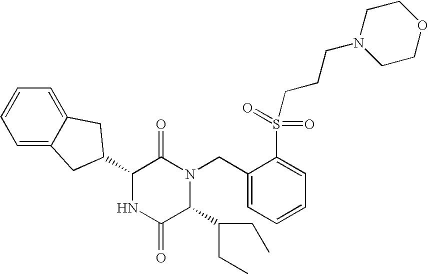 | 581.7 | 2.74(A) | 582;580 | (3R,6R)-3-(2,3-dihydro-1H-inden-2-yl)-6-(1-ethylpropyl)-1-[(2-{[3-(4-morpholinyl)-propyl]sulfonyl}phenyl)methyl]-2,5-piperazinedione |
| 108 | 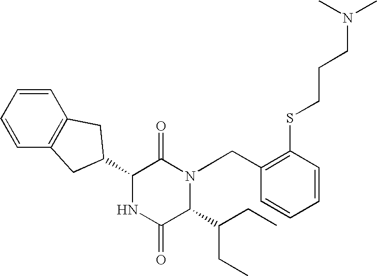 | 507.7 | 2.71(A) | 508;506 | (3R,6R)-3-(2,3-dihydro-1H-inden-2-yl)-1-[(2-{[3-(dimethylamino)propyl]thio}-phenyl)methyl]-6-(1-ethyl-propyl)-2,5-piperazinedione |
| 109 | 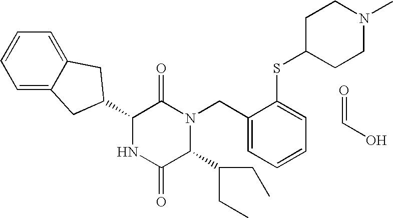 | 565.7 | 2.81(A) | 520;— | (3R,6R)-3-(2,3-dihydro-1H-inden-2-yl)-6-(1-ethylpropyl)-1-({2-[(1-methyl-4-piperidinyl)thio]phenyl}-methyl)-2,5-piperazinedioneformate |
| 110 | 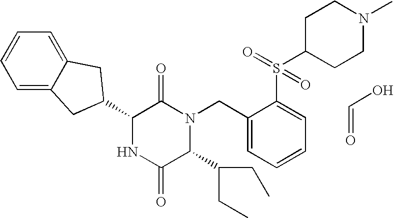 | 597.7 | 2.71(A) | 552;549 | (3R,6R)-3-(2,3-dihydro-1H-inden-2-yl)-6-(1-ethylpropyl)-1-({2-[(1-methyl-4-piperidinyl)sulfonyl]phenyl}-methyl)-2,5-piperazinedioneformate |
| 111 | 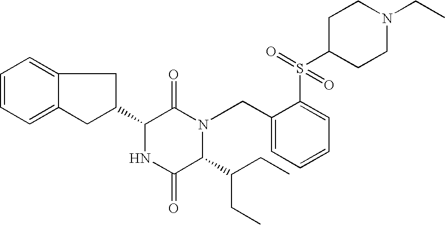 | 565.8 | 2.70(A) | 566;564 | (3R,6R)-3-(2,3-dihydro-1H-inden-2-yl)-1-({2-[(1-ethyl-4-piperidinyl)sulfonyl]phenyl}-methyl)-6-(1-ethylpropyl)-2,5-piperazinedione |

### Example 112

### (3R,6R)-1-[(2-Aminophenyl)methyl]-3-(2,3-dihydro-1H-inden-2-yl)-6-(1-ethylpropyl)-2,5-piperazinedione

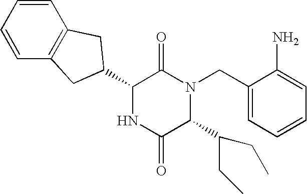

A solution of (3R,6R)-3-(2,3-dihydro-1H-inden-2-yl)-6-(1-ethylpropyl)-1-(2-nitrobenzyl)-piperazine-2,5-dione (Ex. 63) (1.99 g, 4.57 mmol) in ethanol (36 ml) was hydrogenated at room temperature and pressure over 10% Pd/carbon (707 mg) for 2.5 hours. The reaction was filtered through celite and the solvent removed in vacuo to give (3R,6R)-1-[(2-aminophenyl)methyl]-3-(2,3-dihydro-1H-inden-2-yl)-6-(1-ethylpropyl)-2,5-piperazine-dione as a light brown solid (1.79 g, 97%).

HPLC (A) Rt=3.34 minutes; m/z [M+H]⁺=406/407

¹H NMR (CDCl₃) δ 7.25 (m, 1H), 7.18 (m, 3H), 7.13 (dt, 1H), 7.05 (dd, 1H), 6.68 (m, 3H), 5.45 (d, 1H), 4.29 (br s, 2H), 4.07 (dd, 1H), 4.03 (d, 1H), 3.91 (d, 1H), 3.14 (m, 3H), 2.93 (m, 1H), 2.76 (m, 1H), 1.88 (m, 1H), 1.67 (m, 2H), 1.25 (m, 2H), 1.00 (t, 3H), 0.94 (t, 3H).

### Example 113

### N-(2-{[(3R,6R)-3-(2,3-Dihydro-1H-inden-2-yl)-6-(1-ethylpropyl)-2,5-dioxo-1-piperazinyl]methyl}phenyl)methanesulfonamide

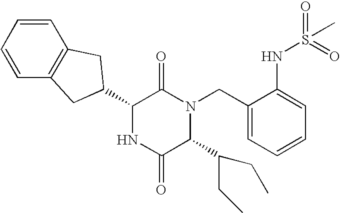

To a solution of (3R,6R)-1-[(2-aminophenyl)methyl]-3-(2,3-dihydro-1H-inden-2-yl)-6-(1-ethylpropyl)-2,5-piperazinedione (Ex. 112) (150 mg, 0.37 mmol) in dry dichloromethane (2.5 ml) at 0° C. was added triethylamine (0.26 ml) and 4-dimethylaminopyridine (450 μg). After five minutes mesyl chloride (34 μl, 0.74 mmol) was added and the reaction stirred at room temperature until absence of starting material was detected by LCMS. The reaction was concentrated and the residue dissolved in tetrahydrofuran (3.5 ml) and treated with 1M sodium hydroxide solution (0.7 ml). After one hour the reaction was neutralised and extracted with ethyl acetate (5 ml). The organic phase was separated, dried (Na₂SO₄) and concentrated. The residue was purified by silica column chromatography to yield N-(2-{[(3R,6R)-3-(2,3-dihydro-1H-inden-2-yl)-6-(1-ethylpropyl)-2,5-dioxo-1-piperazinyl]methyl}phenyl)methanesulfonamide as a white solid (111 mg, 62 HPLC (A) Rt=3.15 minutes; m/z [M+H]⁺=484

¹H NMR (CDCl₃) δ 9.2 (s, 1H), 7.58 (d, 1H), 7.39 (dt, 1H), 7.25 (m, 1H), 7.23-7.13 (m, 5H), 6.38 (br d, 1H), 5.13 (d, 1H), 4.19 (d, 1H), 4.12 (d, 1H), 4.06 (dd, 1H), 3.18 (m, 3H), 3.1 (s, 3H), 2.90 (m, 1H), 2.7 (m, 1H), 1.84 (m, 1H), 1.70 (m, 2H), 1.65 (m, 1H), 1.32 (m, 1H), 1.09 (t, 3H), 0.94 (t, 3H).

Examples 114-115 were prepared by methods analogous to that described for Example 113

| Ex | | | Rt/ | +ve; | |
|---|---|---|---|---|---|
| No | Structure | Mwt | min | −ve | Name |
| 114 | 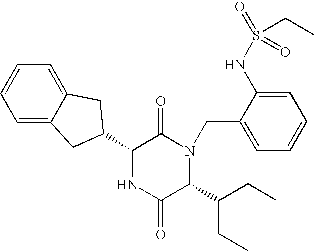 | 497.6 | 3.25(A) | 498;496 | N-(2-{[(3R,6R)-3-(2,3-dihydro-1H-inden-2-yl)-6-(1-ethylpropyl)-2,5-dioxo-1-piperazinyl]-methyl}phenyl)-ethane-sulfonamide |
| 115 | 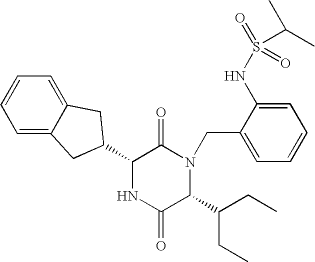 | 511.6 | 3.5 (A) | 512;510 | N-(2-{[(3R,6R)-3-(2,3-dihydro-1H-inden-2-yl)-6-(1-ethylpropyl)-2,5-dioxo-1-piperazinyl]-methyl}phenyl)-2-propanesulfonamide |

### Example 116

### N-(2-{[(3R,6R)-3-(2,3-Dihydro-1H-inden-2-yl)-6-(1-ethylpropyl)-2,5-dioxo-1-piperazinyl]methyl}phenyl)-N-methylmethanesulfonamide

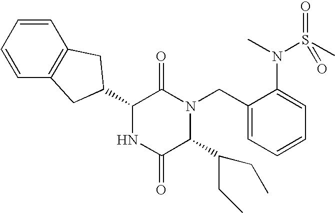

To a solution of N-(2-{[(3R,6R)-3-(2,3-dihydro-1H-inden-2-yl)-6-(1-ethylpropyl)-2,5-dioxo-1-piperazinyl]methyl}phenyl)methanesulfonamide (Ex. 113) (178 mg, 0.37 mmol) in dimethylformamide (1 ml) was added potassium carbonate (102 mg, 0.74 mmol) followed by iodomethane (69 μl, 1.1 mmol) and the reaction stirred at room temperature for 18 hours. The reaction was quenched by the addition of ammonia in methanol solution (2M, 0.74 ml). The reaction was then diluted with dichloromethane (4 ml) and water (4 ml). The organic phase was separated, passed through a hydrophobic frit and concentrated. The residue was purified by silica column chromatography to yield N-(2-{[(3R,6R)-3-(2,3-dihydro-1H-inden-2-yl)-6-(1-ethylpropyl)-2,5-dioxo-1-piperazinyl]-methyl}phenyl)-N-methylmethanesulfonamide as a white solid (62 mg, 34%).

HPLC (A) Rt=3.32 minutes; m/z [M+H]⁺=498

¹H NMR at room temperature showed clear rotomers which coalesced at 120° C. to give the following spectrum: ¹H NMR (DMSO-d6, 120° C.): δ 7.91 (br s 1H), 7.50 (m, 1H), 7.36 (m, 2H), 7.23 (m, 3H), 7.15 (m, 2H), 5.06 (d, 1H), 4.43 (d, 1H), 3.98 (dd, 1H), 3.84 (d, 1H), 3.22 (m, 3H), 3.10-2.98 (m, 8H), 1.6-1.28 (m, 5H), 0.92-0.8 (m, 6H).

### Example 117

### (3R,6R)-3-(2,3-Dihydro-1H-inden-2-yl)-1-{[2-(1,1-dioxido-2-isothiazolidinyl)-phenyl]methyl}-6-(1-ethylpropyl)-2,5-piperazinedione

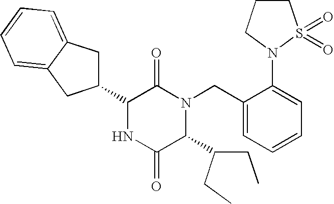

To a solution of (3R,6R)-1-[(2-aminophenyl)methyl]-3-(2,3-dihydro-1H-inden-2-yl)-6-(1-ethylpropyl)-2,5-piperazinedione (Ex. 112) (130 mg, 0.32 mmol) in dichloromethane (1.5 ml) at 0° C. was added triethylamine (0.22 ml) followed by 3-chloropropane sulfonyl chloride (77 ul, 0.64 mmol). Tetrabutyl ammonium iodide (1.2 mg) was added and the reaction stirred at room temperature for 18 hours. The reaction was concentrated and the residue dissolved in ethanol (1 ml), treated with triethylamine (0.11 ml) and heated at reflux for 5 hours. The reaction was concentrated and the residue dissolved in dichloromethane and washed with water. The organic phase was collected via a hydrophobic frit, concentrated and the residue purified by silica column chromatography to give (3R,6R)-3-(2,3-dihydro-1H-inden-2-yl)-1-{[2-(1,1-dioxido-2-isothiazolidinyl)-phenyl]methyl}-6-(1-ethylpropyl)-2,5-piperazinedione as a pale yellow solid (58 mg, 36%).

HPLC (A) Rt=3.3 minutes; m/z [M+H]⁺=510

¹H NMR (CDCl₃) δ 7.51 (d, 1H), 7.35 (m, 3H), 7.12 (m, 5H), 5.28 (d, 1H), 4.31 (d, 1H), 4.06 (m, 2H), 3.69 (m, 2H), 3.37 (m, 2H), 3.15 (m, 3H), 2.93 (m, 1H), 2.79 (m, 1H), 2.57 (m, 2H), 1.77 (m, 1H), 1.64 (m, 3H), δ 1.3 (m, 1H), 0.95 (m, 6H).

### Example 118

### N-(2-{[(3R,6R)-3-(2,3-Dihydro-1H-inden-2-yl)-6-(1-ethylpropyl)-2,5-dioxo-1-piperazinyl]methyl}phenyl)acetamide GSK681884A R11351/189/1

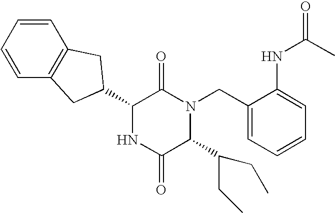

To a solution of (3R,6R)-1-[(2-aminophenyl)methyl]-3-(2,3-dihydro-1H-inden-2-yl)-6-(1-ethylpropyl)-2,5-piperazinedione (Ex. 112) (70 mg, 0.17 mmol) in anhydrous dichloromethane (1 ml) under an atmosphere of nitrogen was added pyridine (32 ul) and acetyl chloride (14.7 ul). After stirring for 16 hr the reaction mixture was partitioned between dichloromethane and water. The phases were separated via a hydrophobic frit and the organic phase was loaded onto a 2 g SCX-SPE cartridge and eluted with methanol. Evaporation of the methanol in vacuo and freeze drying from dioxan gave N-(2-{[(3R,6R)-3-(2,3-dihydro-1H-inden-2-yl)-6-(1-ethylpropyl)-2,5-dioxo-1-piperazinyl]methyl}phenyl)acetamide as a white lyophilate (42 mg).

HPLC (A) Rt=3.21 minutes; m/z [M+H]⁺=448

¹H NMR (CDCl₃) δ 9.59 (s, 1H); 8.28 (d, 1H); 7.37 (t, 1H); 7.27 (t, 1H); 7.20 (m, 4H); 7.07 (t, 1H); 6.85 (s, 1H); 5.25 (d, 1H); 4.16 (d, 1H); 4.04 (m, 1H); 4.01 (d, 1H); 3.15 (m, 3H); 2.91 (m, 1H); 2.78 (m, 1H); 2.26 (s, 3H); 1.88 (m, 1H); 1.70 (m, 2H); 1.56 (m, 1H); 1.31 (m, 1H); 1.1 (t, 3H); 0.96 (t, 3H)

### Example 119

### N¹-(2-{[(3R,6R)-3-(2,3-Dihydro-1H-inden-2-yl)-6-(1-ethylpropyl)-2,5-dioxo-1-piperazinyl]methyl}phenyl)-N³,N³-dimethyl-β-alaninamide formate

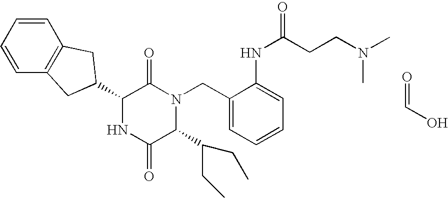

To a solution of (3R,6R)-1-[(2-aminophenyl)methyl]-3-(2,3-dihydro-1H-inden-2-yl)-6-(1-ethylpropyl)-2,5-piperazinedione (Ex. 112) (70 mg, 0.17 mmol) in anhydrous dichloromethane (1 ml) under an atmosphere of nitrogen was added pyridine (32 ul) and N,N-dimethyl-β-alanyl chloride hydrochloride (36 mg). After stirring for 16 hr the reaction mixture was partitioned between dichloromethane and water. The phases were separated via a hydrophobic frit and the organic phase was loaded onto a 2 g SCX-SPE cartridge and eluted with methanol, then 1N ammonia in methanol. The basic fraction was evaporated in vacuo and the residue further purified using the mass-directed autoprep system. Freeze drying from dioxan gave N¹-(2-{[(3R,6R)-3-(2,3-dihydro-1H-inden-2-yl)-6-(1-ethylpropyl)-2,5-dioxo-1-piperazinyl]methyl}phenyl)-N³,N³-dimethyl-β-alaninamide formate (18.8 mg) as a white lyophilate.

HPLC (A) Rt=2.60 minutes; m/z [M+H]⁺=505

¹H NMR (CDCl₃) 10.14 (s, 1H); 8.05 (d, 2H); 7.33 (t, 1H); 7.30-7.14 (m, 5H); 7.10 (t, 1H); 6.47 (s, 1H); 5.16 (d, 1H); 4.08 (m, 3H); 3.15 (m, 3H); 2.93 (m, 1H); 2.80 (m, 3H); 2.67 (t, 2H); 2.40 (s, 6H); 1.83 (m, 2H); 1.68 (m, 2H); 1.57 (m, 1H); 1.03 (t, 3H); 0.92 (t, 3H).

Examples 120-121 were prepared by methods analogous to that described for Example 119

| Ex | | | Rt/ | +ve; | |
|---|---|---|---|---|---|
| No | Structure | Mwt | min | −ve | Name |
| 120 | 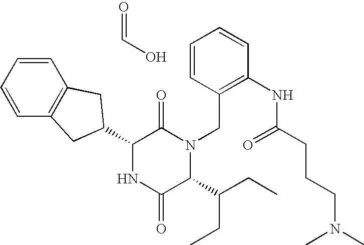 | 518.7 | 2.62(A) | 519;517 | N-(2-{[(3R,6R)-3-(2,3-dihydro-1H-inden-2-yl)-6-(1-ethyl-propyl)-2,5-dioxo-1-piperazinyl]-methyl}-phenyl)-4-(dimethyl-amino)butanamide formate |
| 121 | 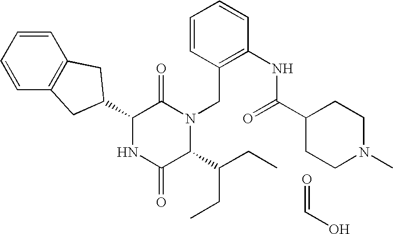 | 530.7 | 2.64(A) | 531;529 | N-(2-{[(3R,6R)-3-(2,3-dihydro-1H-inden-2-yl)-6-(1-ethyl-propyl)-2,5-dioxo-1-piperazinyl]-methyl}phenyl)-1-methyl-4-piperidinecarboxamide formate |

### Example 122

### (3R,6R)-3-(2,3-Dihydro-1H-inden-2-yl)-6-(1-ethylpropyl)-1-{[2-(2-oxo-1-pyrrolidinyl)-phenyl]methyl}-2,5-piperazinedione

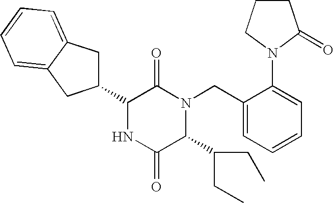

Potassium carbonate (126 mg), cuprous iodide (18 mg), (1R,2R)-(−)-N,N′-dimethyl-cyclohexane-1,2-diamine (48 mg), 2-pyrrolidinone (62 mg) and (3R,6R)-1-[(2-bromophenyl)methyl]-3-(2,3-dihydro-1H-inden-2-yl)-6-(1-ethylpropyl)-2,5-piperazinedione (Ex. 30) (200 mg, 0.42 mmol) and dioxan (0.4 ml) were sequentially added to a 2 ml microwave tube. The mixture was heated with stirring at 15° C. for 4000 seconds in a microwave (Emrys™ Optimizer). The reaction mixture was diluted with dichloromethane and purified on an SPE cartridge (5 g, silica) eluting with methanol: dichloromethane (0 to 3%). The relevant fractions were evaporated in vacuo and further purified on a 2 g SCX-SPE cartridge eluting with dichloromethane then methanol. Evaporation of the methanol in vacuo and freeze drying from dioxan gave (3R,6R)-3-(2,3-dihydro-1H-inden-2-yl)-6-(1-ethylpropyl)-1-{[2-(2-oxo-1-pyrrolidinyl)phenyl]methyl}-2,5-piperazinedione as a white lyophilate (27 mg).

HPLC (A) Rt=3.14 minutes; m/z [M+H]⁺=474

¹H NMR (CDCl₃) δ 7.3 (m, 3H), 7.20 (m, 3H), 7.14 (m, 2H), 5.18 (d, 1H), 4.05 (d, 1H), 4.04 (d, 1H), 3.94 (d, 1H), 3.83 (m, 1H), 3.75 (m, 1H), 3.12 (d, 3H), 2.86 (m, 2H), 2.58 (t, 2H), 2.25 (m, 3H), 1.71 (m, 1H), 1.59 (m, 3H), 1.29 (m, 1H), 0.92 (t, 3H), 0.88 (t, 3H).

### Example 123

### (3R,6R)-3-(2,3-dihydro-1H-inden-2-yl)-1-{[4-{[2-(dimethylamino)ethyl]oxy}-2-(methylsulfonyl)phenyl]methyl}-6-(1-ethylpropyl)-2,5-piperazinedione

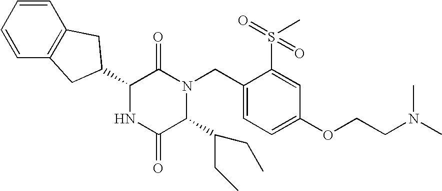

2-{[4-(Aminomethyl)-3-(methylsulfonyl)phenyl]oxy}-N,N-dimethylethanamine (Int. 11) (0.42 g) and 2-ethylbutanal (0.20 mL) were dissolved in 2,2,2-trifluoroethanol (10 mL). Triethylamine (0.20 mL) was then added, and the mixture was stirred overnight then (2R)-2,3-dihydro-1H-inden-2-yl({[(1,1-dimethylethyl)oxy]carbonyl}amino)ethanoic acid (0.45 g) and 4-chlorophenylisonitrile were added and the mixture was stirred for 3 days. The mixture was dissolved in methanol at 0° C. and acetyl chloride (1.7 mL) was added. It was then stirred for 1 hour, but LCMS showed no loss of the Boc group, so acetyl chloride (a further 1.7 mL) was added and the solution was stirred overnight. The mixture was concentrated under reduced pressure and dissolved in chloroform (20 mL). This solution was stirred with aqueous sodium hydrogen carbonate (20 mL) for 1 hour. The organic phase was separated and the aqueous phase was extracted twice with chloroform. The organic extracts were concentrated under reduced pressure then dissolved again in chloroform (20 mL). Acetic acid (12 mL) was added and the mixture was stirred overnight. The mixture was concentrated under reduced pressure to give a yellow solid which was purified using an SPE cartridge followed by mass-directed autoprep and finally by reverse-phase HPLC to give the title compound as a solid

¹H NMR (CDCl₃) δ 7.57 (d, 1H), 7.46 (d, 1H), 7.41 (s, 1H), 7.24-7.14 (m, 4H), 6.89 (s, 1H), 5.11 (d, 1H), 4.51-4.41 (m, 2H), 4.26 (d, 1H), 4.07-3.95 (m, 2H), 3.72-3.46 (m, 2H), 3.18-3.04 (m, 5H), 2.98 (s, 6H), 2.94-2.73 (m, 3H), 1.76-1.52 (m, 4H), 1.43-1.30 (m, 1H), 1.00-0.87 (m, 6H)

### Example 124

### 4-{[(3R,6R)-3-(2,3-Dihydro-1H-inden-2-yl)-6-(2-methylpropyl)-2,5-dioxo-1-piperazinyl]methyl}-N,N-dimethylbenzenesulfonamide

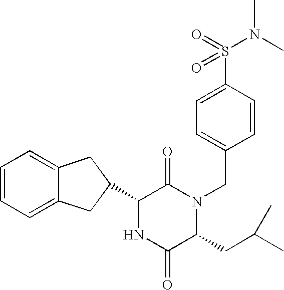

To a solution of 4-(aminomethyl)-N,N-dimethylbenzenesulfonamide (467 mg) in methanol (10 ml) was added (2R)-2,3-dihydro-1H-inden-2-yl({[(1,1-dimethylethyl)-oxy]carbonyl}amino)ethanoic acid (640 mg), 2-{[(1,1-dimethylethyl)(dimethyl)silyl]oxy}-phenyl isocyanide (515 mg) and then 3-methylbutanal (0.24 ml). The reaction was then stirred for 18 hours. The reaction was then cooled to 0° C. and treated with acetyl chloride (0.94 ml) and left to warm to ambient temperature overnight. The reaction was concentrated and the residue partitioned between chloroform (10 ml) and sodium bicarbonate (10 ml) and stirred at room temperature for 72 hours. The organic was collected and the aqueous extracted with further chloroform. The combined organics were then concentrated and the residue dissolved in methanol and passed through a SCX SPE and eluted in methanol. The methanol was concentrated to yield a residue which was purified by Redisep (12 g) column to give the title compound as a yellow solid, 290 mg.

LCMS (A) Rt=3.24 minutes; m/z [M+H]⁺=484; m/z [M−H]⁻=482

### Example 125

### (3R,6R)-3-(2,3-Dihydro-1H-inden-2-yl)-1-[(3-{[ethyl(methyl)amino]methyl}phenyl)-methyl]-6-(1-ethylpropyl)-2,5-piperazinedione

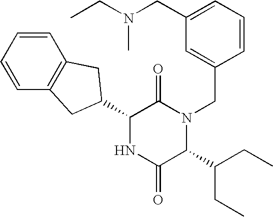

A mixture of N-{[3-(aminomethyl)phenyl]methyl}-N-methylethanamine (0.2 mmol) and 2-ethylbutanal (0.2 mmol) in methanol (1 ml) was treated with diisopropylethylamine (0.3 mmol) then a solution of (2R)-2,3-dihydro-1H-inden-2-yl({[(1,1-dimethylethyl)oxy]carbonyl}amino)ethanoic acid (0.2 mmol) in methanol (1 ml) and then with a solution of 2-{[(1,1-dimethylethyl)(dimethyl)silyl]oxy}phenyl isocyanide (0.2 mmol) in methanol (1 ml). The reaction was then stirred for 2 days. The reaction was then cooled to 0° C. and treated with acetyl chloride (200 ul) and left to warm to ambient temperature overnight. The reaction was concentrated and the residue partitioned between chloroform and sodium bicarbonate and heated to 50° C. for 4 hours. The organic was collected and concentrated and the residue purified by Autoprep HPLC (10-35% CH₃CN). Concentration of the appropriate fractions yielded the title compound as a gum, 10.5 mg. LCMS (A) Rt=2.6 minutes; m/z [M+H]⁺=462

The following Examples were prepared by methods analogous to that described for Example 124

| Ex | | | Rt/ | −ve; | |
|---|---|---|---|---|---|
| No | Structure | MW | min | −ve | Name |
| 126 | 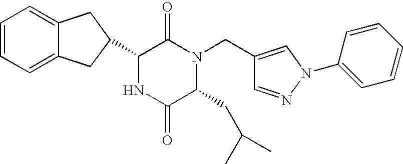 | 442.6 | 3.32(A) | 443;441 | (3R,6R)-3-(2,3-dihydro-1H-inden-2-yl)-6-(2-methylpropyl)-1-[(1-phenyl-1H-pyrazol-4-yl)methyl]-2,5-piperazinedione |
| 127 | 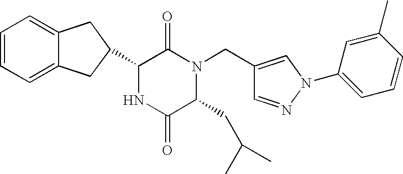 | 456.6 | 3.44(A) | 457;455 | (3R,6R)-3-(2,3-dihydro-1H-inden-2-yl)-1-{[1-(3-methylphenyl)-1H-pyrazol-4-yl]methyl}-6-(2-methylpropyl)-2,5-piperazinedione |
| 128 | 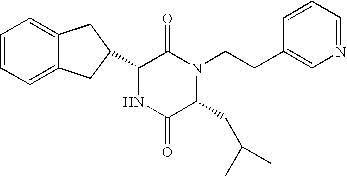 | 391.5 | 2.61(A) | 392;390 | (3R,6R)-3-(2,3-dihydro-1H-inden-2-yl)-6-(2-methylpropyl)-1-[2-(3-pyridinyl)ethyl]-2,5-piperazinedione |
| 129 | 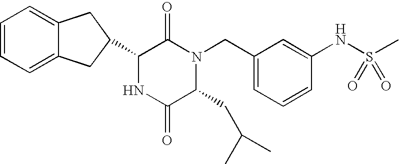 | 469.6 | 3.09(A) | 470;468 | N-(3-{[(3R,6R)-3-(2,3-dihydro-1H-inden-2-yl)-6-(2-methylpropyl)-2,5-dioxo-1-piperazinyl]methyl}phenyl)-methanesulfonamide |
| 130 | 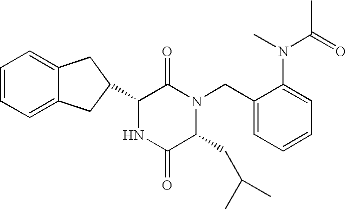 | 447.6 | 3.01(A) | 448;446 | N-(2-{[(3R,6R)-3-(2,3-dihydro-1H-inden-2-yl)-6-(2-methylpropyl)-2,5-dioxo-1-piperazinyl]methyl}-phenyl)-N-methylacetamide |
| 131 | 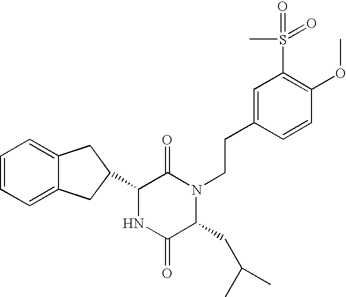 | 498.6 | 3.18(A) | 499;497 | (3R,6R)-3-(2,3-dihydro-1H-inden-2-yl)-1-{2-[4-(methyloxy)-3-(methylsulfonyl)phenyl]ethyl}-6-(2-methylpropyl)-2,5-piperazinedione |

The following Example was prepared by a method analogous to that described for Example 125

| Ex | | | Rt/ | +ve; | |
|---|---|---|---|---|---|
| No | Structure | Mwt | min | −ve | Name |
| 132 | 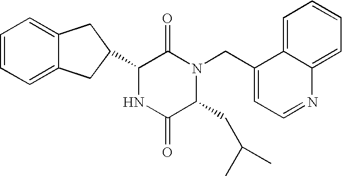 | 427.5 | 3.1(A) | 428;426 | (3R,6R)-3-(2,3-dihydro-1H-inden-2-yl)-6-(2-methylpropyl)-1-(4-quinolinylmethyl)-2,5-piperazinedione |

The following Examples were prepared by methods analogous to that described for Example 1, optionally with the addition of a base such as triethylamine or DIPEA if the hydrochloride salts of amines were used.

| Ex | | | Rt/ | +ve; | |
|---|---|---|---|---|---|
| No | Structure | Mwt | min | −ve | Name |
| 133 | 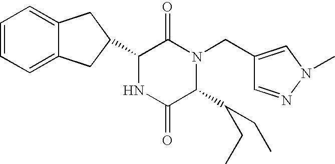 | 394.5 | 2.99(A) | 395;393 | (3R,6R)-3-(2,3-dihydro-1H-inden-2-yl)-6-(1-ethylpropyl)-1-[(1-methyl-1H-pyrazol-4-yl)methyl]-2,5-piperazinedione |
| 134 | 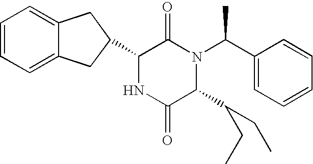 | 404.5 | 3.62(A) | 405;— | (3R,6R)-3-(2,3-dihydro-1H-inden-2-yl)-6-(1-ethylpropyl)-1-[(1S)-1-phenylethyl]-2,5-piperazinedione |
| 135 | 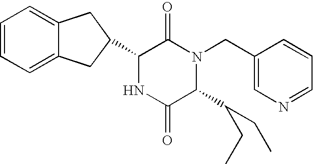 | 391.5 | 2.90(A) | 392;390 | (3R,6R)-3-(2,3-dihydro-1H-inden-2-yl)-6-(1-ethylpropyl)-1-(3-pyridinylmethyl)-2,5-piperazinedione |
| 136 | 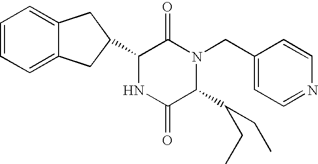 | 391.5 | 2.90(A) | 392;390 | (3R,6R)-3-(2,3-dihydro-1H-inden-2-yl)-6-(1-ethylpropyl)-1-(4-pyridinylmethyl)-2,5-piperazinedione |
| 137 | 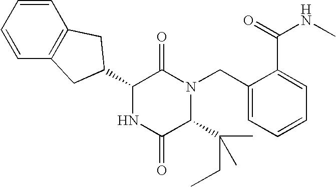 | 447.6 | 3.04(A) | 448;446 | 2-{[(3R,6R)-3-(2,3-dihydro-1H-inden-2-yl)-6-(1,1-dimethylpropyl)-2,5-dioxo-1-piperazinyl]methyl}-N-methylbenzamide |
| 138 | 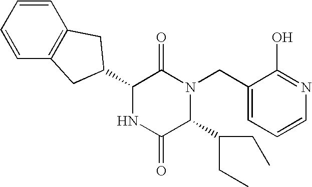 | 407.5 | 2.91(A) | 408;406 | (3R,6R)-3-(2,3-dihydro-1H-inden-2-yl)-6-(1-ethylpropyl)-1-[(2-oxo-1,2-dihydro-3-pyridinyl)methyl]-2,5-piperazinedione |
| 139 | 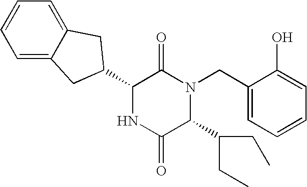 | 406.5 | 3.5 (A) | 407;405 | (3R,6R)-3-(2,3-dihydro-1H-inden-2-yl)-6-(1-ethylpropyl)-1-[(2-hydroxyphenyl)methyl]-2,5-piperazinedione |
| 140 | 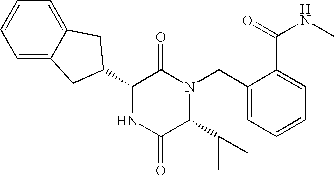 | 419.5 | 2.85(A) | 420;418 | 2-{[(3R,6R)-3-(2,3-dihydro-1H-inden-2-yl)-6-(1-methylethyl)-2,5-dioxo-1-piperazinyl]methyl}-N-methylbenzamide |
| 141 | 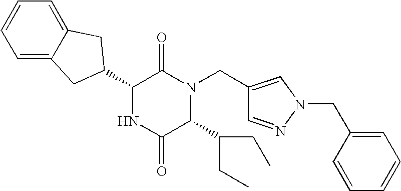 | 470.6 | 3.42(A) | 471;515 | (3R,6R)-3-(2,3-dihydro-1H-inden-2-yl)-6-(1-ethylpropyl)-1-{[1-(phenylmethyl)-1H-pyrazol-4-yl]methyl}-2,5-piperazinedione |
| 142 | 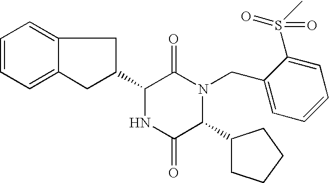 | 466.6 | 3.24(A) | 467;465 | (3R,6R)-6-cyclopentyl-3-(2,3-dihydro-1H-inden-2-yl)-1-{[2-(methylsulfonyl)phenyl]methyl}-2,5-piperazinedione |
| 143 | 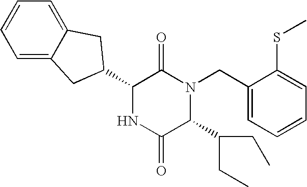 | 436.6 | 3.51(A) | 437;Notseen | (3R,6R)-3-(2,3-dihydro-1H-inden-2-yl)-6-(1-ethylpropyl)-1-{[2-(methylthio)phenyl]methyl}-2,5-piperazinedione |
| 144 | 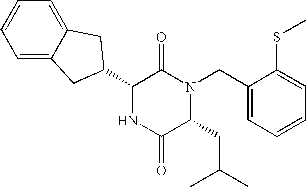 | 422.6 | 3.6 (A) | 423;421 | (3R,6R)-3-(2,3-dihydro-1H-inden-2-yl)-6-(2-methylpropyl)-1-{[2-(methylthio)phenyl]methyl}-2,5-piperazinedione |
| 145 | 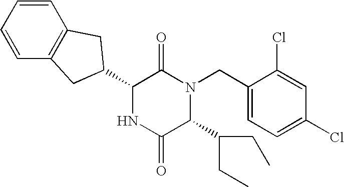 | 459.4 | 3.42(D) | 459 | (3R,6R)-1-[(2,4-dichlorophenyl)-methyl]-3-(2,3-dihydro-1H-inden-2-yl)-6-(1-ethylpropyl)-2,5-piperazinedione |
| 146 | 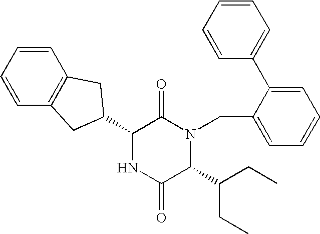 | 466.6 | 3.79(A) | 467 | (3R,6R)-1-(2-biphenylylmethyl)-3-(2,3-dihydro-1H-inden-2-yl)-6-(1-ethylpropyl)-2,5-piperazinedione |
| 147 | 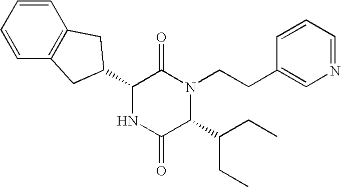 | 405.5 | 2.78(A) | 406 | (3R,6R)-3-(2,3-dihydro-1H-inden-2-yl)-6-(1-ethylpropyl)-1-[2-(3-pyridinyl)ethyl]-2,5-piperazinedione |
| 148 | 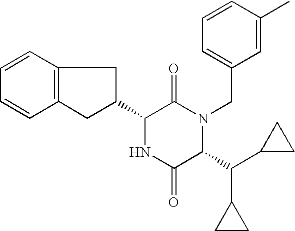 | 428.6 | 3.55(A) | 429 | (3R,6R)-6-(dicyclopropylmethyl)-3-(2,3-dihydro-1H-inden-2-yl)-1-[(3-methylphenyl)methyl]-2,5-piperazinedione |
| 149 | 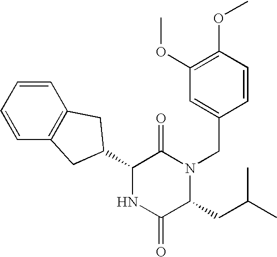 | 436.5 | 3.19(A) | 437 | (3R,6R)-1-{[3,4-bis(methyloxy)-phenyl]methyl}-3-(2,3-dihydro-1H-inden-2-yl)-6-(2-methyl-propyl)-2,5-piperazinedione |
| 150 | 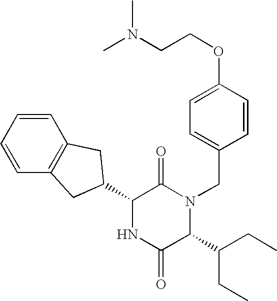 | 477.7 | 2.63(A) | 478 | (3R,6R)-3-(2,3-dihydro-1H-inden-2-yl)-1-[(4-{[2-(dimethylamino)-ethyl]oxy}phenyl)methyl]-6-(1-ethylpropyl)-2,5-piperazinedione |
| 151 | 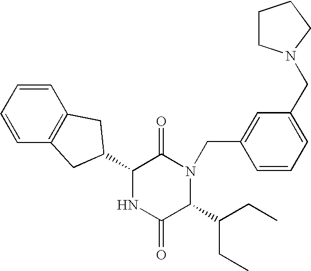 | 473.7 | 2.69(A) | 474 | (3R,6R)-3-(2,3-dihydro-1H-inden-2-yl)-6-(1-ethylpropyl)-1-{[3-(1-pyrrolidinylmethyl)phenyl]methyl}-2,5-piperazinedione |
| 152 | 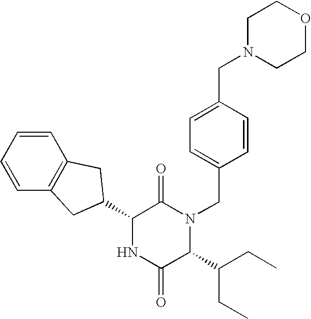 | 489.7 | 2.51(A) | 490 | (3R,6R)-3-(2,3-dihydro-1H-inden-2-yl)-6-(1-ethylpropyl)-1-{[4-(4-morpholinylmethyl)phenyl]methyl}-2,5-piperazinedione |
| 153 | 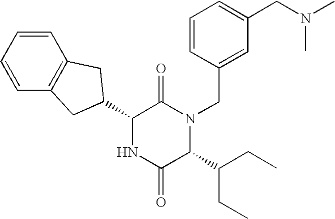 | 447.6 | 2.57(A) | 448 | formic acid-(3R,6R)-3-(2,3-dihydro-1H-inden-2-yl)-1-({3-[(dimethylamino)methyl]phenyl}-methyl)-6-(1-ethylpropyl)-2,5-piperazinedione (1:1) |
| 154 | 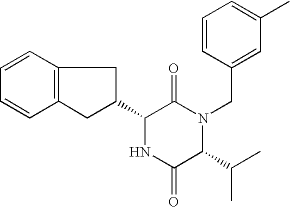 | 376.5 | 3.36(A) | 377 | (3R,6R)-3-(2,3-dihydro-1H-inden-2-yl)-6-(1-methylethyl)-1-[(3-methylphenyl)methyl]-2,5-piperazinedione |
| 155 | 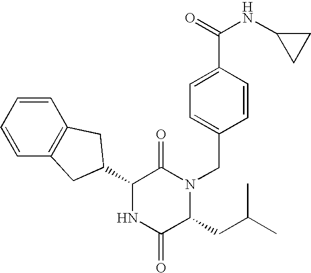 | 459.6 | 3.15(A) | 460 | N-cyclopropyl-4-{[(3R,6R)-3-(2,3-dihydro-1H-inden-2-yl)-6-(2-methylpropyl)-2,5-dioxo-1-piperazinyl]methyl}benzamide |

The following Example was prepared by a method analogous to Example 125 starting from Intermediate 12

| Ex | | | Rt/ | +ve; | |
|---|---|---|---|---|---|
| No | Structure | Mwt | min | −ve | Name |
| 156 | 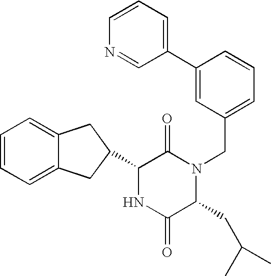 | 453.6 | 3.2(A) | 454;452 | (3R,6R)-3-(2,3-dihydro-1H-inden-2-yl)-6-(2-methylpropyl)-1-{[3-(3-pyridinyl)phenyl]methyl}-2,5-piperazinedione |

The following Examples were prepared by methods analogous to that described for Example 1 using the intermediates indicated, optionally with the addition of a base such as triethylamine or DIPEA if the hydrochloride salts of amines were used

| Ex | Int | | | Rt/ | +ve; | |
|---|---|---|---|---|---|---|
| No | No | Structure | Mwt | min | −ve | Name |
| 157 | 13 | 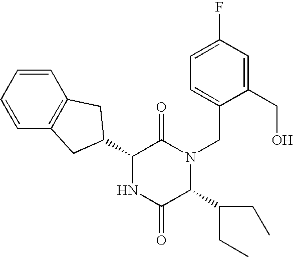 | 438.5 | 3.39(A) | 439;484 | (3R,6R)-3-(2,3-dihydro-1H-inden-2-yl)-6-(1-ethylpropyl)-1-{[4-fluoro-2-(hydroxymethyl)-phenyl]methyl}-2,5-piperazinedione |
| 158 | 23 | 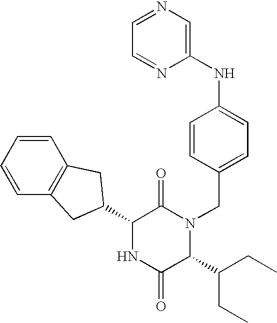 | 483.6 | 0.81(B) | 484 | (3R,6R)-3-(2,3-dihydro-1H-inden-2-yl)-6-(1-ethylpropyl)-1-{[4-(pyrazin-2-yl)aminophenyl]-methyl}-2,5-piperazinedione |
| 159 | 24 | 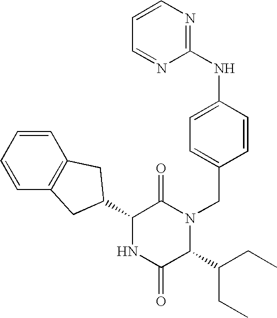 | 483.6 | 0.8 (B) | 484 | (3R,6R)-3-(2,3-dihydro-1H-inden-2-yl)-6-(1-ethylpropyl)-1-{[4-(pyrimid-2-yl)aminophenyl]-methyl}-2,5-piperazinedione |
| 160 | 24 | 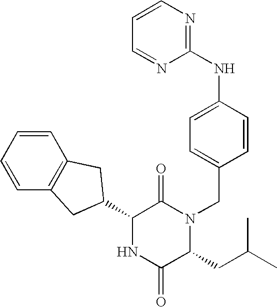 | 469.6 | 0.78(B) | 470 | (3R,6R)-3-(2,3-dihydro-1H-inden-2-yl)-6-(2-methylpropyl)-1-{[4-(pyrimid-2-yl)amino-phenyl]methyl}-2,5-piperazinedione |
| 161 | 25 | 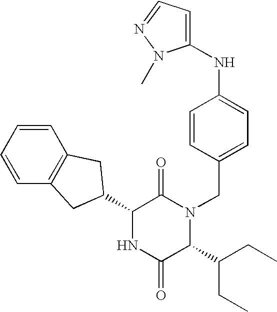 | 485.6 | 0.74(B) | 486 | (3R,6R)-3-(2,3-dihydro-1H-inden-2-yl)-6-(1-ethylpropyl)-1-({4-[(1-methyl-1H-pyrazol-5-yl)amino]phenyl}methyl)-2,5-piperazinedione |
| 162 | 25 | 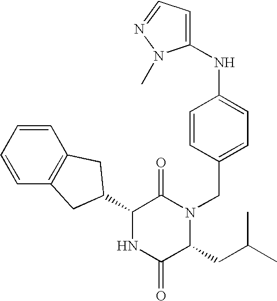 | 471.6 | 0.72(B) | 472 | (3R,6R)-3-(2,3-dihydro-1H-inden-2-yl)-6-(2-methylpropyl)-1-({4-[(1-methyl-1H-pyrazol-5-yl)amino]phenyl}methyl)-2,5-piperazinedione |
| 163 | 26 | 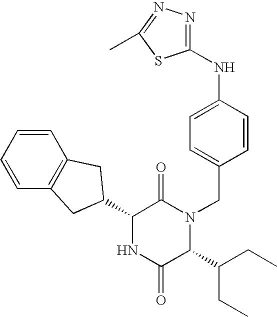 | 503.7 | 0.78(B) | 504 | (3R,6R)-3-(2,3-dihydro-1H-inden-2-yl)-6-(1-ethylpropyl)-1-{[4-(5-methyl-1,3,4-thiadiazol-2-yl)aminophenyl]methyl}-2,5-piperazinedione |
| 164 | 26 | 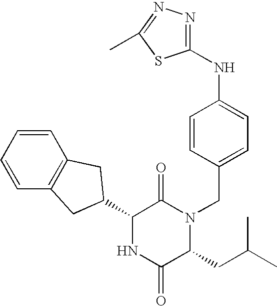 | 489.6 | 0.76(B) | 490 | (3R,6R)-3-(2,3-dihydro-1H-inden-2-yl)-6-(2-methylpropyl)-1-{[4-(5-methyl-1,3,4-thiadiazol-2-yl)aminophenyl]methyl}-2,5-piperazinedione |
| 165 | 27 | 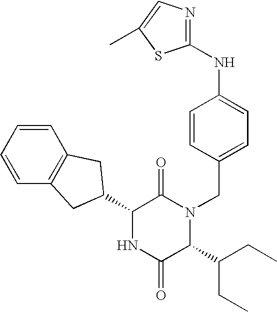 | 502.7 | 0.73(B) | 503 | (3R,6R)-3-(2,3-dihydro-1H-inden-2-yl)-6-(1-ethylpropyl)-1-{[4-(5-methyl-1,3-thiazol-2-yl)aminophenyl]methyl}-2,5-piperazinedione |
| 166 | 27 | 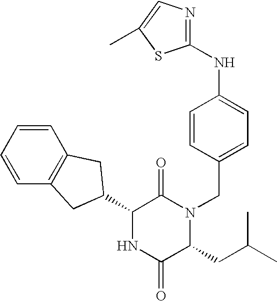 | 488.7 | 0.69(B) | 489 | (3R,6R)-3-(2,3-dihydro-1H-inden-2-yl)-6-(2-methylpropyl)-1-{[4-(5-methyl-1,3-thiazol-2-yl)aminophenyl]methyl}-2,5-piperazinedione |
| 167 | 32 | 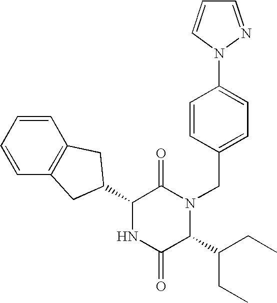 | 456.6 | 0.87(B) | 457 | (3R,6R)-3-(2,3-dihydro-1H-inden-2-yl)-6-(1-ethylpropyl)-1-{[4-(1H-pyrazol-1-yl)phenyl]-methyl}-2,5-piperazinedione |
| 168 | 30 | 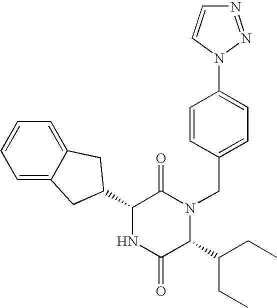 | 457.6 | 0.79(B) | 458 | (3R,6R)-3-(2,3-dihydro-1H-inden-2-yl)-6-(1-ethylpropyl)-1-{[4-(1H-1,2,3-triazol-1-yl)-phenyl]methyl}-2,5-piperazinedione |
| 169 | 33 | 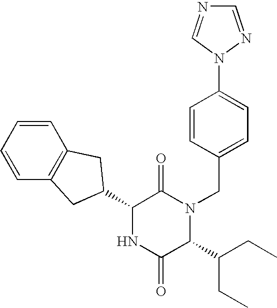 | 457.6 | 0.78(B) | 458 | (3R,6R)-3-(2,3-dihydro-1H-inden-2-yl)-6-(1-ethylpropyl)-1-{[4-(1H-1,2,4-triazol-1-yl)-phenyl]methyl}-2,5-piperazinedione |
| 170 | 31 | 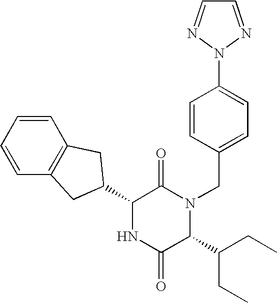 | 457.6 | 0.88(B) | 458 | (3R,6R)-3-(2,3-dihydro-1H-inden-2-yl)-6-(1-ethylpropyl)-1-{[4-(2H-1,2,3-triazol-2-yl)-phenyl]methyl}-2,5-piperazinedione |
| 171 | 37 | 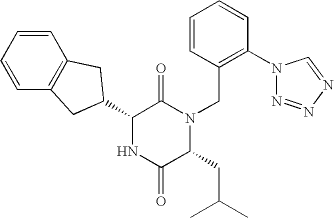 | 444.5 | 0.76(B) | 445 | (3R,6R)-3-(2,3-dihydro-1H-inden-2-yl)-6-(2-methylpropyl)-1-{[2-(1H-tetrazol-1-yl)phenyl]methyl}-2,5-piperazinedione |
| 172 | 37 | 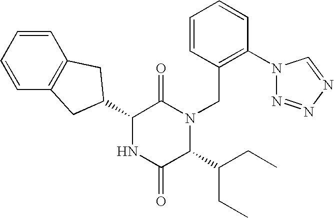 | 458.6 | 0.79(B) | 459 | (3R,6R)-3-(2,3-dihydro-1H-inden-2-yl)-6-(1-ethylpropyl)-1-{[2-(tetrazol-1-yl)phenyl]-methyl}-2,5-piperazinedione |

Example 173 was prepared from Example 157 by methods analogous to those described for Intermediate 51 and Example 65, without isolation of the intermediate aldehyde.

| Ex | | | Rt/ | +ve; | |
|---|---|---|---|---|---|
| No | Structure | MW | min | −ve | Name |
| 173 | 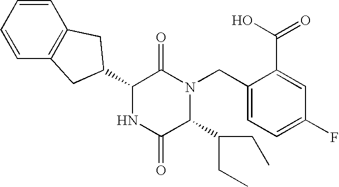 | 452.5 | 3.6(A) | 453;451 | 2-{[(3R,6R)-3-(2,3-dihydro-1H-inden-2-yl)-6-(1-ethylpropyl)-2,5-dioxo-1-piperazinyl]methyl}-5-fluorobenzoic acid |

The following Examples were prepared from Example 173 by methods analogous to that described for Example 66, except using diisopropylethylamine as the base in place of triethylamine.

| Ex | | | Rt/ | +ve; | |
|---|---|---|---|---|---|
| No | Structure | MW | min | −ve | Name |
| 174 | 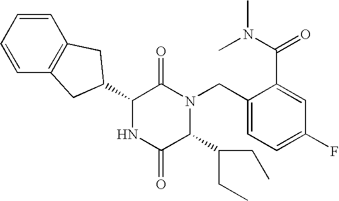 | 479.5 | 3.34(A) | 480524 | 2-{[(3R,6R)-3-(2,3-dihydro-1H-inden-2-yl)-6-(1-ethylpropyl)-2,5-dioxo-1-piperazinyl]methyl}-5-fluoro-N,N-dimethylbenzamide |
| 175 | 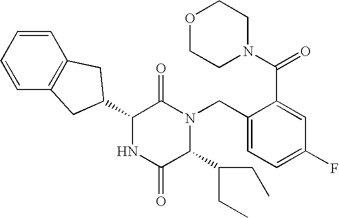 | 521.6 | 3.25(A) | 522566 | (3R,6R)-3-(2,3-dihydro-1H-inden-2-yl)-6-(1-ethylpropyl)-1-{[4-fluoro-2-(4-morpholinylcarbonyl)phenyl]-methyl}-2,5-piperazinedione |
| 176 | 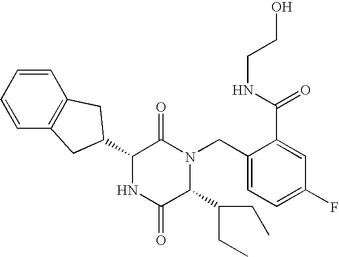 | 495.5 | 2.96(A) | 496494 | 2-{[(3R,6R)-3-(2,3-dihydro-1H-inden-2-yl)-6-(1-ethylpropyl)-2,5-dioxo-1-piperazinyl]methyl}-5-fluoro-N-(2-hydroxyethyl)benzamide |

### Example 177

### 2-{[(3R,6R)-3-(2,3-dihydro-1H-inden-2-yl)-6-(2-methylpropyl)-2,5-dioxo-1-piperazinyl]methyl}benzoic acid

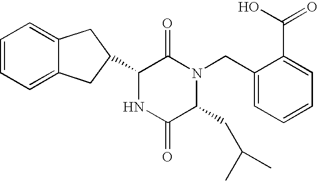

(3R,6R)-3-(2,3-Dihydro-1H-inden-2-yl)-1-{[2-(hydroxymethyl)phenyl]methyl}-6-(2-methylpropyl)-2,5-piperazinedione (Int. 14) (1.488 g, 3.66 mmol) was stirred in acetonitrile (8 ml), water (12 ml) and ethyl acetate (12 ml). Sodium periodate (3.21 g, 15 mmol) was added to the stirred mixture followed by ruthenium(3+) trichloride hydrate (24 mg). The mixture was stirred vigorously for 2.75 hours before it was filtered and the residue washed with ethyl acetate. The filtrate and washings were combined and the phases separated. The aqueous phase was extracted with ethyl acetate (2×10 ml). The organic phases were combined, dried (MgSO₄) and evaporated to leave a brown foam (1.64 g). The brown foam (1.63 g) was stirred in acetonitrile (150 ml) and a solution of sulfamic acid (426 mg, 4.38 mmol) in water (15 ml) was added dropwise, followed after 3 minutes by the dropwise addition of a solution of sodium chlorite (430 mg, 4.75 mmol) in water (15 ml). After the mixture had been stirred at room temperature for 2 hours it was left to stand at room temperature overnight (16.33 hours) before it was evaporated under reduced pressure to remove the organic solvent. The aqueous residue was partitioned between ethyl acetate (100 ml) and water (10 ml). The organic phase was washed with saturated aqueous sodium chloride solution (25 ml), dried (MgSO₄), evaporated under reduced pressure and dried in vacuo to afford 2-{[(3R,6R)-3-(2,3-dihydro-1H-inden-2-yl)-6-(2-methylpropyl)-2,5-dioxo-1-piperazinyl]methyl}benzoic acid as an orange/brown foam (1.608 g). A portion of 2-{[(3R,6R)-3-(2,3-dihydro-1H-inden-2-yl)-6-(2-methylpropyl)-2,5-dioxo-1-piperazinyl]methyl}benzoic acid was purified further by mass directed autoprep to afford a white solid (45 mg).

LCMS (A) Rt=3.22 minutes; m/z [M+H]⁺=421

¹H NMR (CDCl₃) [symbol] 8.36 (br s, 1H), 8.05 (d, 1H), 7.55 (t, 1H), 7.39 (t, 1H), 7.33 (d, 1H), 7.22 (m, 2H), 7.16 (m, 2H), 5.49 (d, 1H), 4.71 (d, 1H), 4.14 (dd, 1H), 3.93 (dd, 1H), 3.14 (m, 3H), 2.90 (m, 2H), 1.93 (m, 1H), 1.82 (m, 1H), 1.71 (m, 1H), 0.91 (t, 6H).

### Example 178

### 2-{[(3R,6R)-3-(2,3-dihydro-1H-inden-2-yl)-6-(2-methylpropyl)-2,5-dioxo-1-piperazinyl]methyl}benzamide

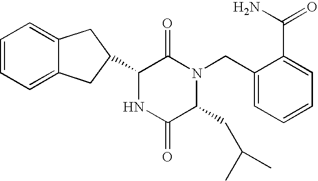

2-{[(3R,6R)-3-(2,3-Dihydro-1H-inden-2-yl)-6-(2-methylpropyl)-2,5-dioxo-1-piperazinyl]-methyl}benzoic acid (Ex. 177) (600 mg, 1.26 mmol) was dissolved in dry dichloromethane (9 ml) and triethylamine (353.4 ul, 2.53 mmol) under nitrogen. 2-(1H-Benzotriazol-1-yl)-1,1,3,3-tetramethyluronium tetrafluoroborate (488 mg, 1.52 mmol) was added to the mixture, which was stirred for 6 hours at room temperature before it was split into 4 and each portion treated with an amine.

To one portion was added a 2M solution of ammonia in methanol (1 ml, 2 mmol). The mixture was left to stand at room temperature over the weekend, before it was diluted with dichloromethane (2 ml) and washed with 1M hydrochloric acid (2 ml) followed by saturated aqueous sodium bicarbonate solution (2 ml). The phases were separated using a hydrophobic frit and the organic phase blown down under nitrogen to leave a brown foam. The foam was purified by mass directed autoprep to afford 2-{[(3R,6R)-3-(2,3-dihydro-1H-inden-2-yl)-6-(2-methylpropyl)-2,5-dioxo-1-piperazinyl]methyl}-benzamide (47 mg) as a white solid.

LCMS (A) Rt=2.94 minutes; m/z [M+H]⁺=420

¹H NMR (CDCl₃) [symbol] 8.15 (brs, 1H), 7.47 (d, 1H), 7.38 (brt, 1H), 7.31 (d, 1H), 7.24 (t, 1H), 7.16 (m, 4H), 6.77 (brs, 2H), 5.42 (d, 1H), 4.28 (d, 1H), 4.03 (m, 1H), 3.91 (m, 1H), 3.06 (m, 3H), 2.82 (m, 2H), 1.92 (m, 1H), 1.73 (m, 2H), 0.93 (d, 6H).

The following Examples were prepared by methods analogous to that described for Example 178

| Ex | | | Rt/ | +ve; | |
|---|---|---|---|---|---|
| No | Structure | MW | min | −ve | Name |
| 179 | 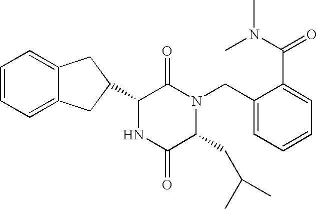 | 477.6 | 3.08(A) | 448;446 | 2-{[(3R,6R)-3-(2,3-dihydro-1H-inden-2-yl)-6-(2-methylpropyl)-2,5-dioxo-1-piperazinyl]methyl}-N,N-dimethylbenzamide |
| 180 | 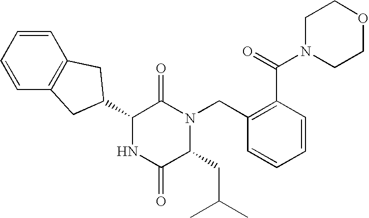 | 489.6 | 3.08(A) | 490;488 | (3R,6R)-3-(2,3-dihydro-1H-inden-2-yl)-6-(2-methylpropyl)-1-{[2-(4-morpholinylcarbonyl)phenyl]meth-yl}-2,5-piperazinedione |
| 181 | 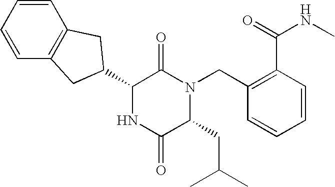 | 433.6 | 3.00(A) | 434;432 | 2-{[(3R,6R)-3-(2,3-dihydro-1H-inden-2-yl)-6-(2-methylpropyl)-2,5-dioxo-1-piperazinyl]methyl}-N-methylbenzamide |

### Example 182

### 3-{[(3R,6R)-3-(2,3-dihydro-1H-inden-2-yl)-6-(1-ethylpropyl)-2,5-dioxo-1-piperazinyl]methyl}benzoic acid

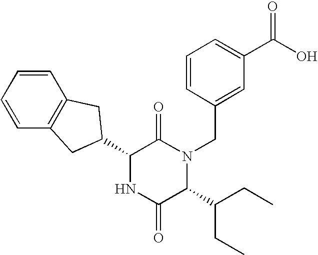

1,1-Dimethylethyl 3-{[(3R,6R)-3-(2,3-dihydro-1H-inden-2-yl)-6-(1-ethylpropyl)-2,5-dioxo-1-piperazinyl]methyl}benzoate (Int. 15) (518 mg, 1.05 mmol) was dissolved in 2M hydrochloric acid in ether (2 ml) and left to stand at room temperature overnight (18.25 hours). Then 4M hydrochloric acid in dioxan (1 ml) was added to the mixture, which was left to stand a further 47 hours before more 4M hydrochloric acid in dioxan (0.4 ml) was added. After a further 5.5 hours the mixture was evaporated under reduced pressure to leave a foam. The foam was dissolved in 4M hydrochloric acid in dioxan (1 ml) and left to stand overnight (22.33 hours) before it was evaporated under reduced pressure to leave a gum, which was evaporated from cyclohexane and dried in vacuo to afford 3-{[(3R,6R)-3-(2,3-dihydro-1H-inden-2-yl)-6-(1-ethylpropyl)-2,5-dioxo-1-piperazinyl]methyl}-benzoic acid as a pale brown foam (540 mg).

LCMS (A) Rt=3.3 minutes; m/z [M+H]⁺=435

### Example 183

### 3-{[(3R,6R)-3-(2,3-dihydro-1H-inden-2-yl)-6-(1-ethylpropyl)-2,5-dioxo-1-piperazinyl]methyl}benzamide

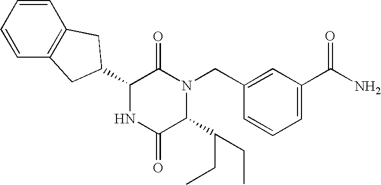

3-{[(3R,6R)-3-(2,3-Dihydro-1H-inden-2-yl)-6-(1-ethylpropyl)-2,5-dioxo-1-piperazinyl]-methyl}benzoic acid (Ex. 182) (493 mg, 0.96 mmol) was dissolved in dry dichloromethane (9 ml) under nitrogen and triethylamine (267.2 ul, 1.92 mmol) added. After 5 minutes 2-(1H-benzotriazol-1-yl)-1,1,3,3-tetramethyluronium tetrafluoroborate (369 mg, 1.15 mmol) was added to the mixture, which was stirred for 2 hours before being left to stand at room temperature overnight (18.25 hours) and then split into 3 parts and each portion treated with an amine.

To one portion was added a 2M solution of ammonia in methanol (1 ml, 2 mmol). The mixture was left to stand at room temperature for 5 hours before it was diluted with dichloromethane (3 ml) and washed with 1M hydrochloric acid (1 ml) followed by saturated aqueous sodium bicarbonate solution (2 ml). The phases were separated using a hydrophobic frit and the organic phase blown down under nitrogen to leave a brown foam. The foam was purified by mass directed autoprep to afford 3-{[(3R,6R)-3-(2,3-dihydro-1H-inden-2-yl)-6-(1-ethylpropyl)-2,5-dioxo-1-piperazinyl]methyl}benzamide as a pale brown foam (80 mg, 56%).

LCMS (A) Rt=3.01 minutes; m/z [M+H]⁺=434

¹H NMR (CDCl₃) δ ? 8.01 (br s, 1H), 7.74 (m, 2H), 7.41 (m, 2H), 7.18 (m, 4H), 6.44 (br s, 2H), 5.35 (d, 1H), 4.10 (m, 2H), 3.93 (d, 1H), 3.14 (m, 3H), 2.88 (m, 2H), 1.73 (m, 1H), 1.66 (m, 1H), 1.58 (m, 2H), 1.31 (m, 1H), 0.92 (t, 3H), 0.87 (t, 3H).

The following Examples were prepared by methods analogous to that described for Example 183

| Ex | | | Rt/ | +ve; | |
|---|---|---|---|---|---|
| No | Structure | MW | min | −ve | Name |
| 184 | 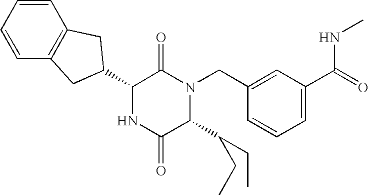 | 447.6 | 3.05(A) | 448;446 | 3-{[(3R,6R)-3-(2,3-dihydro-1H-inden-2-yl)-6-(1-ethylpropyl)-2,5-dioxo-1-piperazinyl]methyl}-N-methylbenzamide |
| 185 | 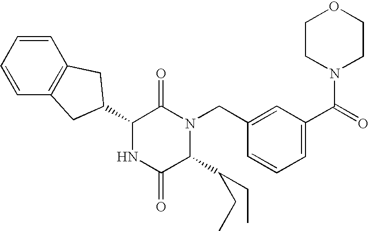 | 503.6 | 3.07(A) | 504;502 | (3R,6R)-3-(2,3-dihydro-1H-inden-2-yl)-6-(1-ethylpropyl)-1-{[3-(4-morpholinylcarbonyl)phenyl]-methyl}-2,5-piperazinedione |

The following Example was prepared from Intermediate 54 by a method analogous to that described for Example 182

| | | | | | |
|---|---|---|---|---|---|
| 186 | 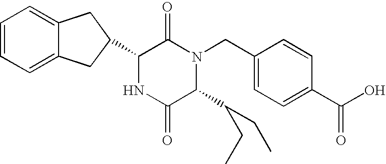 | 434.5 | 3.27(A) | 435;433 | 4-{[(3R,6R)-3-(2,3-dihydro-1H-inden-2-yl)-6-(1-ethylpropyl)-2,5-dioxo-1-piperazinyl]methyl}-benzoic acid |

The following Examples were prepared from Example 186 by methods analogous to that described for Example 183

| Ex | | | Rt/ | +ve; | |
|---|---|---|---|---|---|
| No | Structure | MW | min | −ve | Name |
| 187 | 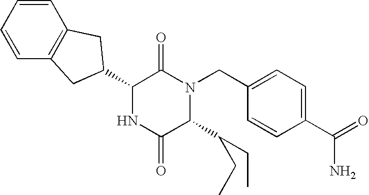 | 433.6 | 2.98(A) | 434;432 | 4-{[(3R,6R)-3-(2,3-dihydro-1H-inden-2-yl)-6-(1-ethylpropyl)-2,5-dioxo-1-piperazinyl]methyl}-benzamide |
| 188 | 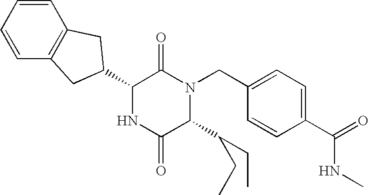 | 447.6 | 3.16(A) | 448;492 | 4-{[(3R,6R)-3-(2,3-dihydro-1H-inden-2-yl)-6-(1-ethylpropyl)-2,5-dioxo-1-piperazinyl]methyl}-N-methylbenzamide |
| 189 | 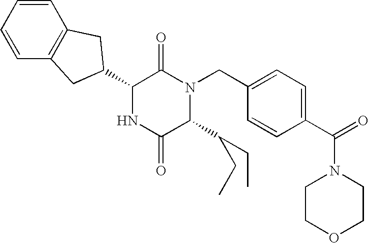 | 503.6 | 3.1 (A) | 504;502 | (3R,6R)-3-(2,3-dihydro-1H-inden-2-yl)-6-(1-ethylpropyl)-1-{[4-(4-morpholinylcarbonyl)-phenyl]methyl}-2,5-piperazine-dione |

### Example 190

### 4-{[(3R,6R)-3-(2,3-Dihydro-1H-inden-2-yl)-6-(1-ethylpropyl)-2,5-dioxo-1-piperazinyl]methyl}-N-(2-hydroxyethyl)benzamide

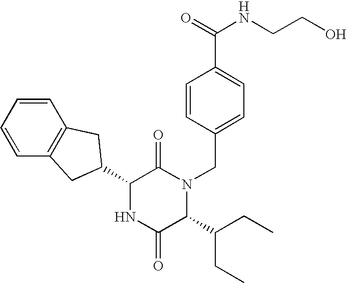

To a solution of the mixed stereoisomers of Intermediate 49 (100 mg, 0.230 mmol) and 2-aminoethanol (30 μL, 0.460 mmol) in CH₂Cl₂ (2 mL) were added diisopropylethylamine (90 μL, 0.506 mmol), 1-(3-dimethylaminopropyl)-3-ethylcarboiimide hydrochloride (EDCI.HCl) (49 mg, 0.253 mmol) and 1-hydroxybenzotriazole hydrate (HOBT) (6 mg, 0.046 mmol). The reaction mixture was stirred at room temperature until judged complete by LCMS. At 16 hours, LCMS indicated about 55% conversion; therefore, additional 2-aminoethanol (10 μL, 0.153 mmol) was added. The reaction mixture was continued to stir for an additional 4 days. LCMS indicated about 65% conversion. The reaction was diluted with EtOAc. The resulting solution was washed with a saturated aqueous NaHCO₃ solution and brine. The organics were dried over MgSO₄, filtered and concentrated to give 80 mg of a white solid. The crude residue was purified by flash chromatography on silica gel [ISCO, 4 g RediSep® column, CH₂Cl₂/MeOH 1%-10%] to give 29 mg of 4-{[(3R,6S)-3-(2,3-dihydro-1H-inden-2-yl)-6-(1-ethylpropyl)-2,5-dioxo-1-piperazinyl]methyl}-N-(2-hydroxyethyl)benzamide (trans-isomer) as an oil and 15 mg of Example 190, 4-{[(3R,6R)-3-(2,3-dihydro-1H-inden-2-yl)-6-(1-ethylpropyl)-2,5-dioxo-1-piperazinyl]methyl}-N-(2-hydroxyethyl)benzamide (cis-isomer) as an oil.

HPLC (B) Rt=0.71 minutes; m/z [M+H]⁺=478

¹H NMR (CDCl₃) δ 7.76 (d, 2H), 7.54 (d, 2H), 7.30-7.15 (m, 2H), 7.05 (t, 1H), 5.67 (br s, 1H), 5.33 (d, 1H), 4.07 (m, 2H), 3.89 (d, 1H), 3.82 (t, 2H), 3.62 (m, 2H), 3.15 (m, 3H), 3.0-2.75 (m, 2H), 2.54 (br s, 2H), 1.80-1.50 (m, 4H), 1.34 (m, 1H), 0.95 (t, 3H), 0.88 (t, 3H).

The following Examples were prepared from Intermediate 49 by methods analogous to that described for Example 190

| Ex | | | Rt/ | +ve; | |
|---|---|---|---|---|---|
| No | Structure | MW | min | −ve | Name |
| 191 | 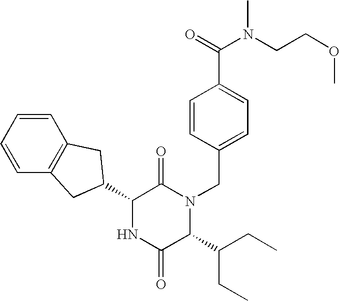 | 505.7 | 0.78(B) | 506 | 4-{[(3R,6R)-3-(2,3-dihydro-1H-inden-2-yl)-6-(1-ethylpropyl)-2,5-dioxo-1-piperazinyl]methyl}-N-methyl-N-[2-(methyloxy)-ethyl]benzamide |
| 192 | 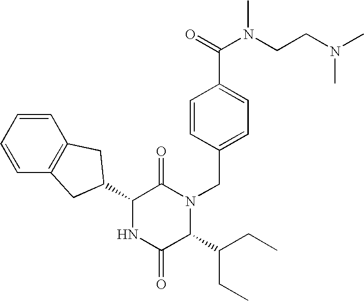 | 518.7 | 0.67(B) | 519 | 4-{[(3R,6R)-3-(2,3-dihydro-1H-inden-2-yl)-6-(1-ethylpropyl)-2,5-dioxo-1-piperazinyl]methyl}-N-[2-(dimethylamino)ethyl]-N-methylbenzamide |
| 193 | 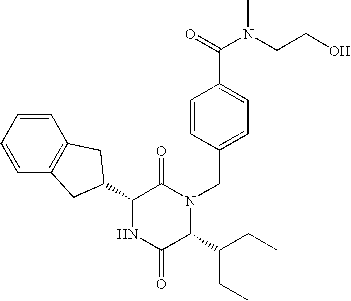 | 491.6 | 0.72(B) | 492 | 4-{[(3R,6R)-3-(2,3-dihydro-1H-inden-2-yl)-6-(1-ethylpropyl)-2,5-dioxo-1-piperazinyl]methyl}-N-(2-hydroxyethyl)-N-methylbenzamide |

### Example 194

### (3R,6R)-3-(2,3-Dihydro-1H-inden-2-yl)-1-({4-[(4-methyl-1-piperazinyl)carbonyl]-phenyl}methyl)-6-(2-methylpropyl)-2,5-piperazinedione

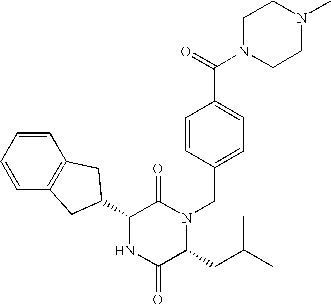

To a solution of the mixed stereoisomers of Intermediate 50 (110 mg, 0.262 mmol) in CH₂Cl₂ (2.3 mL) were added diisopropylethylamine (55 μL, 0.314 mmol), 1-methylpiperizine (32 μL, 0.288 mmol) and O-(7-azabenzotriazol-1-yl)-N,N,N′,N′-tetramethyluronium hexafluorophosphate (HATU) (119 mg, 0.314 mmol). After stirring at room temperature for 2 hours, LCMS indicated complete conversion. The reaction was diluted with EtOAc and washed with a saturated aqueous NaHCO₃ solution and brine. The organics were dried over MgSO₄, filtered and concentrated to give 180 mg of an off-white solid. The crude residue was purified by flash chromatography on silica gel [ISCO, 4 g RediSep® column, CH₂Cl₂/MeOH 1%-10%] to give 53 mg of (3R,6S)-3-(2,3-dihydro-1H-inden-2-yl)-1-({4-[(4-methyl-1-piperazinyl)carbonyl]phenyl}methyl)-6-(2-methylpropyl)-2,5-piperazinedione (trans-isomer) as a white powder and 41 mg of Example 193, (3R,6R)-3-(2,3-dihydro-1H-inden-2-yl)-1-({4-[(4-methyl-1-piperazinyl)-carbonyl]phenyl}methyl)-6-(2-methylpropyl)-2,5-piperazinedione (cis-isomer) as a white powder.

HPLC (B) Rt=0.62 minutes; m/z [M+H]⁺=503

1H NMR (CDCl3) δ 7.40 (d, 2H), 7.32 (d, 2H), 7.28-7.15 (m, 4H), 5.36 (d, 1H), 4.07 (dd, 1H), 3.94 (d, 1H), 3.81 (m, 3H), 3.47 (br s, 2H), 3.12 (m, 3H), 2.86 (m, 2H), 2.60-2.30 (m, 4H), 2.36 (s, 3H), 1.97 (m, 1H), 1.83 (m, 1H), 1.66 (m, 1H), 0.97 (t, 6H).

### Example 195

### 2-{[(3R,6R)-3-(2,3-dihydro-1H-inden-2-yl)-6-(1-ethylpropyl)-2,5-dioxo-1-piperazinyl]methyl}-N-{1-[2-(methyloxy)ethyl]-4-piperidinyl}benzamide

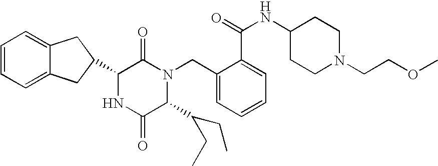

To a stirred solution of 2-{[(3R,6R)-3-(2,3-dihydro-1H-inden-2-yl)-6-(1-ethylpropyl)-2,5-dioxo-1-piperazinyl]methyl}-N-4-piperidinylbenzamide (Example 83) (101 mg, 0.2 mmol) in anhydrous dimethylformamide (2 ml) was added potassium carbonate (27 mg, 0.2 mmol) and 2-bromoethylmethylether (20.2 uL, 0.21 mmol). The reaction was stirred for 20 hours. Further potassium carbonate (13.5 mg, 0.1 mmol) and 2-bromoethylmethyl-ether (15 uL, 0.16 mmol) were added and the reaction was stirred for 2.5 hours before more 2-bromoethylmethylether (15 uL, 0.16 mmol) was added. Then the reaction was stirred at room temperature over the weekend (71 hours) before further 2-bromoethyl-methylether (15 uL, 0.16 mmol) was added. Then the reaction was stirred for 25.5 hours before further potassium carbonate (16 mg, 0.11 mmol) and 2-bromoethylmethylether (15 uL, 0.16 mmol) were added and the reaction was stirred for 18 hours. The reaction mixture was partitioned between dichloromethane (10 ml) and saturated aqueous ammonium chloride (5 ml). The aqueous phase was extracted with dichloromethane (4 ml). The phases were separated using a hydrophobic frit. The combined organic phase was evaporated under reduced pressure to give a pale orange solid. The solid in a small volume of dichloromethane was loaded onto an SCX-SPE column, washed with methanol then eluted with 2M ammonia/methanol. Concentration gave a pale cream foam, which was loaded onto a 12 g flash silica chromatography column (pre-eluted with 0.4% triethylamine in ethyl acetate). The column was eluted with 0% to 100% solvent B in solvent A (solvent A=0.4% triethylamine in ethyl acetate, and solvent B=20% ethanol in ethyl acetate) to afford 2-{[(3R,6R)-3-(2,3-dihydro-1H-inden-2-yl)-6-(1-ethylpropyl)-2,5-dioxo-1-piperazinyl]methyl}-N-{1-[2-(methyloxy)ethyl]-4-piperidinyl}-benzamide as a white solid (68 mg, 60%).

LCMS (A) Rt=2.64 minutes; m/z [M+H]⁺=575

¹H NMR (CDCl₃) [symbol] 7.54 (br d, 1H), 7.43 (br d, 1H), 7.38 (br t, 1H), 7.30 (br t, 1H), 7.24 (m, 2H), 7.17 (m, 3H), 6.78 (d, 1H), 5.11 (d, 1H), 4.43 (d, 1H), 4.12 (d, 1H), 4.03 (dd, 1H), 3.98 (m, 1H), 3.52 (t, 2H), 3.36 (s, 3H), 3.11 (m, 3H), 2.92 (m, 3H), 2.81 (m, 1H), 2.59 (t, 2H), 2.23 (m, 2H), 2.05 (m, 2H), 1.77-1.53 (m, 6H), 1.35 (m, 1H), 0.96 (t, 3H), 0.90 (t, 3H).

### Example 196

### (3R,6R)-1-{[2,4-Bis(hydroxymethyl)phenyl]methyl}-3-(2,3-dihydro-1H-inden-2-yl)-6-(1-ethylpropyl)-2,5-piperazinedione

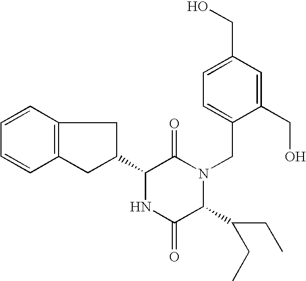

[4-(Aminomethyl)benzene-1,3-diyl]dimethanol (2.22 g, 13.3 mmol) and (2R)-2,3-dihydro-1H-inden-2-yl({[(1,1-dimethylethyl)oxy]carbonyl}amino)ethanoic acid (3.87 g, 13.3 mmol) were dissolved in 15 mL methanol. 2-Ethylbutanal (1.33 g, 13.3 mmol) were added into the solution. The resulting solution was stirred at room temperature for 30 minutes. Then 4-chlorophenyl isocyanide (1.44 g, 13.3 mmol) was added and the resulting mixture was stirred at room temperature for 1 hour. The solvent was removed in vacuo. The residue was redissolved in 20 mL chloroform and cooled to 0° C. The cold solution was then treated with 4M HCl in dioxane. The resulting mixture was then warmed up to room temperature and stirred overnight. The solvent was then removed and redissolved in 150 mL chloroform. The solution was then washed with 30 mL saturated sodium bicarbonate. The aqueous solution was extracted with 2×30 mL chloroform. The combined organics were dried over magnesium sulfate and concentrated. The resulting residue was redissolved in 30 mL chloroform. The solution was treated with 1.5 mL acetic acid and stirred at room temperature overnight. The solution was then concentrated. The residue was purified via silica gel chromatography eluting with 50-100% ethyl acetate in hexanes to give (3R,6R)-1-{[2,4-bis(hydroxymethyl)phenyl]methyl}-3-(2,3-dihydro-1H-inden-2-yl)-6-(1-ethylpropyl)-2,5-piperazinedione (1.05 g, 17%) as an off-white solid.

LCMS (B) Rt=0.71 minutes; m/z [M+H]⁺=451

¹H NMR (CDCl₃) [symbol] 7.39 (s, 1H), 7.16-7.27 (m, 5H), 7.02 (s, 1H), 5.45 (d, 1H), 4.69 (d, 1H), 4.66 (s, 2H), 4.57 (d, 1H), 4.10 (d, 1H), 3.94-4.05 (m, 2H), 3.09-3.14 (m, 2H), 2.87-2.89 (m, 1H), 2.77-2.80 (m, 1H), 2.30 (bs, 3H), 1.70-1.80 (m, 1H), 1.51-1.68 (m, 3H), 1.20-1.30 (m, 1H), 0.80-0.90 (m, 6H).

### Example 197

### ((3R,6R)-3-(2,3-Dihydro-1H-inden-2-yl)-6-(1-ethylpropyl)-1-{[2-(hydroxymethyl)-4-(methylthio)phenyl]methyl}-2,5-piperazinedione

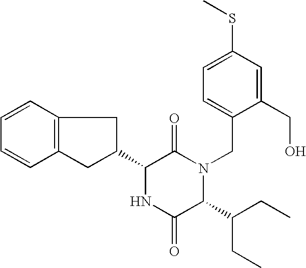

(2R)-2,3-Dihydro-1H-inden-2-yl({[(1,1-dimethylethyl)oxy]carbonyl}amino)ethanoic acid (0.80 g, 2.73 mmol), [2-(aminomethyl)-5-(methylthio)phenyl]methanol (0.50 g, 2.73 mmol) and 2-ethylbutanal (0.36 mL, 2.73 mmol) were dissolved in 10 mL methanol. The resulting solution was stirred at room temperature for 30 minutes. Then 4-chlorophenyl isocyanide (0.30 g, 2.73 mmol) was added and the resulting mixture was stirred at room temperature overnight. The solvent was removed in vacuo. The residue was then purified via isco silica gel chromatography eluting with 10-100% dichloromethane in hexanes to give 1,1-dimethylethyl [(1R)-2-((1-{[(4-chlorophenyl)amino]carbonyl}-2-ethylbutyl){[2-(hydroxymethyl)-4-(methylthio)phenyl]methyl}amino)-1-(2,3-dihydro-1H-inden-2-yl)-2-oxoethyl]carbamate (1.26 g, 67%) as a white solid. This material (0.86 g, 1.24 mmol) was treated with 4 M hydrochloride in dioxane solution (12.4 mL, 2.48 mmol) at room temperature for 2 hours, then was placed in −20° C. freezer for 72 hours. After warming up to room temperature for 2 hours, the solvent was then removed and redissolved in 15 mL chloroform. The solution was then stirred with 10 mL saturated sodium bicarbonate for 20 minutes. The phases were seperated. The aqueous solution was extracted with 2×15 mL chloroform. The combined organics were dried over magnesium sulfate and concentrated. The resulting residue was redissolved in 15 mL chloroform. The solution was treated with 0.75 mL 20% v/v acetic acid in dioxane and stirred at room temperature overnight. The solution was then concentrated. The residue was then redissolved in 50 mL ethyl acetate and washed with 15 mL saturated sodium bicarbonate solution. The organic phase was drive over magnesium sulfate and concentrated. The residue was purified via isco silica gel chromatography eluting with 30-70% ethyl acetate in hexanes to give (3R,6R)-3-(2,3-dihydro-1H-inden-2-yl)-6-(1-ethylpropyl)-1-{[2-(hydroxymethyl)-4-(methylthio)phenyl]methyl}-2,5-piperazinedione (0.17 g, 30%) as an white solid.

LCMS (D) Rt=2.89 minutes; m/z [M+H]⁺=467

¹H NMR (CDCl₃) [symbol] 7.36 (s, 1H), 7.20-7.35 (m, 3H), 7.13-7.17 (m, 3H), 5.25 (d, 1H), 4.61 (dd, 2H), 4.36 (d, 1H), 4.11-4.13 (m, 1H), 3.92 (d, 1H), 3.09-3.11 (m, 3H), 2.88-2.91 (m, 2H), 2.50 (s, 3H), 1.72-1.82 (m, 1H), 1.60-1.65 (m, 3H), 1.20-1.35 (m, 1H), 0.92 (t, 3H), 0.89 (t, 3H).

### Example 199

### (3R,6R)-1-{[2,4-Bis(1-pyrrolidinylcarbonyl)phenyl]methyl}-3-(2,3-dihydro-1H-inden-2-yl)-6-(1-ethylpropyl)-2,5-piperazinedione

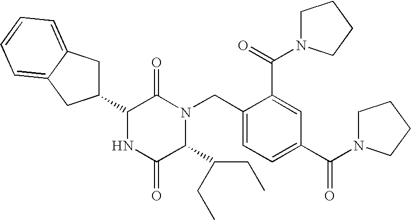

4-{[(3R,6R)-3-(2,3-Dihydro-1H-inden-2-yl)-6-(1-ethylpropyl)-2,5-dioxo-1-piperazinyl]-methyl}-1,3-benzenedicarboxylic acid (Int. 52) (50 mg, 0.105 mmol), HATU (99.4 mg, 0.262 mmol) and pyrrolidine (17.9 mg, 0.251 mmol) were dissolved in 1 mL dichloromethane. diisopropylethylamine (54.1 mg, 0.418 mmol) was then added. The resulting solution was stirred at RT for one hour. The crude reaction mixture was then purified via Gilson HPLC eluting with 0.1% trifluoroacetic acid in acetonitrile/water. The product containing fractions were combined and concentrated. The residue was redissolved in 20 mL ethyl acetate, washed with 5 mL saturated sodium bicarbonate, dried over MgSO₄ and concentrated to give (3R,6R)-1-{[2,4-bis(1-pyrrolidinyl-carbonyl)phenyl]methyl}-3-(2,3-dihydro-1H-inden-2-yl)-6-(1-ethylpropyl)-2,5-piperazinedione (30 mg, 49%) as a white solid.

LCMS (B) Rt=0.77 minutes; m/z [M+H]⁺=585.4

¹H NMR (CDCl₃) [symbol] 7.49 (d, 1H), 7.47 (s, 1H), 7.29 (d, 1H), 7.19 (m, 2H), 7.14 (m, 2H), 5.06 (d, 1H), 4.24 (d, 1H), 3.99 (s, 1H), 3.97 (d, 1H), 3.59 (t, 4H), 3.36-3.41 (m, 4H), 3.06-3.27 (m, 4H), 1.80-2.00 (m, 8H), 1.48-1.70 (m, 4H), 1.20-1.30 (m, 2H), 0.80-0.90 (m, 6H).

The following Examples were prepared by methods analogous to that described for Example 199

| Ex | | | Rt/ | | |
|---|---|---|---|---|---|
| No | Structure | Mwt | min | +ve | Name |
| 200 | 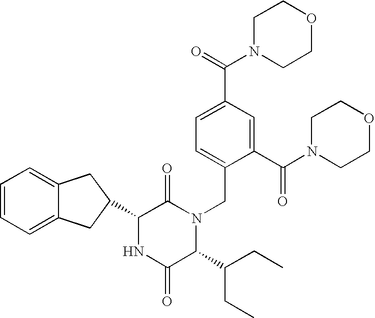 | 616.8 | 0.72(B) | 617 | (3R,6R)-1-{[2,4-bis(4-morpholinylcarbonyl)phenyl]-methyl}-3-(2,3-dihydro-1H-inden-2-yl)-6-(1-ethylpropyl)-2,5-piperazinedione |
| 201 | 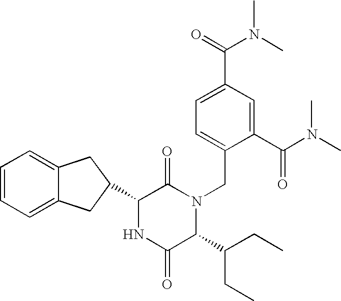 | 532.7 | 0.73(B) | 533 | 4-{[(3R,6R)-3-(2,3-dihydro-1H-inden-2-yl)-6-(1-ethylpropyl)-2,5-dioxo-1-piperazinyl]methyl}-N,N,N′,N′-tetramethyl-1,3-benzenedicarboxamide |
| 202 | 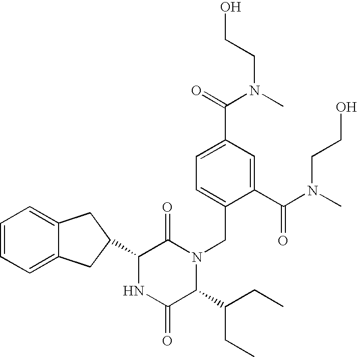 | 592.7 | 0.66(B) | 593 | 4-{[(3R,6R)-3-(2,3-dihydro-1H-inden-2-yl)-6-(1-ethylpropyl)-2,5-dioxo-1-piperazinyl]methyl}-N,N′-bis(2-hydroxyethyl)-N,N′-dimethyl-1,3-benzene-dicarboxamide |
| 203 | 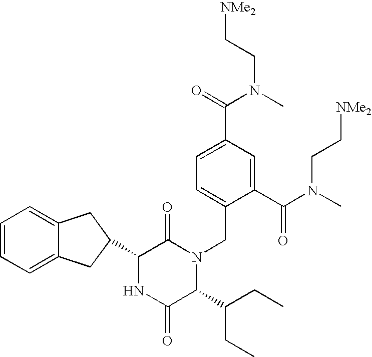 | 646.9 | 0.58(B) | 647 | 4-{[(3R,6R)-3-(2,3-dihydro-1H-inden-2-yl)-6-(1-ethylpropyl)-2,5-dioxo-1-piperazinyl]methyl}-N,N′-bis[2-(dimethylamino)ethyl]-N,N′-dimethyl-1,3-benzene-dicarboxamide |
| 204 | 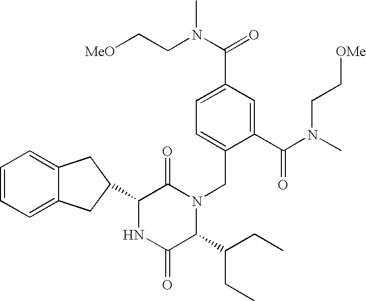 | 620.8 | 0.75(B) | 621 | 4-{[(3R,6R)-3-(2,3-dihydro-1H-inden-2-yl)-6-(1-ethylpropyl)-2,5-dioxo-1-piperazinyl]methyl}-N,N′-dimethyl-N,N′-bis[2-(methyloxy)-ethyl]-1,3-benzene-dicarboxamide |
| 205 | 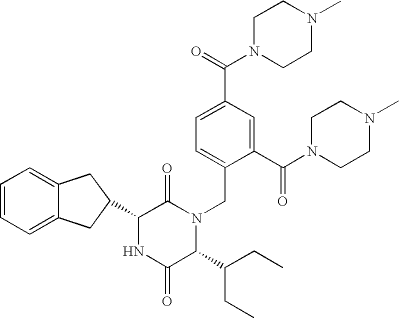 | 641.9 | 0.57(B) | 643 | (3R,6R)-1-({2,4-bis[(4-methyl-1-piperazinyl)carbonyl]phenyl}meth-yl)-3-(2,3-dihydro-1H-inden-2-yl)-6-(1-ethylpropyl)-2,5-piperazinedione |

### Example 206

### (3R,6R)-3-(2,3-Dihydro-1H-inden-2-yl)-6-(1-ethylpropyl)-1-{[2-(hydroxymethyl)-4-(methylsulfonyl)phenyl]methyl}-2,5-piperazinedione

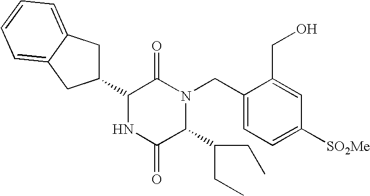

((3R,6R)-3-(2,3-Dihydro-1H-inden-2-yl)-6-(1-ethylpropyl)-1-{[2-(hydroxymethyl)-4-(methylthio)phenyl]methyl}-2,5-piperazinedione (Example 197) (167 mg, 0.36 mmol) was treated with 30% hydrogen peroxide (0.16 mL, 1.44 mmol) and 1 mL acetic acid at 80° C. for 6 hours. The solvent was then removed. The crude material was purified via combiflash silica gel column eluting with 0-5% Methanol in dichloromethane to give (3R,6R)-3-(2,3-dihydro-1H-inden-2-yl)-6-(1-ethylpropyl)-1-{[2-(hydroxymethyl)-4-(methylsulfonyl)phenyl]methyl}-2,5-piperazinedione (0.133 g, 75%) as a white solid.

LCMS (B) Rt=0.73 minutes; m/z [M+H]⁺=499

¹H NMR (CD₃OD) [symbol] 8.08 (s, 1H), 7.92 (d, 1H), 7.47 (d, 1H), 7.19 (m, 2H), 7.12 (m, 2H), 5.28 (d, 1H), 4.78 (dd, 2H), 4.52 (d, 1H), 4.41 (d, 1H), 4.00 (d, 1H), 3.26-3.33 (m, 1H), 3.16 (d, 1H), 3.10-3.16 (m, 1H), 3.00 (dd, 2H), 2.90 (dd, 1H), 1.87 (m, 1H), 1.66 (m, 3H), 1.30-1.41 (m, 1H), 0.90-1.00 (m, 6H).

### Example 208

### (3R,6R)-3-(2,3-Dihydro-1H-inden-2-yl)-6-(1-ethylpropyl)-1-{[4-(methylsulfonyl)-2-(1-pyrrolidinylcarbonyl)phenyl]methyl}-2,5-piperazinedione

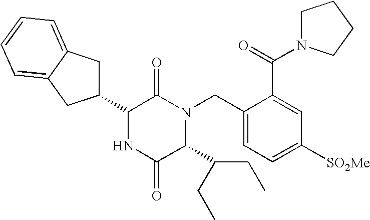

2-{[(3R,6R)-3-(2,3-Dihydro-1H-inden-2-yl)-6-(1-ethylpropyl)-2,5-dioxo-1-piperazinyl]-methyl}-5-(methylsulfonyl)benzoic acid (Int. 53) (50 mg, 0.097 mmol), HATU (48.0 mg, 0.126 mmol) and pyrrolidine (16 μL, 0.194 mmol) were dissolved in 1 mL acetonitrile. diisopropylethylamine (34 μL, 0.194 mmol) was then added. The resulting solution was stirred at RT until no more starting material was left as monitored by LCMS. The crude reaction mixture was then purified via Gilson HPLC eluting with 0.1% trifluoroacetic acid in acetonitrile/water. The product containing fractions were combined and concentrated. The residue was redissolved in 20 mL ethyl acetate, washed with 5 mL saturated sodium bicarbonate, dried over MgSO₄ and concentrated to give (3R,6R)-3-(2,3-dihydro-1H-inden-2-yl)-6-(1-ethylpropyl)-1-{[4-(methylsulfonyl)-2-(1-pyrrolidinyl-carbonyl)phenyl]methyl}-2,5-piperazinedione (8.8 mg, 16%) as a white solid.

LCMS (B) Rt=0.77 minutes; m/z [M+H]⁺=566

¹H NMR (CDCl₃) δ 7.94 (d, 1H), 7.88 (s, 1H), 7.54 (d, 1H), 7.18-7.26 (m, 4H), 6.74 (d, 1H), 5.02 (d, 1H), 4.42 (d, 1H), 4.12 (d, 1H), 4.05 (dd, 1H), 3.66-3.70 (m, 2H), 3.25-3.31 (m, 1H), 3.10-3.23 (m, 4H), 3.07 (s, 3H), 2.83-2.93 (m, 1H), 2.70-2.80 (m, 1H), 1.90-2.10 (m, 4H), 1.50-1.70 (m, 2H), 1.20-1.40 (m, 2H), 0.97 (t, 3H), 0.90 (t, 3H).

The following Examples were prepared by methods analogous to Example 208

| Ex | | | Rt/ | | |
|---|---|---|---|---|---|
| No | Structure | Mwt | min | +ve | Name |
| 209 | 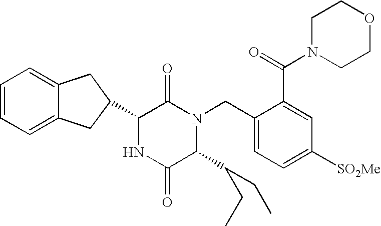 | 581.7 | 0.74(B) | 582 | (3R,6R)-3-(2,3-dihydro-1H-inden-2-yl)-6-(1-ethylpropyl)-1-{[4-(methylsulfonyl)-2-(4-morpholinylcarbonyl)phenyl]meth-yl}-2,5-piperazinedione |
| 210 | 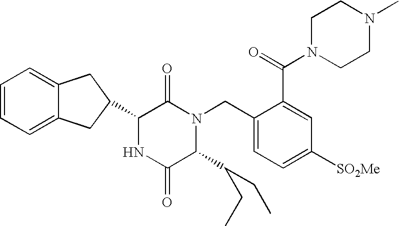 | 594.8 | 0.64(B) | 595 | (3R,6R)-3-(2,3-dihydro-1H-inden-2-yl)-6-(1-ethylpropyl)-1-{[2-[(4-methyl-1-piperazinyl)-carbonyl]-4-(methylsulfonyl)phenyl]methyl}-2,5-piperazine-dione |
| 211 | 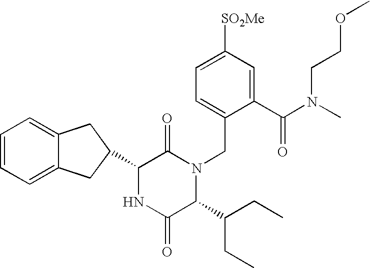 | 583.8 | 0.76(B) | 584 | 2-{[(3R,6R)-3-(2,3-dihydro-1H-inden-2-yl)-6-(1-ethylpropyl)-2,5-dioxo-1-piperazinyl]methyl}-N-methyl-N-[2-(methyloxy)ethyl]-5-(methylsulfonyl)benzamide |
| 211 | 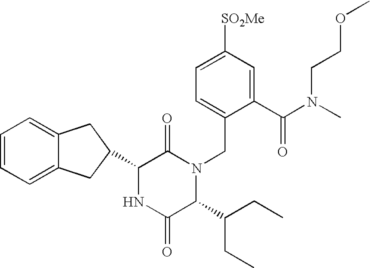 | 583.8 | 0.76(B) | 584 | 2-{[(3R,6R)-3-(2,3-dihydro-1H-inden-2-yl)-6-(1-ethylpropyl)-2,5-dioxo-1-piperazinyl]methyl}-N-methyl-N-(1-methyl-4-piperidinyl)benzamide |
| 212 | 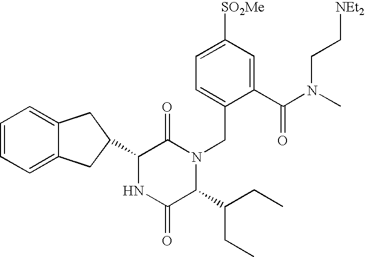 | 624.8 | 0.67(B) | 625 | N-[2-(diethylamino)ethyl]-2-{[(3R,6R)-3-(2,3-dihydro-1H-inden-2-yl)-6-(1-ethylpropyl)-2,5-dioxo-1-piperazinyl]methyl}-N-methyl-5-(methylsulfonyl)-benzamide |
| 213 | 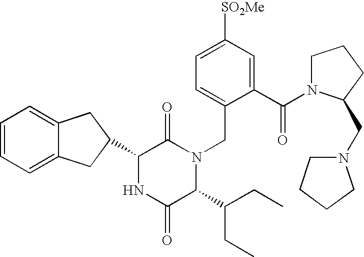 | 648.9 | 0.68(B) | 649 | (3R,6R)-3-(2,3-dihydro-1H-inden-2-yl)-6-(1-ethylpropyl)-1-[(4-(methylsulfonyl)-2-{[(2S)-2-(1-pyrrolidinylmethyl)-1-pyrrolidinyl]carbonyl}phenyl)meth-yl]-2,5-piperazinedione |

The following Examples were prepared by methods analogous to Example 89.

| Ex | | | Rt/ | +ve; | |
|---|---|---|---|---|---|
| No | Structure | MW | min | −ve | Name |
| 214 | 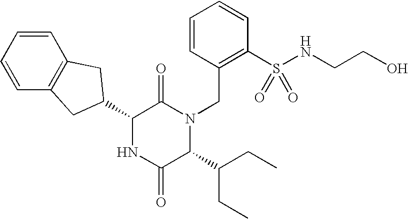 | 513.7 | 3.15(A) | 514;— | 2-{[(3R,6R)-3-(2,3-dihydro-1H-inden-2-yl)-6-(1-ethyl-propyl)-2,5-dioxo-1-piperazinyl]methyl}-N-(2-hydroxyethyl)benzene-sulfonamide |
| 215 | 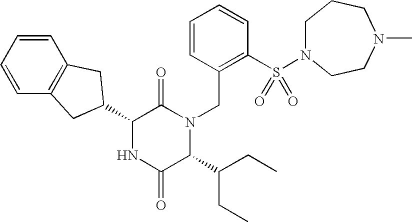 | 566.8 | 2.70(A) | 567;— | (3R,6R)-3-(2,3-dihydro-1H-inden-2-yl)-6-(1-ethylpropyl)-1-({2-[(4-methylhexahydro-1H-1,4-diazepin-1-yl)-sulfonyl]phenyl}methyl)-2,5-piperazinedione |
| 216 | 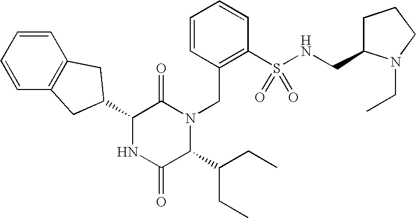 | 580.8 | 2.77(A) | 581;— | 2-{[(3R,6R)-3-(2,3-dihydro-1H-inden-2-yl)-6-(1-ethylpropyl)-2,5-dioxo-1-piperazinyl]methyl}-N-{[(2R)-1-ethyl-2-pyrrolidinyl]methyl}-benzenesulfonamide |
| 217 | 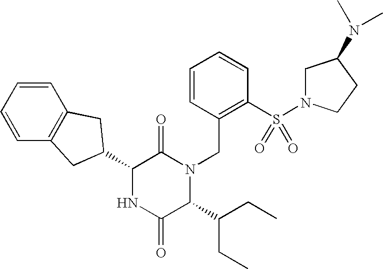 | 566.8 | 2.68(A) | 567;— | (3R,6R)-3-(2,3-dihydro-1H-inden-2-yl)-1-[(2-{[(3S)-3-(dimethylamino)-1-pyrrolidinyl]sulfonyl}phenyl)-methyl]-6-(1-ethylpropyl)-2,5-piperazinedione |
| 218 | 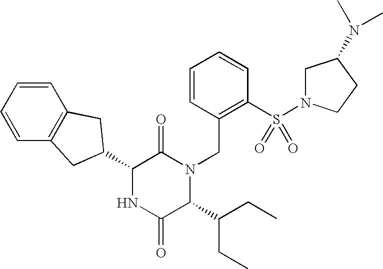 | 566.8 | 2.74(A) | 567;— | (3R,6R)-3-(2,3-dihydro-1H-inden-2-yl)-1-[(2-{[(3R)-3-(dimethylamino)-1-pyrrolidinyl]sulfonyl}phenyl)-methyl]-6-(1-ethylpropyl)-2,5-piperazinedione |

The following Example was prepared by a method analogous to Example 89, starting from Intermediate 47

| Ex | | | Rt/ | +ve; | |
|---|---|---|---|---|---|
| No | Structure | MW | min | −ve | Name |
| 219 | 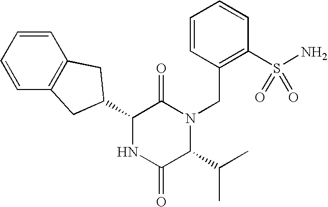 | 441 | 3.0(A) | 442;— | 2-{[(3R,6R)-3-(2,3-dihydro-1H-inden-2-yl)-6-(1-methylethyl)-2,5-dioxo-1-piperazinyl]methyl}benzene-sulfonamide |

### Example 220

### N-[(2-{[(3R,6R)-3-(2,3-dihydro-1H-inden-2-yl)-6-(1-ethylpropyl)-2,5-dioxo-1-piperazinyl]methyl}phenyl)sulfonyl]glycine

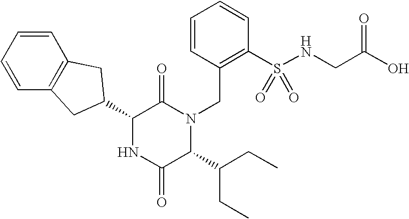

Phenylmethyl N-[(2-{[(3R,6R)-3-(2,3-dihydro-1H-inden-2-yl)-6-(1-ethylpropyl)-2,5-dioxo 1-piperazinyl]methyl}phenyl)sulfonyl]glycinate (Int. 16) (60 mg) was hydrogenated in ethanol (3 mL) at atmospheric pressure in the presence of palladium on carbon (20 mol % Pd) and acetic acid (0.5 mL). The resulting product was purified by MDAP to give 3 mg of pure material.

LCMS (A) Rt=3.28 minutes; m/z [M+H]⁺=528, [M]⁻=526.

¹H NMR δ 8.2 (br.s, 1H), 7.97 (d, 1H), 7.46 (t, 1H), 7.34 (t, 1H), 7.10-7.22 (m, 4H), 6.97 (d, 1H), 6.05 (br.s, 1H), 5.58 (br.d, 1H), 4.88 (br.d, 1H), 4.31 (d, 1H), 3.95 (d, 1H), 3.78 (br.s, 2H), 2.82-3.18 (m, 5H), 1.48-1.73 (m, 3H), 1.27 (m, 1H), 0.86 (m, 6H).

### Example 221

### (3R,6R)-3-(2,3-dihydro-1H-inden-2-yl)-6-(1-ethylpropyl)-1-({2-[(1-methylethyl)-sulfonyl]phenyl}methyl)-2,5-piperazinedione

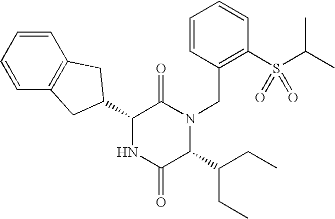

(3R,6R)-3-(2,3-Dihydro-1H-inden-2-yl)-6-(1-ethylpropyl)-1-[(2-mercaptophenyl)-methyl]-2,5-piperazinedione (Int. 19) (100 mg) was stirred with 2-iodopropane (26 uL) and diisopropylethylamine (45 uL) in THF (2 mL) for 18 hours then potassium tert-butoxide (29 mg) was added and the mixture heated to 50° C. for 2 hours and reduced in vacuo. The resulting residue was dissolved in DCM (2 mL) and 3-chloroperoxybenzoic acid (207 mg) was added. The mixture was stirred for 48 hours and passed through a 2 g aminopropyl SPE column eluting the crude product in methanol (10 mL). The eluent was reduced in vacuo and purified by MDAP to give 20 mg of a white solid.

HPLC (A) Rt=3.42 minutes; m/z [M+H]⁺=496.

¹H NMR δ 8.03 (d, 1H), 7.61 (t, 1H), 7.48 (t, 1H), 7.15-7.35 (m, 5H), 6.32 (br.s, 1H), 5.32 (d, 1H), 4.94 (d, 1H), 4.14 (dd, 1H), 4.09 (d, 1H), 3.37 (sex., 1H), 3.15 (m, 2H), 2.98 (sept., 1H), 2.82 (dd, 1H), 1.63 (m, 2H), 1.40 (d, 3H), 1.33 (m, 1H), 1.29 (d, 3H), 0.86 (t, 3H), 0.85 (t, 3H).

### Example 222

### (3R,6R)-1-[(2-Aminophenyl)methyl]-3-(2,3-dihydro-1H-inden-2-yl)-6-(2-methyl-propyl)-2,5-piperazinedione

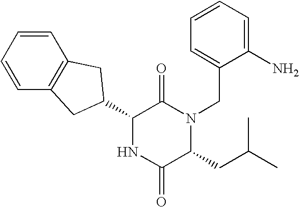

Hydrazine hydrate (0.5 g, 10 mmol) was added dropwise to a mixture of (3R,6R)-3-(2,3-dihydro-1H-inden-2-yl)-6-(2-methylpropyl)-1-[(2-nitrophenyl)methyl]-2,5-piperazinedione (Ex. 35) (1.3 g) and Raney nickel (0.5 g) in THF (20 ml). After 1 hour the catalyst was filtered off and the solution concentrated in vacuo to obtain the desired product (1.3 g, crude).

LCMS (D) Rt=2.63 minutes; m/z [M+H]⁺=392

¹H NMR (CDCl₃) δ 7.27-7.13 (m, 5H), 7.05 (d, 1H), 6.77-6.67 (m, 3H), 5.15 (d, 1H), 4.80 (br s, 2H), 4.09 (d, 1H), 4.02 (dd, 1H), 3.91 (dd, 1H), 3.20-3.06 (m, 3H), 2.94-2.77 (m, 2H), 1.98 (m, 1H), 1.78 (m, 1H), 1.65 (m, 1H), 1.01 (d, 3H), 0.95 (d, 3H).

The following Example was prepared by a method analogous to Example 118

| Ex | | | Rt/ | +ve; | |
|---|---|---|---|---|---|
| No | Structure | MW | min | −ve | Name |
| 223 | 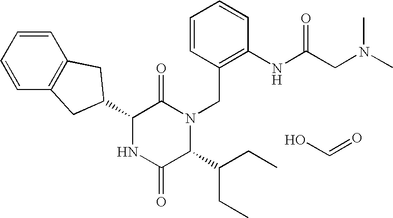 | 490.6 | 2.56(A) | 491;489 | N¹-(2-{[(3R,6R)-3-(2,3-dihydro-1H-inden-2-yl)-6-(1-ethylpropyl)-2,5-dioxo-1-piperazinyl]methyl}phenyl)-N²,N²-dimethylglycinamideformate |

The following Example was prepared by a method analogous to Example 122 except no (1R,2R)-(−)-N,N′-dimethylcyclohexane-1,2-diamine was used

| Ex | | | Rt/ | +ve; | |
|---|---|---|---|---|---|
| No | Structure | MW | min | −ve | Name |
| 224 | 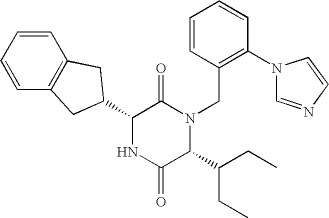 | 456.6 | 2.72(A) | 457;455 | (3R,6R)-3-(2,3-dihydro-1H-inden-2-yl)-6-(1-ethylpropyl)-1-{[2-(1H-imidazol-1-yl)phenyl]-methyl}-2,5-piperazinedione |

The following Example was prepared by a method analogous to Intermediate 21, using N,N-dimethyl-3-chloropropylamine as alkylating agent, and subsequent oxidation by a method analogous to Intermediate 22 without isolation of the intermediate sulfide

| Ex | | | Rt/ | +ve; | |
|---|---|---|---|---|---|
| No | Structure | MW | min | −ve | Name |
| 225 | 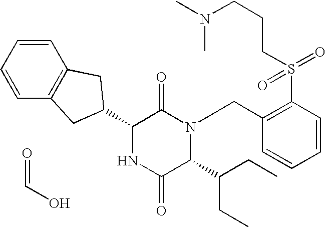 | 585.7 | 2.7(A) | 540;584 | formic acid-(3R,6R)-3-(2,3-dihydro-1H-inden-2-yl)-1-[(2-{[3-(dimethylamino)propyl]sulfonyl}-phenyl)methyl]-6-(1-ethylpropyl)-2,5-piperazinedione (1:1) |

### Example 226

### (3R,6R)-6-Cyclopentyl-3-(2,3-dihydro-1H-inden-2-yl)-1-{[4-(methylsulfonyl)phenyl]-methyl}-2,5-piperazinedione

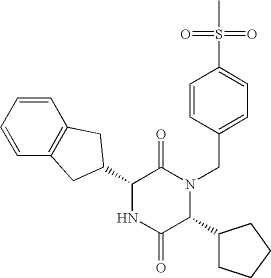

{[4-(Methylsulfonyl)phenyl]methyl}amine hydrochloride (0.85 g, 3.83 mmol) was dissolved in methanol (4 mL) and cyclopentanecarbaldehyde (0.31 mL, 3.83 mmol) added followed by (2R)-2,3-dihydro-1H-inden-2-yl({[(1,1-dimethylethyl)oxy]carbonyl}-amino)ethanoic acid (1.11 g, 3.83 mmol) and diisopropylethyl amine (0.63 mL, 3.61 mmol). The mixture was stirred for 15 minutes before 4-chlorophenylisonitrile (0.52 g, 3.83 mmol) was added. The mixture was stirred for 2.25 hours and then left to stand at room temperature overnight (20 hours) before it was cooled in an ice/water bath. Then acetyl chloride (1.6 mL, 22.80 mmol) was added dropwise, keeping the reaction temperature below 20° C. Then the mixture was stirred in the cooling bath for a further 10 minutes before it was stirred at room temperature. After 18 hours the mixture was evaporated under reduced pressure to leave a dark brown gum. The gum was stirred in chloroform (40 mL) and saturated aqueous sodium bicarbonate solution (40 mL) for 20 minutes before it was diluted with chloroform (40 mL) and the phases separated. The aqueous phase was extracted with chloroform (3×40 mL). The combined organic phase was dried (MgSO₄) and concentrated under reduced pressure. Chloroform (40 mL) was added to the residue and the resulting solution was treated with glacial acetic acid (1.6 mL) and left to stand, at room temperature over the weekend. Then the reaction mixture was washed with 2M hydrochloric acid (40 mL), followed by saturated aqueous sodium bicarbonate solution (40 mL). The organic phase was dried (MgSO₄) and evaporated under reduced pressure to give a brown foam. The crude residue was purified by flash chromatography on silica gel [Isco, 40 g RediSep® column, Hexanes/EtOAc 10% 60%] to give 200 mg of (3R,6R)-6-cyclopentyl-3-(2,3-dihydro-1H-inden-2-yl)-1-{[4-(methylsulfonyl)phenyl]methyl}-2,5-piperazinedione as an colorless oil.

¹H NMR (CDCl₃) δ 7.93 (d, 2H), 7.77 (d, 1H), 7.46 (d, 2H), 7.28-7.15 (m, 3H), 5.47 (d, 1H), 4.14 (m, 2H), 4.06 (dd, 1H), 3.75 (d, 1H), 3.15 (m, 3H), 3.06 (s, 3H), 2.87 (m, 2H), 2.24 (m, 1H), 2.01 (m, 1H), 1.80 (m, 2H), 1.60 (m, 4H).

HPLC (B) Rt=0.77 minutes; m/z [M+H]⁺=467.

### Example 227

### (3R,6R)-3-(2,3-Dihydro-1H-inden-2-yl)-1-[(1,5-dimethyl-1H-pyrazol-4-yl)methyl]-6-(1-ethylpropyl)-2,5-piperazinedione

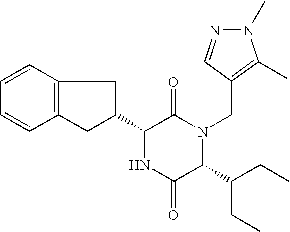

[(1,5-Dimethyl-1H-pyrazol-4-yl)methyl]amine hydrochloride was isolated as the free amine by means of an aminopropyl ion exchange column. To a solution of this material (280 mg) and (2R)-2,3-dihydro-1H-inden-2-yl ({[(1,1-dimethylethyl)oxy]carbonyl}amino)-ethanoic acid (580 mg) in methanol (4 ml) was added 2-{[(1,1-dimethylethyl)(dimethyl)-silyl]oxy}phenyl isocyanide (470 mg) followed by 2-ethylbutanal (250 μl) and molecular sieves. The reaction was then stirred overnight. Acetyl chloride (1.43 ml) was then added and the reaction heated at 50° C. overnight. The reaction was concentrated and the residue partitioned between chloroform and sodium bicarbonate and heated at 50° C. for 6 hours. Triethlyamine (3 eq) was then added and the reaction stirred at room temperature for a week. The organic layer was concentrated in vacuo and the residue purified by MDAP.

LCMS (A) Rt=3.1 minutes; m/z [M+H]⁺=409.

### Examples 228-229

### Isomers of (3R,6R)-3-(2,3-dihydro-1H-inden-2-yl)-6-(1-ethylpropyl)-1-{[2-(methyl-sulfinyl)phenyl]methyl}-2,5-piperazinedione

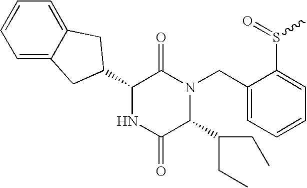

(3R,6R)-3-(2,3-Dihydro-1H-inden-2-yl)-6-(1-ethylpropyl)-1-{[2-(methylthio)phenyl]methyl}-2,5-piperazinedione (Ex. 143) (254 mg, 0.58 mmol) was added to a vigorously stirred suspension of wet alumina (580 mg, for prep see Synlett, 1992, 235) and oxone (357 mg, 0.58 mmol) in dichloromethane (2.9 ml) and the mixture cautiously heated at 40° C. and monitored by LCMS until the reaction was deemed complete. The reaction mixture was then filtered and concentrated to yield a white solid which was purified by silica chromatography (SPE, chloroform, dichloromethane, ether, ethylacetate, acetone and methanol) to yield a 2:1 mixture of the sulfoxide epimers as a white solid (160 mg). A 54 mg portion of this mixture was separated by chiral HPLC (Chiralpak AD, eluent 50% EtOH/heptane, 15 ml/min) to give:

Example 228, isomer 1 (26 mg) with HPLC retention time=11.6 min.

LC/MS (A) Rt=3.15 minutes; m/z [M+H]⁺=453; m/z [M+formate]⁻=497.

Example 229, isomer 2 (14 mg) with HPLC retention time 18.06 min.

LC/MS (A) Rt=3.17 minutes; m/z [M+H]⁺=453; m/z [M+formate]⁻=497.

The absolute stereochemistry at sulfur for each of these isomers is currently unknown.

### Examples 230-231

Isomers of (3R,6R)-3-(2,3-dihydro-1H-inden-2-yl)-6-(2-methylpropyl)-1-{[2-(methyl-sulfinyl)phenyl]ethyl}-2,5-piperazinedione were prepared from Ex. 144 by a method analogous to Examples 228-229, using 40% ethanol/heptane as eluent for the chiral separation.

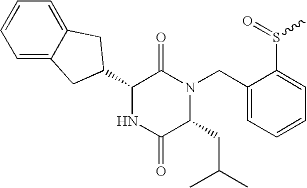

Example 230, isomer 1

LC/MS (A) Rt=3.05 minutes; m/z [M+H]⁺=439; m/z [M+formate]⁻=482.

Example 231, isomer 2

LC/MS (A) Rt=3.07 minutes; m/z [M+H]⁺=439; m/z [M+formate]⁻=482.

The absolute stereochemistry at sulfur for each of these isomers is currently unknown.

### Example 232

### (3R,6R)-3-(2,3-dihydro-1H-inden-2-yl)-6-(1-ethylpropyl)-1-{[2-(1H-pyrazol-1-yl)phenyl]methyl}-2,5-piperazinedione

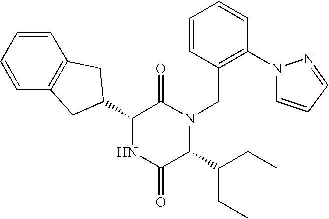

Potassium carbonate (118 mg), cuprous iodide (18 mg), pyrazole (58 mg), (3R,6R)-1-[(2-bromophenyl)methyl]-3-(2,3-dihydro-1H-inden-2-yl)-6-(1-ethylpropyl)-2,5-piperazinedione (Ex. 30) (200 mg, 0.4 mmol) and NMP (0.5 ml) were sequentially added to a 2 ml microwave tube. The mixture was heated with stirring at 190° C. for 2 hours in a microwave (Emrys™ Optimizer). The reaction mixture was diluted with dichloromethane and purified on an SPE cartridge (5 g, SCX2) eluting with ammonia/DCM. The relevant fractions were evaporated in vacuo to a green oil and further purified on a 5 g Si-SPE cartridge eluting with methanol in dichloromethane (0 to 10%). Evaporation of the relevant fraction in vacuo gave after freeze drying from dioxan the title compound (100 mg) as a cream solid.

LC/MS (A) Rt=3.41 minutes; m/z [M+H]⁺=457

### Example 233

(3R,6R)-1-[(2,4-difluorophenyl)methyl]-3-(2,3-dihydro-1H-inden-2-yl)-6-(2-methylpropyl)-2,5-piperazinedione LCMS (A) Rt=5.51 min; m/z [M+H]⁺=413; m/z [M−H]⁻=411.

### Example 234

(3R,6R)-3-(2,3-dihydro-1H-inden-2-yl)-6-(2-methylpropyl)-1-{[4-(methylsulfonyl)phenyl]methyl}-2,5-piperazinedione LCMS (A) Rt=3.01 min; m/z [M+H]⁺=455; m/z [M−H]⁻=453.

### Example 235

(3R,6R)-3-(2,3-dihydro-1H-inden-2-yl)-6-(2-methylpropyl)-1-[(1S)-1-(4-nitrophenyl)ethyl]-2,5-piperazinedione LCMS (A) Rt=3.43 min; m/z [M+H]⁺=436; m/z [M−H]⁻=434.

### Example 236

(3R,6R)-3-(2,3-dihydro-1H-inden-2-yl)-6-(2-methylpropyl)-1-[(4-nitrophenyl)methyl]-2,5-piperazinedione LCMS (A) Rt=3.34 min; m/z [M+H]⁺=422; m/z [M−H]⁻=420.

### Example 237

(3R,6R)-3-(2,3-dihydro-1H-inden-2-yl)-1-[(3-methyl-5-isoxazolyl)methyl]-6-(2-methylpropyl)-2,5-piperazinedione LCMS (A) Rt=3.01 min; m/z [M+H]⁺=382; m/z [M−H]⁻=380.

### Example 238

(3R,6R)-3-(2,3-dihydro-1H-inden-2-yl)-6-(2-methylpropyl)-1-{[2-(4-morpholinyl)phenyl]methyl}-2,5-piperazinedione LCMS (A) Rt=3.31 min; m/z [M+H]⁺=462; m/z [M−H]⁻=460.

### Example 239

(3R,6R)-3-(2,3-dihydro-1H-inden-2-yl)-1-[(1,5-dimethyl-1H-pyrazol-3-yl)methyl]-6-(2-methylpropyl)-2,5-piperazinedione LCMS (A) Rt=2.95 min; m/z [M+H]⁺=395; m/z [M+formate]⁻=439.

### Example 240

(3R,6R)-3-(2,3-dihydro-1H-inden-2-yl)-1-[2-(1-methyl-1H-imidazol-4-yl)ethyl]-6-(2-methylpropyl)-2,5-piperazinedione LCMS (A) Rt=2.33 min; m/z [M+H]⁺=395; m/z [M−H]⁻=393.

### Example 241

(3R,6R)-3-(2,3-dihydro-1H-inden-2-yl)-1-[(1,5-dimethyl-1H-pyrazol-4-yl)methyl]-6-(2-methylpropyl)-2,5-piperazinedione LCMS (A) Rt=2.97 min; m/z [M+H]⁺=395; m/z [M+formate]⁻=439.

### Example 242

(3R,6R)-3-(2,3-dihydro-1H-inden-2-yl)-6-(2-methylpropyl)-1-{[4-(4-morpholinyl)phenyl]methyl}-2,5-piperazinedione LCMS (A) Rt=3.32 min; m/z [M+H]⁺=462.

### Example 243

(3R,6R)-3-(2,3-dihydro-1H-inden-2-yl)-6-(2-methylpropyl)-1-[(2-oxo-1,2-dihydro-3-pyridinyl)methyl]-2,5-piperazinedione LCMS (A) Rt=2.76 min; m/z [M+H]⁺=394; m/z [M−H]⁻=392.

### Example 244

(3R,6R)-3-(2,3-dihydro-1H-inden-2-yl)-1-{[1-(4-methylphenyl)-1H-pyrazol-4-yl]methyl}-6-(2-methylpropyl)-2,5-piperazinedione ¹H NMR (CDCl₃) δ 7.89 (s, 1H), 7.65 (s, 1H), 7.52 (d, 2H), 7.26-7.13 (m, 5H), 6.93 (d, 1H), 5.08 (d, 1H), 4.02-3.90 (m, 3H), 3.10 (m, 3H), 2.95-2.75 (m, 2H), 2.38 (s, 3H), 2.0 (m, 1H), 1.90-1.60 (m, 3H), 1.02 (d, 3H), 0.97 (d, 3H).

### Example 245

N-(3-{[(3R,6R)-3-(2,3-dihydro-1H-inden-2-yl)-6-(2-methylpropyl)-2,5-dioxo-1-piperazinyl]methyl}phenyl)-N-methylacetamide ¹H NMR (CDCl₃) δ 7.50 (d, 1H), 7.39 (t, 1H), 7.25-7.07 (m, 6H), 5.32 (d, 1H), 4.06 (dd, 1H), 3.97 (d, 1H), 3.47 (s, 1H), 3.24 (s, 3H), 3.17-3.07 (m, 3H), 2.95-2.75 (m, 2H), 2.0-1.75 (m, 5H), 1.68-1.57 (m, 1H), 0.95 (d, 3H), 0.93 (d, 3H).

### Example 246

(3R,6R)-3-(2,3-dihydro-1H-inden-2-yl)-6-(2-methylpropyl)-1-{[5-(2-pyridinyl)-2-thienyl]methyl}-2,5-piperazinedione LCMS (A) Rt=3.36 min; m/z [M+H]⁺=460; m/z [M+formate]⁻=504.

### Example 247

(3R,6R)-3-(2,3-dihydro-1H-inden-2-yl)-6-(2-methylpropyl)-1-[(1S)-1-(4-methyl-1,3-thiazol-2-yl)ethyl]-2,5-piperazinedione LCMS (A) Rt=3.21 min; m/z [M+H]⁺=412; m/z [M+formate]⁻=456.

### Example 248

(3R,6R)-3-(2,3-dihydro-1H-inden-2-yl)-1-[(5-methyl-3-phenyl-4-isoxazolyl)methyl]-6-(2-methylpropyl)-2,5-piperazinedione LCMS (A) Rt=3.34 min; m/z [M+H]⁺=458; m/z [M+formate]⁻=502.

### Example 249

(3R,6R)-3-(2,3-dihydro-1H-inden-2-yl)-1-[(1,3-dimethyl-1H-pyrazol-4-yl)methyl]-6-(2-methylpropyl)-2,5-piperazinedione LCMS (A) Rt=2.91 min; m/z [M+H]⁺=395.

### Example 250

(3R,6R)-3-(2,3-dihydro-1H-inden-2-yl)-6-(2-methylpropyl)-1-{[4-(1H-pyrazol-1-yl)phenyl]methyl}-2,5-piperazinedione LCMS (B) Rt=0.83 min; m/z [M+H]⁺=443.

### Example 251

(3R,6R)-3-(2,3-dihydro-1H-inden-2-yl)-1-(3-isoxazolylmethyl)-6-(2-methylpropyl)-2,5-piperazinedione LCMS (A) Rt=3.00 min; m/z [M+H]⁺=368; m/z [M−H]⁻=366.

### Example 252

(3R,6R)-3-(2,3-dihydro-1H-inden-2-yl)-6-(2-methylpropyl)-1-[(5-methyl-2-pyrazinyl)methyl]-2,5-piperazinedione LCMS (A) Rt=2.93 min; m/z [M+H]⁺=393;

### Example 253

(3R,6R)-3-(2,3-dihydro-1H-inden-2-yl)-1-[(1-methyl-1H-benzimidazol-2-yl)methyl]-6-(2-methylpropyl)-2,5-piperazinedione LCMS (A) Rt=2.96 min; m/z [M+H]⁺=431; m/z [M−H]⁻=429.

### Example 254

(3R,6R)-3-(2,3-dihydro-1H-inden-2-yl)-1-[(3-{[(1,1-dimethylethyl)oxy]methyl}phenyl)methyl]-6-(2-methylpropyl)-2,5-piperazinedione LCMS (A) Rt=3.61 min; m/z [M+H]⁺=463.

### Example 255

(3R,6R)-1-[(4-acetylphenyl)methyl]-3-(2,3-dihydro-1H-inden-2-yl)-6-(2-methylpropyl)-2,5-piperazinedione LCMS (A) Rt=3.30 min; m/z [M+H]⁺=419; m/z [M−H]⁻=417.

### Example 256

(3R,6R)-3-(2,3-dihydro-1H-inden-2-yl)-6-(2-methylpropyl)-1-[(2-phenyl-2H-1,2,3-triazol-4-yl)methyl]-2,5-piperazinedione LCMS (A) Rt=3.53 min; m/z [M+H]⁺=444.

### Example 257

(3R,6R)-3-(2,3-dihydro-1H-inden-2-yl)-6-(2-methylpropyl)-1-(phenylmethyl)-2,5-piperazinedione LCMS (A) Rt=3.36 min; m/z [M+H]⁺=377; m/z [M+formate]⁻=421.

### Example 258

(3R,6R)-1-[(4-chlorophenyl)methyl]-3-(2,3-dihydro-1H-inden-2-yl)-6-(2-methylpropyl)-2,5-piperazinedione LCMS (A) Rt=3.52 min; m/z [M+H]⁺=411.

### Example 259

3-{[(3R,6R)-3-(2,3-dihydro-1H-inden-2-yl)-6-(2-methylpropyl)-2,5-dioxo-1-piperazinyl]methyl}-N-methylbenzamide LCMS (A) Rt=2.96 min; m/z [M+H]⁺=434. m/z [M−H]⁻=432.

### Example 260

(3R,6R)-3-(2,3-dihydro-1H-inden-2-yl)-6-(2-methylpropyl)-1-{[3-(methylsulfonyl)phenyl]methyl}-2,5-piperazinedione LCMS (A) Rt=3.03 min; m/z [M+H]⁺=455; m/z [M−H]⁻=453.

### Example 261

4-{[(3R,6R)-3-(2,3-dihydro-1H-inden-2-yl)-6-(2-methylpropyl)-2,5-dioxo-1-piperazinyl]methyl}-N,N-dimethylbenzenesulfonamide LCMS (A) Rt=3.21 min; m/z [M+H]⁺=484; m/z [M−H]⁻=482.

### Example 262

(3R,6R)-3-(2,3-dihydro-1H-inden-2-yl)-6-(2-methylpropyl)-1-{[4-(4-morpholinylcarbonyl)phenyl]methyl}-2,5-piperazinedione LCMS (B) Rt=0.74 min; m/z [M+H]⁺=490.

### Example 263

(3R,6R)-3-(2,3-dihydro-1H-inden-2-yl)-1-[(3-methyl-1,2,4-oxadiazol-5-yl)methyl]-6-(2-methylpropyl)-2,5-piperazinedione LCMS (A) Rt=3.01 min; m/z [M+H]⁺=383; m/z [M−H]⁻=381.

### Example 264

(3R,6R)-3-(2,3-dihydro-1H-inden-2-yl)-6-(2-methylpropyl)-1-({4-[(trifluoromethyl)sulfonyl]phenyl}methyl)-2,5-piperazinedione LCMS (A) Rt=3.49 min; m/z [M+H]⁺=509.

### Example 265

(3R,6R)-1-{[2-chloro-4-(methylsulfonyl)phenyl]methyl}-3-(2,3-dihydro-1H-inden-2-yl)-6-(2-methylpropyl)-2,5-piperazinedione LCMS (A) Rt=3.15 min; m/z [M+H]⁺=489/491; m/z [M−H]⁻=487/489.

### Example 266

(3R,6R)-3-(2,3-dihydro-1H-inden-2-yl)-1-[(5-methyl-1-phenyl-1H-pyrazol-4-yl)methyl]-6-(2-methylpropyl)-2,5-piperazinedione LCMS (A) Rt=3.21 min; m/z [M+H]⁺=457; m/z [M+formate]⁻=501.

### Example 267

(3R,6R)-3-(2,3-dihydro-1H-inden-2-yl)-1-[(4-{[ethyl(methyl)amino]methyl}phenyl)methyl]-6-(1-ethylpropyl)-2,5-piperazinedione LCMS (A) Rt=2.59 min; m/z [M+H]⁺=462; m/z [M+formate]⁻=506.

### Example 268

(3R,6R)-3-(2,3-dihydro-1H-inden-2-yl)-6-(1-ethylpropyl)-1-{[4-(1-pyrrolidinylmethyl)phenyl]methyl}-2,5-piperazinedione LCMS (A) Rt=2.61 min; m/z [M+H]⁺=474.

### Example 269

(3R,6R)-3-(2,3-dihydro-1H-inden-2-yl)-1-{[2-(methylsulfonyl)phenyl]methyl}-6-phenyl-2,5-piperazinedione ¹H NMR (CDCl₃) δ 8.08 (d, 1H), 7.60 (m, 2H), 7.49 (t, 1H), 7.33 (d, 1H), 7.25-7.15 (m, 4H), 5.48 (d, 1H), 4.71 (d, 1H), 4.07 (dd, 1H), 3.87 (d, 1H), 3.25 (s, 3H), 3.19 (dd, 1H), 3.12 (m, 2H), 2.98-2.81 (m, 2H).

### Example 270

(3R,6R)-6-cyclohexyl-3-(2,3-dihydro-1H-inden-2-yl)-1-[(3-methylphenyl)methyl]-2,5-piperazinedione LCMS (A) Rt=3.65 min; m/z [M+H]⁺=417; m/z [M+formate]⁻=461.

### Example 271

(3R,6R)-6-cyclopropyl-3-(2,3-dihydro-1H-inden-2-yl)-1-[(3-methylphenyl)methyl]-2,5-piperazinedione LCMS (A) Rt=3.24 min; m/z [M+H]⁺=375; m/z [M+formate]⁻=419.

### Example 272

(3R,6R)-3-(2,3-dihydro-1H-inden-2-yl)-6-(1-methylethyl)-1-{[2-(1-piperazinylsulfonyl)phenyl]methyl}-2,5-piperazinedione hydrochloride LCMS (A) Rt=3.79 min; m/z [M+H]⁺=491; m/z [M−H]-=489.

### Example 273

1,1-dimethylethyl 3-{[(3R,6R)-3-(2,3-dihydro-1H-inden-2-yl)-6-(1-ethylpropyl)-2,5-dioxo-1-piperazinyl]methyl}benzoate LCMS (A) Rt=3.77 min; m/z [M+H]⁺=491; m/z [M−H]-=489.

### Example 274

(3S,6S)-3-(2,3-dihydro-1H-inden-2-yl)-6-(1-ethylpropyl)-1-{[2-(methylsulfonyl)phenyl]methyl}-2,5-piperazinedione LCMS (A) Rt=3.25 min; m/z [M+H]⁺=469; m/z [M−H]-=467.

### Example 275

(3R,6R)-3-(2,3-dihydro-1H-inden-2-yl)-6-(2-methylpropyl)-1-{[4-(2-pyrazinylamino)phenyl]methyl}-2,5-piperazinedione LCMS (B) Rt=0.78 min; m/z [M+H]⁺=470.

### Example 276

(3R,6R)-3-(2,3-dihydro-1H-inden-2-yl)-6-(2-methylpropyl)-1-{[4-(1H-1,2,3-triazol-1-yl)phenyl]methyl}-2,5-piperazinedione LCMS (B) Rt=0.76 min; m/z [M+H]⁺=444.

### Example 277

(3R,6R)-3-(2,3-dihydro-1H-inden-2-yl)-6-(2-methylpropyl)-1-{[4-(1H-1,2,4-triazol-1-yl)phenyl]methyl}-2,5-piperazinedione LCMS (B) Rt=0.75 min; m/z [M+H]⁺=444.

### Example 278

(3R,6R)-6-cyclopentyl-3-(2,3-dihydro-1H-inden-2-yl)-1-{[4-(4-morpholinylcarbonyl)phenyl]methyl}-2,5-piperazinedione LCMS (B) Rt=0.76 min; m/z [M+H]⁺=502.

### Example 279

N-(2-{[(3R,6R)-3-(2,3-dihydro-1H-inden-2-yl)-6-(1-ethylpropyl)-2,5-dioxo-1-piperazinyl]methyl}phenyl)-4-morpholinecarboxamide HPLC (D) Rt=2.92 min; m/z [M+H]⁺=519.

### Example 280

(3R,6R)-3-(2,3-dihydro-1H-inden-2-yl)-6-(2-methylpropyl)-1-{[4-(2H-1,2,3-triazol-2-yl)phenyl]methyl}-2,5-piperazinedione LCMS (B) Rt=0.85 min; m/z [M+H]⁺=444.

### Example 281

4-{[(3R,6R)-3-(2,3-dihydro-1H-inden-2-yl)-6-(2-methylpropyl)-2,5-dioxo-1-piperazinyl]methyl}-N-(2-hydroxyethyl)benzamide LCMS (B) Rt=0.69 min; m/z [M+H]⁺=464.

### Example 282

4-{[(3R,6R)-3-(2,3-dihydro-1H-inden-2-yl)-6-(2-methylpropyl)-2,5-dioxo-1-piperazinyl]methyl}-N-methyl-N-[2-(methyloxy)ethyl]benzamide LCMS (D) Rt=2.62 min; m/z [M+H]⁺=492.

### Example 283

4-{[(3R,6R)-3-(2,3-dihydro-1H-inden-2-yl)-6-(2-methylpropyl)-2,5-dioxo-1-piperazinyl]methyl}-N-(2-hydroxyethyl)-N-methylbenzamide LCMS (D) Rt=2.39 min; m/z [M+H]⁺=478.

### Example 284

N-(2-{[(3R,6R)-3-(2,3-dihydro-1H-inden-2-yl)-6-(2-methylpropyl)-2,5-dioxo-1-piperazinyl]methyl}phenyl)-4-morpholinecarboxamide LCMS (B) Rt=0.76 min; m/z [M+H]⁺=505.

### Example 285

4-{[(3R,6R)-3-(2,3-dihydro-1H-inden-2-yl)-6-(2-methylpropyl)-2,5-dioxo-1-piperazinyl]methyl}-N-[2-(dimethylamino)ethyl]-N-methylbenzamide LCMS (D) Rt=2.29 min; m/z [M+H]⁺=505.

### Example 286

The preparation of this compound has been described as Intermediate 14: (3R,6R)-3-(2,3-dihydro-1H-inden-2-yl)-1-{[2-(hydroxymethyl)phenyl]methyl}-6-(2-methylpropyl)-2,5-piperazinedione.

### Example 287

The preparation of this compound has been described as Intermediate 15: 1,1-Dimethylethyl 3-{[(3R,6R)-3-(2,3-dihydro-1H-inden-2-yl)-6-(1-ethylpropyl)-2,5-dioxo-1-piperazinyl]methyl}benzoate.

### Example 288

The preparation of this compound has been described as Intermediate 45: (3R,6R)-3-(2,3-dihydro-1H-inden-2-yl)-1-({2-[(1,1-dimethylethyl)thio]phenyl}methyl)-6-(1-methyl ethyl)-2,5-piperazinedione.

### Example 289

The preparation of this compound has been described as Intermediate 52: 4-{[(3R,6R)-3-(2,3-dihydro-1H-inden-2-yl)-6-(1-ethylpropyl)-2,5-dioxo-1-piperazinyl]methyl}-1,3-benzenedicarboxylic acid.

### Example 290

The preparation of this compound has been described as Intermediate 53: 2-{[(3R,6R)-3-(2,3-Dihydro-1H-inden-2-yl)-6-(1-ethylpropyl)-2,5-dioxo-1-piperazinyl]-methyl}-5-(methylsulfonyl)benzoic acid.

### Example 291

The preparation of this compound has been described as Intermediate 54: 1,1-dimethylethyl 4-{[(3R,6R)-3-(2,3-dihydro-1H-inden-2-yl)-6-(1-ethylpropyl)-2,5-dioxo-1-piperazinyl]methyl}benzoate.

### Assay Procedures

### Assay 1) Determination of Antagonist Affinity at Human Oxytocin-1 Receptors Using FLIPR

### Cell Culture

Adherent Chinese Hamster Ovary (CHO) cells, stably expressing the recombinant human Oxytocin-1 (hOT) receptor, were maintained in culture in DMEM:F12 medium (Sigma, cat no D6421), supplemented with 10% heat inactivated foetal calf serum (Gibco/Invitrogen, cat. no. 01000-147), 2 mM L-glutamine (Gibco/Invitrogen, cat. no. 25030-024) and 0.2 mg/ml G418 (Gibco/Invitrogen, cat no. 10131-027). Cells were grown as monolayers under 95%:5% air:CO₂ at 37° C. and passaged every 3-4 days using TrypLE™ Express (Gibco/Invitrogen, cat no. 12604-013).

### Measurement of [Ca²⁺]ᵢ Using the FLIPR™

CHO-hOT cells were seeded into black walled clear-base 384-well plates (Nunc) at a density of 10,000 cells per well in culture medium as described above and maintained overnight (95%:5% air:CO₂ at 37° C.). After removal of culture medium, cells were incubated for 1 h at 37° C. in Tyrode's medium (NaCl, 145 mM; KCl, 2.5 mM; HEPES, 10 mM; Glucose, 10 mM; MgCl₂, 1.2 mM; CaCl₂, 1.5 mM) containing probenacid (0.7 mg/ml), the cytoplasmic calcium indicator, Fluo-4 (4 uM; Teflabs, USA) and the quenching agent Brilliant Black (250 uM; Molecular Devices, UK). Cells were then incubated for an additional 30 min at 37° C. with either buffer alone or buffer containing OT antagonist, before being placed into a FLIPR™ (Molecular Devices, UK) to monitor cell fluorescence (λₑₓ=488 nm, λ_{EM}=540 nm) before and after the addition of a submaximal concentration of oxytocin (EC80).

### Data Analysis

Functional responses using FLIPR were analysed using Activity Base Version 5.0.10.

### Assay 2) Determination of Estimated Antagonist Affinity at Human Oxytocin-1 Receptors Using Fluorescence Polarisation

### Cell Culture

Adherent Chinese Hamster Ovary (CHO) cells, stably expressing the recombinant human Oxytocin-1 (hOT) receptor, were maintained in culture in DMEM:F12 medium (Sigma, cat no D6421), supplemented with 10% foetal calf serum (Gibco/Invitrogen, cat. no. 01000-147), 2 mM L-glutamine (Gibco/Invitrogen, cat. no. 25030-024) and 0.5 mg/ml G418 (Gibco/Invitrogen, cat no. 10131-027). Cells were grown as monolayers under 95%:5% air:CO₂ at 37° C. and passaged every 3-4 days using HBSS+0.6 mM EDTA.

### Membrane Preparation (GSK UK Gene Expression & Protein Biochemistry).

Membranes were prepared from cells cultured in 1800 cm2 roller bottles. Harvested cells (HBSS+0.6 mM EDTA) were centrifuged at 250 g for 5 mins at 4° C. This was repeated after re-suspending the pellets in 200 mls on HBSS+0.6 mM EDTA. All subsequent steps were performed at 4° C. The cells were homogenised for 2×15 secs in 200 mls of 50 mM HEPES+10-4M leupeptin+25 ug/ml bacitracin+1 mM EDTA+1 mM PMSF+2 uM Pepstatin A, (the latter 2 reagents added as fresh ×100 and ×500 stocks respectively in ethanol). The homogenate was plunged onto ice for 5 mins after the first burst and 10-40 mins after the final burst to dissipate the foam. The homogenate was then centrifuged at 500 g for 20 mins and the supernatant centrifuged for 36 mins at 48,000 g. The pellet was re-suspended in the same buffer as above but without PMSF and Pepstatin A. The material was then forced through a 0.6 mm needle, made up to the required volume, (usually x4 the volume of the original cell pellet), aliquoted and stored frozen at −80 deg C.

### Measurement of OT Binding Using Fluorescence Polarisation

Compounds were prepared as 1 in 4 serial dilutions in 100% DMSO. Diluted compound was added to black Greiner 384 microplate at 0.5 μl/well. Bodipy Tamra labelled oxytocin (Perkin Elmer Life Sciences custom request CUS54801) was diluted in assay buffer (10 mM HEPES, 10 mM MgCl2, 0.125 mg/ml bovine serum albumin: pH7.4 with KOH) and added to the microplate using to give a final assay concentration of 0.5 nM @ 20 μl/well, followed by the addition of CHO-hOT membrane, diluted in assay buffer, 20 μl/well, to give a final assay concentration of 2.5 μg/well. The plates were protected from light and incubated at room temperature for 2 hours before being read on an LJL Analyst (Molecular Devices) using Excitation filter wave length 535/22, Emission filter: wave length 580/30.

### Data Analysis

Fluorescence Polarisation units from both the parallel and perpendicular reads were used to calculate the Anisotropy & Total Intensity of the compounds. The anisotropy values under went normalisation conversion then fitted to the 4 parameter logistic equation. As the assay is providing an estimate of receptor affinity, functional pKi correction (Cheng Prusoff relationship) was applied, assuming the pKd of Bodipy Tamra to be 9.9 (4× ligand concentration added). All analysis was conducted using Activity Base version 5.0 (IDBS).

### Activity and Utility of the Compounds of the Invention

Examples 1-185 and 187-232 were found to have at least one of

i) a pKi value of 6.9 or greater in Assay 1;
ii) a pKi value of 7.5 or greater in Assay 2.

Examples 233-285 were found to have measurable activity in at least one of Assay 1 and Assay 2. Examples 233-285 may also have utility as Intermediates in the preparation of other compounds of Formula (I) or Formula (A).

Examples 186 and 286-291 were not tested in the Assays and have utility as Intermediates in the preparation of other compounds of Formula (I) or Formula (A).

What is claimed is:

1. A compound of Formula (I) 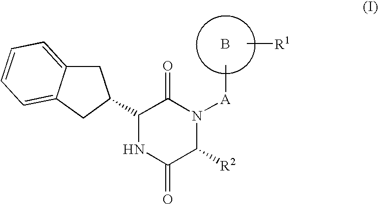
and a pharmaceutically acceptable salt, solvate, or prodrug thereof,
wherein:
A represents a C₁₋₄alkylene group optionally substituted by one or more C₁₋₄alkyl groups;
B represents a mono-, bi- or tricyclic aryl or heteroaryl ring containing one or more heteroatoms independently selected from O, S and N, wherein the aryl or heteroaryl ring is optionally substituted by one or more R¹ groups independently selected from C₁₋₆cycloalkyl, C₁₋₆alkyl, C₁₋₆cycloalkoxy, C₁₋₆alkoxy, aryl, aralkyl, heterocyclyl, heteroaryl, —Oheterocyclyl, —Oheteroaryl, —S(O)ₙheterocyclyl and —S(O)ₙheteroaryl each of which is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴; or each R¹ is independently selected from halo, hydroxyl, —NR³R⁴, nitro, cyano, trifluoroC₁₋₄alkyl, trifluoroC₁₋₄alkoxy, carboxyl, —CONR³R⁴, —COR⁵, —S(O)ₙR⁶, —NR⁷COR⁸, —S(O)ₘNR⁹R¹⁰ and —NR¹¹S(O)ₘR¹²;
R² represents C₃₋₇alkyl, C₃₋₇ cycloalkyl or phenyl, each of which is optionally substituted by one or more groups selected from C₁₋₄alkyl or and C₃₋₇ cycloalkyl;
R³ and R⁴ independently represent H, C₁₋₆alkyl, C₁₋₆cycloalkyl, aryl, heterocyclyl or heteroaryl, wherein the C₁₋₆alkyl, C₁₋₆cycloalkyl, aryl, heterocyclyl or heteroaryl is optionally substituted by one or more groups independently selected from halo, hydroxyl, carboxyl, C₁₋₆alkyl, C₁₋₆alkoxy, C₁₋₃alkoxyC₁₋₆alkyl, trifluoroC₁₋₄alkyl, trifluoroC₁₋₄alkoxy, —S(O)ₙR⁶, COR⁵, heteroaryl, heterocyclyl, aryl and —NR^{3a}R^{4a};
or R³ and R⁴ together form a 5- or 6-membered heteroaryl or a 4- to 7-membered heterocyclyl ring, which ring optionally contains 1 or 2 additional heteroatoms independently selected from O, S and N, wherein the 5- or 6-membered heteroaryl or 4- to 7-membered heterocyclyl ring may be further is optionally substituted by one or more groups selected from C₁₋₄alkyl, C₁₋₄alkoxy, trifluoroC₁₋₄alkyl, trifluoroC₁₋₄alkoxy, —NR^{3a}R^{4a}, COR⁵, hydroxyl, aryl, heteroaryl and heterocyclyl, wherein the C₁₋₄alkyl, C₁₋₄alkoxy, aryl, heteroaryl or heterocyclyl groups on the 5- or 6-membered heteroaryl or 4- to 7-membered heterocyclyl ring may be further is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, COR⁵, heteroaryl, heterocyclyl, aryl and —NR^{3a}R^{4a};
R^{3a} and R^{4a} independently represent H, C₁₋₆alkyl, C₁₋₆cycloalkyl, aryl, heterocyclyl or heteroaryl, wherein the C₁₋₆alkyl, C₁₋₆cycloalkyl, aryl, heterocyclyl or heteroaryl is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, and aryl;
or R^{3a} and R^{4a} together form a 5- or 6-membered heteroaryl or a 4- to 7-membered heterocyclyl ring which ring optionally contains 1 or 2 additional heteroatoms independently selected from O, S and N, wherein the 5- or 6-membered heteroaryl or 4- to 7-membered heterocyclyl ring is optionally substituted by one or more groups selected from C₁₋₄alkyl, C₁₋₄alkoxy, trifluoroC₁₋₄alkyl, trifluoroC₁₋₄alkoxy, hydroxyl, aryl, heteroaryl and heterocyclyl wherein the C₁₋₄alkyl, C₁₋₄alkoxy, aryl, heteroaryl or heterocyclyl groups on the 5- or 6-membered heteroaryl or 4- to 7-membered heterocyclyl ring is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, and aryl;
R⁵ represents C₁₋₆alkyl, C₁₋₆alkoxy, trifluoroC₁₋₆alkyl, aryl, heteroaryl or heterocyclyl, wherein the C₁₋₆alkyl, C₁₋₆alkoxy, trifluoroC₁₋₆alkyl, aryl, heteroaryl or heterocyclyl is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
R⁶ represents C₁₋₆alkyl, C₁₋₆cycloalkyl, trifluoroC₁₋₆alkyl, aryl, heteroaryl, or heterocyclyl, wherein the C₁₋₆alkyl, C₁₋₆cycloalkyl, trifluoroC₁₋₆alkyl, aryl, heteroaryl, or heterocyclyl is optionally substituted by one or more groups independently selected from halo, hydroxyl, carboxyl, C₁₋₆alkyl, C₁₋₆alkoxy, C₁₋₃alkoxyC₁₋₆alkyl, trifluoroC₁₋₄alkyl, trifluoroC₁₋₄alkoxy, heteroaryl, heterocyclyl, aryl and —NR³R⁴;
R⁷ represents H or C₁₋₄alkyl optionally substituted by one or more groups independently selected from by halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
R₈ represents C₁₋₆alkyl, C₁₋₆cycloalkyl, aryl, heterocyclyl or heteroaryl each of which is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
or R⁷ and R⁸ together form a 4- to 7-membered heterocyclyl ring which optionally contains one or more heteroatoms independently selected from O, S and N, and wherein the heterocyclyl ring is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
R⁹ and R¹⁰ independently represent H, C₁₋₆alkyl, C₁₋₆cycloalkyl, aryl, heterocyclyl or heteroaryl, wherein the C₁₋₆alkyl, C₁₋₆cycloalkyl, aryl, heterocyclyl or heteroaryl is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, aryl, —NR³R⁴ and heterocyclyl optionally substituted with C₁₋₆alkyl;
or R⁹ and R¹⁰ form a 5- or 6-membered heteroaryl or a 4- to 7-membered heterocyclyl ring which ring optionally contains 1 or 2 additional heteroatoms independently selected from O, S and N, wherein the 5- or 6-membered heteroaryl or 4- to 7-membered heterocyclyl ring is optionally substituted by one or more groups selected from C₁₋₄alkyl, C₁₋₄alkoxy, trifluoroC₁₋₄alkyl, trifluoroC₁₋₄alkoxy, —NR³R⁴, hydroxyl, aryl, heteroaryl and heterocyclyl, wherein the C₁₋₄alkyl, C₁₋₄alkoxy, aryl, heteroaryl or heterocyclyl groups on the 5- or 6-membered heteroaryl or 4- to 7-membered heterocyclyl ring is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
R¹¹ represents H or C₁₋₄alkyl optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
R¹² represents C₁₋₆alkyl, C₁₋₆cycloalkyl, aryl, heterocyclyl or heteroaryl each of which is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
or R¹¹ and R¹² form a 4- to 7-membered heterocyclyl ring which optionally contains one or more additional heteroatoms independently selected from O, S and N, wherein the heterocyclyl ring is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴;
n represents 0, 1 or 2;
and m represents 1 or 2.

2. A compound of Formula (I) and a pharmaceutically acceptable salt, solvate, or prodrug thereof, provided that the compound of Formula (I) is not one of the following:
(3R,6R)-3-(2,3-dihydro-1H-inden-2-yl)-6-(2-methylpropyl)-1-[2-(2-pyridinyl)ethyl]-2,5-piperazinedione;
(3R,6R)-3-(2,3-dihydro-1H-inden-2-yl)-6-(2-methylpropyl)-1-[2-(4-nitrophenyl)ethyl]-2,5-piperazinedione;
(3R,6R)-3-(2,3-dihydro-1H-inden-2-yl)-6-(2-methylpropyl)-1-[2-(4-pyridinyl)ethyl]-2,5-piperazinedione
(3R,6R)-3-(2,3-dihydro-1H-inden-2-yl)-6-(2-methylpropyl)-1-(4-pyridinylmethyl)-2,5-piperazinedione;
(3R,6R)-3-(2,3-dihydro-1H-inden-2-yl)-6-(2-methylpropyl)-1-[2-(5,6,7,8-tetrahydroimidazo[1,5-a]pyridin-1-yl)ethyl]-2,5-piperazinedione;
(3R,6R)-3-(2,3-dihydro-1H-inden-2-yl)-6-(2-methylpropyl)-1-{[6-(trifluoromethyl)-3-pyridinyl]methyl}-2,5-piperazinedione;
(3R,6R)-3-(2,3-dihydro-1H-inden-2-yl)-1-[2-(1-methyl-1H-imidazol-2-yl)ethyl]-6-(2-methylpropyl)-2,5-piperazinedione;
(3R,6R)-3-(2,3-dihydro-1H-inden-2-yl)-6-(2-methylpropyl)-1-{[4-(3-pyridinyl)phenyl]-methyl}-2,5-piperazinedione;
(3R,6R)-3-(2,3-dihydro-1H-inden-2-yl)-1-[2-(1-methyl-1H-imidazol-5-yl)ethyl]-6-(2-methylpropyl)-2,5-piperazinedione;
(3R,6R)-3-(2,3-dihydro-1H-inden-2-yl)-6-(2-methylpropyl)-1-{2-[4-(methylsulfonyl)phenyl]ethyl}-2,5-piperazinedione;
N-(3-{2-[(3R,6R)-3-(2,3-dihydro-1H-inden-2-yl)-6-(2-methylpropyl)-2,5-dioxo-1-piperazinyl]ethyl}phenyl)methanesulfonamide;
(3R,6R)-3-(2,3-dihydro-1H-inden-2-yl)-6-(2-methylpropyl)-1-[(1-methyl-1H-pyrazol-4-yl)methyl]-2,5-piperazinedione;
(3R,6R)-3-(2,3-dihydro-1H-inden-2-yl)-6-(2-methylpropyl)-1-{[4-(4-morpholinylmethyl)phenyl]methyl}-2,5-piperazinedione;
(3R,6R)-3-(2,3-dihydro-1H-inden-2-yl)-6-(2-methylpropyl)-1-[(2-methyl-2H-tetrazol-5-yl)methyl]-2,5-piperazinedione;
(3R,6R)-3-(2,3-dihydro-1H-inden-2-yl)-1-[(1-ethyl-5-methyl-1H-pyrazol-4-yl)methyl]-6-(2-methylpropyl)-2,5-piperazinedione;
(3R,6R)-3-(2,3-dihydro-1H-inden-2-yl)-1-[(1-ethyl-1H-pyrazol-4-yl)methyl]-6-(2-methylpropyl)-2,5-piperazinedione;
(3R,6R)-3-(2,3-dihydro-1H-inden-2-yl)-6-(2-methylpropyl)-1-{[1-methyl-5-(trifluoromethyl)-1H-pyrazol-4-yl]methyl}-2,5-piperazinedione;
(3R,6R)-3-(2,3-dihydro-1H-inden-2-yl)-1-({4-[(dimethylamino)methyl]phenyl}methyl)-6-(1-ethylpropyl)-2,5-piperazinedione;
(3R,6R)-1-{[3,5-bis(trifluoromethyl)phenyl]methyl}-3-(2,3-dihydro-1H-inden-2-yl)-6-(1-ethylpropyl)-2,5-piperazinedione;
(3R,6R)-6-cyclopropyl-3-(2,3-dihydro-1H-inden-2-yl)-1-{[2-(methylsulfonyl)phenyl]-methyl}-2,5-piperazinedione;
(3R,6R)-3-(2,3-dihydro-1H-inden-2-yl)-1-[(3-methylphenyl)methyl]-6-phenyl-2,5-piperazinedione; and
(3R,6R)-3-(2,3-dihydro-1H-inden-2-yl)-1-[(2-{[3-(dimethylamino)propyl]sulfinyl}phenyl)methyl]-6-(1-ethylpropyl)-2,5-piperazinedione.

3. A compound according to claim 2, wherein A represents CH₂, CH(CH₃) or CH₂CH₂.

4. A compound according to claim 3, wherein the ring B represents phenyl, pyridyl, pyrimidinyl, quinolinyl or pyrazolyl.

5. A compound according to claim 3, wherein the one or more R¹ groups may be independently selected from C₁₋₆alkyl, C₁₋₆alkoxy, aryl, aralkyl, heterocyclyl, and heteroaryl, each of which is optionally substituted by one or more groups independently selected from hydroxyl, C₁₋₆alkyl, C₁₋₆alkoxy, heterocyclyl, aryl and —NR³R⁴); or R¹ may additionally be independently selected from halo, hydroxyl, —NR³R⁴, nitro, trifluoroC₁₋₄alkyl, trifluoroC₁₋₄alkoxy, carboxyl, —CONR³R⁴, —COR⁵, —S(O)ₙR⁶, —NR⁷COR⁸, —S(O)ₘNR⁹R¹⁰ and —NR¹¹S(O)ₘR¹².

6. A compound according to claim 3, wherein R² represents C₃₋₅alkyl, or R² represents C₃₋₅cycloalkyl which may be further optionally substituted by C₁₋₂alkyl, wherein the total number of carbon atoms in the R² group is between 3 and 5.

7. A compound according to claim 3, wherein R³ and R⁴ independently represent H or C₁₋₄alkyl which is optionally substituted by one or more groups independently selected from hydroxyl, C₁₋₂alkyl and —NR^{3a}R^{4a}, or R³ and R⁴ together form a 5- or 6-membered heterocyclyl ring, which ring optionally contain 1 or 2 heteroatoms independently selected from O, S and N, wherein the 5- or 6-membered heterocyclyl ring is optionally substituted by C₁₋₄alkyl.

8. A compound according to claim 3, wherein R^{3a} and R^{4a} independently represent C₁₋₆alkyl.

9. A compound according to claim 3, wherein R⁵ represents C₁₋₆alkoxy which is optionally substituted with hydroxyl, C₁₋₆alkoxy, or —NR³R⁴.

10. A compound according to claim 3, wherein R⁶ represents C₁₋₆alkyl, trifluoroC₁₋₆alkyl or heterocyclyl, each of which is optionally substituted by one or more groups independently selected from C₁₋₆alkyl, C₁₋₃alkoxyC₁₋₆alkyl, heterocyclyl and —NR³R⁴.

11. A compound according to claim 3, wherein R⁷ represents H or C₁₋₄alkyl.

12. A compound according to claim 3, wherein R₈ represents C₁₋₆alkyl or heterocyclyl or heteroaryl each of which is optionally substituted by one or more groups independently selected from C₁₋₆alkyl and —NR³R⁴.

13. A compound according to claim 3, wherein R⁹ and R¹⁰ independently represent H, C₁₋₆alkyl, heterocyclyl or heteroaryl each of which is optionally substituted by one or more groups independently selected from hydroxyl, carboxyl, C₁₋₆alkyl, aryl —NR³R⁴ and heterocyclyl optionally substituted by C₁₋₆alkyl, or R⁹ and R¹⁰ together form a 5-, 6- or 7-membered heterocyclyl ring which ring optionally contains 1 or 2 additional heteroatoms independently selected from O, S and N and wherein the 5-, 6- or 7-membered heterocyclyl ring may be further optionally substituted by one or more groups selected from C₁₋₄alkyl, and —NR³R⁴, wherein the C₁₋₄alkyl group is optionally substituted by C₁₋₆alkoxy.

14. A compound according to claim 3, wherein R¹¹ represents H or C₁₋₄alkyl.

15. A compound according to claim 3, wherein R¹² represents C₁₋₆alkyl.

16. A compound according to claim 3, wherein R¹¹ and R¹² together with the form a 5- or 6-membered heterocyclyl ring which ring may additionally contain one or more heteroatoms independently selected from O, S of and N, wherein the heterocyclyl ring is optionally substituted by one or more groups independently selected from halo, hydroxyl, carboxyl, C₁₋₆alkyl, C₁₋₆alkoxy, trifluoroC₁₋₄alkyl, trifluoroC₁₋₄alkoxy, —S(O)ₙR⁶, heteroaryl, heterocyclyl, aryl and —NR³R⁴.

17. A compound according to claim 3, wherein n represents 2.

18. A compound according to claim 3, wherein m represents 2.

19. A compound according to claim 3, selected from the group consisting of:
(3R,6R)-3-(2,3-dihydro-1H-inden-2-yl)-6-(1-ethylpropyl)-1-[2(hydroxymethyl)benzyl]-piperazine-2,5-dione;
methyl 2-{[(3R,6R)-3-(2,3-dihydro-1H-inden-2-yl)-6-(1-ethylpropyl)-2,5-dioxo-1-piperazinyl]methyl}benzoate;
(3R,6R)-3-(2,3-dihydro-1H-inden-2-yl)-6-(1-ethylpropyl)-1-{[3-(methyloxy)phenyl]methyl}-2,5-piperazinedione;
(3R,6R)-3-(2,3-dihydro-1H-inden-2-yl)-6-(1-ethylpropyl)-1-[(2-methylphenyl)methyl]-2,5-piperazinedione;
(3R,6R)-3-(2,3-dihydro-1H-inden-2-yl)-6-(1-ethylpropyl)-1-{[4-(methyloxy)phenyl]methyl}-2,5-piperazinedione;
(3R,6R)-3-(2,3-dihydro-1H-inden-2-yl)-6-(1-ethylpropyl)-1-{[2-(methyloxy)phenyl]methyl}-2,5-piperazinedione;
(3R,6R)-3-(2,3-dihydro-1H-inden-2-yl)-6-(1-ethylpropyl)-1-[(3-methylphenyl)methyl]-2,5-piperazinedione;
(3R,6R)-3-(2,3-dihydro-1H-inden-2-yl)-6-(1-ethylpropyl)-1-{[3-(trifluoromethyl)phenyl]-methyl}-2,5-piperazinedione;
(3R,6R)-3-(2,3-dihydro-1H-inden-2-yl)-6-(1-ethylpropyl)-1-[(4-methylphenyl)methyl]-2,5-piperazinedione;
(3R,6R)-1-[(3-chlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-[(4-fluorophenyl)methyl]-2,5-piperazinedione;
(3R,6R)-1-[(2-chlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[3-(4-morpholinylmethyl)-phenyl]methyl}-2,5-piperazinedione formate;
(3R,6R)-3-(2,3-dihydro-1H-inden-2-yl)-6-(1-ethylpropyl)-1-{[4-(4-morpholinyl)phenyl]-methyl}-2,5-piperazinedione;
(3R,6R)-3-(2,3-dihydro-1H-inden-2-yl)-6-(1-ethylpropyl)-1-{[3-(1-piperidinylmethyl)-phenyl]methyl}-2,5-piperazinedione formate;
(3R,6R)-3-(2,3-dihydro-1H-inden-2-yl)-1-[(2-{[2-(dimethylamino)ethyl]oxy}phenyl)methyl]-6-(1-ethylpropyl)-2,5-piperazinedione;
(3R,6R)-3-(2,3-dihydro-1H-inden-2-yl)-6-(1-ethylpropyl)-1-[(2-fluorophenyl)methyl]-2,5-piperazinedione;
(3R,6R)-3-(2,3-dihydro-1H-inden-2-yl)-6-(1-ethylpropyl)-1-[(3-fluorophenyl)methyl]-2,5-piperazinedione;
(3R,6R)-3-(2,3-dihydro-1H-inden-2-yl)-6-(1-ethylpropyl)-1-{[2-(trifluoromethyl)phenyl]-methyl}-2,5-piperazinedione;
(3R,6R)-3-(2,3-dihydro-1H-inden-2-yl)-6-(1-ethylpropyl)-1-{[4-(trifluoromethyl)phenyl]-methyl}-2,5-piperazinedione;
(3R,6R)-3-(2,3-dihydro-1H-inden-2-yl)-6-(1-ethylpropyl)-1-({2-[(trifluoromethyl)oxy]-phenyl}methyl)-2,5-piperazinedione;
(3R,6R)-3-(2,3-dihydro-1H-inden-2-yl)-6-(1-ethylpropyl)-1-({3-[(trifluoromethyl)oxy]-phenyl}methyl)-2,5-piperazinedione;
(3R,6R)-1-{[2,6-bis(methyloxy)phenyl]methyl}-3-(2,3-dihydro-1H-inden-2-yl)-6-(1-ethylpropyl)-2,5-piperazinedione;
(3R,6R)-1-[(2,6-dichlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-(2-pyridinylmethyl)-2,5-piperazinedione;
(3R,6R)-3-(2,3-dihydro-1H-inden-2-yl)-6-(2,2-dimethylpropyl)-1-[(3-methylphenyl)methyl]-2,5-piperazinedione;
(3R,6R)-3-(2,3-dihydro-1H-inden-2-yl)-6-(1-ethylpropyl)-1-[(1R)-1-phenylethyl]-2,5-piperazinedione;
(3R,6R)-3-(2,3-dihydro-1H-inden-2-yl)-1-[(2,6-dimethyl-3-pyridinyl)methyl]-6-(1-ethylpropyl)-2,5-piperazinedione;
(3R,6R)-1-{[2,4-bis(methyloxy)phenyl]methyl}-3-(2,3-dihydro-1H-inden-2-yl)-6-(1-ethylpropyl)-2,5-piperazinedione;
(3R,6R)-1-[(2-bromophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[4-(methylsulfonyl)phenyl]-methyl}-2,5-piperazinedione;
4-{[(3R,6R)-3-(2,3-dihydro-1H-inden-2-yl)-6-(1-ethylpropyl)-2,5-dioxo-1-piperazinyl]-methyl}-N,N-dimethyl-benzenesulfonamide;
(3R,6R)-3-(2,3-dihydro-1H-inden-2-yl)-6-(1-ethylpropyl)-1-[(4-hydroxyphenyl)methyl]-2,5-piperazinedione;
(3R,6R)-3-(2,3-dihydro-1H-inden-2-yl)-6-(2-methylpropyl)-1-[(3-nitrophenyl)methyl]-2,5-piperazinedione;
(3R,6R)-3-(2,3-dihydro-1H-inden-2-yl)-6-(2-methylpropyl)-1-[(2-nitrophenyl)methyl]-2,5-piperazinedione;
(3R,6R)-1-({3-[(difluoromethyl)oxy]phenyl}methyl)-3-(2,3-dihydro-1H-inden-2-yl)-6-(2-methylpropyl)-2,5-piperazinedione;
(3R,6R)-3-(2,3-dihydro-1H-inden-2-yl)-6-(2-methylpropyl)-1-{[2-(methylsulfonyl)phenyl]methyl}-2,5-piperazinedione;
(3R,6R)-3-(2,3-dihydro-1H-inden-2-yl)-6-(2-methylpropyl)-1-{[4-(1,2,3-thiadiazol-4-yl)phenyl]methyl}-2,5-piperazinedione;
(3R,6R)-3-(2,3-dihydro-1H-inden-2-yl)-6-(1-ethylpropyl)-1-[(1-phenyl-1H-pyrazol-4-yl)methyl]-2,5-piperazinedione;
(3R,6R)-1-[(3-chlorophenyl)methyl]-3-(2,3-dihydro-1H-inden-2-yl)-6-(2-methylpropyl)-2,5-piperazinedione;
(3R,6R)-1-[(3,4-dichlorophenyl)methyl]-3-(2,3-dihydro-1H-inden-2-yl)-6-(2-methylpropyl)-2,5-piperazinedione;
(3R,6R)-3-(2,3-dihydro-1H-inden-2-yl)-6-(1-ethylpropyl)-1-{[2-(4-morpholinyl)phenyl]-methyl}-2,5-piperazinedione;
(3R,6R)-1-{[3,5-bis(methyloxy)phenyl]methyl}-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-({4-[(trifluoromethyl)oxy]-phenyl}methyl)-2,5-piperazinedione;
(3R,6R)-3-(2,3-dihydro-1H-inden-2-yl)-1-[(3,5-dimethylphenyl)methyl]-6-(1-ethylpropyl)-2,5-piperazinedione;
(3R,6R)-3-(2,3-dihydro-1H-inden-2-yl)-6-(1-ethylpropyl)-1-(phenylmethyl)-2,5-piperazinedione;
(3R,6R)-1-[(4-chlorophenyl)methyl]-3-(2,3-dihydro-1H-inden-2-yl)-6-(1-ethylpropyl)-2,5-piperazinedione;
(3R,6R)-3-(2,3-dihydro-1H-inden-2-yl)-6-(2-methylpropyl)-1-{[3-(2-pyridinyloxy)phenyl]methyl}-2,5-piperazinedione;
(3R,6R)-3-(2,3-dihydro-1H-inden-2-yl)-6-(1-ethylpropyl)-1-{[2-(methylsulfonyl)phenyl]-methyl}-2,5-piperazinedione;
(3R,6R)-6-cyclohexyl-3-(2,3-dihydro-1H-inden-2-yl)-1-{[2-(methylsulfonyl)phenyl]methyl}-2,5-piperazinedione;
(3R,6R)-3-(2,3-dihydro-1H-inden-2-yl)-6-(2-methylphenyl)-1-{[2-(methylsulfonyl)phenyl]methyl}-2,5-piperazinedione;
(3R,6R)-3-(2,3-dihydro-1H-inden-2-yl)-6-(1-methylethyl)-1-{[2-(methylsulfonyl)phenyl]methyl}-2,5-piperazinedione;
(3R,6R)-6-(dicyclopropylmethyl)-3-(2,3-dihydro-1H-inden-2-yl)-1-{[2-(methylsulfonyl)-phenyl]methyl}-2,5-piperazinedione;
(3R,6R)-3-(2,3-dihydro-1H-inden-2-yl)-6-(2,2-dimethylpropyl)-1-{[2-(methylsulfonyl)-phenyl]methyl}-2,5-piperazinedione;
(3R,6R)-3-(2,3-dihydro-1H-inden-2-yl)-6-(1-ethylpropyl)-1-({4-[(trifluoromethyl)-sulfonyl]phenyl}methyl)-2,5-piperazinedione;
(3R,6R)-3-(2,3-dihydro-1H-inden-2-yl)-1-{[2-(methyloxy)-4-(methylsulfonyl)phenyl]-methyl}-6-(2-methylpropyl)-2,5-piperazinedione;
(3R,6R)-3-(2,3-dihydro-1H-inden-2-yl)-6-(1-ethylpropyl)-1-{[2-(methyloxy)-4-(methylsulfonyl)phenyl]methyl}-2,5-piperazinedione;
(3R,6R)-1-{[2-chloro-4-(methylsulfonyl)phenyl]methyl}-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-[(5-methyl-1-phenyl-1H-pyrazol-4-yl)methyl]-2,5-piperazinedione;
(3R,6R)-3-(2,3-dihydro-1H-inden-2-yl)-1-({2-[(1,1-dimethylethyl)thio]phenyl}methyl)-6-(1-ethylpropyl)-2,5-piperazinedione;
(3R,6R)-3-(2,3-dihydro-1H-inden-2-yl)-6-(1-ethylpropyl)-1-{[1-methyl-5-(trifluoromethyl)-1H-pyrazol-4-yl]methyl}-2,5-piperazinedione;
(3R,6R)-3-(2,3-dihydro-1H-inden-2-yl)-6-(1-ethylpropyl)-1-{[3-(methylsulfonyl)-2-pyridinyl]methyl}-2,5-piperazinedione;
(3R,6R)-3-(2,3-dihydro-1H-inden-2-yl)-6-(1-ethylpropyl)-1-(2-nitrobenzyl)piperazine-2,5-dione;
2-{[(3R,6R)-3-(2,3-dihydro-1H-inden-2-yl)-6-(1-ethylpropyl)-2,5-dioxo-1-piperazinyl]-methyl}benzoic acid;
2-{[(3R,6R)-3-(2,3-dihydro-1H-inden-2-yl)-6-(1-ethylpropyl)-2,5-dioxo-1-piperazinyl]-methyl}-N-methylbenzamide;
2-{[(3R,6R)-3-(2,3-dihydro-1H-inden-2-yl)-6-(1-ethylpropyl)-2,5-dioxo-1-piperazinyl]-methyl}benzamide;
2-{[(3R,6R)-3-(2,3-dihydro-1H-inden-2-yl)-6-(1-ethylpropyl)-2,5-dioxo-1-piperazinyl]-methyl}-N,N-dimethylbenzamide;
2-{[(3R,6R)-3-(2,3-dihydro-1H-inden-2-yl)-6-(1-ethylpropyl)-2,5-dioxo-1-piperazinyl]-methyl}-N-methyl-N-(1-methyl-4-piperidinyl)benzamide;
(3R,6R)-3-(2,3-dihydro-1H-inden-2-yl)-1-[(2-{[4-(dimethylamino)-1-piperidinyl]-carbonyl}phenyl)methyl]-6-(1-ethylpropyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-(1-methyl-4-piperidinyl)benzamide formate;
2-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]-N-methylbenzamide;
2-{[(3R,6R)-3-(2,3-dihydro-1H-inden-2-yl)-6-(1-ethylpropyl)-2,5-dioxo-1-piperazinyl]-methyl}-N-[3-(dimethylamino)propyl]-N-methylbenzamide formate;
2-{[(3R,6R)-3-(2,3-dihydro-1H-inden-2-yl)-6-(1-ethylpropyl)-2,5-dioxo-1-piperazinyl]-methyl}-N-[3-(dimethylamino)propyl]benzamide;
2-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]benzamide;
(3R,6R)-3-(2,3-dihydro-1H-inden-2-yl)-6-(1-ethylpropyl)-1-({2-[(4-methyl-1-piperazinyl)carbonyl]phenyl}methyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-[2-(4-morpholinyl)ethyl]benzamide;
2-{[(3R,6R)-3-(2,3-dihydro-1H-inden-2-yl)-6-(1-ethylpropyl)-2,5-dioxo-1-piperazinyl]-methyl}-N-(2-hydroxyethyl)benzamide;
2-{[(3R,6R)-3-(2,3-dihydro-1H-inden-2-yl)-6-(1-ethylpropyl)-2,5-dioxo-1-piperazinyl]-methyl}-N-(2-hydroxyethyl)-N-methylbenzamide;
(3R,6R)-3-(2,3-dihydro-1H-inden-2-yl)-6-(1-ethylpropyl)-1-{[2-(4-morpholinylcarbonyl)phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-N-4-piperidinylbenzamide;
(3R,6R)-3-(2,3-dihydro-1H-inden-2-yl)-6-(1-ethylpropyl)-1-{[2-(1-piperazinylcarbonyl)-phenyl]methyl}-2,5-piperazinedione;
(3R,6R)-3-(2,3-dihydro-1H-inden-2-yl)-6-(1-ethylpropyl)-1-{[2-(4-morpholinylmethyl)-phenyl]methyl}-2,5-piperazinedione;
(3R,6R)-3-(2,3-dihydro-1H-inden-2-yl)-1-({2-[(dimethylamino)methyl]phenyl}methyl)-6-(1-ethylpropyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-methylbenzenesulfonamide;
2-{[(3R,6R)-3-(2,3-dihydro-1H-inden-2-yl)-6-(1-ethyl-propyl)-2,5-dioxo-1-piperazinyl]-methyl}-N,N-dimethyl-benzenesulfonamide;
(3R,6R)-3-(2,3-dihydro-1H-inden-2-yl)-6-(1-ethylpropyl)-1-{[2-(4-morpholinylsulfonyl)-phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}benzene-sulfonamide;
(3R,6R)-3-(2,3-dihydro-1H-inden-2-yl)-6-(1-ethylpropyl)-1-({2-[(4-methyl-1-piperazinyl)sulfonyl]phenyl}-methyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-(1-methyl-4-piperidinyl)-benzenesulfonamide;
2-{[(3R,6R)-3-(2,3-dihydro-1H-inden-2-yl)-6-(1-ethyl-propyl)-2,5-dioxo-1-piperazinyl]-methyl}-N-methyl-N-(1-methyl-4-piperidinyl)benzenesulfonamide;
2-{[(3R,6R)-3-(2,3-dihydro-1H-inden-2-yl)-6-(1-ethylpropyl)-2,5-dioxo-1-piperazinyl]-methyl}-N-[2-(4-morpholinyl)ethyl]benzene-sulfonamide;
2-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]-benzenesulfonamide;
(3R,6R)-3-(2,3-dihydro-1H-inden-2-yl)-1-({2-[(4-ethyl-1-piperazinyl)sulfonyl]phenyl}-methyl)-6-(1-ethylpropyl)-2,5-piperazinedione;
(3R,6R)-3-(2,3-dihydro-1H-inden-2-yl)-6-(1-ethylpropyl)-1-{[2-({4-[2-(methyloxy)ethyl]-1-piperazinyl}sulfonyl)phenyl]methyl}-2,5-piperazinedione;
(3R,6R)-3-(2,3-dihydro-1H-inden-2-yl)-6-(1-ethylpropyl)-1-{[2-(1-piperazinylsulfonyl)-phenyl]methyl}-2,5-piperazinedione hydrochloride;
(3R,6R)-3-(2,3-dihydro-1H-inden-2-yl)-6-(1-ethylpropyl)-1-{[2-(4-piperidinylthio)-phenyl]methyl}-2,5-piperazinedione;
(3R,6R)-3-(2,3-dihydro-1H-inden-2-yl)-6-(1-ethylpropyl)-1-{[2-(4-piperidinylsulfonyl)-phenyl]methyl}-2,5-piperazinedione hydrochloride;
(3R,6R)-3-(2,3-dihydro-1H-inden-2-yl)-6-(1-ethylpropyl)-1-{[2-({1-[2-(methyloxy)-ethyl]-4-piperidinyl}sulfonyl)phenyl]methyl}-2,5-piperazinedione;
(3R,6R)-3-(2,3-dihydro-1H-inden-2-yl)-6-(1-ethylpropyl)-1-[(2-{[3-(4-morpholinyl)propyl]thio}phenyl)methyl]-2,5-piperazinedione;
(3R,6R)-3-(2,3-dihydro-1H-inden-2-yl)-6-(1-ethylpropyl)-1-[(2-{[3-(4-morpholinyl)propyl]sulfonyl}phenyl)methyl]-2,5-piperazinedione;
(3R,6R)-3-(2,3-dihydro-1H-inden-2-yl)-1-[(2-{[3-(dimethylamino)propyl]thio}phenyl)-methyl]-6-(1-ethylpropyl)-2,5-piperazinedione;
(3R,6R)-3-(2,3-dihydro-1H-inden-2-yl)-6-(1-ethylpropyl)-1-({2-[(1-methyl-4-piperidinyl)thio]phenyl}methyl)-2,5-piperazinedione formate;
(3R,6R)-3-(2,3-dihydro-1H-inden-2-yl)-6-(1-ethylpropyl)-1-({2-[(1-methyl-4-piperidinyl)sulfonyl]phenyl}methyl)-2,5-piperazinedione formate;
(3R,6R)-3-(2,3-dihydro-1H-inden-2-yl)-1-({2-[(1-ethyl-4-piperidinyl)sulfonyl]phenyl}-methyl)-6-(1-ethylpropyl)-2,5-piperazinedione;
(3R,6R)-1-[(2-Aminophenyl)methyl]-3-(2,3-dihydro-1H-inden-2-yl)-6-(1-ethylpropyl)-2,5-piperazinedione;
N-(2-{[(3R,6R)-3-(2,3-dihydro-1H-inden-2-yl)-6-(1-ethylpropyl)-2,5-dioxo-1-piperazinyl]methyl}phenyl)methanesulfonamide;
N-(2-{[(3R,6R)-3-(2,3-dihydro-1H-inden-2-yl)-6-(1-ethylpropyl)-2,5-dioxo-1-piperazinyl]-methyl}phenyl)ethane-sulfonamide;
N-(2-{[(3R,6R)-3-(2,3-dihydro-1H-inden-2-yl)-6-(1-ethylpropyl)-2,5-dioxo-1-piperazinyl]-methyl}phenyl)-2-propanesulfonamide;
N-(2-{[(3R,6R)-3-(2,3-dihydro-1H-inden-2-yl)-6-(1-ethylpropyl)-2,5-dioxo-1-piperazinyl]methyl}phenyl)-N-methylmethanesulfonamide;
(3R,6R)-3-(2,3-dihydro-1H-inden-2-yl)-1-{[2-(1,1-dioxido-2-isothiazolidinyl)phenyl]-methyl}-6-(1-ethylpropyl)-2,5-piperazinedione;
N-(2-{[(3R,6R)-3-(2,3-dihydro-1H-inden-2-yl)-6-(1-ethylpropyl)-2,5-dioxo-1-piperazinyl]methyl}phenyl)acetamide;
N-(2-{[(3R,6R)-3-(2,3-dihydro-1H-inden-2-yl)-6-(1-ethylpropyl)-2,5-dioxo-1-piperazinyl]methyl}phenyl)-N³,N³-dimethyl-β-alaninamide formate;
N-(2-{[(3R,6R)-3-(2,3-dihydro-1H-inden-2-yl)-6-(1-ethylpropyl)-2,5-dioxo-1-piperazinyl]methyl}-phenyl)-4-(dimethyl-amino)butanamide formate;
(formic acid —N-(2-{[(3R,6R)-3-(2,3-dihydro-1H-inden-2-yl)-6-(1-ethylpropyl)-2,5-dioxo-1-piperazinyl]methyl}phenyl)-1-methyl-4-piperidinecarboxamide;
(3R,6R)-3-(2,3-dihydro-1H-inden-2-yl)-6-(1-ethylpropyl)-1-{[2-(2-oxo-1-pyrrolidinyl)-phenyl]methyl}-2,5-piperazinedione;
4-{[(3R,6R)-3-(2,3-dihydro-1H-inden-2-yl)-6-(2-methylpropyl)-2,5-dioxo-1-piperazinyl]methyl}-N,N-dimethylbenzenesulfonamide;
4-{[(3R,6R)-3-(2,3-dihydro-1H-inden-2-yl)-6-(2-methylpropyl)-2,5-dioxo-1-piperazinyl]methyl}-N,N-dimethylbenzenesulfonamide;
(3R,6R)-3-(2,3-dihydro-1H-inden-2-yl)-1-[(3-{[ethyl(methyl)amino]methyl}phenyl)methyl]-6-(1-ethylpropyl)-2,5-piperazinedione;
(3R,6R)-3-(2,3-dihydro-1H-inden-2-yl)-6-(2-methylpropyl)-1-[(1-phenyl-1H-pyrazol-4-yl)methyl]-2,5-piperazinedione;
(3R,6R)-3-(2,3-dihydro-1H-inden-2-yl)-1-{[1-(3-methylphenyl)-1H-pyrazol-4-yl]methyl}-6-(2-methylpropyl)-2,5-piperazinedione;
(3R,6R)-3-(2,3-dihydro-1H-inden-2-yl)-6-(2-methylpropyl)-1-[2-(3-pyridinyl)ethyl]-2,5-piperazinedione;
N-(3-{[(3R,6R)-3-(2,3-dihydro-1H-inden-2-yl)-6-(2-methylpropyl)-2,5-dioxo-1-piperazinyl]methyl}phenyl)methanesulfonamide;
N-(2-{[(3R,6R)-3-(2,3-dihydro-1H-inden-2-yl)-6-(2-methylpropyl)-2,5-dioxo-1-piperazinyl]methyl}phenyl)-N-methylacetamide;
(3R,6R)-3-(2,3-dihydro-1H-inden-2-yl)-1-{2-[4-(methyloxy)-3-(methylsulfonyl)phenyl]-ethyl}-6-(2-methylpropyl)-2,5-piperazinedione;
(3R,6R)-3-(2,3-dihydro-1H-inden-2-yl)-6-(1-ethylpropyl)-1-[(1-methyl-1H-pyrazol-4-yl)methyl]-2,5-piperazinedione;
(3R,6R)-3-(2,3-dihydro-1H-inden-2-yl)-6-(1-ethylpropyl)-1-[(1S)-1-phenylethyl]-2,5-piperazinedione;
(3R,6R)-3-(2,3-dihydro-1H-inden-2-yl)-6-(1-ethylpropyl)-1-(3-pyridinylmethyl)-2,5-piperazinedione;
(3R,6R)-3-(2,3-dihydro-1H-inden-2-yl)-6-(1-ethylpropyl)-1-(4-pyridinylmethyl)-2,5-piperazinedione;
2-{[(3R,6R)-3-(2,3-dihydro-1H-inden-2-yl)-6-(1,1-dimethylpropyl)-2,5-dioxo-1-piperazinyl]methyl}-N-methylbenzamide;
(3R,6R)-3-(2,3-dihydro-1H-inden-2-yl)-6-(1-ethylpropyl)-1-[(2-oxo-1,2-dihydro-3-pyridinyl)methyl]-2,5-piperazinedione;
(3R,6R)-3-(2,3-dihydro-1H-inden-2-yl)-6-(1-ethylpropyl)-1-[(2-hydroxyphenyl)methyl]-2,5-piperazinedione;
2-{[(3R,6R)-3-(2,3-dihydro-1H-inden-2-yl)-6-(1-methylethyl)-2,5-dioxo-1-piperazinyl]methyl}-N-methylbenzamide;
(3R,6R)-3-(2,3-dihydro-1H-inden-2-yl)-6-(1-ethylpropyl)-1-{[1-(phenylmethyl)-1H-pyrazol-4-yl]methyl}-2,5-piperazinedione;
(3R,6R)-6-cyclopentyl-3-(2,3-dihydro-1H-inden-2-yl)-1-{([2-(methylsulfonyl)phenyl]-methyl}-2,5-piperazinedione;
(3R,6R)-3-(2,3-dihydro-1H-inden-2-yl)-6-(1-ethylpropyl)-1-{[2-(methylthio)phenyl]methyl}-2,5-piperazinedione;
(3R,6R)-3-(2,3-dihydro-1H-inden-2-yl)-6-(2-methylpropyl)-1-{[2-(methylthio)phenyl]-methyl}-2,5-piperazinedione;
(3R,6R)-1-[(2,4-dichlorophenyl)methyl]-3-(2,3-dihydro-1H-inden-2-yl)-6-(1-ethylpropyl)-2,5-piperazinedione;
(3R,6R)-1-(2-biphenylylmethyl)-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-[2-(3-pyridinyl)ethyl]-2,5-piperazinedione;
(3R,6R)-6-(dicyclopropylmethyl)-3-(2,3-dihydro-1H-inden-2-yl)-1-[(3-methylphenyl)-methyl]-2,5-piperazinedione;
(3R,6R)-1-{[3,4-bis(methyloxy)phenyl]methyl}-3-(2,3-dihydro-1H-inden-2-yl)-6-(2-methylpropyl)-2,5-piperazinedione;
(3R,6R)-3-(2,3-dihydro-1H-inden-2-yl)-1-[(4-{[2-(dimethylamino)ethyl]oxy}phenyl)methyl]-6-(1-ethylpropyl)-2,5-piperazinedione;
(3R,6R)-3-(2,3-dihydro-1H-inden-2-yl)-6-(1-ethylpropyl)-1-{[3-(1-pyrrolidinylmethyl)-phenyl]methyl}-2,5-piperazinedione;
(3R,6R)-3-(2,3-dihydro-1H-inden-2-yl)-6-(1-ethylpropyl)-1-{[4-(4-morpholinylmethyl)-phenyl]methyl}-2,5-piperazinedione;
formic acid-(3R,6R)-3-(2,3-dihydro-1H-inden-2-yl)-1-({3-[(dimethylamino)methyl]-phenyl}methyl)-6-(1-ethylpropyl)-2,5-piperazinedione (1:1);
(3R,6R)-3-(2,3-dihydro-1H-inden-2-yl)-6-(1-methylethyl)-1-[(3-methylphenyl)methyl]-2,5-piperazinedione;
N-cyclopropyl-4-{[(3R,6R)-3-(2,3-dihydro-1H-inden-2-yl)-6-(2-methylpropyl)-2,5-dioxo-1-piperazinyl]methyl}benzamide;
(3R,6R)-3-(2,3-dihydro-1H-inden-2-yl)-6-(2-methylpropyl)-1-{[3-(3-pyridinyl)phenyl]-methyl}-2,5-piperazinedione;
(3R,6R)-3-(2,3-dihydro-1H-inden-2-yl)-6-(1-ethylpropyl)-1-{[4-fluoro-2-(hydroxymethyl)phenyl]methyl}-2,5-piperazinedione;
(3R,6R)-3-(2,3-dihydro-1H-inden-2-yl)-6-(1-ethylpropyl)-1-{[4-(2-pyrazinylamino)-phenyl]methyl}-2,5-piperazinedione;
(3R,6R)-3-(2,3-dihydro-1H-inden-2-yl)-6-(1-ethylpropyl)-1-{[4-(2-pyrimidinylamino)-phenyl]methyl}-2,5-piperazinedione;
(3R,6R)-3-(2,3-dihydro-1H-inden-2-yl)-6-(2-methylpropyl)-1-{[4-(2-pyrimidinylamino)-phenyl]methyl}-2,5-piperazinedione;
(3R,6R)-3-(2,3-dihydro-1H-inden-2-yl)-6-(1-ethylpropyl)-1-({4-[(1-methyl-1H-imidazol-2-yl)amino]phenyl}methyl)-2,5-piperazinedione;
(3R,6R)-3-(2,3-dihydro-1H-inden-2-yl)-1-({4-[(1-methyl-1H-imidazol-2-yl)amino]phenyl}methyl)-6-(2-methylpropyl)-2,5-piperazinedione;
(3R,6R)-3-(2,3-dihydro-1H-inden-2-yl)-6-(1-ethylpropyl)-1-({4-[(5-methyl-1,3,4-thiadiazol-2-yl)amino]phenyl}methyl)-2,5-piperazinedione;
(3R,6R)-3-(2,3-dihydro-1H-inden-2-yl)-6-(2-methylpropyl)-1-({4-[(5-methyl-1,3,4-thiadiazol-2-yl)amino]phenyl}methyl)-2,5-piperazinedione;
(3R,6R)-3-(2,3-dihydro-1H-inden-2-yl)-6-(1-ethylpropyl)-1-({4-[(5-methyl-1,3-thiazol-2-yl)amino]phenyl}methyl)-2,5-piperazinedione;
(3R,6R)-3-(2,3-dihydro-1H-inden-2-yl)-6-(2-methylpropyl)-1-({4-[(5-methyl-1,3-thiazol-2-yl)amino]phenyl}methyl)-2,5-piperazinedione;
(3R,6R)-3-(2,3-dihydro-1H-inden-2-yl)-6-(1-ethylpropyl)-1-{[4-(1H-pyrazol-1-yl)phenyl]methyl}-2,5-piperazinedione;
(3R,6R)-3-(2,3-dihydro-1H-inden-2-yl)-6-(1-ethylpropyl)-1-{[4-(1H-1,2,3-triazol-1-yl)phenyl]methyl}-2,5-piperazinedione;
(3R,6R)-3-(2,3-dihydro-1H-inden-2-yl)-6-(1-ethylpropyl)-1-{[4-(1H-1,2,4-triazol-1-yl)phenyl]methyl}-2,5-piperazinedione;
(3R,6R)-3-(2,3-dihydro-1H-inden-2-yl)-6-(1-ethylpropyl)-1-{[4-(2H-1,2,3-triazol-2-yl)phenyl]methyl}-2,5-piperazinedione;
(3R,6R)-3-(2,3-dihydro-1H-inden-2-yl)-6-(2-methylpropyl)-1-{[2-(1H-tetrazol-1-yl)phenyl]methyl}-2,5-piperazinedione;
(3R,6R)-3-(2,3-dihydro-1H-inden-2-yl)-6-(1-ethylpropyl)-1-{[2-(1H-tetrazol-1-yl)phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-5-fluorobenzoic acid;
2-{[(3R,6R)-3-(2,3-dihydro-1H-inden-2-yl)-6-(1-ethylpropyl)-2,5-dioxo-1-piperazinyl]methyl}-5-fluoro-N,N-dimethylbenzamide;
(3R,6R)-3-(2,3-dihydro-1H-inden-2-yl)-6-(1-ethylpropyl)-1-{[4-fluoro-2-(4-morpholinylcarbonyl)phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-5-fluoro-N-(2-hydroxyethyl)benzamide;
2-{[(3R,6R)-3-(2,3-dihydro-1H-inden-2-yl)-6-(2-methylpropyl)-2,5-dioxo-1-piperazinyl]methyl}benzoic acid;
2-{[(3R,6R)-3-(2,3-dihydro-1H-inden-2-yl)-6-(2-methylpropyl)-2,5-dioxo-1-piperazinyl]methyl}benzamide;
2-{[(3R,6R)-3-(2,3-dihydro-1H-inden-2-yl)-6-(2-methylpropyl)-2,5-dioxo-1-piperazinyl]methyl}-N,N-dimethylbenzamide;
(3R,6R)-3-(2,3-dihydro-1H-inden-2-yl)-6-(2-methylpropyl)-1-{[2-(4-morpholinylcarbonyl)phenyl]methyl}-2,5-piperazinedione;
2-{[(3R,6R)-3-(2,3-dihydro-1H-inden-2-yl)-6-(2-methylpropyl)-2,5-dioxo-1-piperazinyl]methyl}-N-methylbenzamide;
3-{[(3R,6R)-3-(2,3-dihydro-1H-inden-2-yl)-6-(1-ethylpropyl)-2,5-dioxo-1-piperazinyl]-methyl}benzoic acid;
3-{[(3R,6R)-3-(2,3-dihydro-1H-inden-2-yl)-6-(1-ethylpropyl)-2,5-dioxo-1-piperazinyl]-methyl}benzamide;
3-{[(3R,6R)-3-(2,3-dihydro-1H-inden-2-yl)-6-(1-ethylpropyl)-2,5-dioxo-1-piperazinyl]methyl}-N-methylbenzamide;
(3R,6R)-3-(2,3-dihydro-1H-inden-2-yl)-6-(1-ethylpropyl)-1-{[3-(4-morpholinylcarbonyl)phenyl]methyl}-2,5-piperazinedione;
4-{[(3R,6R)-3-(2,3-dihydro-1H-inden-2-yl)-6-(1-ethylpropyl)-2,5-dioxo-1-piperazinyl]-methyl}benzoic acid;
4-{[(3R,6R)-3-(2,3-dihydro-1H-inden-2-yl)-6-(1-ethylpropyl)-2,5-dioxo-1-piperazinyl]-methyl}benzamide;
4-{[(3R,6R)-3-(2,3-dihydro-1H-inden-2-yl)-6-(1-ethylpropyl)-2,5-dioxo-1-piperazinyl]-methyl}-N-methylbenzamide;
(3R,6R)-3-(2,3-dihydro-1H-inden-2-yl)-6-(1-ethylpropyl)-1-{[4-(4-morpholinylcarbonyl)phenyl]methyl}-2,5-piperazinedione;
4-{[(3R,6R)-3-(2,3-dihydro-1H-inden-2-yl)-6-(1-ethylpropyl)-2,5-dioxo-1-piperazinyl]-methyl}-N-(2-hydroxyethyl)benzamide;
4-{[(3R,6R)-3-(2,3-dihydro-1H-inden-2-yl)-6-(1-ethylpropyl)-2,5-dioxo-1-piperazinyl]-methyl}-N-methyl-N-[2-(methyloxy)ethyl]benzamide;
4-{[(3R,6R)-3-(2,3-dihydro-1H-inden-2-yl)-6-(1-ethylpropyl)-2,5-dioxo-1-piperazinyl]-methyl}-N-[2-(dimethylamino)ethyl]-N-methylbenzamide;
4-{[(3R,6R)-3-(2,3-dihydro-1H-inden-2-yl)-6-(1-ethylpropyl)-2,5-dioxo-1-piperazinyl]methyl}-N-(2-hydroxyethyl)-N-methylbenzamide;
(3R,6R)-3-(2,3-dihydro-1H-inden-2-yl)-1-({4-[(4-methyl-1-piperazinyl)carbonyl]-phenyl}methyl)-6-(2-methylpropyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-{1-[2-(methyloxy)ethyl]-4-piperidinyl}benzamide;
(3R,6R)-1-{[2,4-bis(hydroxymethyl)phenyl]methyl}-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[2-(hydroxymethyl)-4-(methylthio)phenyl]methyl}-2,5-piperazinedione;
(3R,6R)-1-{[2,4-bis(1-pyrrolidinylcarbonyl)phenyl]methyl}-3-(2,3-dihydro-1H-inden-2-yl)-6-(1-ethylpropyl)-2,5-piperazinedione;
(3R,6R)-1-{[2,4-bis(4-morpholinylcarbonyl)phenyl]methyl}-3-(2,3-dihydro-1H-inden-2-yl)-6-(1-ethylpropyl)-2,5-piperazinedione;
4-{[(3R,6R)-3-(2,3-dihydro-1H-inden-2-yl)-6-(1-ethylpropyl)-2,5-dioxo-1-piperazinyl]-methyl}-N,N,N′,N′-tetramethyl-1,3-benzenedicarboxamide;
4-{[(3R,6R)-3-(2,3-dihydro-1H-inden-2-yl)-6-(1-ethylpropyl)-2,5-dioxo-1-piperazinyl]-methyl}-N,N′-bis(2-hydroxyethyl)-N,N′-dimethyl-1,3-benzenedicarboxamide;
4-{[(3R,6R)-3-(2,3-dihydro-1H-inden-2-yl)-6-(1-ethylpropyl)-2,5-dioxo-1-piperazinyl]-methyl}-N,N′-bis[2-(dimethylamino)ethyl]-N,N′-dimethyl-1,3-benzenedicarboxamide;
4-{[(3R,6R)-3-(2,3-dihydro-1H-inden-2-yl)-6-(1-ethylpropyl)-2,5-dioxo-1-piperazinyl]-methyl}-N,N′-dimethyl-N,N′-bis[2-(methyloxy)ethyl]-1,3-benzenedicarboxamide;
(3R,6R)-1-({2,4-bis[(4-methyl-1-piperazinyl)carbonyl]phenyl}methyl)-3-(2,3-dihydro-1H-inden-2-yl)-6-(1-ethylpropyl)-2,5-piperazinedione;
(3R,6R)-3-(2,3-dihydro-1H-inden-2-yl)-6-(1-ethylpropyl)-1-{[2-(hydroxymethyl)-4-(methylsulfonyl)phenyl]methyl}-2,5-piperazinedione;
(3R,6R)-3-(2,3-dihydro-1H-inden-2-yl)-6-(1-ethylpropyl)-1-{[4-(methylsulfonyl)-2-(4-morpholinylcarbonyl)phenyl]methyl}-2,5-piperazinedione;
(3R,6R)-3-(2,3-dihydro-1H-inden-2-yl)-6-(1-ethylpropyl)-1-{[2-[(4-methyl-1-piperazinyl)carbonyl]-4-(methylsulfonyl)phenyl]methyl}-2,5-piperazinedione;
2-{[(3R,6R)-3-(2,3-dihydro-1H-inden-2-yl)-6-(1-ethylpropyl)-2,5-dioxo-1-piperazinyl]-methyl}-N-methyl-N-[2-(methyloxy)ethyl]-5-(methylsulfonyl)benzamide;
N-[2-(diethylamino)ethyl]-2-{[(3R,6R)-3-(2,3-dihydro-1H-inden-2-yl)-6-(1-ethylpropyl)-2,5-dioxo-1-piperazinyl]methyl}-N-methyl-5-(methylsulfonyl)benzamide;
(3R,6R)-3-(2,3-dihydro-1H-inden-2-yl)-6-(1-ethylpropyl)-1-[(4-(methylsulfonyl)-2-{[(2S)-2-(1-pyrrolidinylmethyl)-1-pyrrolidinyl]carbonyl}phenyl)methyl]-2,5-piperazinedione;
2-{[(3R,6R)-3-(2,3-dihydro-1H-inden-2-yl)-6-(1-ethylpropyl)-2,5-dioxo-1-piperazinyl]methyl}-N-(2-hydroxyethyl)benzenesulfonamide;
(3R,6R)-3-(2,3-dihydro-1H-inden-2-yl)-6-(1-ethylpropyl)-1-({2-[(4-methylhexahydro-1H-1,4-diazepin-1-yl)sulfonyl]phenyl}methyl)-2,5-piperazinedione;
2-{[(3R,6R)-3-(2,3-dihydro-1H-inden-2-yl)-6-(1-ethylpropyl)-2,5-dioxo-1-piperazinyl]-methyl}-N-{[(2R)-1-ethyl-2-pyrrolidinyl]methyl}benzenesulfonamide;
(3R,6R)-3-(2,3-dihydro-1H-inden-2-yl)-1-[(2-{[(3S)-3-(dimethylamino)-1-pyrrolidinyl]-sulfonyl}phenyl)methyl]-6-(1-ethylpropyl)-2,5-piperazinedione;
(3R,6R)-3-(2,3-dihydro-1H-inden-2-yl)-1-[(2-{[(3R)-3-(dimethylamino)-1-pyrrolidinyl]-sulfonyl}phenyl)methyl]-6-(1-ethylpropyl)-2,5-piperazinedione;
2-{[(3R,6R)-3-(2,3-dihydro-1H-inden-2-yl)-6-(1-methylethyl)-2,5-dioxo-1-piperazinyl]-methyl}benzenesulfonamide;
N-[(2-{[(3R,6R)-3-(2,3-dihydro-1H-inden-2-yl)-6-(1-ethylpropyl)-2,5-dioxo-1-piperazinyl]methyl}phenyl)sulfonyl]glycine;
(3R,6R)-3-(2,3-dihydro-1H-inden-2-yl)-6-(1-ethylpropyl)-1-({2-[(1-methylethyl)sulfonyl]phenyl}methyl)-2,5-piperazinedione;
(3R,6R)-1-[(2-aminophenyl)methyl]-3-(2,3-dihydro-1H-inden-2-yl)-6-(2-methylpropyl)-2,5-piperazinedione;
formic acid —N-(2-{[(3R,6R)-3-(2,3-dihydro-1H-inden-2-yl)-6-(1-ethylpropyl)-2,5-dioxo-1-piperazinyl]methyl}phenyl)-N²,N²-dimethylglycinamide (1:1);
(3R,6R)-3-(2,3-dihydro-1H-inden-2-yl)-6-(1-ethylpropyl)-1-{[2-(1H-imidazol-1-yl)phenyl]methyl}-2,5-piperazinedione;
formic acid-(3R,6R)-3-(2,3-dihydro-1H-inden-2-yl)-1-[(2-{[3-(dimethylamino)propyl]-sulfonyl}phenyl)methyl]-6-(1-ethylpropyl)-2,5-piperazinedione (1:1);
(3R,6R)-6-cyclopentyl-3-(2,3-dihydro-1H-inden-2-yl)-1-{[4-(methylsulfonyl)phenyl]-methyl}-2,5-piperazinedione;
(3R,6R)-3-(2,3-dihydro-1H-inden-2-yl)-1-[(1,5-dimethyl-1H-pyrazol-4-yl)methyl]-6-(1-ethylpropyl)-2,5-piperazinedione;
(3R,6R)-3-(2,3-dihydro-1H-inden-2-yl)-6-(1-ethylpropyl)-1-{[2-(methylsulfinyl)phenyl]-methyl}-2,5-piperazinedione;
(3R,6R)-3-(2,3-dihydro-1H-inden-2-yl)-6-(1-ethylpropyl)-1-{[2-(methylsulfinyl)phenyl]-methyl}-2,5-piperazinedione;
(3R,6R)-3-(2,3-dihydro-1H-inden-2-yl)-6-(2-methylpropyl)-1-{[2-(methylsulfinyl)phenyl]methyl}-2,5-piperazinedione;
(3R,6R)-3-(2,3-dihydro-1H-inden-2-yl)-6-(2-methylpropyl)-1-{[2-(methylsulfinyl)phenyl]methyl}-2,5-piperazinedione;
(3R,6R)-3-(2,3-dihydro-1H-inden-2-yl)-6-(1-ethylpropyl)-1-{[2-(1H-pyrazol-1-yl)phenyl]methyl}-2,5-piperazinedione;
and a pharmaceutically acceptable solvate, salt, or prodrug thereof.

20. A pharmaceutical composition comprising a compound of Formula (I) according to claim 1 or a pharmaceutically acceptable salt, solvate, or prodrug thereof.

21-24. (canceled)

25. A method of treating or preventing diseases or conditions mediated through the action of oxytocin, which comprises administering to a mammal in need thereof an effective amount of a compound at least one chemical of Formula (I) according to claim 1 or a pharmaceutically acceptable salt, solvate, or prodrug thereof.

26. A method according to claim 25 wherein the disease or condition is selected from pre-term labour, dysmenorrhea and endometriosis.

27. A process for the preparation of a compound of Formula (I) according to claim 1 comprising cyclisation of a compound of Formula (VII), wherein A, B, R¹ and R² are as defined for Formula (I), and J is an optional substituent, optionally in the presence of a suitable acid or base. [chemical structure]
